(19) United States
(12) Patent Application Publication
(10) Pub. No.: US 2026/0285863 A1
(43) Pub. Date: Sep. 24, 2026

(54) COMPOUNDS CAPABLE OF MODULATING GPR65

(71) Applicant: Pathios Therapeutics Limited, Milton, Abingdon, Oxford Oxfordshire (GB)

(72) Inventors: Alan Naylor, Milton, Abingdon, Oxford Oxfordshire (GB); Gavin Milne, Milton, Abingdon, Oxford Oxfordshire (GB); David Miller, Milton, Abingdon, Oxford Oxfordshire (GB)

(73) Assignee: Pathios Therapeutics Limited, Milton, Abingdon, Oxford Oxfordshire (GB)

(21) Appl. No.: 19/472,323

(22) PCT Filed: Apr. 24, 2024
(86) PCT No.: PCT/GB2024/051071

§ 371 Date: Oct. 3, 2025

(30) Foreign Application Priority Data
Apr. 25, 2023 (GB) 2306073.4

(51) Int. Cl. C07D 471/18; A61K 31/439; A61K 31/444; A61K 31/504

(52) U.S. Cl. CPC C07D 471/18; A61K 31/439; A61K 31/444; A61K 31/504

(57) ABSTRACT

One aspect of the invention relates to a compound of formula (I), or a pharmaceutically acceptable salt or solvate thereof, (I). Wherein pring A is selected from: Y is CR₁₀R_{10′}, wherein R₁₀ and R_{10′} are each independently selected from H, F, alkyl, and haloalkyl; Ra and Rb are each independently selected from H and alkyl; ring B is: a monocyclic aryl group; or a monocyclic heteroaryl group, each of which is substituted by: (i) a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR¹¹R₁₁, CON₁₂R_{12′}, alkyl-N₁₃R_{13′}, alkoxy-alkyl, alkoxy-alkoxy and NHSO₂-alkyl; wherein said —(CH₂)_{q}-heterocycloalkyl group is further substituted by one or more substituents selected from CO₂R₁₄, halo, haloalkyl, haloalkoxy and alkyl; and (ii) optionally one or more substituents selected from halo, haloalkyl, haloalkoxy, alkyl and alkoxy; q is an integer from 0 to 3; R₆ is selected from H and alkyl, more preferably H; R₇, R₈ and R₉ are each independently selected from H, halo and alkyl; each R₁₁, R_{11′}, R₁₂, R_{12′}, R₁₃ and R_{13′} is independently selected from H, alkyl, and alkoxyalkyl; R₁₄ is selected from alkyl and haloalkyl; R₁₅ is selected from H and alkyl; and R₁₆ is selected from alkyl, haloalkyl and CO-haloalkyl. Further aspects of the invention relate to compounds for use as a medicament, particularly in the field of immuno-oncology, immunology, and related applications.

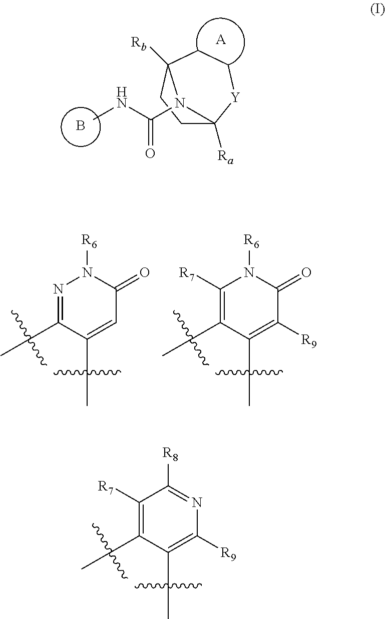

The present invention relates to compounds that are capable of modulating GPR65. The compounds have potential therapeutic applications in the treatment of a variety of disorders including proliferative and immune disorders.

### BACKGROUND TO THE INVENTION

GPR65 is a Gs-coupled G protein-coupled receptor (GPCR) that is primarily expressed in immune cells and is activated by acidic extracellular pH to cause increases in cytoplasmic cyclic adenosine monophosphate (cAMP) (Wang, 2004). It has long been known that tumours typically undergo a switch in cellular metabolism from oxidative phosphorylation to aerobic glycolysis, which in turn results in an acidic extracellular microenvironment (Damaghi, 2013). Recently, it has been shown that this acidic microenvironment causes GPR65 activation in tumour-associated macrophages, resulting in an increase in cytoplasmic cAMP leading to transcription of the inducible cAMP early repressor (ICER). This, in turn, suppresses the secretion of tumour necrosis factor alpha (TNFα) to bias the macrophages toward an anti-inflammatory, tumour-permissive phenotype (Bohn, 2018). This GPR65-dependent pathway therefore appears to represent a mechanism by which tumours exploit their acidic microenvironment to evade detection by the immune system.

Autoimmune diseases are also often associated with an acidic local microenvironment (for instance, an inflamed joint). Recent studies also suggest that GPR65 acts through ICER in CD4+ T cells, to suppress IL-2 and hence bias cells toward an inflammatory Th17 phenotype, which is associated with increased pathogenicity in the context of autoimmune disease (Korn, 2009). Supporting this is the recent finding that ICER is required for Th17 differentiation (Yoshida, 2016) as well as that agonism of GPR65 leads to an increase in Th17 differentiation (Hernandez, 2018). Indeed, mutations in the GPR65 locus are associated with several autoimmune diseases, such as multiple sclerosis, ankylosing spondylitis, inflammatory bowel disease, and Crohn's disease (Gaublomme, 2015). One recent study found that mice with CD4+ T cells lacking GPR65 were protected from developing the disease autoimmune encephalomyelitis (EAE) (Gaublomme, 2015).

Thus, GPR65 appears to act through ICER to promote an anti-inflammatory and tumour-permissive phenotype in tumour associated macrophages and an inflammatory Th17 phenotype in CD4+ T cells that is associated with autoimmune disease. GPR65 signalling, therefore, represents an attractive pathway for therapeutic intervention for the treatment of both cancer and autoimmune diseases. There is therefore an ongoing need to develop new small molecule GPR65 modulators.

WO 2021/245427 and WO 2022136844 (Pathios Therapeutics Limited) disclose a series of small molecule GPR65 modulators. The present invention seeks to provide further compounds that are capable of modulating GPR65. As made clear from the above discussion, such compounds have potential therapeutic applications in the treatment of a variety of disorders, including proliferative disorders and immune disorders, as well as asthma and chronic obstructive pulmonary disease. Advantageously, the presently claimed compounds may also exhibit one or more of the following properties: enhanced activity against GPR65 (also in native cells), better in vitro selectivity and toxicity profiles and/or enhanced oral pharmacokinetic profiles.

### STATEMENT OF INVENTION

Another aspect of the invention relates to a compound of formula (1), or a pharmaceutically acceptable salt or solvate thereof,

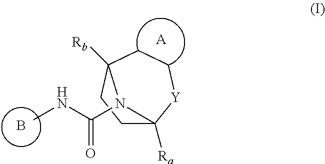

wherein:
ring A is selected from:

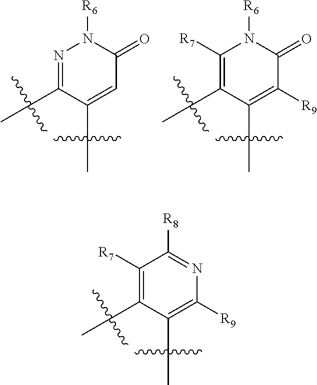

Y is CR₁₀R_{10′}, wherein R₁₀ and R_{10′} are each independently selected from H, F, alkyl, and haloalkyl;
Rₐ and R_{b} are each independently selected from H and alkyl;
ring B is:
   a monocyclic aryl group; or
   a monocyclic heteroaryl group,
each of which is substituted by:
   (i) a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
      wherein said —(CH₂)_{q}-heterocycloalkyl group is further substituted by one or more substituents selected from CO₂R₁₄, halo, haloalkyl, haloalkoxy and alkyl;
   (ii) optionally one or more substituents selected from halo, haloalkyl, haloalkoxy, alkyl and alkoxy;
q is an integer from 0 to 3;
R₆ is selected from H and alkyl, more preferably H;
R₇, Rₐ and R₉ are each independently selected from H, halo and alkyl;
each R₁₁, R_{11′}, R₁₂, R_{12′}, R₁₃ and R_{13′} is independently selected from H, alkyl, and alkoxyalkyl;
R₁₄ is selected from alkyl and haloalkyl;
R₁₅ is selected from H and alkyl; and
R₁₆ is selected from alkyl, haloalkyl and CO-haloalkyl.

A second aspect of the invention relates to a compound of formula (II), or a pharmaceutically acceptable salt or solvate thereof,

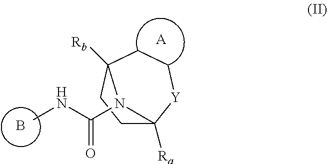

wherein:
ring A is selected from:

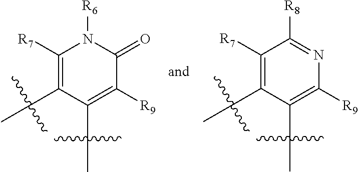

Y is CR₁₀R_{10′}, wherein R₁₀ and R_{10′} are each independently selected from H, F, alkyl, and haloalkyl;
Rₐ and R_{b} are each independently selected from H and alkyl;
ring B is a monocyclic heteroaryl group which is substituted by:
   (i) a bicyclic heteroaryl group, wherein said bicyclic heteroaryl group is optionally further substituted by one or more substituents selected from halo, haloalkyl, alkyl, alkoxy, NHCO-alkyl, NR₁₁R_{11′}, SO₂-alkyl, CN, hydroxyalkyl, CONR₁₂R_{12′}, alkyl-NR₁₃R_{13′}, alkylamino-alkoxy, dialkylamino-alkoxy, heterocycloalkyl, alkyl-heterocycloalkyl, alkyl-cycloalkyl, aryl, (CH₂)ₘ-NHSO₂-alkyl, CO₂R₁₄, alkoxy-alkyl, haloalkoxy, heteroaryl, alkoxy-alkoxy, O—(CH₂)ₚ-cycloalkyl, and O—(CH₂)_{q}-heterocycloalkyl, wherein said cycloalkyl and heterocycloalkyl groups in each of the above substituents are optionally further substituted by one or more halo, haloalkyl, alkyl or alkoxy groups; and
   (ii) optionally one or more substituents selected from halo, haloalkyl, haloalkoxy, alkyl, alkoxy, OH and CN;
each of m, p and q is an integer from 0 to 3;
R₆ is selected from H and alkyl, more preferably H;
R₇, R₈ and R₉ are each independently selected from H, halo and alkyl;
R₁₁, R_{11′}, R₁₂, R_{12′}, R₁₃ and R_{13′} are each independently selected from H, alkyl, and alkoxyalkyl; and
R₁₄ is selected from alkyl and haloalkyl.

Another aspect of the invention relates to a compound selected from compounds (1)-(92) described herein, or a pharmaceutically acceptable salt or solvate thereof.

Advantageously, the presently claimed compounds are capable of modulating GPR65, thereby rendering the compounds of therapeutic interest in the treatment of various disorders, for example, in the field of oncology, immuno-oncology, and immunology.

Another aspect of the invention relates to a compound or pharmaceutically acceptable salt or solvate thereof, as described herein for use as a medicament.

Another aspect of the invention relates to a compound or pharmaceutically acceptable salt or solvate thereof, as described herein for use in treating or preventing a disorder selected from a proliferative disorder, an immune disorder, asthma, chronic obstructive pulmonary disease (COPD) and acute respiratory distress syndrome (ARDS).

Another aspect of the invention relates to a pharmaceutical composition comprising a compound as described herein and a pharmaceutically acceptable diluent, excipient, or carrier.

Another aspect of the invention relates to a pharmaceutical composition as described herein for use as a medicament.

Another aspect of the invention relates to a pharmaceutical composition as described herein for use in treating or preventing a disorder selected from a proliferative disorder, an immune disorder, asthma, chronic obstructive pulmonary disease (COPD) and acute respiratory distress syndrome (ARDS).

Another aspect of the invention relates to a method of treating a disorder, comprising administering to a subject a compound or a pharmaceutical composition as described herein.

### DETAILED DESCRIPTION

The present invention relates to compounds that are capable of modulating GPR65. “Alkyl” is defined herein as a straight-chain or branched alkyl radical, preferably C₁₋₂₀ alkyl, more preferably C₁₋₁₂ alkyl, even more preferably C₁₋₁₀ alkyl or C₁₋₆ alkyl, for example, methyl, ethyl, propyl, isopropyl, butyl, isobutyl, tert-butyl, pentyl, hexyl. More preferably, the alkyl is a C₁₋₃ alkyl.

“Cycloalkyl” is defined herein as a monocyclic alkyl ring, preferably, C₃₇-cycloalkyl, more preferably C₃.s-cycloalkyl. Preferred examples include cyclopropyl, cyclobutyl, cyclopentyl, cyclohexyl or cycloheptyl, or a fused bicyclic ring system such as norbornane.

As used herein, the term “aryl” refers to a C₆₋₁₂ aromatic group, which may be a monocyclic or fused bicyclic group, including benzocondensed groups. Examples include phenyl and naphthyl.

“Haloalkyl” is defined herein as a straight-chain or branched alkyl radical as defined above, for example, methyl, ethyl, propyl, isopropyl, butyl, isobutyl, tert-butyl, pentyl, hexyl, that is substituted with one or more halogen atoms (that may be the same or different), such as fluorine, chlorine, bromine, and iodine. Preferably, the haloalkyl is a C₁₋₂₀ haloalkyl, more preferably a C₁₋₁₂ haloalkyl, even more preferably a C₁₋₁₀ haloalkyl or a C₁₋₆ haloalkyl, or a C₁₋₃ haloalkyl. Preferred examples are CF₃ and CHF₂, with CF₃ being particularly preferred.

“Alkoxy” is defined herein as an oxygen atom bonded to an alkyl group as defined above, for example methoxy, ethoxy, propoxy, isopropoxy, butoxy, isobutoxy, tert-butoxy, pentoxy and hexoxy. Preferably, the alkoxy is a C₁₋₂₀ alkoxy, more preferably a C₁₋₁₂ alkoxy, even more preferably C₁₋₁₀ alkoxy or a C₁₋₆ alkoxy, or a C₁₋₃ alkoxy. A particularly preferred example is methoxy (—OCH₃). “Alkoxy-alkyl” is defined as an alkyl group as that is substituted by one or more alkoxy groups, e.g. MeOCH₂CH₂—. “Alkoxy-alkoxy” is defined as an alkoxy group that is substituted by one or more further alkoxy groups, e.g. MeOCH₂CH₂O— (also referred to as an ether group).

“Haloalkoxy” is defined herein as an alkoxy group as described above that is substituted with one or more halogen atoms (that may be the same or different), such as fluorine, chlorine, bromine, and iodine.

“Heteroaryl” or “heteroaromatic” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulphur, which is optionally interrupted by one or more —(CO)-groups in the ring and/or which optionally contains one or more double bonds in the ring. Preferably, the heterocycloalkyl group is monocyclic or bicyclic. Preferably, the heterocycloalkyl group is a C₃₋₄-heterocycloalkyl, more preferably a C₃₋₆-heterocycloalkyl.

Alternatively, the heterocycloalkyl group is a C₄₇-heterocycloalkyl, more preferably a C₄₋₆-heterocycloalkyl. Preferred heterocycloalkyl groups include, but are not limited to, azetidinyl, piperazinyl, piperidinyl, morpholinyl, thiomorpholinyl, pyrrolidinyl, tetrahydrofuranyl and tetrahydropyranyl. Examples of heterocycloalkyl groups containing a CO group and one or more double bonds include 3-oxo-3,4-dihydro-2H-benzo[b][1,4]oxazin-6-yl, oxoisoindolinyl, oxoindolinyl, 1-oxo-1,2,3,4-tetrahydroiso-quinolin-6-yl, 1-oxo-1,2,3,4-tetrahydroisoquinolin-6-yl and the like.

“Aralkyl” is defined herein as an alkyl group as defined above substituted by one or more aryl groups as defined above.

Preferably alkyl is C₁-C₆ alkyl, haloalkyl is C₁-C₆ haloalkyl, alkoxy is C₁-C₆ alkoxy and haloalkoxy is C₁-C₆ haloalkoxy.

### Structural Representation of the Compounds

The compounds of the invention comprise a structure wherein an optionally substituted ring A is fused to a bicyclic nitrogen-containing moiety to form a tricyclic structure. The resulting tricyclic structure can exist in two different configurations as depicted below:

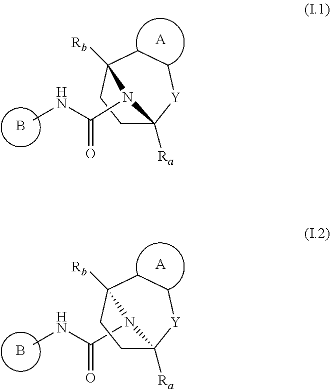

For the avoidance of doubt, the invention encompasses the compounds in either of the above configurations, as well as mixtures thereof, including racemic mixtures.

Alternatively, the structure can, for example, be represented, as follows (where Rₐ and R_{b} groups are omitted for clarity):

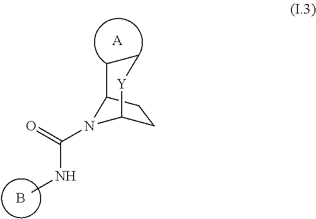

For the avoidance of doubt, the invention encompasses the compounds in the above configuration, as well the corresponding enantiomers thereof, and mixtures thereof, including racemic mixtures. As used throughout, and for ease of representation, specific examples of compounds according to the invention depicted in the above configuration (1.3) refer to mixtures of both enantiomers (in particular, the racemate), whereas the respective enantiomers—where these have been synthesised or separated—are depicted as either configuration (1.1) or configuration (1.2) with wedged bonds or dashed bonds respectively.

Enantiomeric forma (1.1) and (1.2) apply equally to all of the various subformulae described herein.

In one preferred embodiment, the compound is in enantiomerically pure form. In one preferred embodiment, the compound is in the form of a mixture that is enantiomerically enriched with a compound of formula (1.2).

In one preferred embodiment, the compound is in the form of a mixture comprising a compound of formula (1.1) and its corresponding enantiomer of formula (1.2). In one preferred embodiment, the mixture is a racemic mixture, i.e. a 50:50 mixture of a compound of formula (1.1) and its corresponding enantiomer of formula (1.2).

Racemic mixtures can be used to prepare enantiomerically pure compounds of formula (1.1) or (1.2) by separating the compounds of formula (1.1) or (1.2) by standard methods, for example by chemical resolution using optically active acid or by the use of column chromatography or reverse-phase column chromatography using a substantially optically active (or “chiral”) stationary phase as known to those skilled in the art. Racemic mixtures can also be used to prepare enantiomerically enriched mixtures of compounds of formula (1.1) or (1.2). Mixtures enriched with either a compound of formula (1.1) or (1.2) can also be obtained from the appropriate enantiomerically enriched precursors.

In one preferred embodiment of the invention, the compound is in the form of a mixture comprising enantiomers wherein the weight:weight ratio is at least approximately 2:1 or greater, preferably at least approximately 5:1 or greater, most preferably at least approximately 10:1 or greater in favour of the enantiomer that displays significant in vitro and/or in vivo activity (the eutomer).

In one particularly preferred embodiment, the compound is in the form of a mixture comprising a compound of formula (1.1) and its corresponding enantiomer of formula (1.2), wherein the weight:weight ratio of said compound of formula (1.1) to said compound of formula (1.2) is greater than 1.05:1, more preferably, greater than 2:1, even more preferably greater than 5:1, even more preferably greater than 10:1.

In one particularly preferred embodiment, the compound is in the form of a mixture comprising a compound of formula (1.1) and its corresponding enantiomer of formula (1.2), which is substantially enriched with said compound of formula (1.1).

In one embodiment, the compound is in the form of a mixture comprising a compound of formula (1.1) and its corresponding enantiomer of formula (1.2), wherein the weight:weight ratio of said compound of formula (1.2) to said compound of formula (1.1) is greater than 1.05:1, more preferably, greater than 2:1, even more preferably greater than 5:1, even more preferably greater than 10:1.

In one embodiment, the compound is in the form of a mixture comprising a compound of formula (1.1) and its corresponding enantiomer of formula (1.2), which is substantially enriched with said compound of formula (1.2).

Some of the compounds described comprise an A ring which contains a C═O group and which can exist in more than one tautomeric form. For example, in some embodiments, the A ring can exist in the following tautomeric forms:

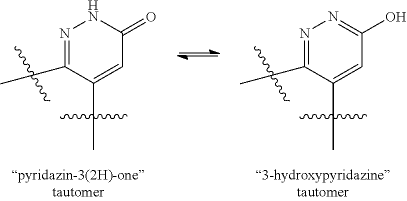 [SMILES: *c1cc(=O)[nH]nc1* ; *c1cc(O)nnc1*]

The pyradazin-3(2H)-one tautomer is believed to be the predominant solid state form. In solution, the energy difference between the two tautomeric forms is understood to be very small and is dependent on the polarity of the solvent. The skilled person would understand that other hydroxy substituted A rings (e.g. pyridinyl and the like) can exist in similar tautomeric forms. The invention encompasses all tautomeric forms of the compounds described herein.

### Compounds of Formula (1)

A further aspect of the invention relates to a compound of formula (I), or a pharmaceutically acceptable salt or solvate thereof,

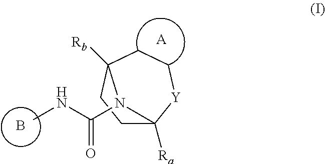

wherein:
ring A is selected from:

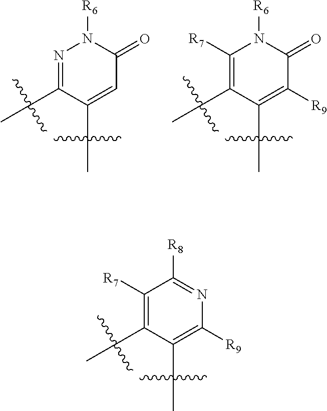

Y is CR₁₀R_{10′}, wherein R₁₀ and R_{10′} are each independently selected from H, F, alkyl, and haloalkyl;
Rₐ and R_{b} are each independently selected from H and alkyl;
ring B is:
   a monocyclic aryl group; or
   a monocyclic heteroaryl group,
each of which is substituted by:
   (i) a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
      wherein said —(CH₂)_{q}-heterocycloalkyl group is further substituted by one or more substituents selected from CO₂R₁₄, halo, haloalkyl, haloalkoxy and alkyl;
   (ii) optionally one or more substituents selected from halo, haloalkyl, haloalkoxy, alkyl and alkoxy;
q is an integer from 0 to 3;
R₆ is selected from H and alkyl, more preferably H;
R₇, R₈ and R₉ are each independently selected from H, halo and alkyl;
each R₁₁, R_{11′}, R₁₂, R_{12′}, R₁₃ and R_{13′} is independently selected from H, alkyl, and alkoxyalkyl;
R₁₄ is selected from alkyl and haloalkyl;
R₁₅ is selected from H and alkyl; and
R₁₆ is selected from alkyl, haloalkyl and CO-haloalkyl.

In one embodiment, the heterocycloalkyl of said —(CH₂)_{q}-heterocycloalkyl group is further substituted by one or more substituents selected from CO₂R₁₄, halo, haloalkyl, haloalkoxy and alkyl.

In one particularly preferred embodiment, the compound is of formula (Ia) or a pharmaceutically acceptable salt or solvate thereof,

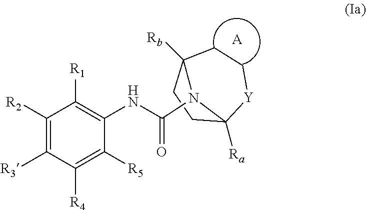

wherein:
R₁, R₂, R₄ and R₅ are each independently selected from H, halo, haloalkyl, haloalkoxy, alkyl and alkoxy; and
R_{3′} is a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
   wherein said —(CH₂)_{q}-heterocycloalkyl group is further substituted by one or more substituents selected from CO₂R₁₄, halo, haloalkyl, haloalkoxy and alkyl.

In one preferred embodiment, R₁₁, R_{11′}, R₁₂, R_{12′}, R₁₃ and R_{13′} are each independently selected from H and alkyl, more preferably H and Me.

In one preferred embodiment, R₁₄ is alkyl, more preferably Me, Et, ⁱPr, ⁿPr, ⁿBu, ⁱBu or ^{t}Bu.

In one preferred embodiment, the compound is of formula (Ib) or a pharmaceutically acceptable salt or solvate thereof,

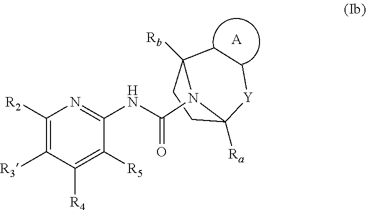

wherein:
R₂, R₄ and R₅ are each independently selected from H, halo, haloalkyl, haloalkoxy, alkyl and alkoxy; and
R_{3′} is a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
   wherein said —(CH₂)_{q}-heterocycloalkyl group is further substituted by one or more substituents selected from CO₂R₁₄, halo, haloalkyl, haloalkoxy and alkyl.

In one preferred embodiment, in formula (Ia) or (Ib), R_{3′} is a pyridinyl group substituted by at least one group selected from —NR₁₅R₁₆, and —O—(CH₂)_{q}-heterocycloalkyl, wherein the heterocycloalkyl of said —(CH₂)_{q}-heterocycloalkyl group is further substituted by one or more substituents selected from CO₂R₁₄, halo, haloalkyl, haloalkoxy and alkyl.

In one preferred embodiment, in formula (Ia) or (Ib), R_{3′} is a pyridinyl group substituted by a group —NR₁₅R₁₆, wherein R₁₅ is H or alkyl, and R₁₆ is a group selected from alkyl, haloalkyl and CO-haloalkyl, more preferably selected from haloalkyl and CO-haloalkyl.

In one preferred embodiment, in formula (Ia) or (Ib), R_{3′} is a pyridinyl group substituted by a group —O—(CH₂)_{q}-heterocycloalkyl, wherein said heterocycloalkyl group is selected from piperazinyl, azetidinyl, piperidinyl, morpholinyl, thiomorpholinyl, pyrrolidinyl, tetrahydrofuranyl and tetrahydropyranyl, 2-azabicyclo[2.1.1]hexanyl and 2-oxabicyclo[2.1.1]hexanyl, each of which is further substituted by one or more substituents selected from CO₂R₁₄, halo, haloalkyl, haloalkoxy and alkyl.

In one preferred embodiment, in formula (Ia) or (Ib), R_{3′} is a pyridinyl group substituted by a group —O—(CH₂)_{q}-heterocycloalkyl, wherein said heterocycloalkyl group is selected from pyrrolidinyl, azetidinyl, tetrahydrofuranyl, 2-azabicyclo[2.1.1]hexanyl and 2-oxabicyclo[2.1.1]hexanyl, each of which is further substituted by one or more substituents selected from CO₂-alkyl, haloalkyl and alkyl.

In one preferred embodiment, in formula (Ia) or (Ib), R_{3′} is a pyridinyl group substituted by a group —O—(CH₂)_{q}-heterocycloalkyl, wherein said heterocycloalkyl group is selected from pyrrolidinyl, azetidinyl, tetrahydrofuranyl, 2-azabicyclo[2.1.1]hexanyl and 2-oxabicyclo[2.1.1]hexanyl, each of which is further substituted by one or more substituents selected from CO₂—Bu, Me, CF₃, and CF₃CH₂.

In one preferred embodiment, q is 0 or 1.

In one preferred embodiment, q is 0.

In one preferred embodiment, q is 1.

In one preferred embodiment, R₂ and R₅ are both H.

In one preferred embodiment, said compound is of formula (Ia) and R₁ is selected from H, F, Me, MeO and Cl, and is preferably H or F.

In one preferred embodiment, R₄ is selected from C₁, Br, and CF₃, more preferably Cl.

In one preferred embodiment, R₂ is H, R₄ is Cl and R₅ is H.

In one preferred embodiment, ring A is of formula:

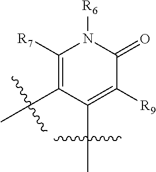

wherein R₆, R₇ and R₉ are all H, i.e. the compound is of the formula:

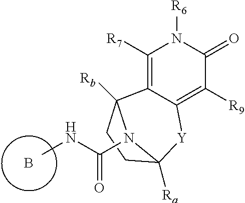

In one preferred embodiment, ring A is of formula:

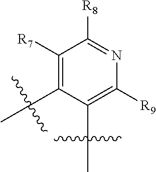

wherein R₇ and R₃ are H, and R₉ is F, i.e. the compound is of the formula:

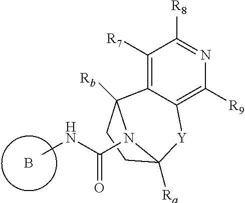

In one preferred embodiment, ring A is of formula:

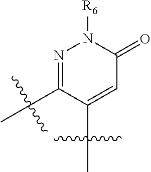

wherein R₆ is H, i.e. the compound is of the formula:

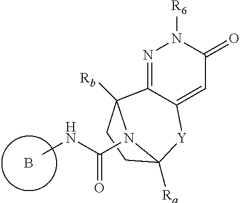

In one preferred embodiment, Rₐ and R_{b} are both H.

In one preferred embodiment, Y is selected from CH₂, CHF and CF₂, more preferably CH₂.

In one preferred embodiment, the compound of formula (I) is selected from the following:

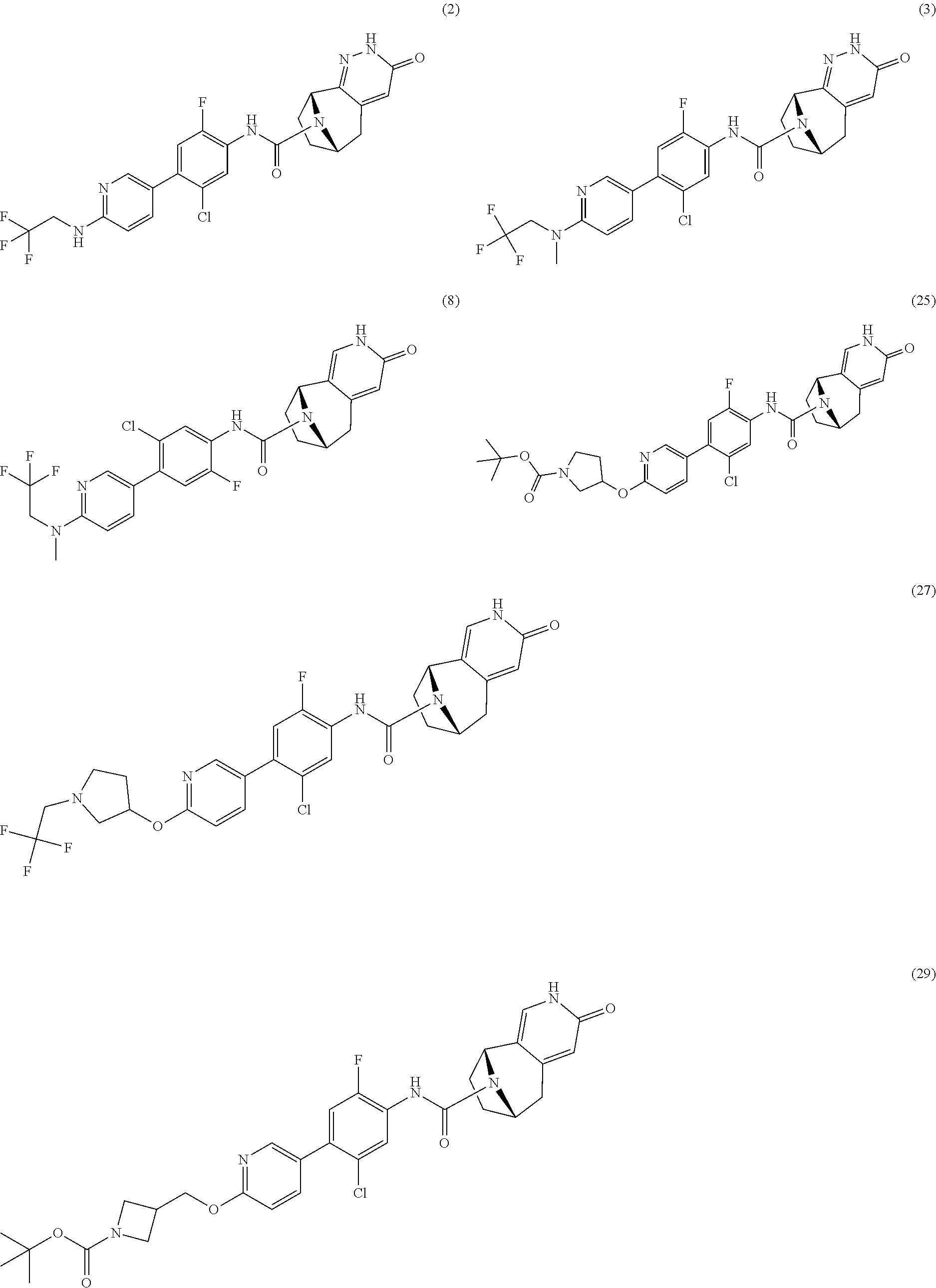

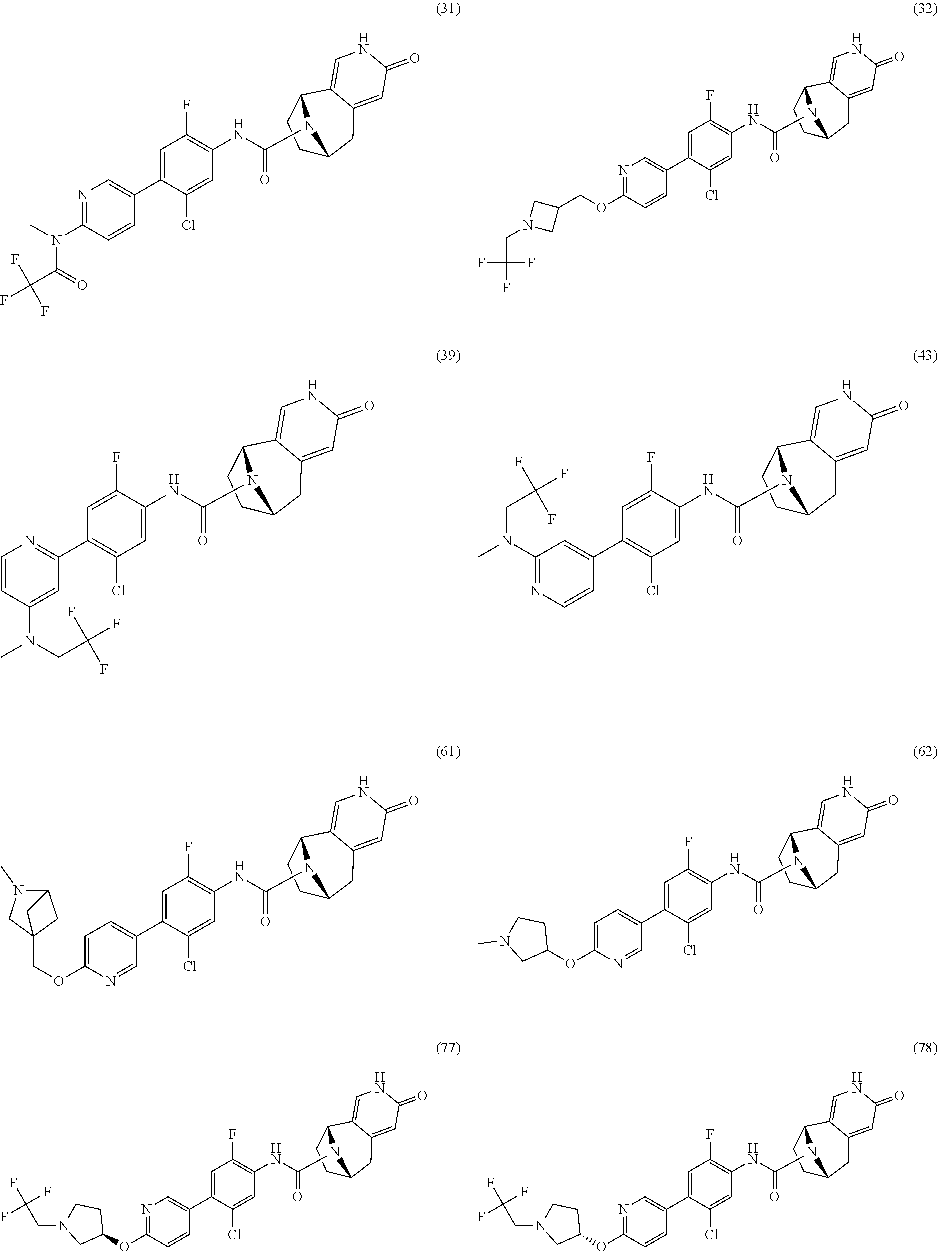

and enantiomers thereof, and mixtures of enantiomers thereof, including racemic mixtures, and pharmaceutically acceptable salts and solvates thereof.

### Compounds of Formula (II)

The present invention relates to compounds of formula (11), or a pharmaceutically acceptable salt or solvate thereof,

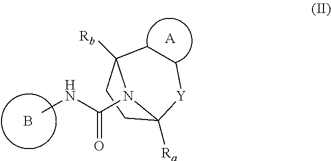

wherein:
ring A is selected from:

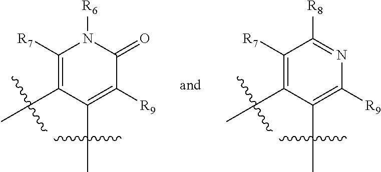

Y is CR₁₀R_{10′}, wherein R₁₀ and R_{10′} are each independently selected from H, F, alkyl, and haloalkyl;
Rₐ and R_{b} are each independently selected from H and alkyl;
ring B is a monocyclic heteroaryl group which is substituted by:
   (i) a bicyclic heteroaryl group, wherein said bicyclic heteroaryl group is optionally further substituted by one or more substituents selected from halo, haloalkyl, alkyl, alkoxy, NHCO-alkyl, NR₁₁R_{11′}, SO₂-alkyl, CN, hydroxyalkyl, CONR₁₂R_{12′}, alkyl-NR₁₃R_{13′}, alkylamino-alkoxy, dialkylamino-alkoxy, heterocycloalkyl, alkyl-heterocycloalkyl, alkyl-cycloalkyl, aryl, (CH₂)ₘ-NHSO₂-alkyl, CO₂R₁₄, alkoxy-alkyl, haloalkoxy, heteroaryl, alkoxy-alkoxy, O—(CH₂)ₚ-cycloalkyl, and O—(CH₂)_{q}—heterocycloalkyl,
      wherein said cycloalkyl and heterocycloalkyl groups in each of the above substituents are optionally further substituted by one or more halo, haloalkyl, alkyl or alkoxy groups; and
   (ii) optionally one or more substituents selected from halo, haloalkyl, haloalkoxy, alkyl, alkoxy, OH and CN;
each of m, p and q is an integer from 0 to 3;
R₆ is selected from H and alkyl, more preferably H;
R₇, R₈ and R₉ are each independently selected from H, halo and alkyl;
R₁₁, R_{11′}, R₁₂, R_{12′}, R₁₃ and R_{13′} are each independently selected from H, alkyl, and alkoxyalkyl; and
R₁₄ is selected from alkyl and haloalkyl.

In one preferred embodiment, ring B is a substituted monocyclic 5- or 6-membered heteroaryl group. Preferably, the substituted monocyclic 5- or 6-membered heteroaryl group comprises at least one nitrogen atom. In one preferred embodiment, the substituted monocyclic 5- or 6-membered heteroaryl is selected from pyridin-2-yl, pyridin-3-yl, pyridin-4-yl, pyrazin-2-yl, pyrazin-3-yl, pyrimidin-2-yl, pyrimidin-4-yl, pyrimidin-5-yl, 1,2,3-triazinyl, 1,2,4-triazinyl, 1,3,5-triazinyl, 1,2,4,5-tetrazinyl, 1,2,3,4-tetrazinyl, oxadiazoyl, thiadiazolyl, imidazolyl, pyrrolyl, pyrazolyl, diazolyl, triazolyl, isoxazolyl, isothiazolyl, tetrazolyl, oxazolyl, and thiazolyl.

In one preferred embodiment, ring B is a pyridinyl group which is substituted by a bicyclic heteroaryl group, wherein said bicyclic heteroaryl group is optionally further substituted by one or more substituents selected from halo, haloalkyl, alkyl, alkoxy, NHCO-alkyl, NR₁₁R_{11′}, SO₂-alkyl, CN, hydroxyalkyl, CONR₁₂R_{12′}, alkyl-NR₁₃R_{13′}, alkylamino-alkoxy, dialkylamino-alkoxy, heterocycloalkyl, alkyl-heterocycloalkyl, alkyl-cycloalkyl, aryl, (CH₂)ₘ—NHSO₂-alkyl, CO₂R₁₄, alkoxy-alkyl, haloalkoxy, heteroaryl, alkoxy-alkoxy, O—(CH₂)ₚ-cycloalkyl, and O—(CH₂)_{q}-heterocycloalkyl,
wherein said cycloalkyl and heterocycloalkyl groups (or portions of groups) in each of the above substituents are optionally further substituted by one or more halo, haloalkyl, alkyl or alkoxy groups.

In one preferred embodiment, the compound is of formula (IIb), or a pharmaceutically acceptable salt or solvate thereof,

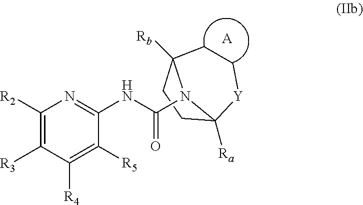

wherein:
R₂, R₄ and R₅ are each independently selected from H, halo, haloalkyl, haloalkoxy, alkyl, alkoxy, OH and CN; and
R₃ is a bicyclic heteroaryl group, wherein said bicyclic heteroaryl group is optionally further substituted by one or more substituents selected from halo, haloalkyl, alkyl, alkoxy, NHCO-alkyl, NR₁₁R_{11′}, SO₂-alkyl, CN, hydroxyalkyl, CONR₁₂R_{12′}, alkyl-NR₁₃R_{13′}, alkylamino-alkoxy, dialkylamino-alkoxy, heterocycloalkyl, alkyl-heterocycloalkyl, alkyl-cycloalkyl, aryl, (CH₂)ₘ NHSO₂-alkyl, CO₂R₁₄, alkoxy-alkyl, haloalkoxy, heteroaryl, alkoxy-alkoxy, O—(CH₂)ₚ-cycloalkyl, and O—(CH₂)_{q}-heterocycloalkyl,
   wherein said cycloalkyl and heterocycloalkyl groups in each of the above substituents are optionally further substituted by one or more halo, haloalkyl, alkyl or alkoxy groups.

In one preferred embodiment, R₁₁, R_{11′}, R₁₂, R_{12′}, R₁₃ and R_{13′} are each independently selected from H and alkyl, more preferably H and Me.

In one preferred embodiment, R₁₄ is alkyl, more preferably Me, Et, ⁱPr, ⁿPr, ⁿBu, ⁱBu or ^{t}Bu.

In one preferred embodiment, the bicyclic heteroaryl group is a 9- or 10-membered bicyclic heteroaryl group, preferably containing at least one nitrogen.

In one preferred embodiment, R₃ is a 9-membered bicyclic heteroaryl group selected from the following:

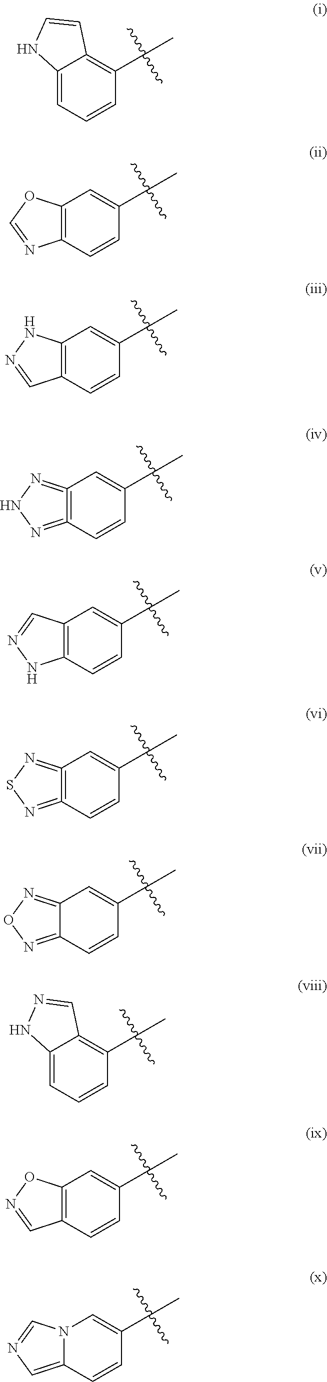

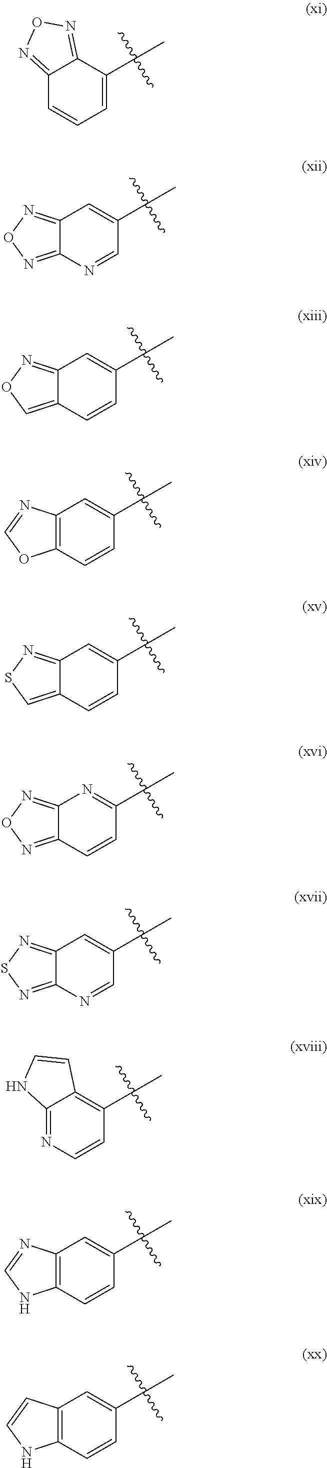

each of which is optionally substituted by one or more substituents selected from alkyl, haloalkyl and halo.

In one preferred embodiment, R₃ is selected from the following groups:

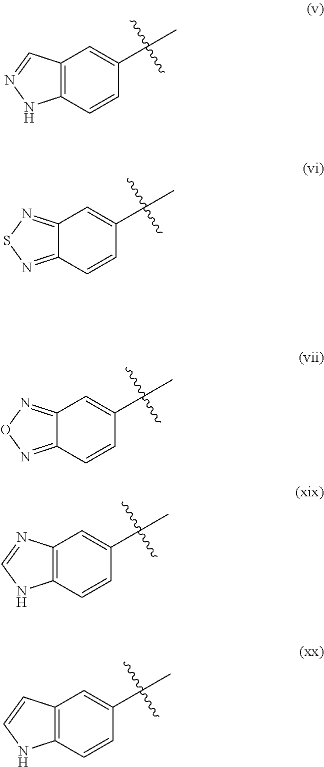 [SMILES: *c1ccc2[nH]ncc2c1 ; *c1ccc2nsnc2c1 ; *c1ccc2nonc2c1 ; *c1ccc2[nH]cnc2c1 ; *c1ccc2[nH]ccc2c1]

In one preferred embodiment, R₃ is a group of formula (vii).

In one preferred embodiment, R₂ and R₅ are both H.

In one preferred embodiment, R₄ is selected from C₁, Br, and CF₃, more preferably Cl.

In one preferred embodiment, m is 0 or 1.

In one preferred embodiment, p is 0 or 1.

In one preferred embodiment, q is 0 or 1.

In one preferred embodiment, ring A is of formula:

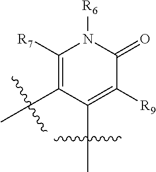

wherein R₆, R₇ and R₉ are all H, i.e. the compound is of the formula:

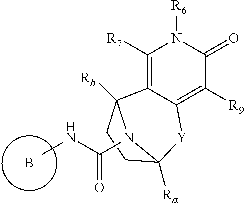

In one preferred embodiment, ring A is of formula:

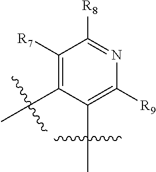

wherein R₇ and R₃ are H, and R₉ is F, i.e. the compound is of the formula:

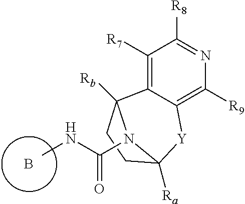

In one preferred embodiment, Rₐ and R_{b} are both H.

In one preferred embodiment, Y is selected from CH₂, CHF and CF₂, more preferably CH₂.

In one preferred embodiment, the compound of formula (II) is selected from the following:

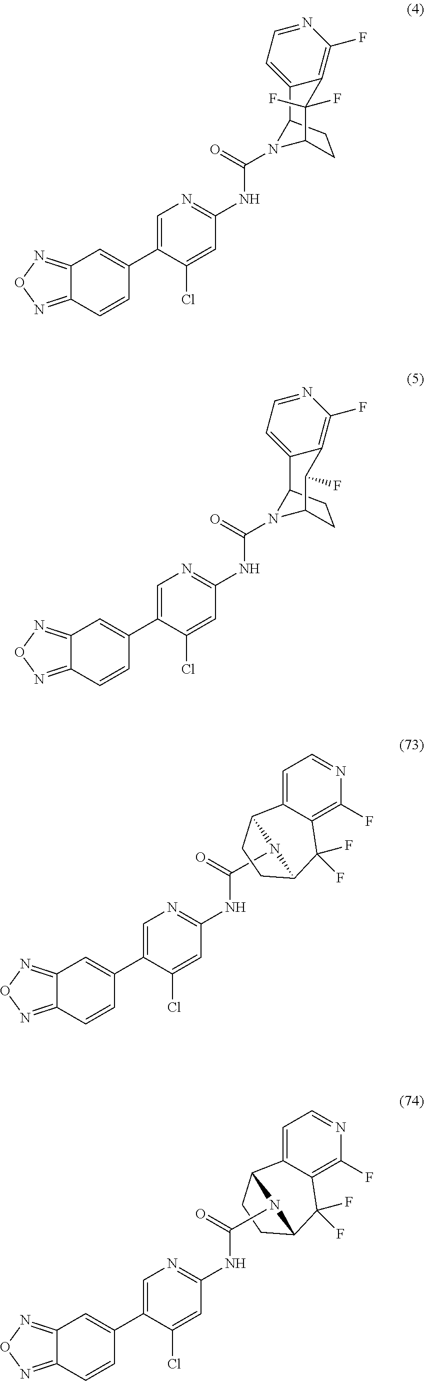 [SMILES: O=C(Nc1cc(Cl)c(-c2ccc3nonc3c2)cn1)N1C2CCC1C(F)(F)c1c2ccnc1F ; O=C(Nc1cc(Cl)c(-c2ccc3nonc3c2)cn1)N1C2CCC1[C@H](F)c1c2ccnc1F ; O=C(Nc1cc(Cl)c(-c2ccc3nonc3c2)cn1)N1[C@H]2CC[C@@H]1C(F)(F)c1c2ccnc1F ; O=C(Nc1cc(Cl)c(-c2ccc3nonc3c2)cn1)N1[C@@H]2CC[C@H]1C(F)(F)c1c2ccnc1F]

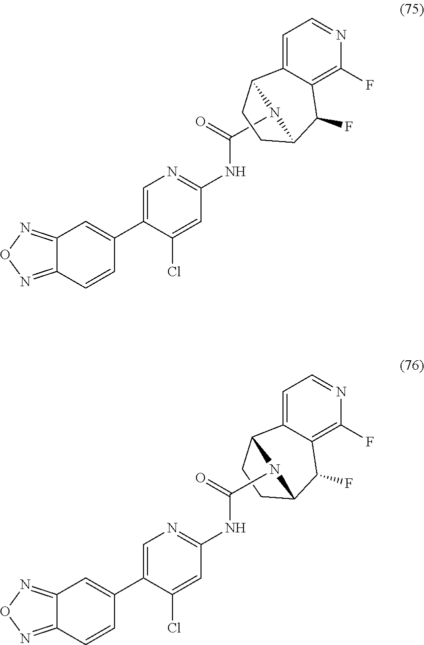 [SMILES: O=C(Nc1cc(Cl)c(-c2ccc3nonc3c2)cn1)N1[C@@H]2CC[C@H]1c1ccnc(F)c1[C@H]2F ; O=C(Nc1cc(Cl)c(-c2ccc3nonc3c2)cn1)N1[C@@H]2CC[C@H]1[C@@H](F)c1c2ccnc1F]

and enantiomers thereof, and mixtures of enantiomers thereof, including racemic mixtures, and pharmaceutically acceptable salts and solvates thereof.

### Further Compounds According to the Invention

Another aspect of the invention relates to a compound selected from the following:

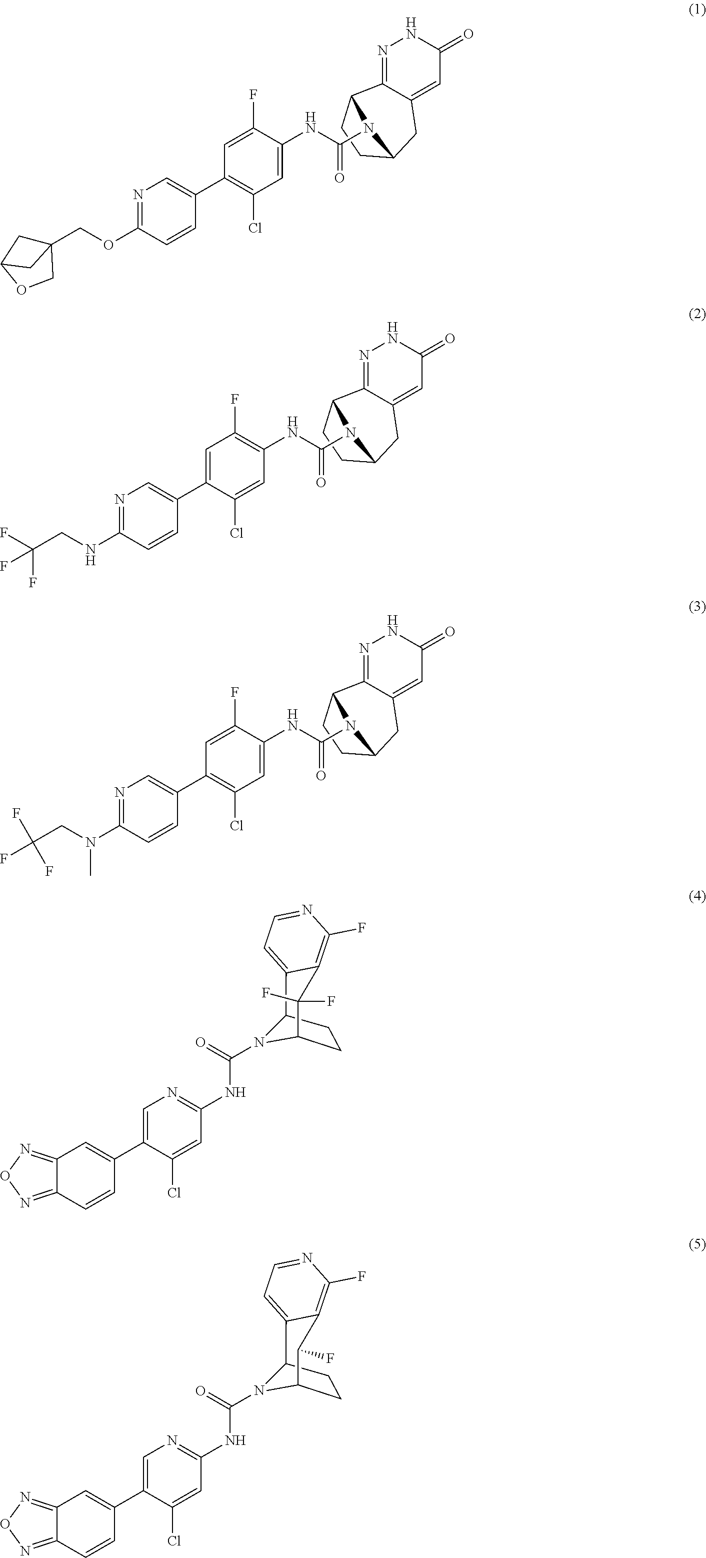 [SMILES: O=C(Nc1cc(Cl)c(-c2ccc(OCC34COC(C3)C4)nc2)cc1F)N1[C@H]2CC[C@@H]1c1n[nH]c(=O)cc1C2 ; O=C(Nc1cc(Cl)c(-c2ccc(NCC(F)(F)F)nc2)cc1F)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccc3nonc3c2)cn1)N1C2CCC1C(F)(F)c1c2ccnc1F ; O=C(Nc1cc(Cl)c(-c2ccc3nonc3c2)cn1)N1C2CCC1[C@H](F)c1c2ccnc1F]

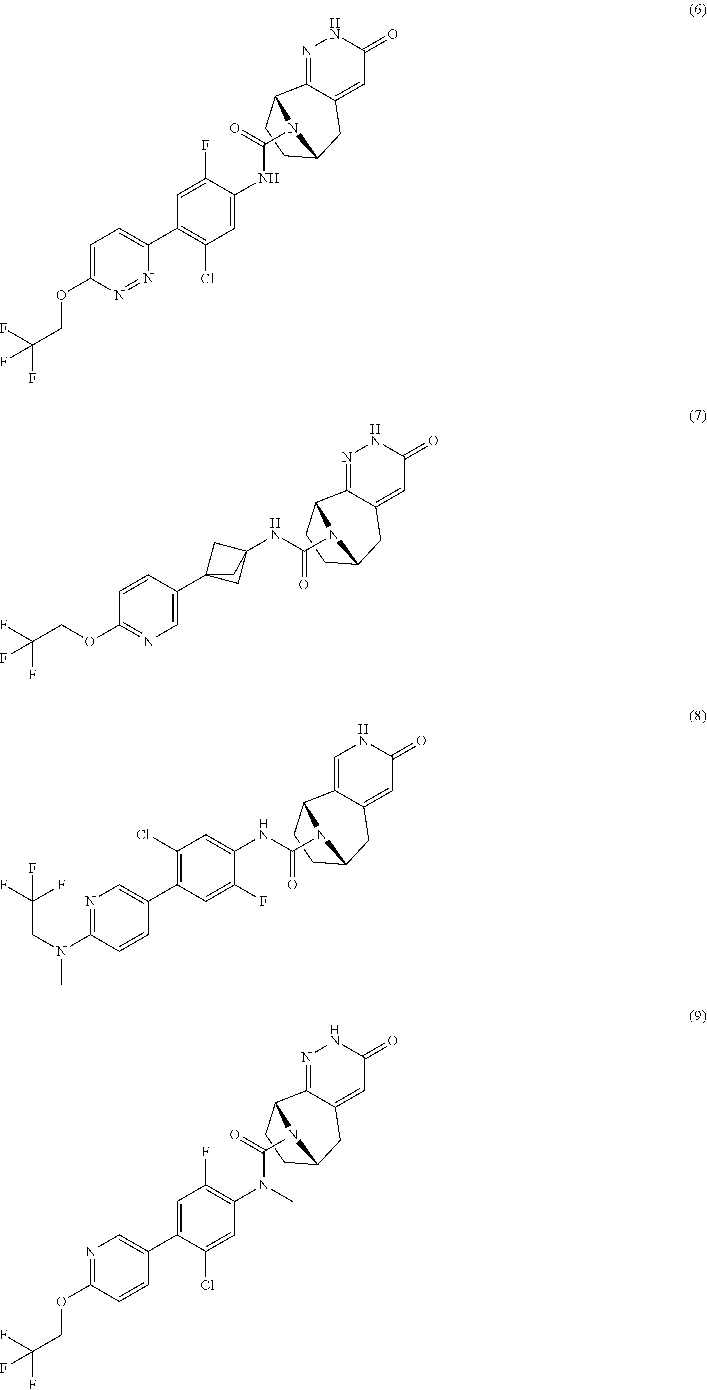 [SMILES: O=C(Nc1cc(Cl)c(-c2ccc(OCC(F)(F)F)nn2)cc1F)N1[C@H]2CC[C@@H]1c1n[nH]c(=O)cc1C2 ; O=C(NC12CC(c3ccc(OCC(F)(F)F)nc3)(C1)C2)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3c[nH]c(=O)cc3C4)cc2Cl)cn1 ; CN(C(=O)N1[C@H]2CC[C@@H]1c1n[nH]c(=O)cc1C2)c1cc(Cl)c(-c2ccc(OCC(F)(F)F)nc2)cc1F]

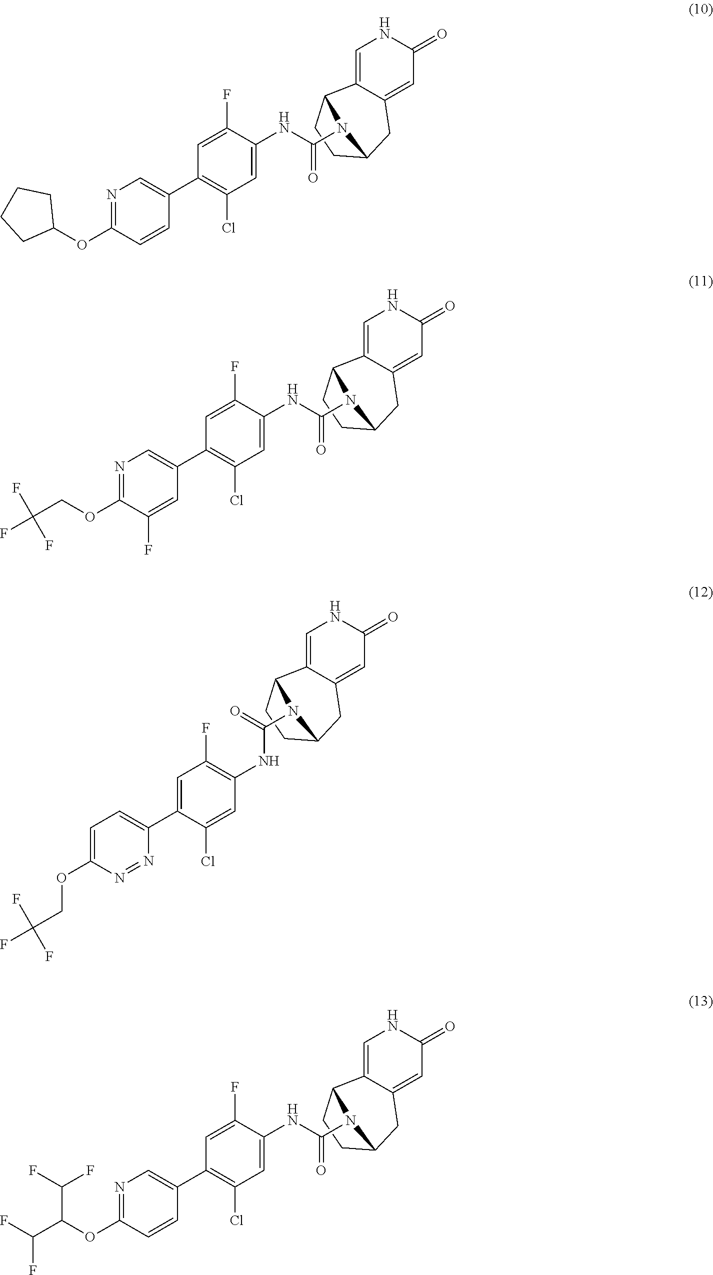 [SMILES: O=C(Nc1cc(Cl)c(-c2ccc(OC3CCCC3)nc2)cc1F)N1[C@H]2CC[C@@H]1c1c[nH]c(=O)cc1C2 ; O=C(Nc1cc(Cl)c(-c2cnc(OCC(F)(F)F)c(F)c2)cc1F)N1[C@H]2CC[C@@H]1c1c[nH]c(=O)cc1C2 ; O=C(Nc1cc(Cl)c(-c2ccc(OCC(F)(F)F)nn2)cc1F)N1[C@H]2CC[C@@H]1c1c[nH]c(=O)cc1C2 ; O=C(Nc1cc(Cl)c(-c2ccc(OC(C(F)F)C(F)F)nc2)cc1F)N1[C@H]2CC[C@@H]1c1c[nH]c(=O)cc1C2]

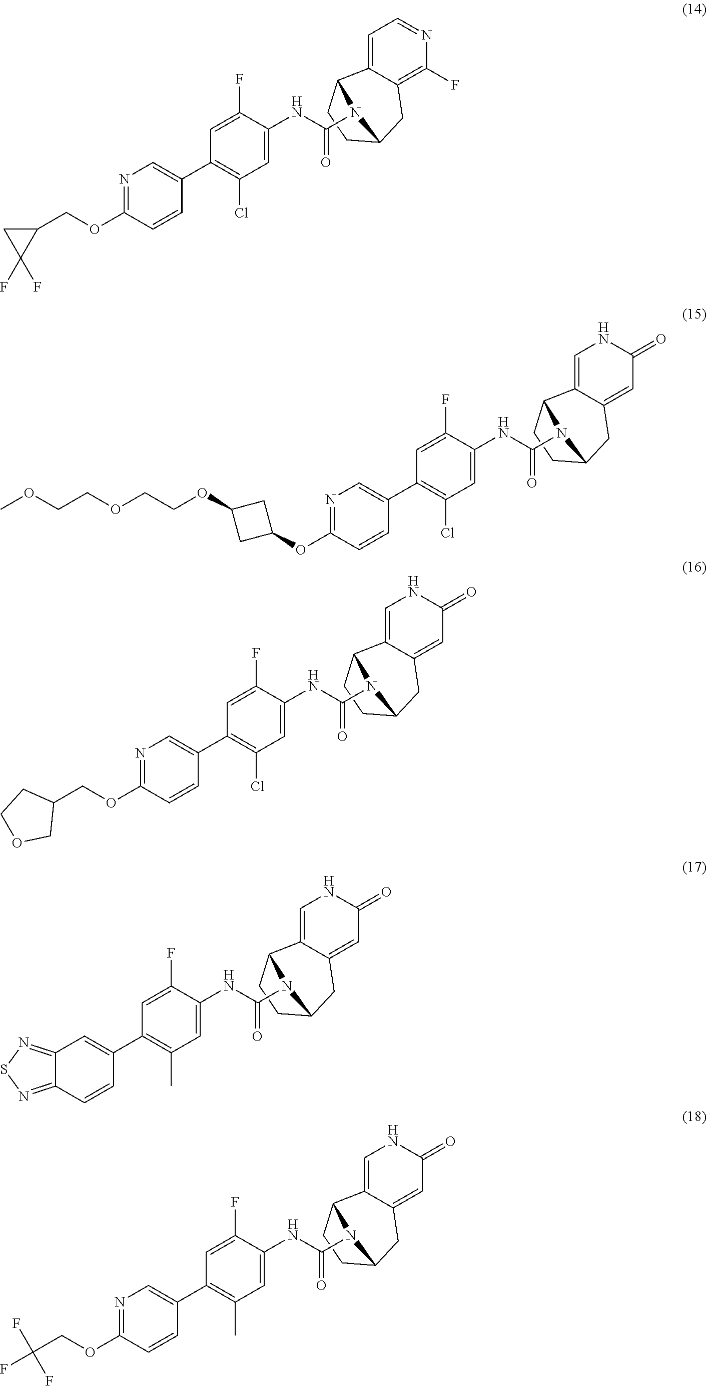 [SMILES: O=C(Nc1cc(Cl)c(-c2ccc(OCC3CC3(F)F)nc2)cc1F)N1[C@H]2CC[C@@H]1c1ccnc(F)c1C2 ; COCCOCCO[C@H]1C[C@@H](Oc2ccc(-c3cc(F)c(NC(=O)N4[C@H]5CC[C@@H]4c4c[nH]c(=O)cc4C5)cc3Cl)cn2)C1 ; O=C(Nc1cc(Cl)c(-c2ccc(OCC3CCOC3)nc2)cc1F)N1[C@H]2CC[C@@H]1c1c[nH]c(=O)cc1C2 ; Cc1cc(NC(=O)N2[C@H]3CC[C@@H]2c2c[nH]c(=O)cc2C3)c(F)cc1-c1ccc2nsnc2c1 ; Cc1cc(NC(=O)N2[C@H]3CC[C@@H]2c2c[nH]c(=O)cc2C3)c(F)cc1-c1ccc(OCC(F)(F)F)nc1]

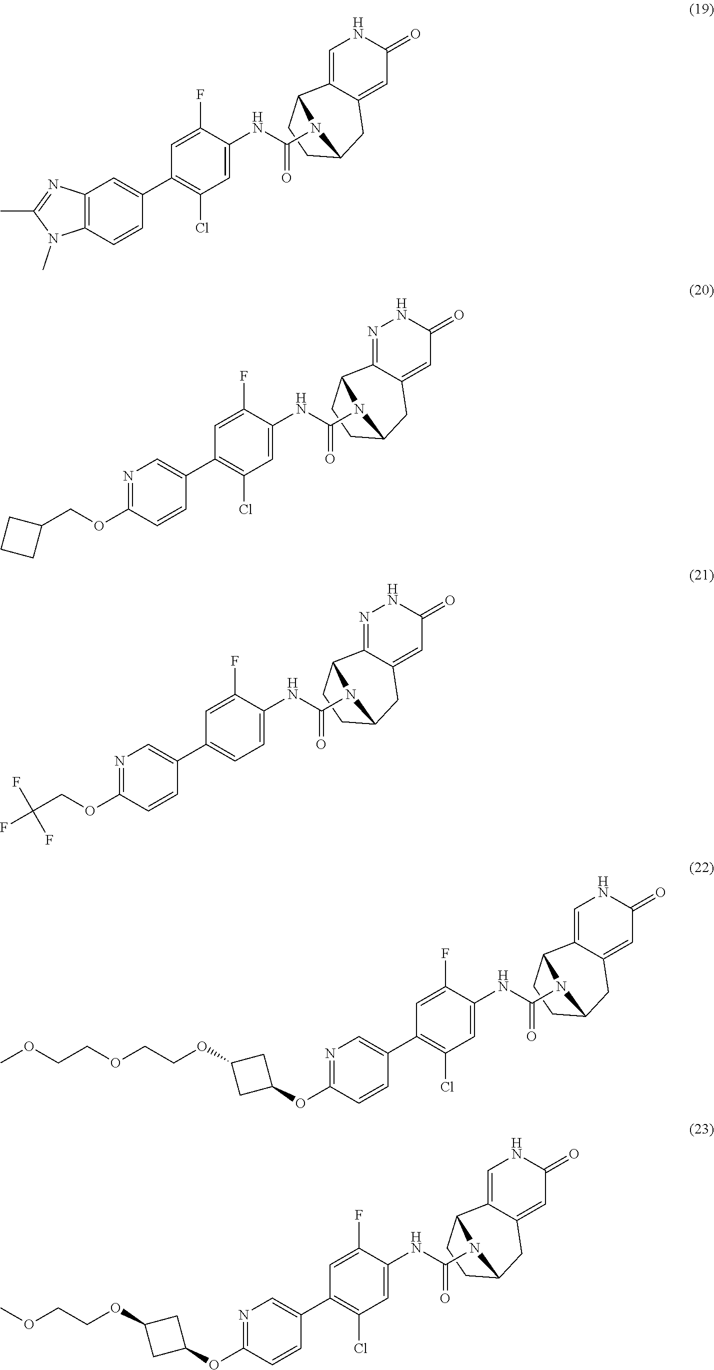 [SMILES: Cc1nc2cc(-c3cc(F)c(NC(=O)N4[C@H]5CC[C@@H]4c4c[nH]c(=O)cc4C5)cc3Cl)ccc2n1C ; O=C(Nc1cc(Cl)c(-c2ccc(OCC3CCC3)nc2)cc1F)N1[C@H]2CC[C@@H]1c1n[nH]c(=O)cc1C2 ; O=C(Nc1ccc(-c2ccc(OCC(F)(F)F)nc2)cc1F)N1[C@H]2CC[C@@H]1c1n[nH]c(=O)cc1C2 ; COCCOCCO[C@H]1C[C@H](Oc2ccc(-c3cc(F)c(NC(=O)N4[C@H]5CC[C@@H]4c4c[nH]c(=O)cc4C5)cc3Cl)cn2)C1 ; COCCO[C@H]1C[C@@H](Oc2ccc(-c3cc(F)c(NC(=O)N4[C@H]5CC[C@@H]4c4c[nH]c(=O)cc4C5)cc3Cl)cn2)C1]

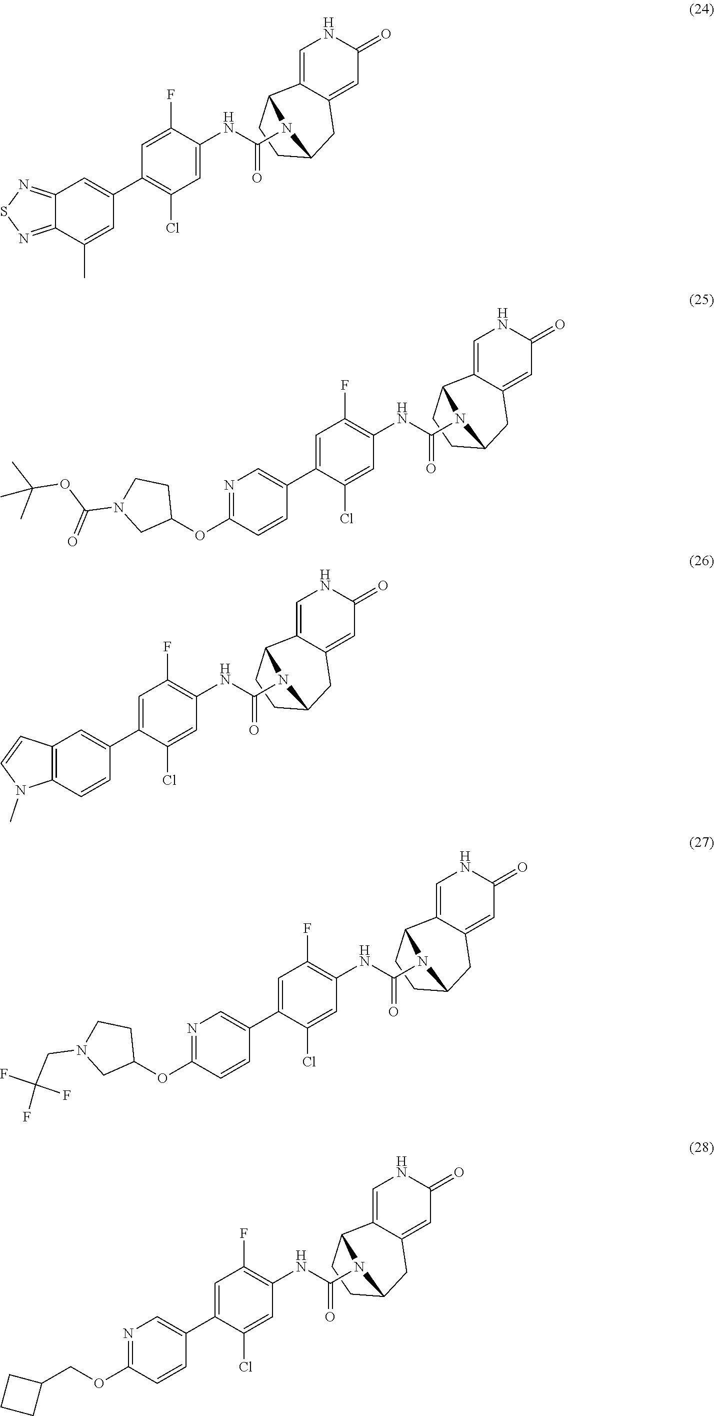 [SMILES: Cc1cc(-c2cc(F)c(NC(=O)N3[C@H]4CC[C@@H]3c3c[nH]c(=O)cc3C4)cc2Cl)cc2nsnc12 ; CC(C)(C)OC(=O)N1CCC(Oc2ccc(-c3cc(F)c(NC(=O)N4[C@H]5CC[C@@H]4c4c[nH]c(=O)cc4C5)cc3Cl)cn2)C1 ; Cn1ccc2cc(-c3cc(F)c(NC(=O)N4[C@H]5CC[C@@H]4c4c[nH]c(=O)cc4C5)cc3Cl)ccc21 ; O=C(Nc1cc(Cl)c(-c2ccc(OC3CCN(CC(F)(F)F)C3)nc2)cc1F)N1[C@H]2CC[C@@H]1c1c[nH]c(=O)cc1C2 ; O=C(Nc1cc(Cl)c(-c2ccc(OCC3CCC3)nc2)cc1F)N1[C@H]2CC[C@@H]1c1c[nH]c(=O)cc1C2]

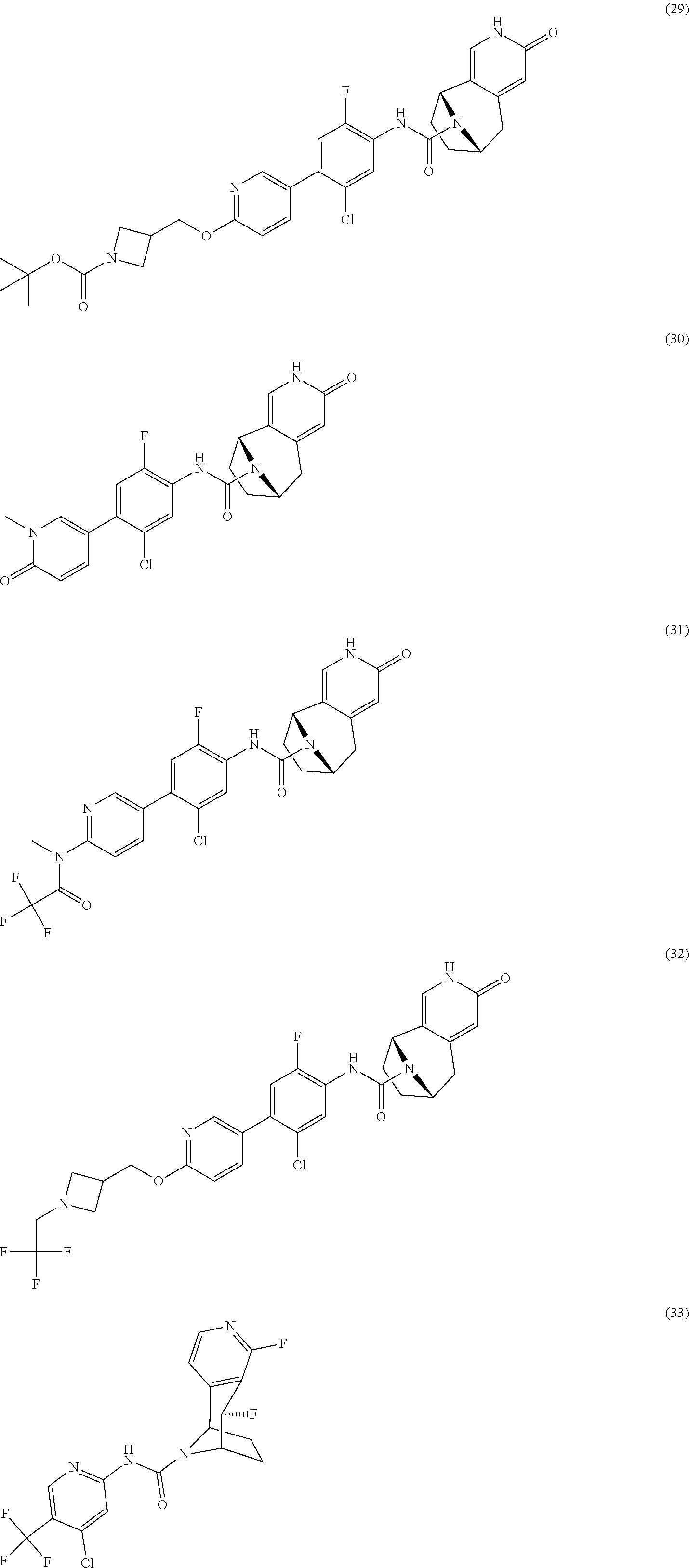 [SMILES: CC(C)(C)OC(=O)N1CC(COc2ccc(-c3cc(F)c(NC(=O)N4[C@H]5CC[C@@H]4c4c[nH]c(=O)cc4C5)cc3Cl)cn2)C1 ; Cn1cc(-c2cc(F)c(NC(=O)N3[C@H]4CC[C@@H]3c3c[nH]c(=O)cc3C4)cc2Cl)ccc1=O ; CN(C(=O)C(F)(F)F)c1ccc(-c2cc(F)c(NC(=O)N3[C@H]4CC[C@@H]3c3c[nH]c(=O)cc3C4)cc2Cl)cn1 ; O=C(Nc1cc(Cl)c(-c2ccc(OCC3CN(CC(F)(F)F)C3)nc2)cc1F)N1[C@H]2CC[C@@H]1c1c[nH]c(=O)cc1C2 ; O=C(Nc1cc(Cl)c(C(F)(F)F)cn1)N1C2CCC1[C@H](F)c1c2ccnc1F]

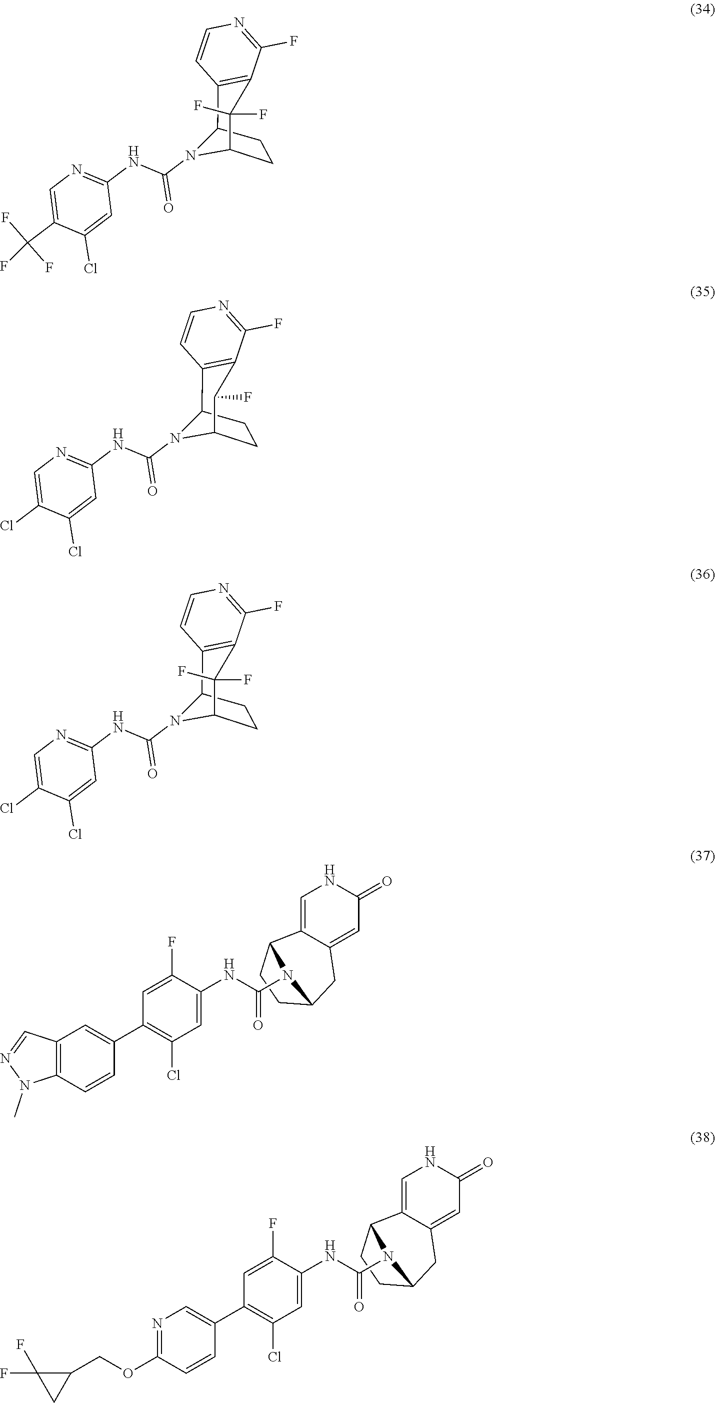 [SMILES: O=C(Nc1cc(Cl)c(C(F)(F)F)cn1)N1C2CCC1C(F)(F)c1c2ccnc1F ; O=C(Nc1cc(Cl)c(Cl)cn1)N1C2CCC1[C@H](F)c1c2ccnc1F ; O=C(Nc1cc(Cl)c(Cl)cn1)N1C2CCC1C(F)(F)c1c2ccnc1F ; Cn1ncc2cc(-c3cc(F)c(NC(=O)N4[C@H]5CC[C@@H]4c4c[nH]c(=O)cc4C5)cc3Cl)ccc21 ; O=C(Nc1cc(Cl)c(-c2ccc(OCC3CC3(F)F)nc2)cc1F)N1[C@H]2CC[C@@H]1c1c[nH]c(=O)cc1C2]

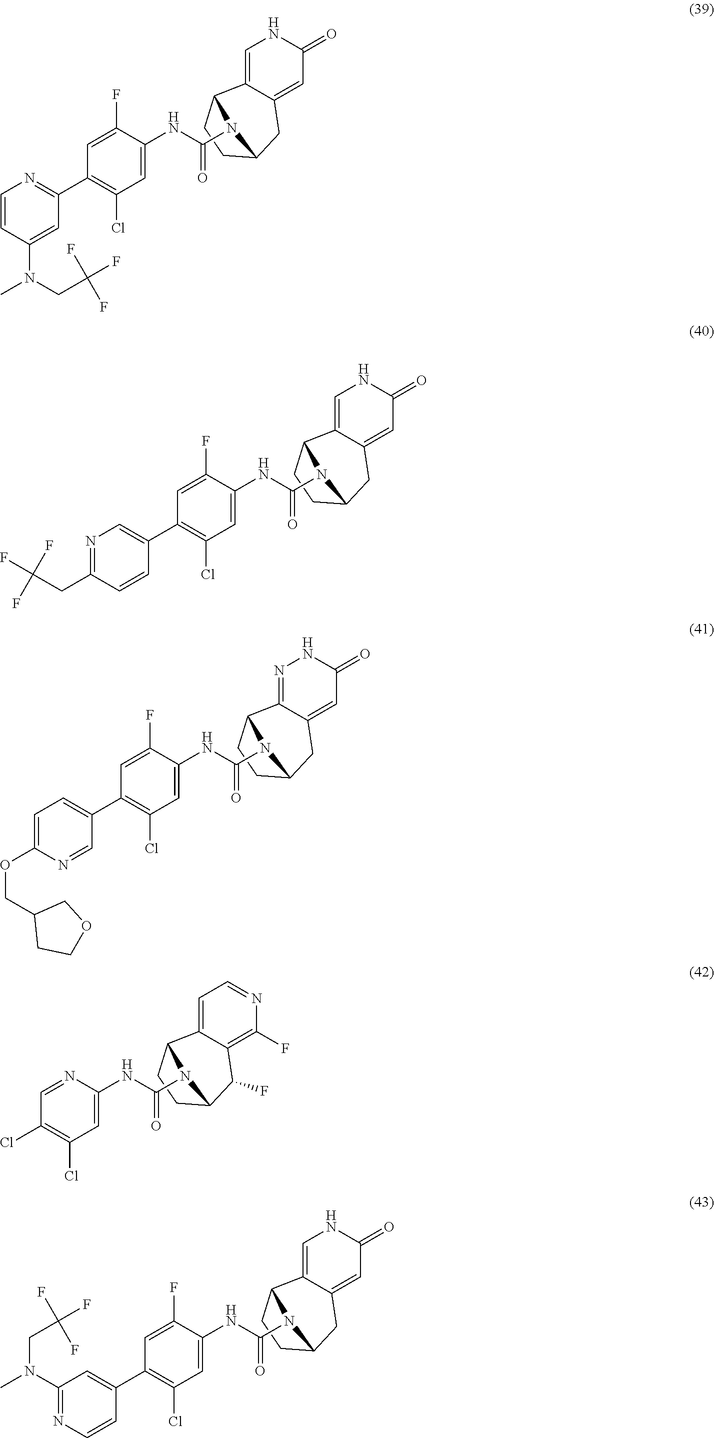 [SMILES: CN(CC(F)(F)F)c1ccnc(-c2cc(F)c(NC(=O)N3[C@H]4CC[C@@H]3c3c[nH]c(=O)cc3C4)cc2Cl)c1 ; O=C(Nc1cc(Cl)c(-c2ccc(CC(F)(F)F)nc2)cc1F)N1[C@H]2CC[C@@H]1c1c[nH]c(=O)cc1C2 ; O=C(Nc1cc(Cl)c(-c2ccc(OCC3CCOC3)nc2)cc1F)N1[C@H]2CC[C@@H]1c1n[nH]c(=O)cc1C2 ; O=C(Nc1cc(Cl)c(Cl)cn1)N1[C@@H]2CC[C@H]1[C@@H](F)c1c2ccnc1F ; CN(CC(F)(F)F)c1cc(-c2cc(F)c(NC(=O)N3[C@H]4CC[C@@H]3c3c[nH]c(=O)cc3C4)cc2Cl)ccn1]

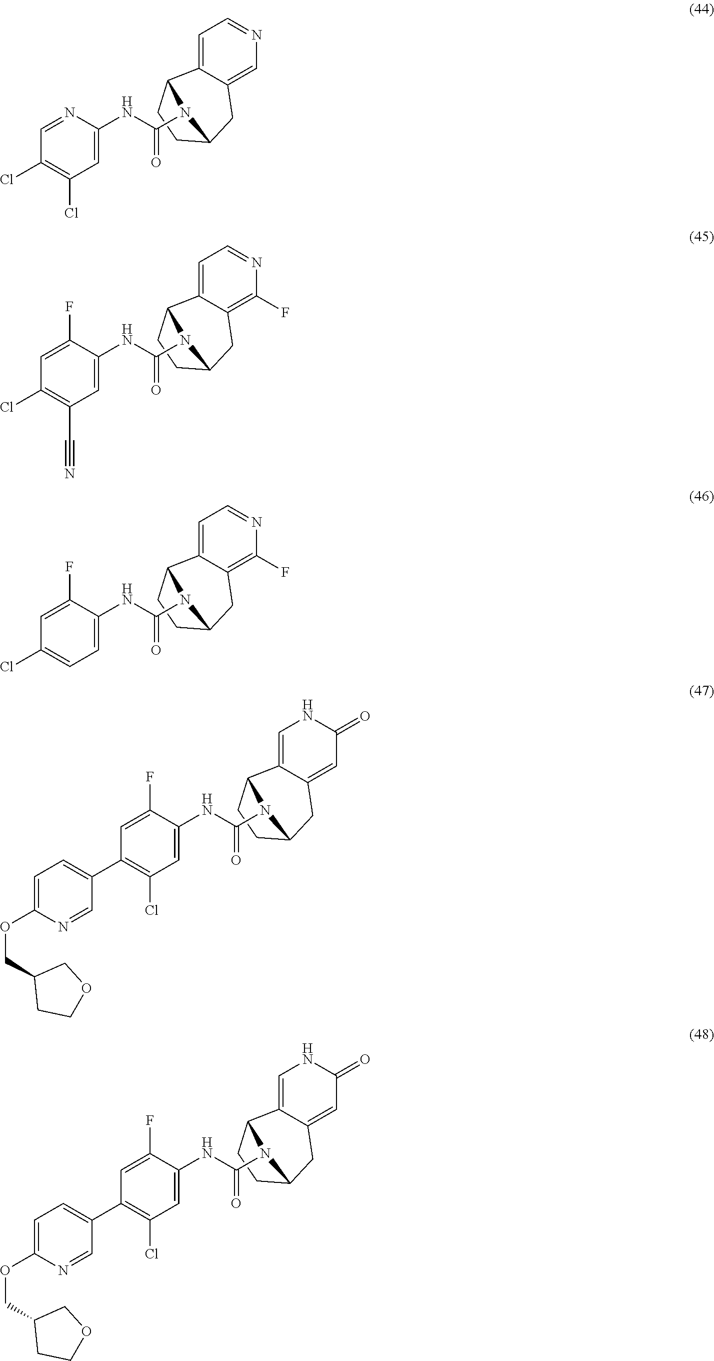 [SMILES: O=C(Nc1cc(Cl)c(Cl)cn1)N1[C@H]2CC[C@@H]1c1ccncc1C2 ; N#Cc1cc(NC(=O)N2[C@H]3CC[C@@H]2c2ccnc(F)c2C3)c(F)cc1Cl ; O=C(Nc1ccc(Cl)cc1F)N1[C@H]2CC[C@@H]1c1ccnc(F)c1C2 ; O=C(Nc1cc(Cl)c(-c2ccc(OC[C@@H]3CCOC3)nc2)cc1F)N1[C@H]2CC[C@@H]1c1c[nH]c(=O)cc1C2 ; O=C(Nc1cc(Cl)c(-c2ccc(OC[C@H]3CCOC3)nc2)cc1F)N1[C@H]2CC[C@@H]1c1c[nH]c(=O)cc1C2]

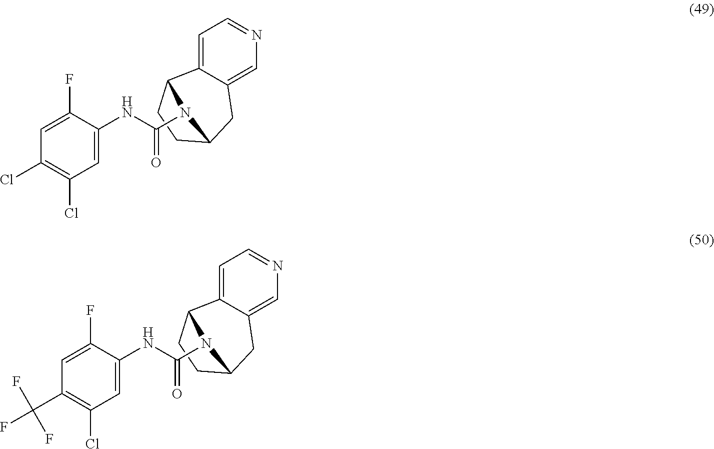 [SMILES: O=C(Nc1cc(Cl)c(Cl)cc1F)N1[C@H]2CC[C@@H]1c1ccncc1C2 ; O=C(Nc1cc(Cl)c(C(F)(F)F)cc1F)N1[C@H]2CC[C@@H]1c1ccncc1C2]

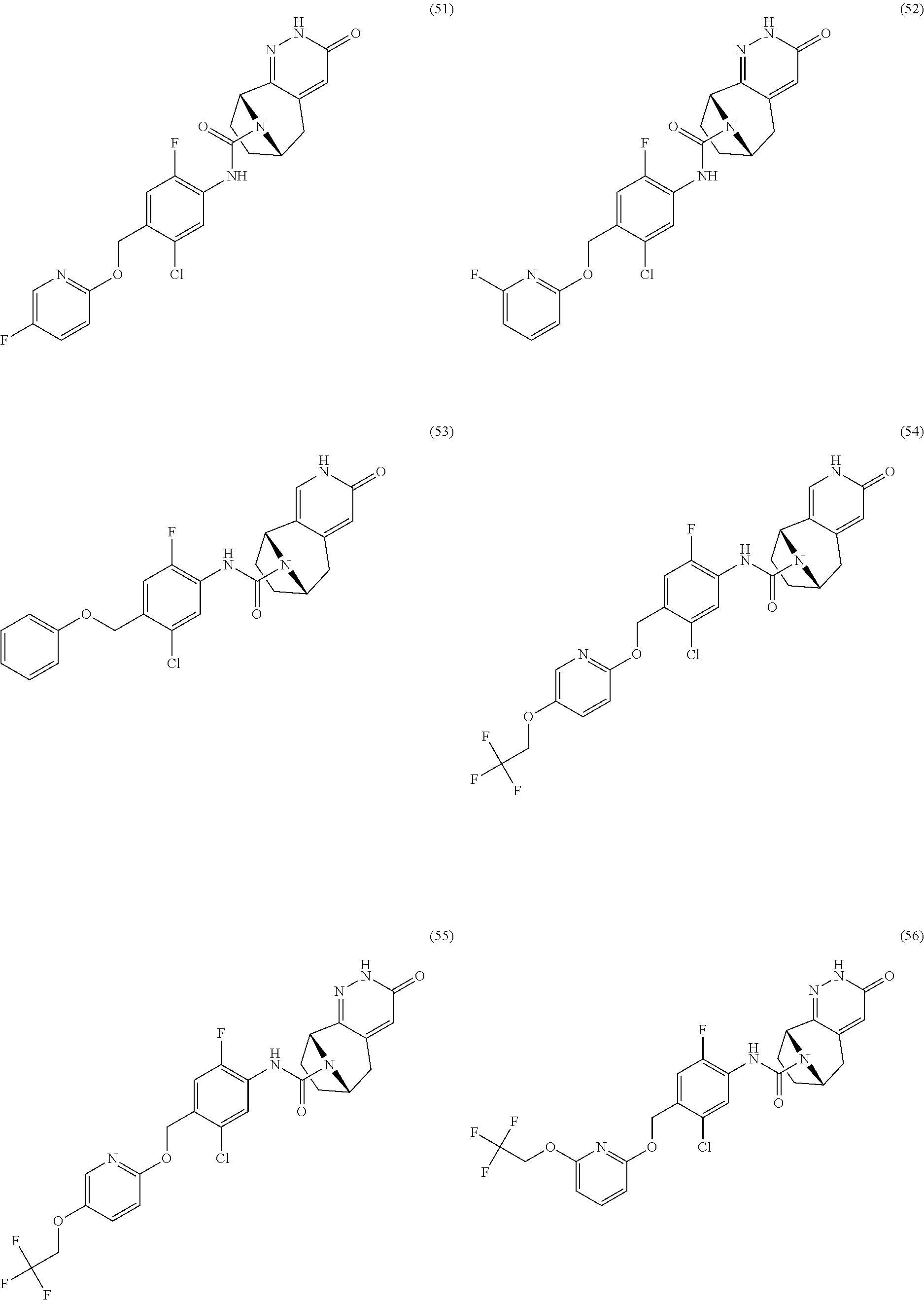 [SMILES: O=C(Nc1cc(Cl)c(COc2ccc(F)cn2)cc1F)N1[C@H]2CC[C@@H]1c1n[nH]c(=O)cc1C2 ; O=C(Nc1cc(Cl)c(COc2cccc(F)n2)cc1F)N1[C@H]2CC[C@@H]1c1n[nH]c(=O)cc1C2 ; O=C(Nc1cc(Cl)c(COc2ccccc2)cc1F)N1[C@H]2CC[C@@H]1c1c[nH]c(=O)cc1C2 ; O=C(Nc1cc(Cl)c(COc2ccc(OCC(F)(F)F)cn2)cc1F)N1[C@H]2CC[C@@H]1c1c[nH]c(=O)cc1C2 ; O=C(Nc1cc(Cl)c(COc2ccc(OCC(F)(F)F)cn2)cc1F)N1[C@H]2CC[C@@H]1c1n[nH]c(=O)cc1C2 ; O=C(Nc1cc(Cl)c(COc2cccc(OCC(F)(F)F)n2)cc1F)N1[C@H]2CC[C@@H]1c1n[nH]c(=O)cc1C2]

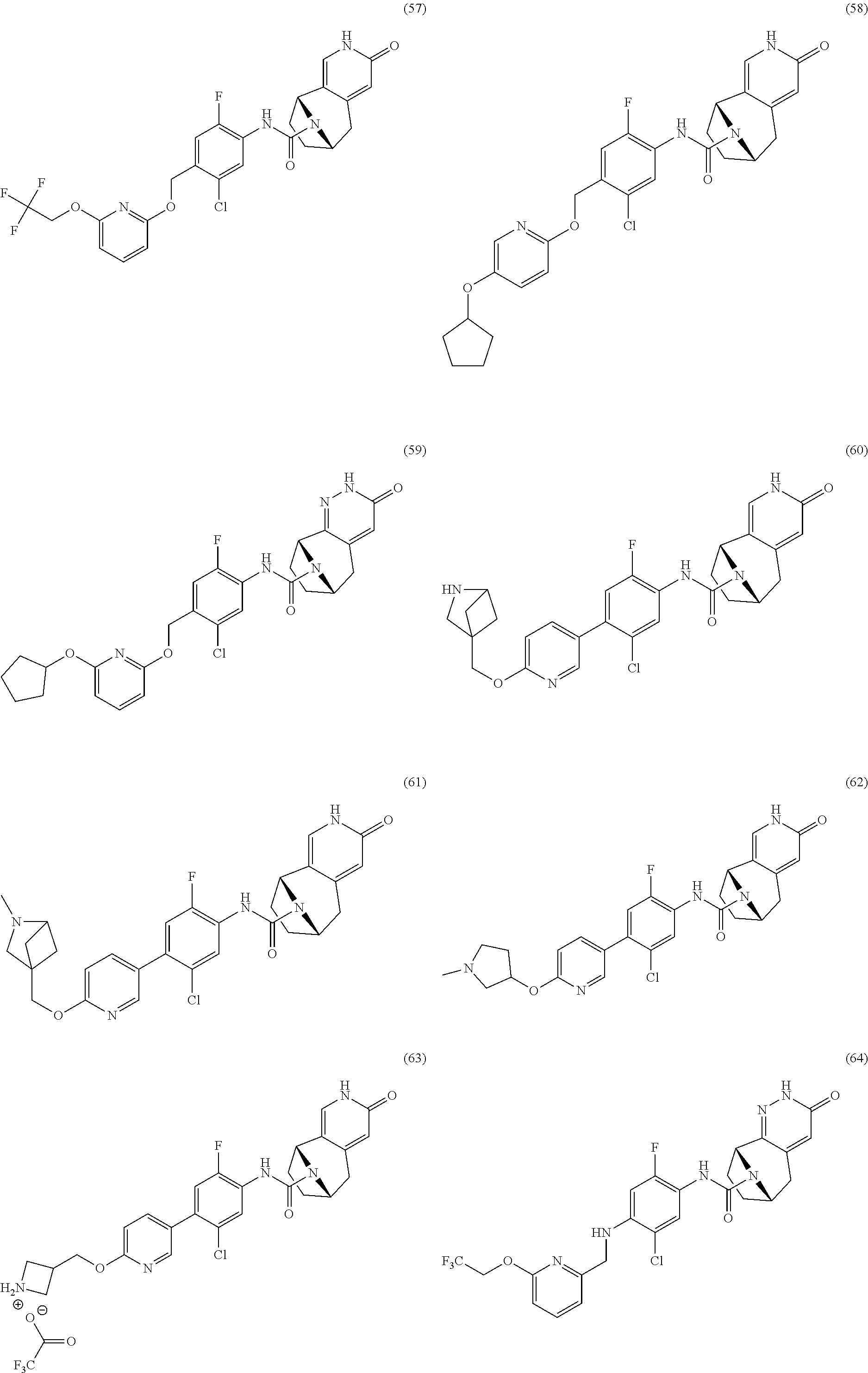

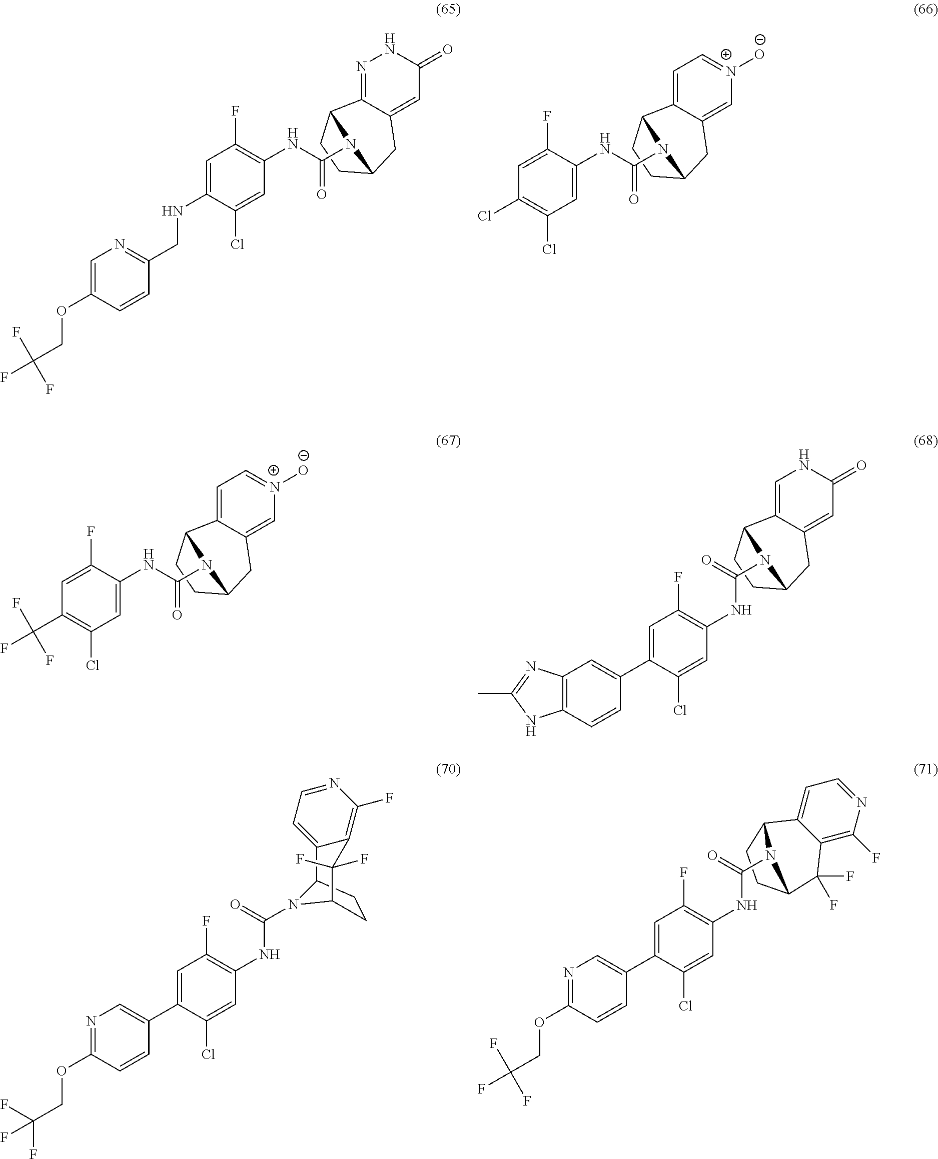 [SMILES: O=C(Nc1cc(Cl)c(NCc2ccc(OCC(F)(F)F)cn2)cc1F)N1[C@H]2CC[C@@H]1c1n[nH]c(=O)cc1C2 ; O=C(Nc1cc(Cl)c(Cl)cc1F)N1[C@H]2CC[C@@H]1c1cc[n+]([O-])cc1C2 ; O=C(Nc1cc(Cl)c(C(F)(F)F)cc1F)N1[C@H]2CC[C@@H]1c1cc[n+]([O-])cc1C2 ; Cc1nc2cc(-c3cc(F)c(NC(=O)N4[C@H]5CC[C@@H]4c4c[nH]c(=O)cc4C5)cc3Cl)ccc2[nH]1 ; O=C(Nc1cc(Cl)c(-c2ccc(OCC(F)(F)F)nc2)cc1F)N1C2CCC1C(F)(F)c1c2ccnc1F ; O=C(Nc1cc(Cl)c(-c2ccc(OCC(F)(F)F)nc2)cc1F)N1[C@@H]2CC[C@H]1C(F)(F)c1c2ccnc1F]

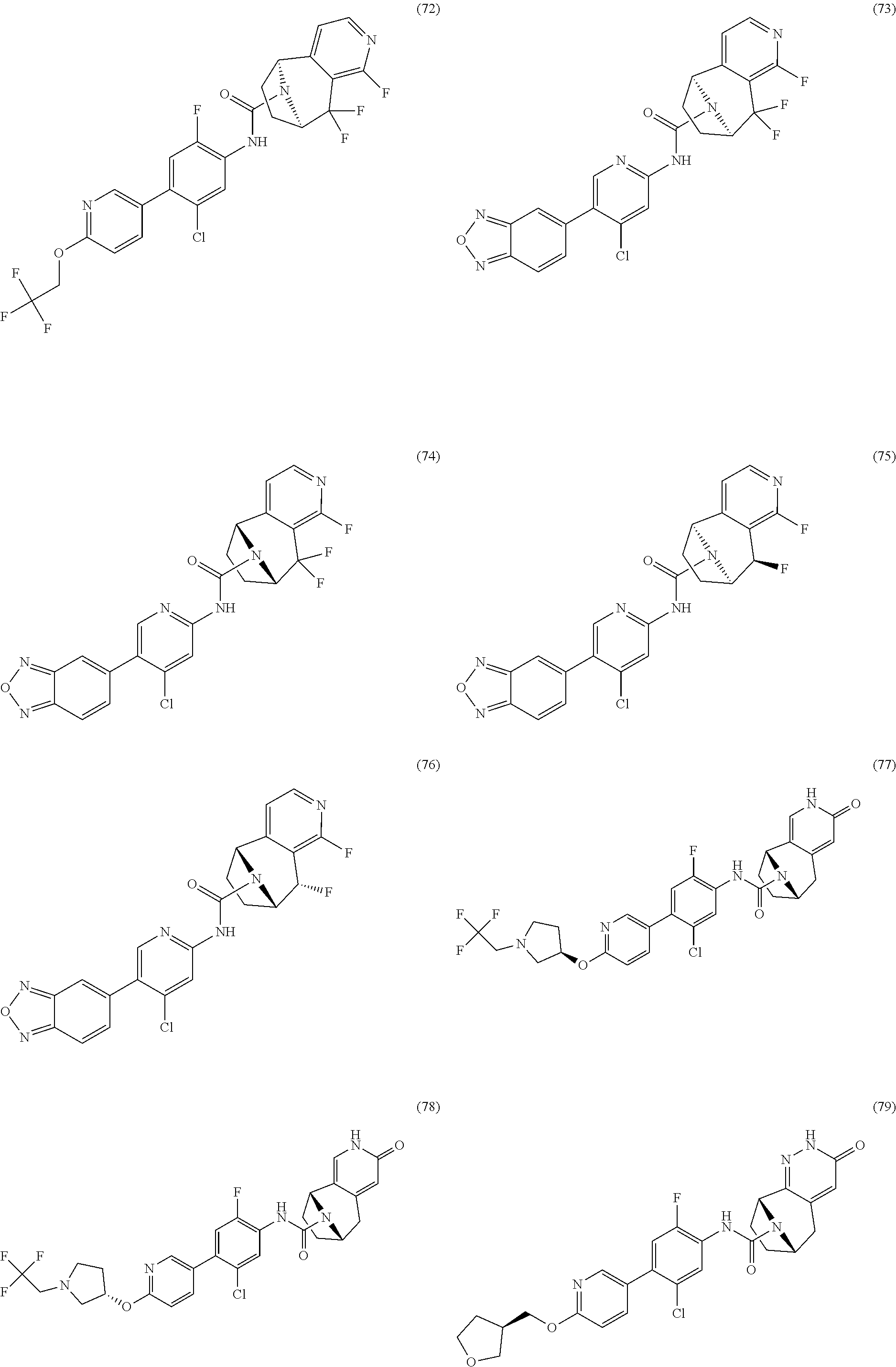

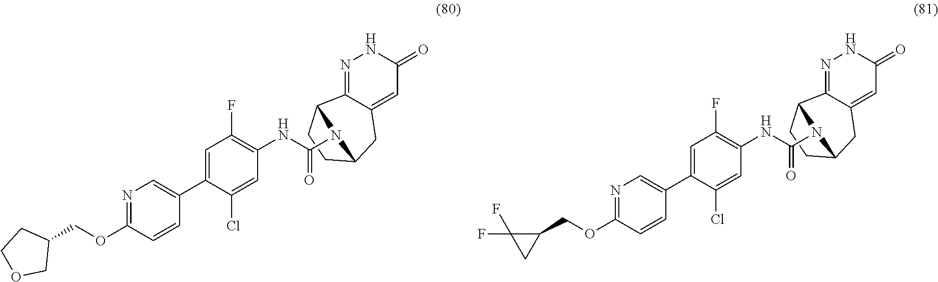 [SMILES: O=C(Nc1cc(Cl)c(-c2ccc(OC[C@H]3CCOC3)nc2)cc1F)N1[C@H]2CC[C@@H]1c1n[nH]c(=O)cc1C2 ; O=C(Nc1cc(Cl)c(-c2ccc(OC[C@H]3CC3(F)F)nc2)cc1F)N1[C@H]2CC[C@@H]1c1n[nH]c(=O)cc1C2]

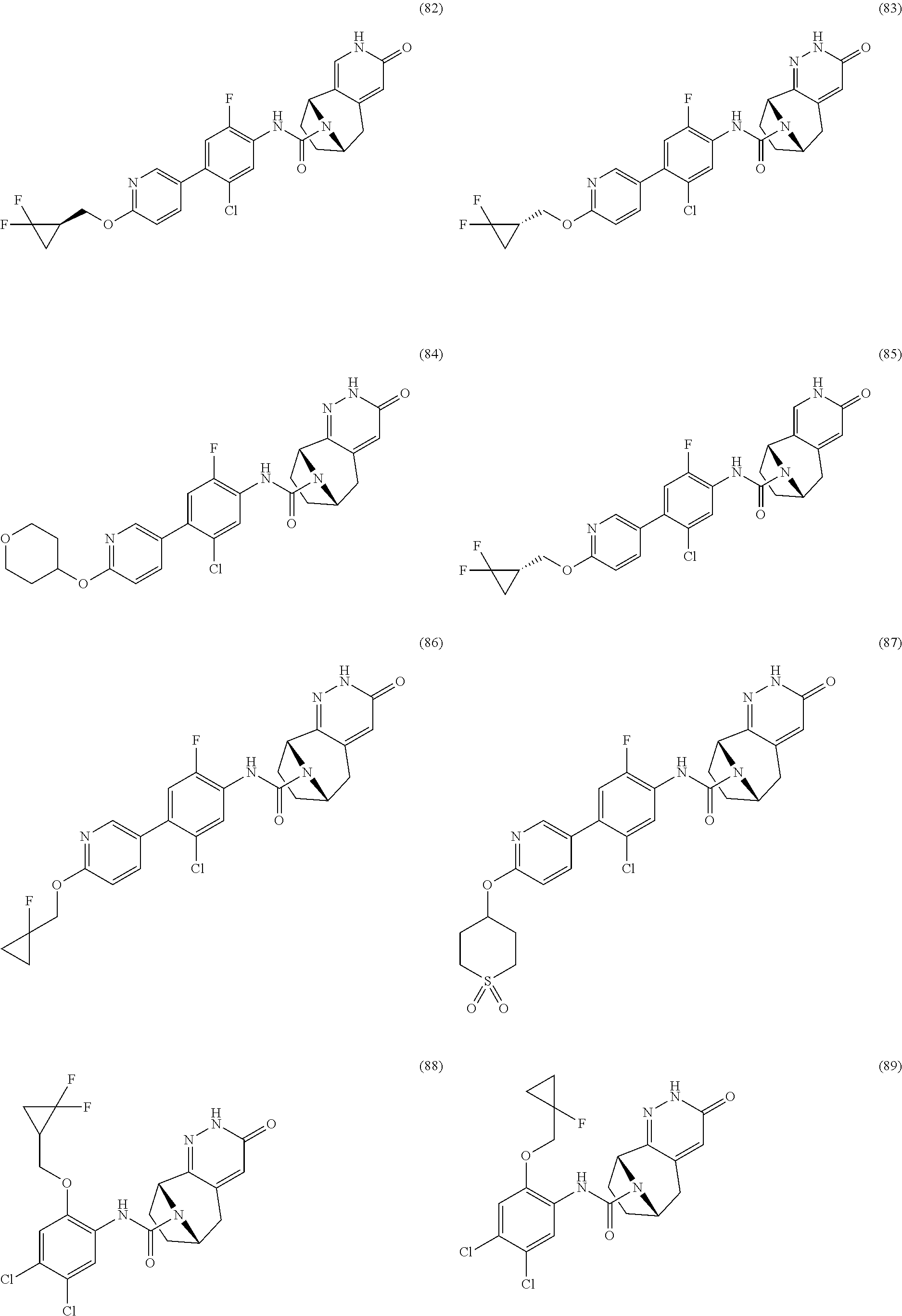

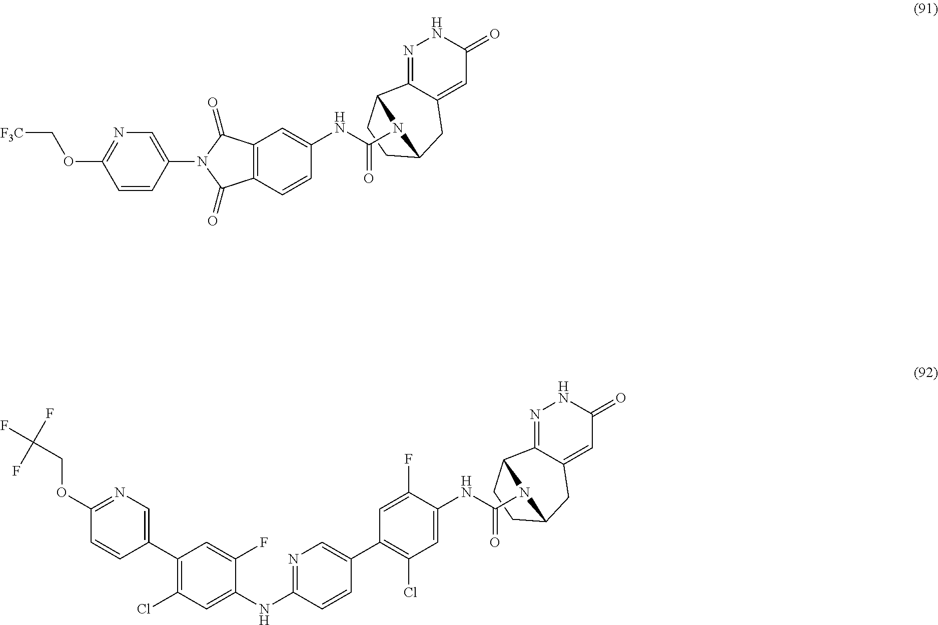 [SMILES: O=C1c2ccc(NC(=O)N3[C@H]4CC[C@@H]3c3n[nH]c(=O)cc3C4)cc2C(=O)N1c1ccc(OCC(F)(F)F)nc1 ; O=C(Nc1cc(Cl)c(-c2ccc(Nc3cc(Cl)c(-c4ccc(OCC(F)(F)F)nc4)cc3F)nc2)cc1F)N1[C@H]2CC[C@@H]1c1n[nH]c(=O)cc1C2]

and enantiomers thereof, and mixtures of enantiomers thereof, including racemic mixtures, and pharmaceutically acceptable salts and solvates thereof.

In one preferred embodiment, the compound of the invention is selected from the following:

| | |
|---|---|
| (1) | (6S,9R)-N-(4-(6-((2-oxabicyclo[2.1.1]hexan-4-yl)methoxy)pyridin-3-yl)-5-chloro-2- |
| | fluorophenyl)-3-oxo-3,5,6,7,8,9-hexahydro-2H-6,9- |
| | epiminocyclohepta[c]pyridazine-10-carboxamide |
| (2) | (6S,9R)-N-(5-chloro-2-fluoro-4-(6-((2,2,2-trifluoroethyl)amino)pyridin-3-yl)phenyl)- |
| | 3-oxo-3,5,6,7,8,9-hexahydro-2H-6,9-epiminocyclohepta[c]pyridazine-10- |
| | carboxamide |
| (3) | (6S,9R)-N-(5-chloro-2-fluoro-4-(6-(methyl(2,2,2-trifluoroethyl)amino)pyridin-3- |
| | yl)phenyl)-3-oxo-3,5,6,7,8,9-hexahydro-2H-6,9-epiminocyclohepta[c]pyridazine- |
| | 10-carboxamide |
| (4) | (5R,8S)-N-(5-(benzo[c][1,2,5]oxadiazol-5-yl)-4-chloropyridin-2-yl)-1,9,9-trifluoro- |
| | 6,7,8,9-tetrahydro-5H-5,8-epiminocyclohepta[c]pyridine-10-carboxamide |
| (5) | (±,9S)-N-(5-(benzo[c][1,2,5]oxadiazol-5-yl)-4-chloropyridin-2-yl)-1,9-difluoro- |
| | 6,7,8,9-tetrahydro-5H-5,8-epiminocyclohepta[c]pyridine-10-carboxamide |
| (6) | (6S,9R)-N-(5-chloro-2-fluoro-4-(6-(2,2,2-trifluoroethoxy)pyridazin-3-yl)phenyl)-3- |
| | oxo-3,5,6,7,8,9-hexahydro-2H-6,9-epiminocyclohepta[c]pyridazine-10- |
| | carboxamide |
| (7) | (6S,9R)-3-Oxo-N-(3-(6-(2,2,2-trifluoroethoxy)pyridin-3-yl)bicyclo[1.1.1]pentan-1- |
| | yl)-3,5,6,7,8,9-hexahydro-2H-6,9-epiminocyclohepta[c]pyridazine-10-carboxamide |
| (8) | (6S,9R)-N-(5-chloro-2-fluoro-4-(6-(methyl(2,2,2-trifluoroethyl)amino)pyridin-3- |
| | yl)phenyl)-3-oxo-3,5,6,7,8,9-hexahydro-2H-6,9-epiminocyclohepta[c]pyridine-10- |
| | carboxamide |
| (9) | (6S,9R)-N-(5-chloro-2-fluoro-4-(6-(2,2,2-trifluoroethoxy)pyridin-3-yl)phenyl)-N- |
| | methyl-3-oxo-3,5,6,7,8,9-hexahydro-2H-6,9-epiminocyclohepta[c]pyridazine-10- |
| | carboxamide |
| (10) | (6S,9R)-N-(5-chloro-4-(6-(cyclopentyloxy)pyridin-3-yl)-2-fluorophenyl)-3-oxo- |
| | 3,5,6,7,8,9-hexahydro-2H-6,9-epiminocyclohepta[c]pyridine-10-carboxamide |
| (11) | (6S,9R)-N-(5-chloro-2-fluoro-4-(5-fluoro-6-(2,2,2-trifluoroethoxy)pyridin-3- |
| | yl)phenyl)-3-oxo-3,5,6,7,8,9-hexahydro-2H-6,9-epiminocyclohepta[c]pyridine-10- |
| | carboxamide |
| (12) | (6S,9R)-N-(5-chloro-2-fluoro-4-(6-(2,2,2-trifluoroethoxy)pyridazin-3-yl)phenyl)-3- |
| | oxo-3,5,6,7,8,9-hexahydro-2H-6,9-epiminocyclohepta[c]pyridine-10-carboxamide |
| (13) | (6S,9R)-N-(5-chloro-2-fluoro-4-(6-((1,1,3,3-tetrafluoropropan-2-yl)oxy)pyridin-3- |
| | yl)phenyl)-3-oxo-3,5,6,7,8,9-hexahydro-2H-6,9-epiminocyclohepta[c]pyridine-10- |
| | carboxamide |
| (14) | (5R,8S)-N-(5-chloro-4-(6-((2,2-difluorocyclopropyl)methoxy)pyridin-3-yl)-2- |
| | fluorophenyl)-1-fluoro-6,7,8,9-tetrahydro-5H-5,8-epiminocyclohepta[c]pyridine-10- |
| | carboxamide |
| (15) | (6S,9R)-N-(5-chloro-2-fluoro-4-(6-((1s,3S)-3-(2-(2- |
| | methoxyethoxy)ethoxy)cyclobutoxy)pyridin-3-yl)phenyl)-3-oxo-3,5,6,7,8,9- |
| | hexahydro-2H-6,9-epiminocyclohepta[c]pyridine-10-carboxamide |
| (16) | (6S,9R)-N-(5-chloro-2-fluoro-4-(6-((tetrahydrofuran-3-yl)methoxy)pyridin-3- |
| | yl)phenyl)-3-oxo-3,5,6,7,8,9-hexahydro-2H-6,9-epiminocyclohepta[c]pyridine-10- |
| | carboxamide |
| (17) | (6S,9R)-N-(4-(benzo[c][1,2,5]thiadiazol-5-yl)-2-fluoro-5-methylphenyl)-3-oxo- |
| | 3,5,6,7,8,9-hexahydro-2H-6,9-epiminocyclohepta[c]pyridine-10-carboxamide |
| (18) | (6S,9R)-N-(2-fluoro-5-methyl-4-(6-(2,2,2-trifluoroethoxy)pyridin-3-yl)phenyl)-3- |
| | oxo-3,5,6,7,8,9-hexahydro-2H-6,9-epiminocyclohepta[c]pyridine-10-carboxamide |
| (19) | (6S,9R)-N-(5-chloro-4-(1,2-dimethyl-1H-benzo[d]imidazol-5-yl)-2-fluorophenyl)-3- |
| | oxo-3,5,6,7,8,9-hexahydro-2H-6,9-epiminocyclohepta[c]pyridine-10-carboxamide |
| (20) | (6S,9R)-N-(5-chloro-4-(6-(cyclobutylmethoxy)pyridin-3-yl)-2-fluorophenyl)-3-oxo- |
| | 3,5,6,7,8,9-hexahydro-2H-6,9-epiminocyclohepta[c]pyridazine-10-carboxamide |
| (21) | (6S,9R)-N-(2-fluoro-4-(6-(2,2,2-trifluoroethoxy)pyridin-3-yl)phenyl)-3-oxo- |
| | 3,5,6,7,8,9-hexahydro-2H-6,9-epiminocyclohepta[c]pyridazine-10-carboxamide |
| (22) | (6S,9R)-N-(5-chloro-2-fluoro-4-(6-((1r,3R)-3-(2-(2- |
| | methoxyethoxy)ethoxy)cyclobutoxy)pyridin-3-yl)phenyl)-3-oxo-3,5,6,7,8,9- |
| | hexahydro-2H-6,9-epiminocyclohepta[c]pyridine-10-carboxamide |
| (23) | (6S,9R)-N-(5-chloro-2-fluoro-4-(6-((1s,3S)-3-(2- |
| | methoxyethoxy)cyclobutoxy)pyridin-3-yl)phenyl)-3-oxo-3,5,6,7,8,9-hexahydro-2H- |
| | 6,9-epiminocyclohepta[c]pyridine-10-carboxamide |
| (24) | (6S,9R)-N-(5-chloro-2-fluoro-4-(7-methylbenzo[c][1,2,5]thiadiazol-5-yl)phenyl)-3- |
| | oxo-3,5,6,7,8,9-hexahydro-2H-6,9-epiminocyclohepta[c]pyridine-10-carboxamide |
| (25) | tert-Butyl 3-((5-(2-chloro-5-fluoro-4-((6S,9R)-3-oxo-3,5,6,7,8,9-hexahydro-2H-6,9- |
| | epiminocyclohepta[c]pyridine-10-carboxamido)phenyl)pyridin-2-yl)oxy)pyrrolidine- |
| | 1-carboxylate |
| (26) | (6S,9R)-N-(5-chloro-2-fluoro-4-(1-methyl-1H-indol-5-yl)phenyl)-3-oxo-3,5,6,7,8,9- |
| | hexahydro-2H-6,9-epiminocyclohepta[c]pyridine-10-carboxamide |
| (27) | (6S,9R)-N-(5-chloro-2-fluoro-4-(6-((1-(2,2,2-trifluoroethyl)pyrrolidin-3- |
| | yl)oxy)pyridin-3-yl)phenyl)-3-oxo-3,5,6,7,8,9-hexahydro-2H-6,9- |
| | epiminocyclohepta[c]pyridine-10-carboxamide |
| (28) | (6S,9R)-N-(5-chloro-4-(6-(cyclobutylmethoxy)pyridin-3-yl)-2-fluorophenyl)-3-oxo- |
| | 3,5,6,7,8,9-hexahydro-2H-6,9-epiminocyclohepta[c]pyridine-10-carboxamide |
| (29) | tert-Butyl 3-(((5-(2-chloro-5-fluoro-4-((6S,9R)-3-oxo-3,5,6,7,8,9-hexahydro-2H-6,9- |
| | epiminocyclohepta[c]pyridine-10-carboxamido)phenyl)pyridin-2- |
| | yl)oxy)methyl)azetidine-1-carboxylate |
| (30) | (6S,9R)-N-(5-chloro-2-fluoro-4-(1-methyl-6-oxo-1,6-dihydropyridin-3-yl)phenyl)-3- |
| | oxo-3,5,6,7,8,9-hexahydro-2H-6,9-epiminocyclohepta[c]pyridine-10-carboxamide |
| (31) | (6S,9R)-N-(5-chloro-2-fluoro-4-(6-(2,2,2-trifluoro-N-methylacetamido)pyridin-3- |
| | yl)phenyl)-3-oxo-3,5,6,7,8,9-hexahydro-2H-6,9-epiminocyclohepta[c]pyridine-10- |
| | carboxamide |
| (32) | (6S,9R)-N-(5-chloro-2-fluoro-4-(6-((1-(2,2,2-trifluoroethyl)azetidin-3- |
| | yl)methoxy)pyridin-3-yl)phenyl)-3-oxo-3,5,6,7,8,9-hexahydro-2H-6,9- |
| | epiminocyclohepta[c]pyridine-10-carboxamide |
| (33) | (±)(9S)-N-(4-chloro-5-(trifluoromethyl)pyridin-2-yl)-1,9-difluoro-6,7,8,9-tetrahydro- |
| | 5H-5,8-epiminocyclohepta[c]pyridine-10-carboxamide |
| (34) | (±)-N-(4-chloro-5-(trifluoromethyl)pyridin-2-yl)-1,9,9-trifluoro-6,7,8,9-tetrahydro- |
| | 5H-5,8-epiminocyclohepta[c]pyridine-10-carboxamide |
| (35) | (±)(9S)-N-(4,5-dichloropyridin-2-yl)-1,9-difluoro-6,7,8,9-tetrahydro-5H-5,8- |
| | epiminocyclohepta[c]pyridine-10-carboxamide |
| (36) | (±)-N-(4,5-dichloropyridin-2-yl)-1,9,9-trifluoro-6,7,8,9-tetrahydro-5H-5,8- |
| | epiminocyclohepta[c]pyridine-10-carboxamide |
| (37) | (6S,9R)-N-(5-chloro-2-fluoro-4-(1-methyl-1H-indazol-5-yl)phenyl)-3-oxo- |
| | 3,5,6,7,8,9-hexahydro-2H-6,9-epiminocyclohepta[c]pyridine-10-carboxamide |
| (38) | (6S,9R)-N-(5-chloro-4-(6-((2,2-difluorocyclopropyl)methoxy)pyridin-3-yl)-2- |
| | fluorophenyl)-3-oxo-3,5,6,7,8,9-hexahydro-2H-6,9-epiminocyclohepta[c]pyridine- |
| | 10-carboxamide |
| (39) | (6S,9R)-N-(5-chloro-2-fluoro-4-(4-(methyl(2,2,2-trifluoroethyl)amino)pyridin-2- |
| | yl)phenyl)-3-oxo-3,5,6,7,8,9-hexahydro-2H-6,9-epiminocyclohepta[c]pyridine-10- |
| | carboxamide |
| (40) | (6S,9R)-N-(5-chloro-2-fluoro-4-(6-(2,2,2-trifluoroethyl)pyridin-3-yl)phenyl)-3-oxo- |
| | 3,5,6,7,8,9-hexahydro-2H-6,9-epiminocyclohepta[c]pyridine-10-carboxamide |
| (41) | (6S,9R)-N-(5-chloro-2-fluoro-4-(6-((tetrahydrofuran-3-yl)methoxy)pyridin-3- |
| | yl)phenyl)-3-oxo-3,5,6,7,8,9-hexahydro-2H-6,9-epiminocyclohepta[c]pyridazine- |
| | 10-carboxamide |
| (42) | (5R,8S,9S)-N-(4,5-dichloropyridin-2-yl)-1,9-difluoro-6,7,8,9-tetrahydro-5H-5,8- |
| | epiminocyclohepta[c]pyridine-10-carboxamide |
| (43) | (6S,9R)-N-(5-chloro-2-fluoro-4-(2-(methyl(2,2,2-trifluoroethyl)amino)pyridin-4- |
| | yl)phenyl)-3-oxo-3,5,6,7,8,9-hexahydro-2H-6,9-epiminocyclohepta[c]pyridine-10- |
| | carboxamide |
| (44) | (5R,8S)-N-(4,5-dichloropyridin-2-yl)-6,7,8,9-tetrahydro-5H-5,8- |
| | epiminocyclohepta[c]pyridine-10-carboxamide |
| (45) | (5R,8S)-N-(4-chloro-5-cyano-2-fluorophenyl)-1-fluoro-6,7,8,9-tetrahydro-5H-5,8- |
| | epiminocyclohepta[c]pyridine-10-carboxamide |
| (46) | (5R,8S)-N-(4-chloro-2-fluorophenyl)-1-fluoro-6,7,8,9-tetrahydro-5H-5,8- |
| | epiminocyclohepta[c]pyridine-10-carboxamide |
| (47) | (6S,9R)-N-(5-chloro-2-fluoro-4-(6-(((tetrahydrofuran-3-yl)methoxy)pyridin-3- |
| | yl)phenyl)-3-oxo-3,5,6,7,8,9-hexahydro-2H-6,9-epiminocyclohepta[c]pyridine-10- |
| | carboxamide Isomer 1 |
| (48) | (6S,9R)-N-(5-chloro-2-fluoro-4-(6-(((tetrahydrofuran-3-yl)methoxy)pyridin-3- |
| | yl)phenyl)-3-oxo-3,5,6,7,8,9-hexahydro-2H-6,9-epiminocyclohepta[c]pyridine-10- |
| | carboxamide Isomer 2 |
| (49) | (5R,8S)-N-(4,5-dichloro-2-fluorophenyl)-6,7,8,9-tetrahydro-5H-5,8- |
| | epiminocyclohepta[c]pyridine-10-carboxamide |
| (50) | (5R,8S)-N-(5-chloro-2-fluoro-4-(trifluoromethyl)phenyl)-6,7,8,9-tetrahydro-5H-5,8- |
| | epiminocyclohepta[c]pyridine-10-carboxamide |
| (51) | (6S,9R)-N-(5-Chloro-2-fluoro-4-(((5-fluoropyridin-2-yl)oxy)methyl)phenyl)-3-oxo- |
| | 3,5,6,7,8,9-hexahydro-2H-6,9-epiminocyclohepta[c]pyridazine-10-carboxamide |
| (52) | (6S,9R)-N-(5-Chloro-2-fluoro-4-(((6-fluoropyridin-2-yl)oxy)methyl)phenyl)-3-oxo- |
| | 3,5,6,7,8,9-hexahydro-2H-6,9-epiminocyclohepta[c]pyridazine-10-carboxamide |
| (53) | (6S,9R)-N-(5-chloro-2-fluoro-4-(phenoxymethyl)phenyl)-3-oxo-3,5,6,7,8,9- |
| | hexahydro-2H-6,9-epiminocyclohepta[c]pyridine-10-carboxamide |
| (54) | (6S,9R)-N-(5-chloro-2-fluoro-4-(((5-(2,2,2-trifluoroethoxy)pyridin-2- |
| | yl)oxy)methyl)phenyl)-3-oxo-3,5,6,7,8,9-hexahydro-2H-6,9- |
| | epiminocyclohepta[c]pyridine-10-carboxamide |
| (55) | (6S,9R)-N-(5-Chloro-2-fluoro-4-(((5-(2,2,2-trifluoroethoxy)pyridin-2- |
| | yl)oxy)methyl)phenyl)-3-oxo-3,5,6,7,8,9-hexahydro-2H-6,9- |
| | epiminocyclohepta[c]pyridazine-10-carboxamide |
| (56) | (6S,9R)-N-(5-chloro-2-fluoro-4-(((6-(2,2,2-trifluoroethoxy)pyridin-2- |
| | yl)oxy)methyl)phenyl)-3-oxo-3,5,6,7,8,9-hexahydro-2H-6,9- |
| | epiminocyclohepta[c]pyridazine-10-carboxamide |
| (57) | (6S,9R)-N-(5-chloro-2-fluoro-4-(((6-(2,2,2-trifluoroethoxy)pyridin-2- |
| | yl)oxy)methyl)phenyl)-3-oxo-3,5,6,7,8,9-hexahydro-2H-6,9- |
| | epiminocyclohepta[c]pyridine-10-carboxamide |
| (58) | (6S,9R)-N-(5-chloro-4-(((5-(cyclopentyloxy)pyridin-2-yl)oxy)methyl)-2- |
| | fluorophenyl)-3-oxo-3,5,6,7,8,9-hexahydro-2H-6,9-epiminocyclohepta[c]pyridine- |
| | 10-carboxamide |
| (59) | (6S,9R)-N-(5-chloro-4-(((6-(cyclopentyloxy)pyridin-2-yl)oxy)methyl)-2- |
| | fluorophenyl)-3-oxo-3,5,6,7,8,9-hexahydro-2H-6,9- |
| | epiminocyclohepta[c]pyridazine-10-carboxamide |
| (60) | (6S,9R)-N-(4-(6-((2-azabicyclo[2.1.1]hexan-4-yl)methoxy)pyridin-3-yl)-5-chloro-2- |
| | fluorophenyl)-3-oxo-3,5,6,7,8,9-hexahydro-2H-6,9-epiminocyclohepta[c]pyridine- |
| | 10-carboxamide |
| (61) | (6S,9R)-N-(5-Chloro-2-fluoro-4-(6-((2-methyl-2-azabicyclo[2.1.1]hexan-4- |
| | yl)methoxy)pyridin-3-yl)phenyl)-3-oxo-3,5,6,7,8,9-hexahydro-2H-6,9- |
| | epiminocyclohepta[c]pyridine-10-carboxamide |
| (62) | (6S,9R)-N-(5-Chloro-2-fluoro-4-(6-((2-methyl-2-azabicyclo[2.1.1]hexan-4- |
| | yl)methoxy)pyridin-3-yl)phenyl)-3-oxo-3,5,6,7,8,9-hexahydro-2H-6,9- |
| | epiminocyclohepta[c]pyridine-10-carboxamide |
| (63) | (6S,9R)-N-(4-(6-(Azetidin-3-ylmethoxy)pyridin-3-yl)-5-chloro-2-fluorophenyl)-3- |
| | oxo-3,5,6,7,8,9-hexahydro-2H-6,9-epiminocyclohepta[c]pyridine-10-carboxamide |
| | trifluoroacetate salt |
| (64) | (6S,9R)-N-(5-chloro-2-fluoro-4-(((6-(2,2,2-trifluoroethoxy)pyridin-2- |
| | yl)methyl)amino)phenyl)-3-oxo-3,5,6,7,8,9-hexahydro-2H-6,9- |
| | epiminocyclohepta[c]pyridazine-10-carboxamide |
| (65) | (6S,9R)-N-(5-chloro-2-fluoro-4-(((5-(2,2,2-trifluoroethoxy)pyridin-2- |
| | yl)methyl)amino)phenyl)-3-oxo-3,5,6,7,8,9-hexahydro-2H-6,9- |
| | epiminocyclohepta[c]pyridazine-10-carboxamide |
| (66) | (5R,8S)-10-((4,5-dichloro-2-fluorophenyl)carbamoyl)-6,7,8,9-tetrahydro-5H-5,8- |
| | epiminocyclohepta[c]pyridine 2-oxide |
| (67) | (5R,8S)-10-((5-chloro-2-fluoro-4-(trifluoromethyl)phenyl)carbamoyl)-6,7,8,9- |
| | tetrahydro-5H-5,8-epiminocyclohepta[c]pyridine 2-oxide |
| (68) | (6S,9R)-N-(5-chloro-2-fluoro-4-(2-methyl-1H-benzo[d]imidazol-5-yl)phenyl)-3-oxo- |
| | 3,5,6,7,8,9-hexahydro-2H-6,9-epiminocyclohepta[c]pyridine-10-carboxamide |
| (70) | (±)-N-(5-Chloro-2-fluoro-4-(6-(2,2,2-trifluoroethoxy)pyridin-3-yl)phenyl)-1,9,9- |
| | trifluoro-6,7,8,9-tetrahydro-5H-5,8-epiminocyclohepta[c]pyridine-10-carboxamide |
| (71) | (5R,8S)-N-(5-Chloro-2-fluoro-4-(6-(2,2,2-trifluoroethoxy)pyridin-3-yl)phenyl)-1,9,9- |
| | trifluoro-6,7,8,9-tetrahydro-5H-5,8-epiminocyclohepta[c]pyridine-10-carboxamide |
| (72) | (5S,8R)-N-(5-Chloro-2-fluoro-4-(6-(2,2,2-trifluoroethoxy)pyridin-3-yl)phenyl)-1,9,9- |
| | trifluoro-6,7,8,9-tetrahydro-5H-5,8-epiminocyclohepta[c]pyridine-10-carboxamide |
| (73) | (5S,8R)-N-(5-(Benzo[c][1,2,5]oxadiazol-5-yl)-4-chloropyridin-2-yl)-1,9,9-trifluoro- |
| | 6,7,8,9-tetrahydro-5H-5,8-epiminocyclohepta[c]pyridine-10-carboxamide |
| (74) | (5R,8S)-N-(5-(Benzo[c][1,2,5]oxadiazol-5-yl)-4-chloropyridin-2-yl)-1,9,9-trifluoro- |
| | 6,7,8,9-tetrahydro-5H-5,8-epiminocyclohepta[c]pyridine-10-carboxamide |
| (75) | (5S,8R,9R)-N-(5-(Benzo[c][1,2,5]oxadiazol-5-yl)-4-chloropyridin-2-yl)-1,9-difluoro- |
| | 6,7,8,9-tetrahydro-5H-5,8-epiminocyclohepta[c]pyridine-10-carboxamide |
| (76) | (5R,8S,9S)-N-(5-(Benzo[c][1,2,5]oxadiazol-5-yl)-4-chloropyridin-2-yl)-1,9-difluoro- |
| | 6,7,8,9-tetrahydro-5H-5,8-epiminocyclohepta[c]pyridine-10-carboxamide |
| (77) | (6S,9R)-N-(5-Chloro-2-fluoro-4-(6-((1-(2,2,2-trifluoroethyl)pyrrolidin-3- |
| | yl)oxy)pyridin-3-yl)phenyl)-3-oxo-3,5,6,7,8,9-hexahydro-2H-6,9- |
| | epiminocyclohepta[c]pyridine-10-carboxamide Isomer 1 |
| (78) | (6S,9R)-N-(5-Chloro-2-fluoro-4-(6-((1-(2,2,2-trifluoroethyl)pyrrolidin-3- |
| | yl)oxy)pyridin-3-yl)phenyl)-3-oxo-3,5,6,7,8,9-hexahydro-2H-6,9- |
| | epiminocyclohepta[c]pyridine-10-carboxamide Isomer 2 |
| (79) | (6S,9R)-N-(5-Chloro-2-fluoro-4-(6-(((R)-tetrahydrofuran-3-yl)methoxy)pyridin-3- |
| | yl)phenyl)-3-oxo-3,5,6,7,8,9-hexahydro-2H-6,9-epiminocyclohepta[c]pyridazine- |
| | 10-carboxamide |
| (80) | (6S,9R)-N-(5-Chloro-2-fluoro-4-(6-(((S)-tetrahydrofuran-3-yl)methoxy)pyridin-3- |
| | yl)phenyl)-3-oxo-3,5,6,7,8,9-hexahydro-2H-6,9-epiminocyclohepta[c]pyridazine- |
| | 10-carboxamide |
| (81) | (6S,9R)-N-(5-Chloro-4-(6-(((R)-2,2-difluorocyclopropyl)methoxy)pyridin-3-yl)-2- |
| | fluorophenyl)-3-oxo-3,5,6,7,8,9-hexahydro-2H-6,9- |
| | epiminocyclohepta[c]pyridazine-10-carboxamide |
| (82) | (6S,9R)-N-(5-Chloro-4-(6-(((R)-2,2-difluorocyclopropyl)methoxy)pyridin-3-yl)-2- |
| | fluorophenyl)-3-oxo-3,5,6,7,8,9-hexahydro-2H-6,9-epiminocyclohepta[c]pyridine- |
| | 10-carboxamide |
| (83) | (6S,9R)-N-(5-Chloro-4-(6-(((S)-2,2-difluorocyclopropyl)methoxy)pyridin-3-yl)-2- |
| | fluorophenyl)-3-oxo-3,5,6,7,8,9-hexahydro-2H-6,9- |
| | epiminocyclohepta[c]pyridazine-10-carboxamide |
| (84) | (6S,9R)-N-(5-Chloro-2-fluoro-4-(6-((tetrahydro-2H-pyran-4-yl)oxy)pyridin-3- |
| | yl)phenyl)-3-oxo-3,5,6,7,8,9-hexahydro-2H-6,9-epiminocyclohepta[c]pyridazine- |
| | 10-carboxamide |
| (85) | (6S,9R)-N-(5-Chloro-4-(6-(((S)-2,2-difluorocyclopropyl)methoxy)pyridin-3-yl)-2- |
| | fluorophenyl)-3-oxo-3,5,6,7,8,9-hexahydro-2H-6,9-epiminocyclohepta[c]pyridine- |
| | 10-carboxamide |
| (86) | (6S,9R)-N-(5-Chloro-2-fluoro-4-(6-((1-fluorocyclopropyl)methoxy)pyridin-3- |
| | yl)phenyl)-3-oxo-3,5,6,7,8,9-hexahydro-2H-6,9-epiminocyclohepta[c]pyridazine- |
| | 10-carboxamide |
| (87) | (6S,9R)-N-(5-Chloro-4-(6-((1,1-dioxidotetrahydro-2H-thiopyran-4-yl)oxy)pyridine- |
| | 3-yl)-2-fluorophenyl)-3-oxo-3,5,6,7,8,9-hexahydro-2H-6,9- |
| | epiminocyclohepta[c]pyridazine-10-carboxamide |
| (88) | (6S,9R)-N-(4,5-Dichloro-2-((2,2-difluorocyclopropyl)methoxy)phenyl)-3-oxo- |
| | 3,5,6,7,8,9-hexahydro-2H-6,9-epiminocyclohepta[c]pyridazine-10-carboxamide |
| (89) | (6S,9R)-N-(4,5-Dichloro-2-((1-fluorocyclopropyl)methoxy)phenyl)-3-oxo- |
| | 3,5,6,7,8,9-hexahydro-2H-6,9-epiminocyclohepta[c]pyridazine-10-carboxamide |
| (91) | (6S,9R)-N-(1,3-Dioxo-2-(6-(2,2,2-trifluoroethoxy)pyridin-3-yl)isoindolin-5-yl)-3-oxo- |
| | 3,5,6,7,8,9-hexahydro-2H-6,9-epiminocyclohepta[c]pyridazine-10-carboxamide |
| (92) | (6S,9R)-N-(5-Chloro-4-(6-((5-chloro-2-fluoro-4-(6-(2,2,2-trifluoroethoxy)pyridin-3- |
| | yl)phenyl)amino)pyridin-3-yl)-2-fluorophenyl)-3-oxo-3,5,6,7,8,9-hexahydro-2H-6,9- |
| | epiminocyclohepta[c]pyridazine-10-carboxamide |

and enantiomers thereof, and mixtures of enantiomers thereof, including racemic mixtures, and pharmaceutically acceptable salts and solvates thereof.

In one preferred embodiment, the compound is selected from the following: 1-6, 8, 10-18, 20-29, 32-59, 64, 66, 67, 70-72, 74-87 and 92.

### Process

A further aspect of the invention relates to a process for preparing a compound as defined herein, said process comprising reacting a compound of formula (III) with a compound of formula (IV), where A, B, Y, Rₐ and R_{b} are as defined above, to form a compound of formula (I) (or II):

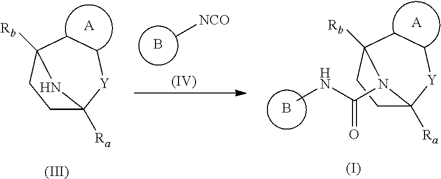

In one preferred embodiment, the reaction takes place in the presence of a base, preferably, N,N-diisopropylethylamine (DIPEA) or triethylamine. Preferably, the reaction takes place in an organic solvent. Suitable organic solvents include, but are not limited to, dichloromethane, tetrahydrofuran and dimethylformamide, or mixtures of two or more thereof. The skilled person would understand that other bases and solvents would also be suitable. The isocyanate intermediate (IV) can be generated in situ from the corresponding amine by reacting with triphosgene (bis(trichloromethyl) carbonate) in the presence of a suitable base.

A further aspect of the invention relates to a process for preparing a compound as defined herein, said process comprising reacting a compound of formula (III) with a compound of formula (V), where A, B, Y, Rₐ and R_{b} are as defined above, in the presence of triphosgene to form a compound of formula (I) (or II):

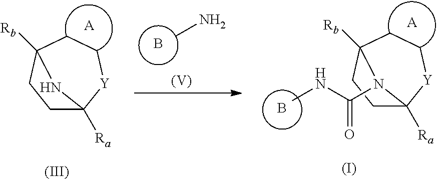

In one preferred embodiment, the reaction takes place in the presence of a base, preferably, N,N-diisopropylethylamine (DIPEA) or triethylamine. Preferably, the reaction takes place in an organic solvent. Suitable organic solvents include, but are not limited to, dichloromethane, tetrahydrofuran and dimethylformamide, or mixtures of two or more thereof. The skilled person would understand that other bases and solvents would also be suitable. Preferably, the organic solvent is dichloromethane and the base is trimethylamine.

A further aspect of the invention relates to a process for preparing a compound as defined herein, said process comprising the steps of:

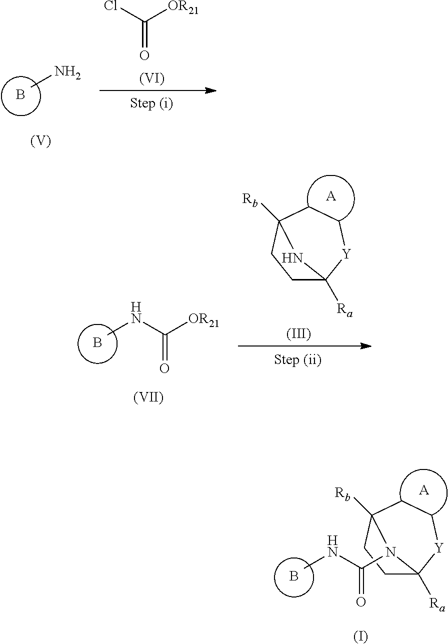

(i) treating a compound of formula (V), where B is defined as above, with a compound of formula (VI), where R₂₁ is phenyl optionally substituted with 1 to 5 fluorine atoms, to form a compound of formula (VII); and
(ii) treating said compound of formula (VII) with a compound of formula (III), or a pharmaceutically acceptable salt thereof, where A, Y, Rₐ, R_{b} and R₆ are as described above, to form a compound of formula (I) or (II);

In one preferred embodiment, step (i) is carried out in a solvent. Preferred solvents include organic solvents such as tetrahydrofuran, dichloromethane, DMSO, and 2-methyltetrahydrofuran, and mixtures thereof.

Further details of the above syntheses are set out in the accompanying examples.

### Therapeutic Applications

A further aspect of the invention relates to compounds as described herein for use in medicine. The compounds have particular use in the field of oncology, immuno-oncology, and immunology as described in more detail below. In a preferred embodiment, the compound of the invention modulates GPR65, and more preferably inhibits GPR65 signalling.

Yet another aspect of the invention relates to compounds as described herein for use as a medicament, preferably for use in treating or preventing a disorder selected from a proliferative disorder and an immune disorder.

Another aspect of the invention relates to compounds as described herein for use in treating or preventing asthma and/or chronic obstructive pulmonary disease (COPD). GPR65 variant/SNP (rs6574978) has been shown to be associated with asthma/COPD syndrome with almost GWAS significant p value (1.18×10e-7) (Hardin, 2014). Furthermore, GPR65 activation by pH (pH is low/acidic in asthmatic lungs) promotes eosinophil viability in a cAMP-dependent manner, contributing to disease progression/exacerbation. It is further known that GPR65 KO mice have attenuated asthma symptoms (Kottyan, 2009).

Another aspect of the invention relates to compounds as described herein for use in treating or preventing acute respiratory distress syndrome (ARDS). GPR65 has been shown to be protective in a model of LPS-induced acute lung injury model (Tsurumaki, 2015).

One aspect of the invention relates to a compound as described herein for use in treating a proliferative disorder. Preferably, the proliferative disorder is a cancer or leukemia.

In one preferred embodiment, the cancer is a solid tumour and/or metastases thereof.

In another preferred embodiment, the cancer is selected from melanoma, renal cell carcinoma (RCC), gastric cancer, acute myeloid leukaemia (AML), triple negative breast cancer (TNBC), colorectal cancer, head and neck cancer, colorectal adenocarcinoma, pancreatic adenocarcinoma, lung cancer, sarcoma, ovarian cancer, and gliomas, preferably glioblastoma (GBM).

Without wishing to be bound by theory, it is understood that GPR65 modulators are capable of preventing the increase in cytoplasmic cAMP in tumour-associated macrophages (TAMs), natural killer (NK) cells and subsets of T cells that would typically result from their exposure to the acidic tumour microenvironment and concomitant GPR65 activation. This reduction in the level of cytoplasmic cAMP in turn reduces the levels of ICER and pro-inflammatory mediators such as CXCL10 and TNFα, preventing the polarization of TAMs and alteration of other immune cells that are associated with a non-inflammatory and tumour-permissive environment. Therefore, GPR65 modulators are expected to result in an increase in the visibility of the tumour to the immune system leading to increased immune-mediated tumour clearance. This suggests that modulation of GPR65 activity could be an effective treatment for cancer as stand-alone therapy or in combination with cancer immunotherapies (vaccines, agents that promote T cell mediated immune responses) or in patients that do not respond to immunomodulatory approaches such as PD1/PDL-1 blockade.

Another aspect of the invention relates to a compound as described herein for use in treating or preventing an immune disorder, preferably an autoimmune disease.

In one embodiment, the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, autoimmune polyendocrinopathies, autoimmune myocarditis, type I diabetes and atopic dermatitis.

In a particularly preferred embodiment, the autoimmune disease is selected from psoriasis, psoriatic arthritis, ankylosing spondylitis, Crohn's disease, and multiple sclerosis (MS).

Without wishing to be bound by theory, it is understood that GPR65 modulators will prevent the upregulation of ICER in CD4+ T cells. This, in turn, is expected to prevent the ICER-associated suppression of IL-2 that biases CD4+ T cells toward the inflammatory Th17 phenotype associated with increased pathogenicity in the context of autoimmune disease.

This is supported by the fact that mutations in the GPR65 locus are associated with several autoimmune diseases, such as multiple sclerosis, ankylosing spondylitis, inflammatory bowel disease, and Crohn's disease (Gaublomme, 2015). This suggests that modulation of GPR65 activity could be an effective treatment for autoimmune diseases.

Another aspect relates to a compound as described herein for use in treating or preventing a disorder caused by, associated with or accompanied by abnormal activity against GPR65.

Another aspect relates to a compound as described herein for use in treating or preventing a GPR65-associated disease or disorder.

Another aspect of the invention relates to a method of treating a disorder as described above comprising administering a compound as described herein to a subject. Another aspect of the invention relates to a method of treating a GPR65-associated disease or disorder in a subject. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a subject having a disease state alleviated by modulation of GPR65 wherein the method comprises administering to the subject a therapeutically effective amount of a compound according to the invention.

Another aspect relates to a method of treating a disease state alleviated by modulation of GPR65, wherein the method comprises administering to a subject a therapeutically effective amount of a compound according to the invention.

Preferably, the subject is a mammal, more preferably a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately to determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀.

Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see, e.g., Fingl et al, 1975, The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/day, commonly from about 100-1000 mg/day, preferably from about 150-700 mg/day and most preferably from about 250-500 mg/day or from 50-100 mg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “GPR65-related disease or disorder” refers to a disease or disorder characterized by inappropriate GPR65 activity. Inappropriate GPR65 activity refers to either an increase or decrease in GPR65 activity as measured by enzyme or cellular assays, for example, compared to the activity in a healthy subject. Inappropriate activity could also be due to overexpression of GPR65 in diseased tissue compared with healthy adjacent tissue.

Preferred diseases or disorders that the compounds described herein may be useful in preventing include proliferative disorders and immune disorders as described hereinbefore, as well as asthma and chronic obstructive pulmonary disease.

Thus, the present invention further provides use of compounds as defined herein in the preparation of a medicament for the treatment of a disease where it is desirable to modulate GPR65. Such diseases include proliferative disorders and immune disorders as described hereinbefore, as well as asthma and chronic obstructive pulmonary disease.

As used herein the phrase “preparation of a medicament” includes the use of the components of the invention directly as the medicament in addition to their use in any stage of the preparation of such a medicament.

In one preferred embodiment, the compound prevents the increase in cytoplasmic cAMP levels expected following GPR65 activation at acidic pH. This prevention of cAMP accumulation in turn prevents downstream signalling through ICER, as described above.

The “Human GPR65 cyclic adenosine monophosphate (cAMP) Homogeneous Time Resolved Fluorescence (HTRF) antagonist assay”, or simply “cAMP assay”, as described in the accompanying examples, can be used to measure the potency of GPR65 modulators, which is expressed as the concentration of compound required to reduce the increase in cAMP concentration upon GPR65 activation by 50% (i.e. an IC₅₀).

In one preferred embodiment, the compound exhibits an IC₅₀ value in the cAMP assay of less than about 25 μM. More preferably, the compound exhibits an IC₅₀ value in the cAMP assay of less than about 10 μM, more preferably, less than about 5 μM, even more preferably, less than about 1 μM, even more preferably, less than about 0.1 μM.

In another preferred embodiment, the compound exhibits an hGPR65 IC₅₀ value of less than <5 μM, more preferably less than <500 nM in the aforementioned assay.

In one preferred embodiment, the compound, or compound for use, according to the invention exhibits an IC₅₀ of >500 nM and <5 μM in a Human GPR65 cyclic adenosine monophosphate (cAMP) Homogeneous Time Resolved Fluorescence (HTRF) antagonist assay as described in the accompanying examples. In one preferred embodiment, the compound is selected from those denoted “high” or “medium” in Table 1.

In a more preferred embodiment, the compound, or compound for use, according to the invention exhibits an IC₅₀ of <500 nM in a Human GPR65 cAMP HTRF antagonist assay as described in the accompanying examples. In one preferred embodiment, the compound is selected from those denoted “high” in Table 1.

### Pharmaceutical Compositions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers, excipients or diluents therefor and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and PJ Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet.

Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline.

Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like. Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein into conjunction or association with a pharmaceutically or veterinarily acceptable carrier or vehicle.

### Salts/Esters

The compounds of the invention can be present in free base form, or as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹ P ³²P 3S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formulae (I) and (II) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Atropisomers

Some of the compounds of the invention may exist as atropisomers. Atropisomers are stereoisomers arising because of hindered rotation about a single bond, where energy differences due to steric strain or other contributors create a barrier to rotation that is high enough to allow for isolation of individual conformers. The invention encompasses all such atropisomers. The invention also covers rotamers of the compounds.

### Prodrugs

The invention further includes the compounds of the present invention in prodrug form, i.e. covalently bonded compounds which release the active parent drug in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms. Preferably, the solvate is a hydrate.

### Combinations

A further aspect of the inventiont relates to a combination comprising a compound as described herein and one or more additional active agents. In a particularly preferred embodiment, the one or more compounds of the invention are administered in combination with one or more additional active agents, for example, existing drugs available on the market. In such cases, the compounds of the invention may be administered consecutively, simultaneously or sequentially with the one or more other active agents. Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the activity of the test compounds with agents known or suspected of being valuable in the treatment of a particular disorder.

This procedure can also be used to determine the order of administration of the agents, i.e. before, simultaneously, or after delivery. Such scheduling may be a feature of all the active agents identified herein.

In the context of cancer, compounds of the invention can be used in combination with immunotherapies such as cancer vaccines and/or with other immune-modulators such as agents that block the PD1/PDL-1 interaction. Other examples of agents for use in combination with the presently claimed compounds include immune modulators that block CTLA-4 or LAG-3. Thus, in one preferred embodiment, the additional active agent is an immunotherapy agent, more preferably a cancer immunotherapy agent. An “immunotherapy agent” refers to a treatment that uses the subject's own immune system to fight diseases such as cancer. For other disorders the compounds of the invention can be used in combination agents that block or decrease inflammation such as antibodies that target pro-inflammatory cytokines. The compounds of the invention can also be used in combination with other chemotherapy agents and/or in conjunction with radiotherapy.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation.

The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The dosage amount will further be modified according to the mode of administration of the compound. For example, to achieve an “effective amount” for acute therapy, parenteral administration of a compound is typically preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg; preferably between 0.1 and 20 mg, in a manner to maintain the concentration of drug in the plasma at a concentration effective to modulate GPR65. The compounds may be administered one to four times daily at a level to achieve a total daily dose of about 0.4 to about 400 mg. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 500 mg or about 0.1 to about 50 mg in a manner consistent with the condition of the patient. Preferably the oral dose would be about 0.5 to about 50 mg or about 0.5 to about 20 mg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

The invention is further described with reference to the following non-limiting examples.

### Examples

Where the preparation of starting materials is not described, these are commercially available, known in the literature, or readily obtainable by those skilled in the art using standard procedures. Where it is indicated that compounds were prepared analogously to earlier examples or intermediates, it will be appreciated by the skilled person that the reaction time, number of equivalents of reagents, solvent, concentration and temperature can be modified for each specific reaction and that it may be necessary or desirable to employ different work-up or purification techniques.

### General Schemes

### Abbreviations

A list of some common abbreviations is shown below—where other abbreviations are used which are not listed, these will be understood by the person skilled in the art.

AcOH: acetic acid; BINAP: (2,2′-bis(diphenylphosphino)-1,1′-binaphthyl); Boc: tert-butyloxycarbonyl; br.: broad; CAN: Ceric ammonium nitrate; CBS: Corey-Bakshi-Shibata catalyst; d: doublet; DAST: diethylaminosulfur trifluoride; DCM: dichloromethane; DIAD: diisopropyl azodicarboxylate; DIBAL: diisobutylaluminium hydride; DIPEA: N,N-diisopropylethylamine; DMF: N,N-dimethylformamide; DMSO: dimethylsulfoxide; dppf: 1,1′-bis(diphenylphosphino)ferrocene; (ES+): electrospray ionization positive mode; Et₃N: triethylamine; EtOAc: ethyl acetate; EtOH: ethanol; h: hours; hept: heptet; HPLC: high performance liquid chromatography; HCl: hydrochloric acid; Hz: hertz; J: coupling constant; I: litre; M: molar; m: multiplet; [M+H]+: protonated molecular ion; MeCN: acetonitrile; MeOH: methanol; MHz: megahertz; min: minutes; mL: millilitres; MS: mass spectrometry; MTBE: methyl tert-butyl ether; m/z: mass-to-charge ratio; NMR: nuclear magnetic resonance; p: pentet; PDA: photodiode array; Pd(dppf)Cl₂: [1,1′-Bis(diphenylphosphino)ferrocene]dichloropalladium(II); Pd-161: AmPhos Pd(crotyl)CI; Pd-178: P(Cy)3 Pd(crotyl)CI; PIDA: (Diacetoxyiodo)benzene; prep TLC: preparative thin layer chromatography; q: quintet; RT: room temperature; Rt: retention time; s: singlet; SFC: supercritical fluid chromatography; t: triplet; THF: tetrahydrofuran; TLC: thin layer chromatography; UPLC: ultra performance liquid chromatography; UV: ultra-violet; Other abbreviations are intended to convey their generally accepted meaning.

### General Experimental Conditions

All starting materials and solvents were obtained either from commercial sources or prepared according to literature methods. The appropriate isocyanate and aniline starting materials were either commercially available and were obtained from, for example, Sigma Aldrich, Fluorochem or Enamine store, or were synthesised as described herein. The appropriate tricyclic amine starting materials were synthesised as described herein.

Reaction mixtures were magnetically stirred and reactions performed at room temperature (approximately 20° C.) unless otherwise indicated.

Silica gel chromatography was performed on an automated flash chromatography system, such as CombiFlash Companion, CombiFlash Rf system or Reveleris X2 flash system using RediSep® Rf or Reveleris® or the GraceResolv™ pre-packed silica (230-400 mesh, 40-63 μm) cartridges.

Analytical UPLC-MS experiments to determine retention times and associated mass ions were performed using a Waters ACQUITY UPLC® H-Class system, equipped with ACQUITY PDA Detector and ACQUITY QDa mass spectrometer or Waters SQD mass spectrometer, running the analytical method described below.

Analytical LC-MS experiments to determine retention times and associated mass ions were performed using an Agilent 1200 series HPLC system coupled to an Agilent 1956, 6100 or 6120 series single quadrupole mass spectrometer running one of the analytical methods described below or a Shimadzu-2020-P2 system consisting of a Shimadzu LC-20AD series LC system and a Shimadzu-2020, single quadrupole mass spectrometer running one of the analytical methods described below Analytical SFC experiments to determine retention times were performed using a Waters SFC system UPC2 system with a column temperature of 40° C. and a back pressure (ABPR) of 1750 psi using one of the analytical methods described.

Preparative HPLC purifications were performed either using a Waters Xbridge Prep OBD C18, 10 μm, 40×150 mm column using a gradient of MeCN and 0.1% ammonia in water or a gradient of MeCN and 0.1% formic acid in water. Fractions were collected following UV detection across all wavelengths with PDA and in some cases an SQD2 or ACQUITY QDa mass spectrometer.

Preparative SFC purifications were performed using either a Waters SFC prep 100 system or a Sepiatec Prep SFC 50 with either a: Phenomenex Lux® Cellulose-4, Column 1×25 cm, 5 μm particle size column or a a Phenomenex Lux® Al 5 μm, LC Column 250×10 mm or a Chiralpak IH 10×250 mm, flow rate 15-65 ml/min eluting with a mixture of CO₂ and co-solvent (MeOH, EtOH or IPA). Fractions were collected following UV detection at 210-400 nm using a PDA.

NMR spectra were recorded using either a Bruker Avance Ill HD 500 MHz instrument, a Bruker Avance Neo 400 MHz, Bruker Avance III 400 MHz instrument or a QOne AS400 400 MHz spectrometer using either residual non-deuterated solvent, or tetra-methylsilane as a reference

In the absence of the absolute stereochemistry being explicitly indicated through wedged and dashed bonds, chemical structures disclosed throughout the examples (for example, in the configuration (1.3) described hereinabove) are to be interpreted as depicting the racemate. For the avoidance of doubt, the invention encompasses the compounds in either configuration, as well as mixtures thereof.

### Separation of Enantiomers by Chiral Chromatography

It will be appreciated that the enantiomers of the compounds described above can be isolated using techniques well known in the art, including, but not limited to, chiral chromatography. For example, a racemic mixture can be dissolved in a solvent, for example, methanol, followed by separation by chiral SFC on a Waters prep 15 with UV detection by DAD at 210-400 nm, 40° C., 120 bar. The column was Chiralpak IG 10×250 mm, 5 m, flow rate 15 ml/min at 45% MeOH (0.1% DEA), 55% CO₂ to afford both enantiomers as the separated pure compounds.

### Analytical Methods

Method 1—Basic 3 min method
Column: Waters ACQUITY UPLCO BEH C18,1.7 μm, 2.1×30 mm at 40° C.
Detection: UV at 210-400 nm unless otherwise indicated, MS by electrospray ionisation
Solvents: A: 0.1% Ammonia in water, B: MeCN
Gradient:

| Time | % A | % B | Flow rate (ml/min) |
|---|---|---|---|
| 0.00 | 98 | 2 | 0.77 |
| 2.50 | 0 | 100 | 0.77 |
| 3.00 | 0 | 100 | 0.77 |

### Method 2—Acidic 3 Min Method 1

Column: Waters CSH C18, 1.7 μm, 2.1×30 mm at 40° C.
Detection: UV at 210-400 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 98 | 2 | 0.77 |
| 2.5 | 0 | 100 | 0.77 |
| 3 | 0 | 100 | 0.77 |

### Method 3—Acidic 5 Min Method 1

Column: Agilent EclipsePlus RRHD C18, 1.8 μm, 3.0×50 mm at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 90 | 10 | 0.8 |
| 0.50 | 90 | 10 | 0.8 |
| 4.00 | 10 | 90 | 0.8 |
| 4.50 | 0 | 100 | 0.8 |
| 4.51 | 90 | 10 | 0.8 |
| 5.00 | 90 | 10 | 0.8 |

### Method 4—Acidic 5 Min Method 2

Column: Waters Sunfire, 3.5 μm, 4.6×50 mm column at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 85 | 15 | 1.0 |
| 0.50 | 85 | 15 | 1.0 |
| 4.00 | 0 | 100 | 1.0 |
| 4.50 | 0 | 100 | 1.0 |
| 4.51 | 85 | 15 | 1.0 |
| 5.00 | 85 | 15 | 1.0 |

### Method 5—Acidic 3 Min Method 2

Column: Waters CORTECS UPLC,C18, 1.6 μm, 2.1×50 mm column at 25° C.
Detection: UV at 210-400 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 80 | 20 | 0.6 |
| 1.80 | 20 | 80 | 0.6 |
| 2.65 | 20 | 80 | 0.6 |
| 2.80 | 80 | 20 | 0.6 |
| 3.00 | 80 | 20 | 0.6 |

### Method 6—Acidic 3 Min Method 1

Column: Waters Sunfire, 3.5 μm, 4.6×50 mm column at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 80 | 20 | 1 |
| 2.00 | 15 | 85 | 1 |
| 2.50 | 15 | 85 | 1 |
| 2.51 | 80 | 20 | 1 |
| 3.00 | 80 | 20 | 1 |

### Method 7—Acidic 3 Min Method 1

Column: Agilent EclipsePlus RRHD C18, 1.8 μm, 3.0×50 mm at 25° C.
Detection: UV at 214 and 254 nm unless otherwise indicated, MS by electrospray ionisation
Solvents: A: 0.05% formic acid in water, B: 0.05% formic acid in MeCN Gradient:

| Time | % A | % B | Flow rate (ml/min) |
|---|---|---|---|
| 0.00 | 92 | 8 | 0.8 |
| 2.50 | 40 | 60 | 0.8 |
| 3.00 | 20 | 80 | 0.8 |

### Method 8—Acidic 3 Min Method 2

Column: Waters CORTECS, C18, 2.7 μm, 2.1×30 mm column at 40° C.
Detection: UV at 260 nm+/−90 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 98 | 2 | 1.35 |
| 2.50 | 0 | 100 | 1.35 |
| 3.00 | 0 | 100 | 1.35 |

### Experimental Scheme 1

### Compound 1 (6S,9R)-N-(4-(6-((2—Oxabicyclo[2.1.1]hexan-4-yl)methoxy)pyridin-3-yl)-5-chloro-2-fluorophenyl)-3-oxo-3,5,6,7,8,9-hexahydro-2H-6,9-epiminocyclohepta[c]pyridazine-10-carboxamide

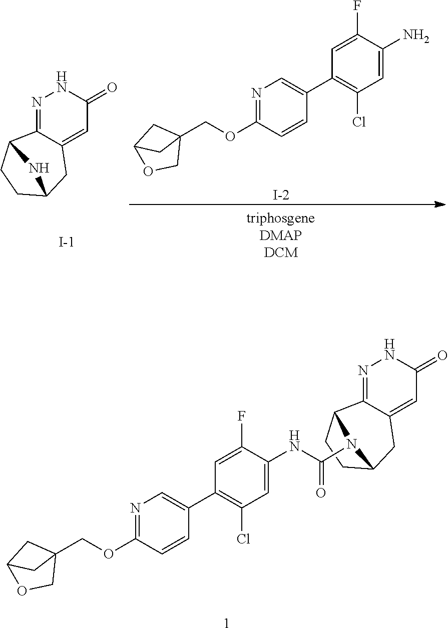

To a solution of triphosgene (11.6 mg, 39.0 μmol) in DCM (1.50 mL) was added a solution of 4-(6-((2-oxabicyclo[2.1.1]hexan-4-yl)methoxy)pyridin-3-yl)-5-chloro-2-fluoroaniline 1-2 (34.0 mg, 97.5 μmol) and DMAP (35.7 mg, 292 μmol) in DCM (1.50 mL) and the resulting mixture was stirred at RT for 5 min. The reaction mixture was added dropwise to a suspension of (6S,9R)-2,5,6,7,8,9-hexahydro-3H-6,9-epiminocyclohepta[c]pyridazin-3-one 1-1 (17.5 mg, 97.5 μmol) in DCM (1.50 mL) and the resulting solution was left to stir at RT for 16 h. The whole was diluted with DCM (5 mL) and washed with 1M HCl (2×2 mL) after which the organic phase was passed through a hydrophobic frit and the filtrate was concentrated in vacuo. The product was purified by chromatography on silica gel (0-85% (3:1 EtOAc:EtOH)/isohexane) to afford (6S,9R)-N-(4-(6-((2-oxabicyclo[2.1.1]hexan-4-yl)methoxy)pyridin-3-yl)-5-chloro-2-fluorophenyl)-3-oxo-3,5,6,7,8,9-hexahydro-2H-6,9-epiminocyclohepta[c]pyridazine-10-carboxamide 1 as a colourless solid. LCMS (Method 1) m/z 538.3, 540.3 (M+H)+(ES+), at 1.22 min; ¹H NMR (400 MHz, DMSO-d6) δ 12.72 (s, 1H), 8.88 (s, 1H), 8.20 (dd, J=2.5, 0.8 Hz, 1H), 7.81 (dd, J=8.6, 2.5 Hz, 1H), 7.76 (d, J=7.3 Hz, 1H), 7.38 (d, J=11.1 Hz, 1H), 6.92 (dd, J=8.6, 0.8 Hz, 1H), 6.69 (s, 1H), 5.08 (d, J=5.8 Hz, 1H), 4.62 (s, 3H), 4.53-4.48 (m, 1H), 3.59 (s, 2H), 3.20 (dd, J=18.2, 5.4 Hz, 1H), 2.65 (d, J=18.1 Hz, 1H), 2.27-2.11 (m, 2H), 1.82 (dt, J=4.7, 1.5 Hz, 3H), 1.68 (t, J=7.9 Hz, 1H), 1.49 (dd, J=4.5, 1.8 Hz, 2H).

The following compounds were prepared using appropriate starting materials in an analogous procedure to that described in Experimental Scheme 1. Where the starting materials are not described in the literature, their synthesis is described below.

| | | Method | |
|---|---|---|---|
| | | LCMS Rt | |
| Compound | Structure | [M + H]⁺ | NMR |
| 2 | 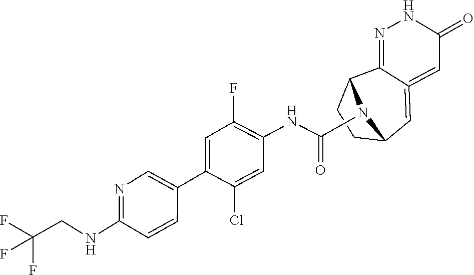 | Method 1 523.2, 525.3 at 1.44 min | ¹H NMR (400 MHz, DMSO- d6) δ 12.72 (s, 1H), 8.87 (s, 1H), 8.11 (d, J = 2.4 Hz, 1H), 7.85 (s, 1H), 7.77-7.67 (m, 2H), 7.35 (d, J = 11.1 Hz, 1H), 6.86 (d, J = 8.8 Hz, 1H), 6.69 (s, 1H), 5.07 (d, J = 5.8 Hz, 1H), 4.64 (t, J = 6.5 Hz, 1H), 4.33-4.22 (m, 2H), 3.23-3.19 (m, 1H), 2.68- 2.61 (m, 1H), 2.25-2.12 (m, 2H), 1.84-1.75 (m, 1H), 1.72-1.63 (m, 1H). |
| | (6S,9R)-N-(5-Chloro-2-fluoro- | | |
| | 4-(6-((2,2,2- | | |
| | trifluoroethyl)amino)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 3 | 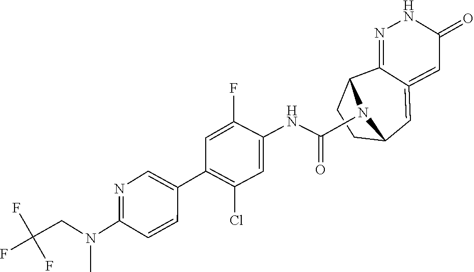 | Method 1 537.3, 539.3 at 1.37 min | ¹H NMR (400 MHz, DMSO- d6) δ 12.72 (d, J = 2.2 Hz, 1H), 8.84 (s, 1H), 8.20 (d, J = 2.5 Hz, 1H), 7.75-7.68 (m, 2H), 7.34 (d, J = 11.1 Hz, 1H), 6.87 (d, J = 8.8 Hz, 1H), 6.69 (s, 1H), 5.07 (d, J = 5.7 Hz, 1H), 4.64 (d, J = 6.4 Hz, 1H), 4.53 (q, J = 9.6 Hz, 2H), 3.19 (dd, J = 18.4, 5.1 Hz, 1H), 3.13 (s, 3H), 2.65 (d, J = 18.1 Hz, 1H), 2.19 (dd, J = 11.7, 6.5 Hz, 2H), 1.86- 1.75 (m, 1H), 1.73-1.60 (m, 1H) |
| | (6S,9R)-N-(5-Chloro-2-fluoro- | | |
| | 4-(6-(methyl(2,2,2- | | |
| | trifluoroethyl)amino)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 4^{(a)(b)} | 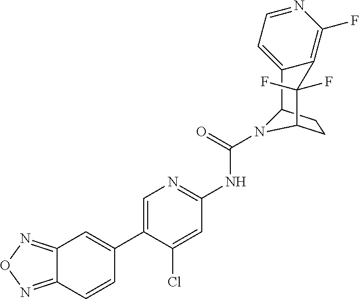 [SMILES: O=C(Nc1cc(Cl)c(-c2ccc3nonc3c2)cn1)N1C2CCC1C(F)(F)c1c2ccnc1F] | Method 6 487.1 at 1.11 min | ¹H NMR (400 MHz, DMSO- d6) δ 10.27 (s, 1H), 8.47 (s, 1H), 8.38 (d, J = 4.8 Hz, 1H), 8.21 (s, 1H), 8.14 (d, J = 9.2 Hz, 1H), 8.07 (s, 1H), 7.75 (d, J = 9.2 Hz, 1H), 7.51 (d, J = 9.2 Hz, 1H), 5.56 (d, J = 6.0 Hz, 1H), 5.41 (t, J = 9.2 Hz, 1H), 2.38-2.31 (m, 1H), 2.25-2.16 (m, 1H), 1.97- 1.90 (m, 1H), 1.75-1.70 (m, 1H). |
| | (5R,8S)-N-(5- | | |
| | (Benzo[c][1,2,5]oxadiazol-5-yl)- | | |
| | 4-chloropyridin-2-yl)-1,9,9- | | |
| | trifluoro-6,7,8,9-tetrahydro-5H- | | |
| | 5,8- | | |
| | epiminocyclohepta[c]pyridine- | | |
| | 10-carboxamide | | |
| 5^{(b)(c)} | 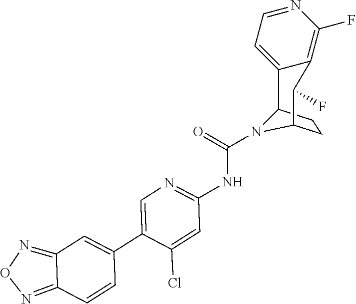 [SMILES: O=C(Nc1cc(Cl)c(-c2ccc3nonc3c2)cn1)N1C2CCC1[C@H](F)c1c2ccnc1F] | Method 7 467.1 at 2.32 min | ¹H NMR (400 MHz, DMSO- d6) δ 10.24 (s, 1H), 8.46 (s, 1H), 8.19 (s, 2H), 8.15 (d, J = 9.6 Hz, 1H), 8.10 (s, 1H), 7.73 (d, J = 10.8 Hz, 1H), 7.35 (d, J = 5.2 Hz, 1H), 6.33 (dd, J = 51.2, 6.0 Hz, 1H), 5.48 (d, J = 6.0 Hz, 1H), 5.10 (m, 1H), 2.20-2.12 (m, 3H), 1.85-1.81 (m, 1H). |
| | (±,9S)-N-(5- | | |
| | (Benzo[c][1,2,5]oxadiazol-5-yl)- | | |
| | 4-chloropyridin-2-yl)-1,9- | | |
| | difluoro-6,7,8,9-tetrahydro-5H- | | |
| | 5,8- | | |
| | epiminocyclohepta[c]pyridine- | | |
| | 10-carboxamide | | |
| 6^{(a)(b)} | 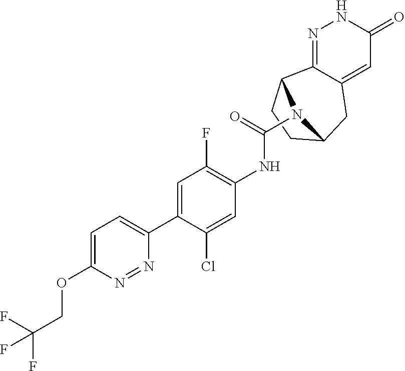 [SMILES: O=C(Nc1cc(Cl)c(-c2ccc(OCC(F)(F)F)nn2)cc1F)N1[C@H]2CC[C@@H]1c1n[nH]c(=O)cc1C2] | Method 4 525.1 at 1.50 min | ¹H NMR (400 MHz, DMSO- d6) δ 12.72 (s, 1H), 8.96 (s, 1H), 8.01 (d, J = 9.6 Hz, 1H), 7.85 (d, J = 7.2 Hz, 1H), 7.55 (s, 1H), 7.53 (d, J = 3.2 Hz, 1H), 6.69 (s, 1H), 5.25 (q, J = 9.6 Hz, 2H), 5.09 (d, J = 5.2 Hz, 1H), 4.66 (t, J = 5.6 Hz, 1H), 3.20 (dd, J = 5.6 Hz, J = 18.8 Hz, 1H), 2.65 (d, J = 18.0 Hz, 1H), 2.24-2.17 (m, 2H), 1.83-1.66 (m, 2H) |
| | (6S,9R)-N-(5-Chloro-2-fluoro- | | |
| | 4-(6-(2,2,2- | | |
| | trifluoroethoxy)pyridazin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 7 | 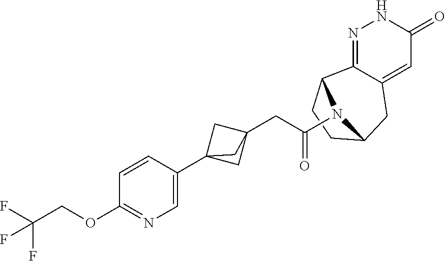 [SMILES: O=C(CC12CC(c3ccc(OCC(F)(F)F)nc3)(C1)C2)N1[C@H]2CC[C@@H]1c1n[nH]c(=O)cc1C2] | Method 2 462.4 at 1.62 min | ¹H NMR (400 MHz, DMSO- d6) δ 12.68 (s, 1H), 8.02 (d, J = 1.7 Hz, 1H), 7.66 (dd, J = 8.5, 2.4 Hz, 1H), 7.44 (s, 1H), 6.91 (d, J = 8.5 Hz, 1H), 6.65 (s, 1H), 4.95 (q, J = 9.2 Hz, 2H), 4.92 (d, J = 5.9 Hz, 1H), 4.44 (t, J = 6.2 Hz, 1H), 3.04 (dd, J = 18.1, 5.2 Hz, 1H), 2.55 (d, J = 18.3 Hz, 1H), 2.19 (s, 6H), 2.14-2.01 (m, 2H), 1.76-1.66 (m, 1H), 1.65-1.55 (m, 1H). |
| | (6S,9R)-3-Oxo-N-(3-(6-(2,2,2- | | |
| | trifluoroethoxy)pyridin-3- | | |
| | yl)bicyclo[1.1.1]pentan-1-yl)- | | |
| | 3,5,6,7,8,9-hexahydro-2H-6,9- | | |
| | epiminocyclohepta[c]pyridazine- | | |
| | 10-carboxamide | | |
| 8^{(c)} | 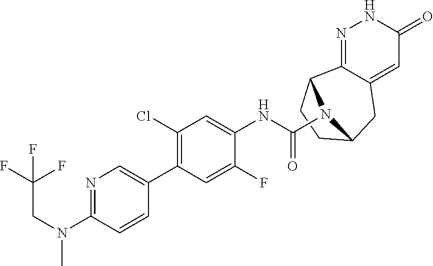 [SMILES: CN(CC(F)(F)F)c1ccc(-c2cc(F)c(NC(=O)N3[C@H]4CC[C@@H]3c3n[nH]c(=O)cc3C4)cc2Cl)cn1] | Method 1 535.6, 538.6 at 1.37 min | ¹H NMR (400 MHz, DMSO- d6) δ 11.27 (s, 1H), 8.60 (s, 1H), 8.20 (d, J = 2.5 Hz, 1H), 7.79-7.64 (m, 2H), 7.33 (d, J = 11.2 Hz, 1H), 7.20 (s, 1H), 6.87 (d, J = 9.0 Hz, 1H), 6.11 (s, 1H), 5.06 (d, J = 5.9 Hz, 1H), 4.59 (t, J = 6.4 Hz, 1H), 4.53 (q, J = 9.5 Hz, 2H), 3.20-3.09 (m, 4H), 2.55 (d, J = 17.7 Hz, 1H), 2.24-2.14 (m, 1H), 2.14-2.01 (m, 1H), 1.77-1.59 (m, 2H). |
| | (6S,9R)-N-(5-Chloro-2-fluoro- | | |
| | 4-(6-(methyl(2,2,2- | | |
| | trifluoroethyl)amino)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 9 | 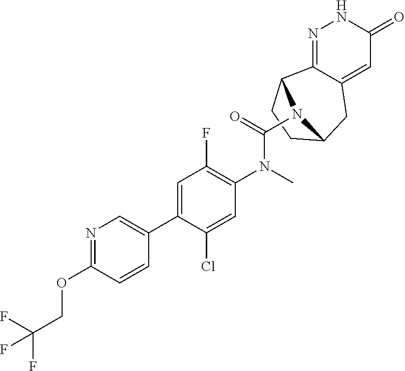 [SMILES: CN(C(=O)N1[C@H]2CC[C@@H]1c1n[nH]c(=O)cc1C2)c1cc(Cl)c(-c2ccc(OCC(F)(F)F)nc2)cc1F] | 538.1, 540.2 at 1.48 min | ¹H NMR (400 MHz, DMSO- d6) δ 12.60 (s, 1H), 8.26 (dd, J = 2.5, 0.8 Hz, 1H), 7.90 (dd, J = 8.6, 2.5 Hz, 1H), 7.66 (d, J = 7.4 Hz, 1H), 7.41 (d, J = 11.1 Hz, 1H), 7.10 (dd, J = 8.6, 0.8 Hz, 1H), 6.62 (s, 1H), 5.06 (q, J = 9.1 Hz, 2H), 4.53-4.47 (m, 1H), 4.35 (d, J = 6.5 Hz, 1H), 3.13 (s, 3H), 2.89 (dd, J = 18.5, 5.2 Hz, 1H), 2.56 (d, J = 18.1 Hz, 1H), 2.08- 1.96 (m, 2H), 1.68 (t, J = 9.6 Hz, 1H), 1.59-1.50 (m, 1H). |
| | (6S,9R)-N-(5-Chloro-2-fluoro- | | |
| | 4-(6-(2,2,2- | | |
| | trifluoroethoxy)pyridin-3- | | |
| | yl)phenyl)-N-methyl-3-oxo- | | |
| | 3,5,6,7,8,9-hexahydro-2H-6,9- | | |
| | epiminocyclohepta[c]pyridazine- | | |
| | 10-carboxamide | | |
| 10 | 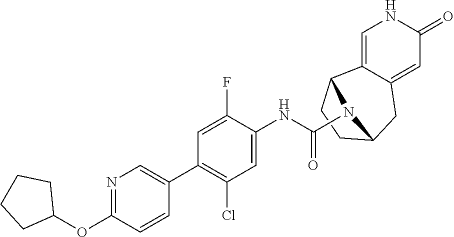 [SMILES: O=C(Nc1cc(Cl)c(-c2ccc(OC3CCCC3)nc2)cc1F)N1[C@H]2CC[C@@H]1c1c[nH]c(=O)cc1C2] | Method 1 509.3, 511.4 at 1.63 min | ¹H NMR (400 MHz, DMSO- d6) δ 11.27 (s, 1H), 8.62 (s, 1H), 8.20 (d, J = 2.6 Hz, 1H), 7.86-7.70 (m, 2H), 7.36 (d, J = 11.1 Hz, 1H), 7.20 (s, 1H), 6.83 (d, J = 8.5 Hz, 1H), 6.11 (s, 1H), 5.48-5.33 (m, 1H), 5.06 (d, J = 5.9 Hz, 1H), 4.59 (t, J = 6.3 Hz, 1H), 3.15 (dd, J = 17.6, 5.1 Hz, 1H), 2.55 (d, J = 17.8 Hz, 1H), 2.25-2.13 (m, 1H), 2.13- 2.02 (m, 1H), 2.02-1.88 (m, 2H), 1.79-1.53 (m, 8H). |
| | (6S,9R)-N-(5-Chloro-4-(6- | | |
| | (cyclopentyloxy)pyridin-3-yl)-2- | | |
| | fluorophenyl)-3-oxo- | | |
| | 3,5,6,7,8,9-hexahydro-2H-6,9- | | |
| | epiminocyclohepta[c]pyridine- | | |
| | 10-carboxamide | | |
| 11 | 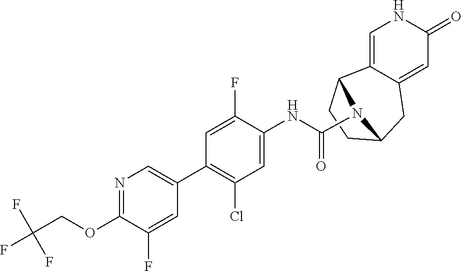 [SMILES: O=C(Nc1cc(Cl)c(-c2cnc(OCC(F)(F)F)c(F)c2)cc1F)N1[C@H]2CC[C@@H]1c1c[nH]c(=O)cc1C2] | Method 1 541.3, 543.2 at 1.53 min | ¹H NMR (400 MHz, DMSO- d6) δ 11.28 (s, 1H), 8.67 (s, 1H), 8.11 (d, J = 1.9 Hz, 1H), 8.00 (dd, J = 11.2, 2.0 Hz, 1H), 7.81 (d, J = 7.3 Hz, 1H), 7.45 (d, J = 11.1 Hz, 1H), 7.21 (s, 1H), 6.11 (s, 1H), 5.15 (q, J = 9.0 Hz, 2H), 5.07 (d, J = 5.8 Hz, 1H), 4.60 (t, J = 6.3 Hz, 1H), 3.15 (dd, J = 17.6, 5.0 Hz, 1H), 2.56 (d, J = 17.9 Hz, 1H), 2.26-2.02 (m, 2H), 1.80- 1.59 (m, 2H). |
| | (6S,9R)-N-(5-Chloro-2-fluoro- | | |
| | 4-(5-fluoro-6-(2,2,2- | | |
| | trifluoroethoxy)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 12 | 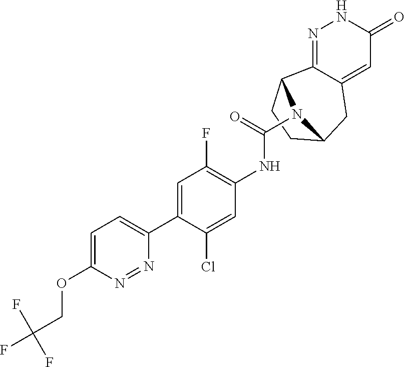 [SMILES: O=C(Nc1cc(Cl)c(-c2ccc(OCC(F)(F)F)nn2)cc1F)N1[C@H]2CC[C@@H]1c1n[nH]c(=O)cc1C2] | Method 3 524.1 at 2.24 min | ¹H NMR (400 MHz, DMSO- d6) δ 11.27 (s, 1H), 8.72 (s, 1H), 8.01 (d, J = 9.6 Hz, 1H), 7.86 (d, J = 7.2 Hz, 1H), 7.55 (s, 1H), 7.51 (d, J = 3.6 Hz, 1H), 7.21 (s, 1H), 6.11 (s, 1H), 5.25 (q, J = 8.8 Hz, 2H), 5.08 (d, J = 6.0 Hz, 1H), 4.61 (t, J = 6.4 Hz, 1H), 3.15 (dd, J = 5.2 Hz, J = 12.8 Hz, 1H), 2.55 (d, J = 18.0 Hz, 1H), 2.20-2.07 (m, 2H), 1.75- 1.62 (m, 2H). |
| | (6S,9R)-N-(5-Chloro-2-fluoro- | | |
| | 4-(6-(2,2,2- | | |
| | trifluoroethoxy)pyridazin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 13 | 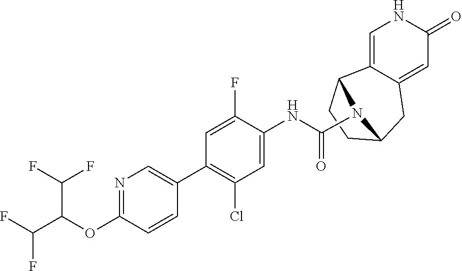 [SMILES: O=C(Nc1cc(Cl)c(-c2ccc(OC(C(F)F)C(F)F)nc2)cc1F)N1[C@H]2CC[C@@H]1c1c[nH]c(=O)cc1C2] | Method 2 555.3, 557.0 at 1.80 min | ¹H NMR (400 MHz, DMSO- d6) δ 11.27 (s, 1H), 8.66 (s, 1H), 8.26 (dd, J = 2.5, 0.8 Hz, 1H), 7.93 (dd, J = 8.6, 2.5 Hz, 1H), 7.80 (d, J = 7.3 Hz, 1H), 7.42 (d, J = 11.1 Hz, 1H), 7.20 (s, 1H), 7.12 (dd, J = 8.5, 0.7 Hz, 1H), 6.83-6.23 (m, 2H), 6.10 (d, J = 7.3 Hz, 2H), 5.06 (d, J = 5.9 Hz, 1H), 4.60 (t, J = 6.3 Hz, 1H), 3.15 (dd, J = 17.9, 5.0 Hz, 1H), 2.56 (d, J = 17.9 Hz, 1H), 2.28-2.00 (m, 2H), 1.69 (ddd, J = 21.8, 11.4, 7.2 Hz, 2H). |
| | (6S,9R)-N-(5-Chloro-2-fluoro- | | |
| | 4-(6-((1,1,3,3- | | |
| | tetrafluoropropan-2- | | |
| | yl)oxy)pyridin-3-yl)phenyl)-3- | | |
| | oxo-3,5,6,7,8,9-hexahydro-2H- | | |
| | 6,9- | | |
| | epiminocyclohepta[c]pyridine- | | |
| | 10-carboxamide | | |
| 14 | 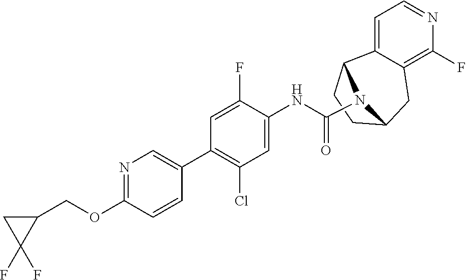 [SMILES: O=C(Nc1cc(Cl)c(-c2ccc(OCC3CC3(F)F)nc2)cc1F)N1[C@H]2CC[C@@H]1c1ccnc(F)c1C2] | Method 2 531.1, 533.3 at 2.20 min | ¹H NMR (400 MHz, DMSO- d6) δ 8.83 (s, 1H), 8.21 (dd, J = 2.5, 0.8 Hz, 1H), 8.01 (d, J = 5.0 Hz, 1H), 7.82 (dd, J = 8.6, 2.5 Hz, 1H), 7.75 (d, J = 7.3 Hz, 1H), 7.37 (d, J = 11.1 Hz, 1H), 7.21 (dd, J = 5.1, 1.6 Hz, 1H), 6.95 (dd, J = 8.6, 0.7 Hz, 1H), 5.23 (d, J = 5.9 Hz, 1H), 4.80 (t, J = 6.1 Hz, 1H), 4.50 (ddd, J = 11.3, 5.2, 2.1 Hz, 1H), 4.24 (ddd, J = 11.4, 8.6, 1.8 Hz, 1H), 3.20 (dd, J = 17.4, 5.0 Hz, 1H), 2.59 (d, J = 17.4 Hz, 1H), 2.37- 2.13 (m, 3H), 1.91-1.80 (m, 1H), 1.76-1.69 (m, 2H), |
| | (5R,8S)-N-(5-Chloro-4-(6-((2,2- | | 1.63-1.46 (m, 1H). |
| | difluorocyclopropyl)methoxy) | | |
| | pyridin-3-yl)-2-fluorophenyl)-1- | | |
| | fluoro-6,7,8,9-tetrahydro-5H- | | |
| | 5,8- | | |
| | epiminocyclohepta[c]pyridine- | | |
| | 10-carboxamide | | |
| 15 | 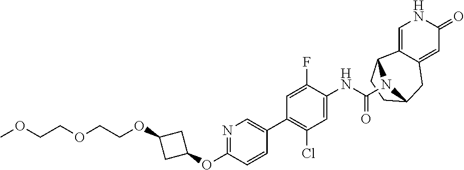 [SMILES: COCCOCCO[C@H]1C[C@@H](Oc2ccc(-c3cc(F)c(NC(=O)N4[C@H]5CC[C@@H]4c4c[nH]c(=O)cc4C5)cc3Cl)cn2)C1] | Method 1 613.4, 615.6 at 1.27 min | ¹H NMR (400 MHz, DMSO- d6) δ 11.30 (s, 1H), 8.66 (s, 1H), 8.18 (dd, J = 2.5, 0.8 Hz, 1H), 7.80 (dd, J = 8.6, 2.5 Hz, 1H), 7.76 (d, J = 7.3 Hz, 1H), 7.38 (d, J = 11.1 Hz, 1H), 7.21 (s, 1H), 6.92-6.85 (m, 1H), 6.11 (s, 1H), 5.05 (d, J = 5.9 Hz, 1H), 4.83 (p, J = 7.2 Hz, 1H), 4.59 (t, J = 6.4 Hz, 1H), 3.75 (p, J = 7.0 Hz, 1H), 3.55-3.47 (m, 4H), |
| | (6S,9R)-N-(5-Chloro-2-fluoro- | | 3.46-3.41 (m, 4H), 3.23 (s, |
| | 4-(6-((1s,3S)-3-(2-(2- | | 3H), 3.14 (dd, J = 17.8, 5.0 |
| | methoxyethoxy)ethoxy) | | Hz, 1H), 2.90-2.78 (m, 2H), |
| | cyclobutoxy)pyridin-3-yl) | | 2.55 (d, J = 18.1 Hz, 1H), |
| | phenyl)-3-oxo- | | 2.24-2.03 (m, 2H), 2.00- |
| | 3,5,6,7,8,9-hexahydro-2H-6,9- | | 1.89 (m, 2H), 1.77-1.59 (m, |
| | epiminocyclohepta[c]pyridine- | | 2H). |
| | 10-carboxamide | | |
| 16 | 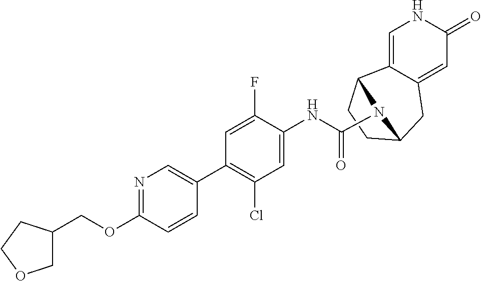 [SMILES: O=C(Nc1cc(Cl)c(-c2ccc(OCC3CCOC3)nc2)cc1F)N1[C@H]2CC[C@@H]1c1c[nH]c(=O)cc1C2] | Method 2 526.3 at 1.57 min | ¹H NMR (400 MHz, DMSO- d6) δ 11.27 (s, 1H), 8.63 (s, 1H), 8.20 (dd, J = 2.5, 0.8 Hz, 1H), 7.80 (dd, J = 8.6, 2.5 Hz, 1H), 7.77 (d, J = 7.3 Hz, 1H), 7.37 (d, J = 11.1 Hz, 1H), 7.20 (s, 1H), 6.90 (d, J = 8.6 Hz, 1H), 6.11 (s, 1H), 5.06 (d, J = 5.8 Hz, 1H), 4.59 (t, J = 6.4 Hz, 1H), 4.39- 4.04 (m, 2H), 3.83-3.72 (m, 2H), 3.71-3.61 (m, 1H), 3.54 (dd, J = 8.6, 5.5 Hz, 1H), 3.15 (dd, J = 17.8, 5.0 Hz, 1H), 2.69 (dq, J = 13.7, 6.7 Hz, 1H), 2.58 (s, 1H), 2.54 (d, J = 2.5 Hz, 1H), 2.24-1.96 (m, 3H), 1.77- |
| | (6S,9R)-N-(5-Chloro-2-fluoro- | | 1.60 (m, 3H). |
| | 4-(6-((tetrahydrofuran-3- | | |
| | yl)methoxy)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 17 | 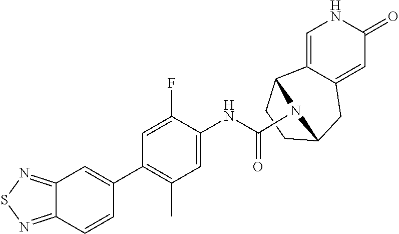 [SMILES: Cc1cc(NC(=O)N2[C@H]3CC[C@@H]2c2c[nH]c(=O)cc2C3)c(F)cc1-c1ccc2nsnc2c1] | Method 2 462.3, 464.3 at 1.61 min | ¹H NMR (400 MHz, DMSO- d6) δ 11.27 (s, 1H), 8.46 (s, 1H), 8.13 (dd, J = 9.0, 0.8 Hz, 1H), 8.04 (dd, J = 1.8, 0.8 Hz, 1H), 7.73 (dd, J = 9.0, 1.7 Hz, 1H), 7.45 (d, J = 8.1 Hz, 1H), 7.29-7.13 (m, 2H), 6.12 (s, 1H), 5.06 (d, J = 5.8 Hz, 1H), 4.60 (t, J = 6.3 Hz, 1H), 3.22-3.11 (m, 1H), 2.55 (d, J = 17.9 Hz, 1H), 2.22 (s, 3H), 2.21-2.04 (m, 2H), 1.70 (ddd, J = 21.5, 11.6, 7.2 Hz, 2H). |
| | (6S,9R)-N-(4- | | |
| | (Benzo[c][1,2,5]thiadiazol-5-yl)- | | |
| | 2-fluoro-5-methylphenyl)-3- | | |
| | oxo-3,5,6,7,8,9-hexahydro-2H- | | |
| | 6,9- | | |
| | epiminocyclohepta[c]pyridine- | | |
| | 10-carboxamide | | |
| 18 | 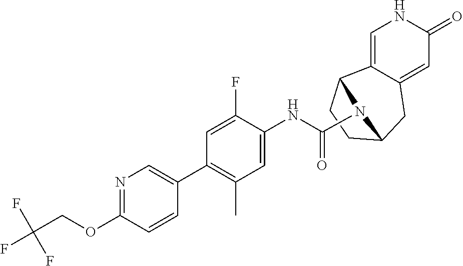 [SMILES: Cc1cc(NC(=O)N2[C@H]3CC[C@@H]2c2c[nH]c(=O)cc2C3)c(F)cc1-c1ccc(OCC(F)(F)F)nc1] | Method 2 504.4 505.4 at 1.78 min | ¹H NMR (400 MHz, DMSO- d6) δ 11.26 (s, 1H), 8.41 (s, 1H), 8.18 (d, J = 2.5 Hz, 1H), 7.83 (dd, J = 8.5, 2.5 Hz, 1H), 7.39 (d, J = 8.2 Hz, 1H), 7.18 (s, 1H), 7.09 (d, J = 11.3 Hz, 1H), 7.05 (d, J = 8.5 Hz, 1H), 6.11 (s, 1H), 5.14- 4.93 (m, 3H), 4.58 (t, J = 6.3 Hz, 1H), 3.14 (dd, J = 18.5, 5.0 Hz, 1H), 2.56 (s, 1H), 2.27-2.01 (m, 5H), 1.81- 1.57 (m, 2H). |
| | (6S,9R)-N-(2-Fluoro-5-methyl- | | |
| | 4-(6-(2,2,2- | | |
| | trifluoroethoxy)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 19 | 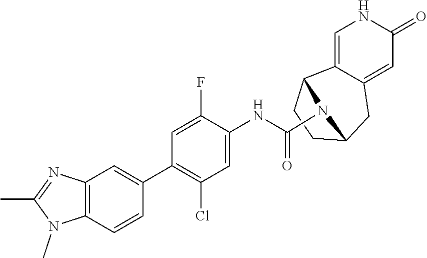 [SMILES: Cc1nc2cc(-c3cc(F)c(NC(=O)N4[C@H]5CC[C@@H]4c4c[nH]c(=O)cc4C5)cc3Cl)ccc2n1C] | Method 2 492.3, 494.3 at 1.00 min | ¹H NMR (400 MHz, DMSO- d6) δ 7.74 (d, J = 7.4 Hz, 1H), 7.56-7.50 (m, 2H), 7.30 (d, J = 11.1 Hz, 1H), 7.23 (dd, J = 8.4, 1.6 Hz, 1H), 7.21 (s, 1H), 6.11 (s, 1H), 5.06 (d, J = 5.9 Hz, 1H), 4.68-4.50 (m, 1H), 3.76 (s, 3H), 3.16 (dd, J = 17.8, 5.3 Hz, 1H), 2.54 (s, 4H), 2.25- 2.03 (m, 2H), 1.78-1.60 (m, 2H). (2 exchangeable protons not observed) |
| | (6S,9R)-N-(5-Chloro-4-(1,2- | | |
| | dimethyl-1H-benzo[d]imidazol- | | |
| | 5-yl)-2-fluorophenyl)-3-oxo- | | |
| | 3,5,6,7,8,9-hexahydro-2H-6,9- | | |
| | epiminocyclohepta[c]pyridine- | | |
| | 10-carboxamide | | |
| 20 | 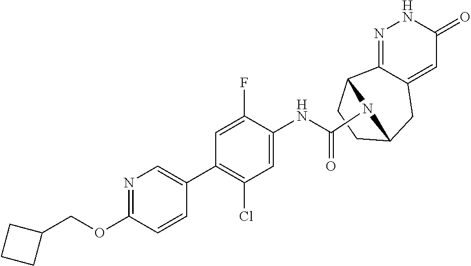 [SMILES: O=C(Nc1cc(Cl)c(-c2ccc(OCC3CCC3)nc2)cc1F)N1[C@H]2CC[C@@H]1c1n[nH]c(=O)cc1C2] | Method 2 510.3, 512.3 at 2.01 min | ¹H NMR (400 MHz, DMSO- d6) δ 12.72 (s, 1H), 8.87 (s, 1H), 8.20 (dd, J = 2.5, 0.8 Hz, 1H), 7.79 (dd, J = 8.6, 2.6 Hz, 1H), 7.76 (d, J = 7.4 Hz, 1H), 7.37 (d, J = 11.1 Hz, 1H), 6.88 (dd, J = 8.6, 0.8 Hz, 1H), 6.69 (s, 1H), 5.08 (d, J = 5.7 Hz, 1H), 4.64 (t, J = 6.0 Hz, 1H), 4.28 (d, J = 6.9 Hz, 2H), 3.20 (dd, J = 18.5, 5.3 Hz, 1H), 2.74 (p, J = 7.3 Hz, 1H), 2.65 (d, J = 18.2 Hz, 1H), 2.29-2.12 (m, 2H), 2.13-2.00 (m, 2H), 1.96- 1.76 (m, 5H), 1.72-1.63 (m, 1H). |
| | (6S,9R)-N-(5-Chloro-4-(6- | | |
| | (cyclobutylmethoxy)pyridin-3- | | |
| | yl)-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 21 | 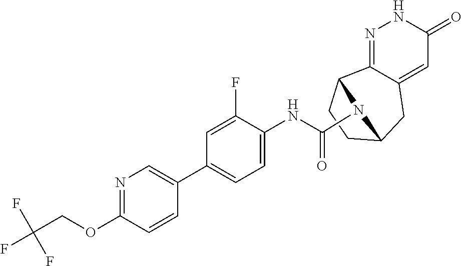 [SMILES: O=C(Nc1ccc(-c2ccc(OCC(F)(F)F)nc2)cc1F)N1[C@H]2CC[C@@H]1c1n[nH]c(=O)cc1C2] | Method 2 490.3 at 1.73 min | ¹H NMR (400 MHz, DMSO- d6) δ 12.71 (s, 1H), 8.71 (s, 1H), 8.53 (d, J = 0.8 Hz, 1H), 8.13 (dd, J = 8.6, 2.6 Hz, 1H), 7.63-7.50 (m, 2H), 7.46 (dd, J = 8.4, 2.1 Hz, 1H), 7.07 (d, J = 0.7 Hz, 1H), 6.69 (s, 1H), 5.09-4.98 (m, 3H), 4.63 (t, J = 6.1 Hz, 1H), 3.20 (dd, J = 18.3, 5.3 Hz, 1H), 2.65 (d, J = 18.0 Hz, 1H), 2.28-2.11 (m, 2H), 1.87-1.75 (m, 1H), 1.72- 1.64 (m, 1H). |
| | (6S,9R)-N-(2-Fluoro-4-(6- | | |
| | (2,2,2-trifluoroethoxy)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 22 | 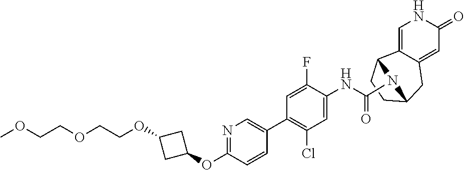 [SMILES: COCCOCCO[C@H]1C[C@H](Oc2ccc(-c3cc(F)c(NC(=O)N4[C@H]5CC[C@@H]4c4c[nH]c(=O)cc4C5)cc3Cl)cn2)C1] | Method 1 613.4, 615.5 at 1.36 min | ¹H NMR (400 MHz, DMSO- d6) δ 11.26 (s, 1H), 8.63 (s, 1H), 8.19 (dd, J = 2.6, 0.8 Hz, 1H), 7.80 (dd, J = 8.6, 2.5 Hz, 1H), 7.76 (d, J = 7.4 Hz, 1H), 7.36 (d, J = 11.1 Hz, 1H), 7.20 (s, 1H), 6.88 (dd, J = 8.5, 0.7 Hz, 1H), 6.11 (s, 1H), 5.28 (tt, J = 7.0, 4.5 Hz, 1H), 5.06 (d, J = 5.8 Hz, 1H), 4.63-4.55 (m, 1H), 4.21 (tt, J = 6.9, 4.4 Hz, 1H), 3.58- |
| | (6S,9R)-N-(5-Chloro-2-fluoro- | | 3.47 (m, 4H), 3.48-3.40 (m, |
| | 4-(6-((1r,3R)-3-(2-(2- | | 4H), 3.24 (s, 3H), 3.15 (dd, |
| | methoxyethoxy)ethoxy) | | J = 17.9, 5.1 Hz, 1H), 2.55 (d, |
| | cyclobutoxy)pyridin-3-yl) | | J = 18.0 Hz, 1H), 2.45-2.38 |
| | phenyl)-3-oxo- | | (m, 2H), 2.37-2.28 (m, 2H), |
| | 3,5,6,7,8,9-hexahydro-2H-6,9- | | 2.24-2.02 (m, 2H), 1.77- |
| | epiminocyclohepta[c]pyridine- | | 1.60 (m, 2H). |
| | 10-carboxamide | | |
| 23 | 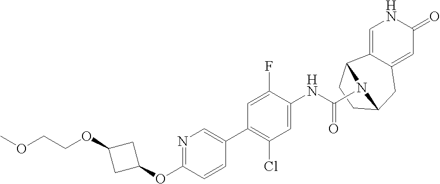 [SMILES: COCCO[C@H]1C[C@@H](Oc2ccc(-c3cc(F)c(NC(=O)N4[C@H]5CC[C@@H]4c4c[nH]c(=O)cc4C5)cc3Cl)cn2)C1] | Method 1 569.3, 571.3 at 1.26 min | ¹H NMR (400 MHz, DMSO- d6) δ 11.27 (s, 1H), 8.63 (s, 1H), 8.18 (dd, J = 2.5, 0.8 Hz, 1H), 7.80 (dd, J = 8.6, 2.6 Hz, 1H), 7.76 (d, J = 7.4 Hz, 1H), 7.37 (d, J = 11.1 Hz, 1H), 7.20 (s, 1H), 6.88 (dd, J = 8.6, 0.7 Hz, 1H), 6.11 (s, 1H), 5.06 (d, J = 5.8 Hz, 1H), 4.84 (p, J = 7.2 Hz, 1H), 4.59 (t, J = 6.0 Hz, 1H), 3.75 (p, J = 6.9 Hz, 1H), 3.46-3.41 |
| | | | (m, 4H), 3.24 (s, 3H), 3.15 |
| | (6S,9R)-N-(5-Chloro-2-fluoro- | | (dd, J = 17.9, 5.0 Hz, 1H), |
| | 4-(6-((1s,3S)-3-(2- | | 2.90-2.78 (m, 2H), 2.55 (d, |
| | methoxyethoxy)cyclobutoxy) | | J = 18.0 Hz, 1H), 2.24-2.02 |
| | pyridin-3-yl)phenyl)-3-oxo- | | (m, 2H), 2.01-1.88 (m, 2H), |
| | 3,5,6,7,8,9-hexahydro-2H-6,9- | | 1.77-1.59 (m, 2H). |
| | epiminocyclohepta[c]pyridine- | | |
| | 10-carboxamide | | |
| 24 | 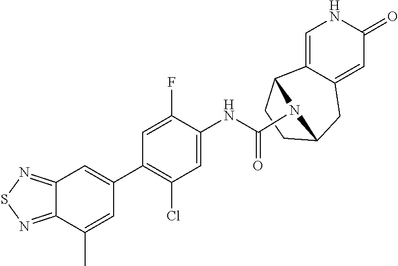 [SMILES: Cc1cc(-c2cc(F)c(NC(=O)N3[C@H]4CC[C@@H]3c3c[nH]c(=O)cc3C4)cc2Cl)cc2nsnc12] | Method 1 496.3, 498.2 at 1.44 min | ¹H NMR (400 MHz, DMSO- d6) δ 11.28 (s, 1H), 8.69 (s, 1H), 7.95 (d, J = 1.6 Hz, 1H), 7.85 (dd, J = 7.3, 4.1 Hz, 1H), 7.59 (t, J = 1.5 Hz, 1H), 7.49 (d, J = 11.0 Hz, 1H), 7.22 (s, 1H), 6.12 (s, 1H), 5.08 (d, J = 5.8 Hz, 1H), 4.62 (t, J = 6.4 Hz, 1H), 3.16 (dd, J = 18.0, 5.1 Hz, 1H), 2.72 (s, 3H), 2.57 (d, J = 17.9 Hz, 1H), 2.24-2.00 (m, 2H), 1.83-1.57 (m, 2H). |
| | (6S,9R)-N-(5-Chloro-2-fluoro- | | |
| | 4-(7- | | |
| | methylbenzo[c][1,2,5]thiadiazol- | | |
| | 5-yl)phenyl)-3-oxo-3,5,6,7,8,9- | | |
| | hexahydro-2H-6,9- | | |
| | epiminocyclohepta[c]pyridine- | | |
| | 10-carboxamide | | |
| 25^{(d)} | 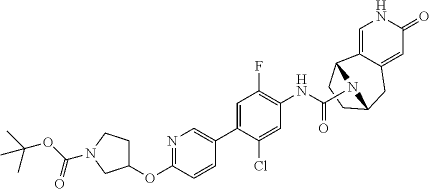 [SMILES: CC(C)(C)OC(=O)N1CCC(Oc2ccc(-c3cc(F)c(NC(=O)N4[C@H]5CC[C@@H]4c4c[nH]c(=O)cc4C5)cc3Cl)cn2)C1] | Method 7 610.2, 612.2 at 1.61 min | ¹H NMR (400 MHz, DMSO- d6) δ 12.26 (s, 1H), 8.63 (s, 1H), 8.22 (d, J =2.4 Hz, 1H), 7.83-7.77 (m, 2H), 7.38 (d, J = 10.8 Hz, 1H), 7.20 (s, 1H), 6.90 (d, J = 8.4 Hz, 1H), 6.11 (s, 1H), 5.56-5.48 (m, 1H), 5.06 (d, J = 5.6 Hz, 1H), 4.60 (d, J = 6.0 Hz, 1H), 3.65- 3.57 (m, 1H), 3.47-3.33 (m, 3H), 3.14 (dd, J = 5.2 Hz, J = 17.6 Hz, 1H), 2.55 (d, J = 18.0 Hz, 1H), 2.22-2.06 (m, |
| | tert-Butyl 3-((5-(2-chloro-5- | | 4H), 1.75-1.62 (m, 2H), |
| | fluoro-4-((6S,9R)-3-oxo- | | 1.39 (d, J = 5.2 Hz, 9H). |
| | 3,5,6,7,8,9-hexahydro-2H-6,9- | | |
| | epiminocyclohepta[c]pyridine- | | |
| | 10- | | |
| | carboxamido)phenyl)pyridin-2- | | |
| | yl)oxy)pyrrolidine-1-carboxylate | | |
| 26^{(c)} | 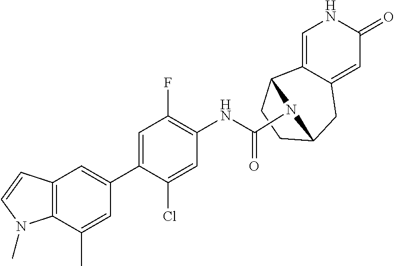 [SMILES: Cc1cc(-c2cc(F)c(NC(=O)N3[C@H]4CC[C@@H]3c3c[nH]c(=O)cc3C4)cc2Cl)cc2ccn(C)c12] | Method 7 477.1 at 1.71 min | ¹H NMR (400 MHz, DMSO- d6) δ 11.32 (s, 1H), 8.57 (s, 1H), 7.73-7.70 (m, 1H), 7.58 (s, 1H), 7.50 (d, J = 8.8 Hz, 1H), 7.38 (d, J = 3.2 Hz, 1H), 7.29 (d, J = 11.2 Hz, 1H), 7.21-7.183 (m, 2H), 6.47 (d, J = 3.2 Hz, 1H), 6.12 (s, 1H), 5.07 (d, J = 5.6 Hz, 1H), 4.60 (t, J = 6.4 Hz, 1H), 3.82 (s, 3H), 3.17 (dd, J = 5.2, 18.0 Hz, 1H), 2.58- 2.50 (m, 1H), 2.20-2.06 (m, 2H), 1.75-1.62 (m, 2H). |
| | (6S,9R)-N-(5-Chloro-2-fluoro- | | |
| | 4-(1-methyl-1H-indol-5- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 27^{(c)} | 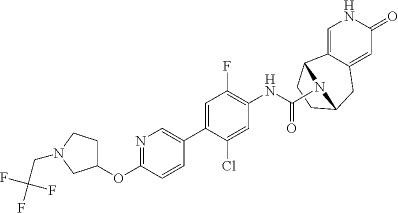 [SMILES: O=C(Nc1cc(Cl)c(-c2ccc(OC3CCN(CC(F)(F)F)C3)nc2)cc1F)N1[C@H]2CC[C@@H]1c1c[nH]c(=O)cc1C2] | Method 7 592.1 at 1.82 min | ¹H NMR (400 MHz, DMSO) δ 11.25 (s, 1H), 8.62 (s, 1H), 8.20 (d, J = 2.5 Hz, 1H), 7.83- 7.73 (m, 2H), 7.36 (d, J = 11.1 Hz, 1H), 7.19 (s, 1H), 6.88 (d, J = 8.6 Hz, 1H), 6.10 (s, 1H), 5.42-5.38 (m, 1H), 5.05 (d, J = 5.8 Hz, 1H), 4.62- 4.56 (m, 1H), 3.27 (d, J = 10.2 Hz, 1H), 3.19-3.06 (m, 2H), 2.93 (q, J = 7.6 Hz, 1H), 2.85 (dd, J = 10.9, 2.7 Hz, 1H), 2.67 (q, J = 7.5 Hz, 1H), 2.55 (d, J = 17.9 Hz, 2H), |
| | (6S,9R)-N-(5-Chloro-2-fluoro- | | 2.34-2.21 (m, 1H), 2.21- |
| | 4-(6-((1-(2,2,2- | | 2.02 (m, 2H), 1.91-1.83 (m, |
| | trifluoroethyl)pyrrolidin-3- | | 1H), 1.76-1.59 (m, 2H). |
| | yl)oxy)pyridin-3-yl)phenyl)-3- | | |
| | ox0-3,5,6,7,8,9-hexahydro-2H- | | |
| | 6,9-epiminocyclohepta[c]pyridine- | | |
| | 10-carboxamide | | |
| 28^{(a)} | 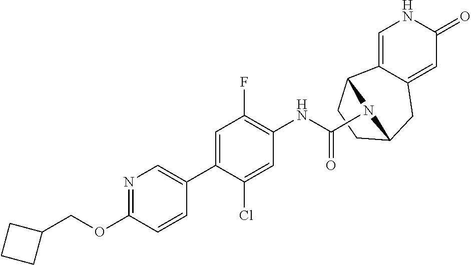 [SMILES: O=C(Nc1cc(Cl)c(-c2ccc(OCC3CCC3)nc2)cc1F)N1[C@H]2CC[C@@H]1c1c[nH]c(=O)cc1C2] | Method 7 min 509.1 at 2.23 min | ¹H NMR (400 MHz, DMSO- d6) δ 8.65 (s, 1H), 8.19 (d, J = 2.0 Hz, 1H), 7.80-7.77 (m, 2H), 7.36 (d, J = 11.2 Hz, 1H), 7.28 (s, 1H), 6.88 (d, J = 8.8 Hz, 1H), 6.18 (s, 1H), 5.09 (d, J = 5.6 Hz, 1H), 4.61 (t, J = 5.6 Hz, 1H), 4.28 (d, J = 6.8 Hz, 2H), 3.17 (dd, J = 4.0, 18.0 Hz, 1H), 2.77- 2.70 (m, 1H), 2.59 (d, J = 18.0 Hz, 1H), 2.23-2.04 (m, 4H), 1.92-1.65 (m, 6H). 1 exchangeable proton not observed |
| | (6S,9R)-N-(5-Chloro-4-(6- | | |
| | (cyclobutylmethoxy)pyridin-3- | | |
| | yl)-2-fluorophenyl)-3-oxo- | | |
| | 3,5,6,7,8,9-hexahydro-2H-6,9- | | |
| | epiminocyclohepta[c]pyridine- | | |
| | 10-carboxamide | | |
| 29^{(c)} | 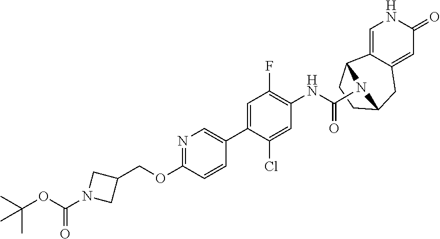 [SMILES: CC(C)(C)OC(=O)N1CC(COc2ccc(-c3cc(F)c(NC(=O)N4[C@H]5CC[C@@H]4c4c[nH]c(=O)cc4C5)cc3Cl)cn2)C1] | Method 3 610.2 at 1.99 min | ¹H NMR (400 MHz, DMSO- d6) δ 8.63 (s, 1H), 8.20 (d, J = 2.4 Hz, 1H), 7.82-7.76 (m, 2H), 7.36 (d, J = 10.8 Hz, 1H), 7.20 (s, 1H), 6.89 (d, J = 8.8 Hz, 1H), 6.11 (s, 1H), 5.06 (d, J = 5.6 Hz, 1H), 4.60 (t, J = 6.4 Hz, 1H), 4.43 (d, J = 6.4 Hz, 2H), 3.96 (t, J = 9.2 Hz, 2H), 3.68 (t, J = 6.4 Hz, 2H), 3.18-3.12 (m, 1H), 3.01-2.94 (m, 1H), 2.58- 2.53 (m, 1H), 2.22-2.04 (m, 2H), 1.75-1.62 (m, 2H), 1.38 (s, 9H). 1 exchangeable proton not observed |
| | tert-Butyl 3-(((5-(2-chloro-5- | | |
| | fluoro-4-((6S,9R)-3-oxo- | | |
| | 3,5,6,7,8,9-hexahydro-2H-6,9- | | |
| | epiminocyclohepta[c]pyridine- | | |
| | 10-carboxamido)phenyl)pyridin-2- | | |
| | yl)oxy)methyl)azetidine-1- | | |
| | carboxylate | | |
| 30 | 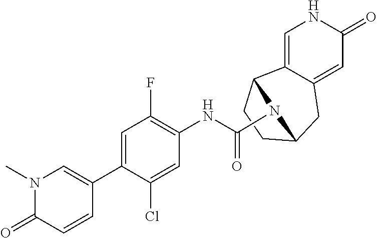 [SMILES: Cn1cc(-c2cc(F)c(NC(=O)N3[C@H]4CC[C@@H]3c3c[nH]c(=O)cc3C4)cc2Cl)ccc1=O] | Method 7 455.1 at 0.88 min | ¹H NMR (400 MHz, DMSO- d6) δ 8.60 (s, 1H), 7.87 (d, J = 2.4 Hz, 1H), 7.75-7.72 (m, 1H), 7.56 (dd, J = 2.4, 9.6 Hz, 1H), 7.24 (d, J = 11.2 Hz, 1H), 7.20 (s, 1H), 6.44 (d, J = 9.6 Hz, 1H), 6.11 (s, 1H), 5.05 (d, J = 5.6 Hz, 1H), 4.59 (t, J = 5.6 Hz, 1H), 3.47 (s, 3H), 3.14 (dd, J = 4.4, 17.6 Hz, 1H), 2.58-2.52 (m, 1H), 2.22-2.04 (m, 2H), 1.75- 1.62 (m, 2H). (1 Exchangeable proton not observed) |
| | (6S,9R)-N-(5-Chloro-2-fluoro- | | |
| | 4-(1-methyl-6-oxo-1,6- | | |
| | dihydropyridin-3-yl)phenyl)-3- | | |
| | oxo-3,5,6,7,8,9-hexahydro-2H- | | |
| | 6,9- | | |
| | epiminocyclohepta[c]pyridine- | | |
| | 10-carboxamide | | |
| 31^{(a)} | 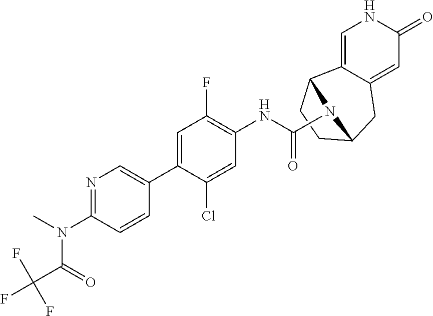 [SMILES: CN(C(=O)C(F)(F)F)c1ccc(-c2cc(F)c(NC(=O)N3[C@H]4CC[C@@H]3c3c[nH]c(=O)cc3C4)cc2Cl)cn1] | Method 3 550.1 at 1.46 min | ¹H NMR (400 MHz, DMSO- d6) δ 11.27 (s, 1H), 8.69 (s, 1H), 8.59 (d, J = 1.6 Hz, 1H), 8.32 (d, J = 9.2 Hz, 1H), 8.18- 8.15 (m, 1H), 7.84 (d, J = 7.2 Hz, 1H), 7.48 (d, J = 11.2 Hz, 1H), 7.20 (s, 1H), 6.11 (s, 1H), 5.07 (d, J = 6.0 Hz, 1H), 4.60 (t, J = 6.0 Hz, 1H), 3.93 (s, 3H), 3.17-3.11 (m, 1H), 2.58-2.54(m, 1H), 2.25-2.03 (m, 2H), 1.75- 1.62 (m, 2H). |
| | (6S,9R)-N-(5-Chloro-2-fluoro- | | |
| | 4-(6-(2,2,2-trifluoro-N- | | |
| | methylacetamido)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 32^{(c)} | 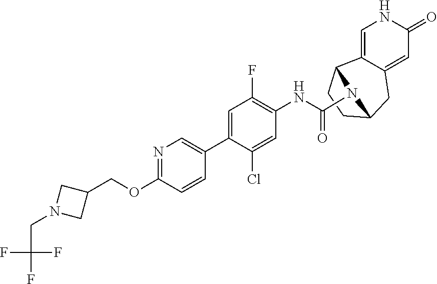 [SMILES: O=C(Nc1cc(Cl)c(-c2ccc(OCC3CN(CC(F)(F)F)C3)nc2)cc1F)N1[C@H]2CC[C@@H]1c1c[nH]c(=O)cc1C2] | Method 7 592.1 at 0.82 min | ¹H NMR (400 MHz, DMSO- d6) δ 11.26 (s, 1H), 8.63 (s, 1H), 8.20 (d, J = 2.8 Hz, 1H), 7.82-7.76 (m, 2H), 7.36 (d, J = 11.2 Hz, 1H), 7.20 (s, 1H), 6.90 (d, J = 8.8 Hz, 1H), 6.11 (s, 1H), 5.06 (d, J = 5.6 Hz, 1H), 4.60 (t, J = 6.0 Hz, 1H), 4.42 (d, J = 6.8 Hz, 2H), 3.51 (t, J = 7.6 Hz, 2H), 3.31- 3.12 (m, 5H), 2.92-2.85 (m, 1H), 2.58-2.53 (m, 1H), 2.22-2.05 (m, 2H), 1.75- 1.62 (m, 2H). |
| | 32(c) (6S,9R)-N-(5-Chloro-2-fluoro- | | |
| | 4-(6-((1-(2,2,2- | | |
| | trifluoroethyl)azetidin-3- | | |
| | yl)methoxy)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 33^{(c)} | 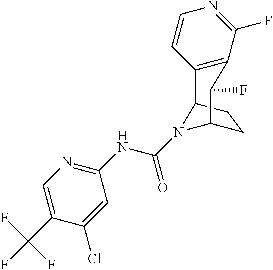 [SMILES: O=C(Nc1cc(Cl)c(C(F)(F)F)cn1)N1C2CCC1[C@H](F)c1c2ccnc1F] | Method 7 417.1 at 2.25 min | ¹H NMR (400 MHz, DMSO- d6) δ 10.50 (s, 1H), 8.67 (s, 1H), 8.19 (d, J = 5.2 Hz, 1H), 8.13 (s, 1H), 7.34 (d, J = 5.2 Hz, 1H), 6.30 (dd, J = 51.2, 6.0 Hz, 1H), 5.45 (d, J = 6.0 Hz, 1H), 5.08 (m, 1H), 2.20- 2.09 (m, 3H), 1.84-1.78 (m, 1H). |
| | (±)(9S)-N-(4-Chloro-5- | | |
| | (trifluoromethyl)pyridin-2-yl)- | | |
| | 1,9-difluoro-6,7,8,9-tetrahydro- | | |
| | 5H-5,8- | | |
| | epiminocyclohepta[c]pyridine- | | |
| | 10-carboxamide | | |
| 34^{(e)} | 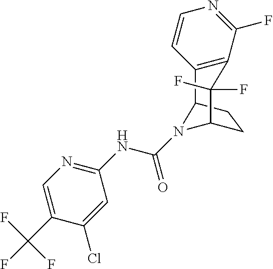 [SMILES: O=C(Nc1cc(Cl)c(C(F)(F)F)cn1)N1C2CCC1C(F)(F)c1c2ccnc1F] | Method 7 437.1 at 2.29 min | ¹H NMR (400 MHz, DMSO- d6) δ 10.55 (s, 1H), 8.68 (s, 1H), 8.38 (d, J = 5.2 Hz, 1H), 8.11 (s, 1H), 7.50 (d, J = 5.2 Hz, 1H), 5.56 (d, J = 6.0 Hz, 1H), 5.39 (t, J = 10 Hz, 1H), 2.33-2.17 (m, 2H), 1.95- 1.87 (m, 1H), 1.74-1.68 (m, 1H). |
| | (±)-N-(4-Chloro-5- | | |
| | (trifluoromethyl)pyridin-2-yl)- | | |
| | 1,9,9-trifluoro-6,7,8,9- | | |
| | tetrahydro-5H-5,8- | | |
| | epiminocyclohepta[c]pyridine- | | |
| | 10-carboxamide | | |
| 35^{(c)} | 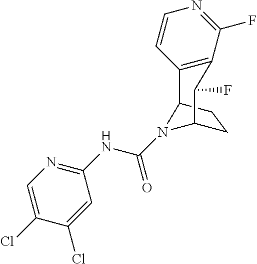 [SMILES: O=C(Nc1cc(Cl)c(Cl)cn1)N1C2CCC1[C@H](F)c1c2ccnc1F] | Method 7 383.1 at 2.14 min | ¹H NMR (400 MHz, DMSO- d6) δ 10.13 (s, 1H), 8.46 (s, 1H), 8.19 (d, J = 4.8 Hz, 1H), 8.09 (s, 1H), 7.33 (d, J = 4.8 Hz, 1H), 6.30 (dd, J = 51.2, 6.0 Hz, 1H), 5.44 (d, J = 6.0 Hz, 1H), 5.05 (m, 1H), 2.18- 2.08 (m, 3H), 1.83-1.79 (m, 1H). |
| | (±)(9S)-N-(4,5-Dichloropyridin- | | |
| | 2-yl)-1,9-difluoro-6,7,8,9- | | |
| | tetrahydro-5H-5,8- | | |
| | epiminocyclohepta[c]pyridine- | | |
| | 10-carboxamide | | |
| 36^{(e)} | 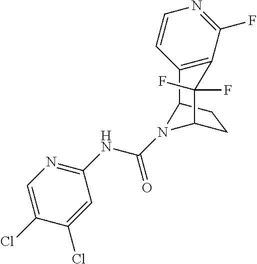 [SMILES: O=C(Nc1cc(Cl)c(Cl)cn1)N1C2CCC1C(F)(F)c1c2ccnc1F] | Method 7 403 at 2.18 min | ¹H NMR (400 MHz, DMSO- d6) δ 10.17 (s, 1H), 8.47 (s, 1H), 8.37 (d, J = 5.2 Hz, 1H), 8.07 (s, 1H), 7.48 (d, J = 5.2 Hz, 1H), 5.55 (d, J = 6.4 Hz, 1H), 5.39 (t, J = 10.4 Hz, 1H), 2.33-2.28 (m, 1H), 2.21- 2.15 (m, 1H), 1.91-1.86 (m, 1H), 1.73-1.67 (m, 1H). |
| | (±)-N-(4,5-Dichloropyridin-2-yl)- | | |
| | 1,9,9-trifluoro-6,7,8,9- | | |
| | tetrahydro-5H-5,8- | | |
| | epiminocyclohepta[c]pyridine- | | |
| | 10-carboxamide | | |
| 37^{(d)} | 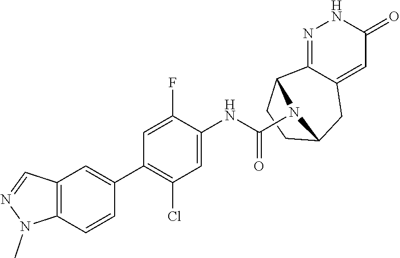 [SMILES: Cn1ncc2cc(-c3cc(F)c(NC(=O)N4[C@H]5CC[C@@H]4c4n[nH]c(=O)cc4C5)cc3Cl)ccc21] | Method 1 478.3, 480.3 at 1.17 min | ¹H NMR (400 MHz, DMSO) δ 11.28 (s, 1H), 8.61 (s, 1H), 8.10 (d, J = 0.9 Hz, 1H), 7.81- 7.78 (m, 1H), 7.76 (d, J = 7.4 Hz, 1H), 7.73-7.67 (m, 1H), 7.45 (dd, J = 8.7, 1.6 Hz, 1H), 7.34 (d, J = 11.2 Hz, 1H), 7.21 (s, 1H), 6.11 (s, 1H), 5.07 (d, J = 5.9 Hz, 1H), 4.60 (t, J = 6.3 Hz, 1H), 4.08 (s, 3H), 3.16 (dd, J = 17.9, 5.1 Hz, 1H), 2.56 (d, J = 17.9 Hz, 1H), 2.25-2.03 (m, 2H), 1.77-1.60 (m, 2H). |
| | (6S,9R)-N-(5-Chloro-2-fluoro- | | |
| | 4-(1-methyl-1H-indazol-5- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 38^{(d)} | 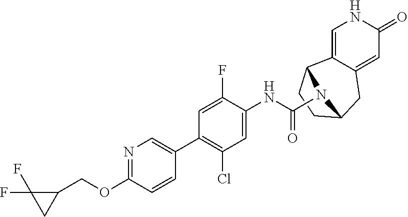 [SMILES: O=C(Nc1cc(Cl)c(-c2ccc(OCC3CC3(F)F)nc2)cc1F)N1[C@H]2CC[C@@H]1c1c[nH]c(=O)cc1C2] | Method 1 531.2 & 533.3 at 1.42 min | ¹H NMR (400 MHz, DMSO- d6) δ 11.27 (s, 1H), 8.63 (s, 1H), 8.22 (dd, J = 2.5, 0.8 Hz, 1H), 7.83 (dd, J = 8.6, 2.5 Hz, 1H), 7.77 (d, J = 7.3 Hz, 1H), 7.38 (d, J = 11.1 Hz, 1H), 7.20 (s, 1H), 6.95 (d, J = 8.5 Hz, 1H), 6.11 (s, 1H), 5.06 (d, J = 5.8 Hz, 1H), 4.60 (t, J = 6.4 Hz, 1H), 4.55- 4.45 (m, 1H), 4.29-4.19 (m, 1H), 3.15 (dd, J = 17.9, 5.1 Hz, 1H), 2.56 (d, J = 17.5 Hz, 1H), 2.38-1.94 (m, 3H), |
| | (6S,9R)-N-(5-Chloro-4-(6-((2,2- | | 1.80-1.60 (m, 3H), 1.59- |
| | difluorocyclopropyl)methoxy) | | 1.40 (m, 1H). |
| | pyridin-3-yl)-2-fluorophenyl)-3- | | |
| | oxo-3,5,6,7,8,9-hexahydro-2H- | | |
| | 6,9-epiminocyclohepta[c]pyridine- | | |
| | 10-carboxamide | | |
| 39^{(c)} | 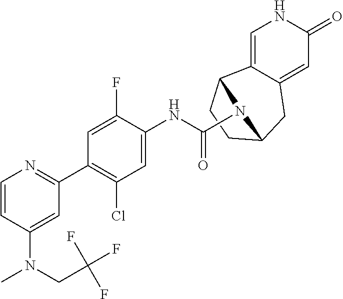 [SMILES: CN(CC(F)(F)F)c1ccnc(-c2cc(F)c(NC(=O)N3[C@H]4CC[C@@H]3c3c[nH]c(=O)cc3C4)cc2Cl)c1] | Method 1 536.3, 538.3 at 0.8 min | ¹H NMR (400 MHz, DMSO- d6) δ 11.27 (s, 1H), 8.63 (s, 1H), 8.26 (d, J = 6.0 Hz, 1H), 7.74 (d, J = 7.2 Hz, 1H), 7.39 (d, J = 11.1 Hz, 1H), 7.20 (s, 1H), 7.02 (d, J = 2.6 Hz, 1H), 6.83 (dd, J = 6.0, 2.7 Hz, 1H), 6.11 (s, 1H), 5.07 (d, J = 5.8 Hz, 1H), 4.60 (t, J = 6.4 Hz, 1H), 4.39 (q, J = 9.4 Hz, 2H), 3.15 (dd, J = 17.7, 4.8 Hz, 1H), 3.08 (s, 3H), 2.56 (d, J = 17.9 Hz, 1H), 2.25- 2.02 (m, 2H), 1.77-1.60 (m, 2H). |
| | (6S,9R)-N-(5-Chloro-2-fluoro- | | |
| | 4-(4-(methyl(2,2,2- | | |
| | trifluoroethyl)amino)pyridin-2- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 40^{(c)} | 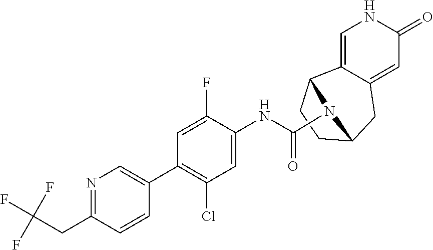 [SMILES: O=C(Nc1cc(Cl)c(-c2ccc(CC(F)(F)F)nc2)cc1F)N1[C@H]2CC[C@@H]1c1c[nH]c(=O)cc1C2] | Method 1 507.6, 509.4 at 1.22 min | ¹H NMR (400 MHz, DMSO- d6) δ 11.27 (s, 1H), 8.67 (s, 1H), 8.64 (dd, J = 2.4, 0.8 Hz, 1H), 7.94 (dd, J = 8.1, 2.4 Hz, 1H), 7.82 (d, J = 7.3 Hz, 1H), 7.56 (d, J = 8.1 Hz, 1H), 7.46 (d, J = 11.1 Hz, 1H), 7.21 (s, 1H), 6.11 (s, 1H), 5.07 (d, J = 5.8 Hz, 1H), 4.60 (t, J = 6.3 Hz, 1H), 3.88 (q, J = 11.5 Hz, 2H), 3.15 (dd, J = 17.9, 5.0 Hz, 1H), 2.56 (d, J = 17.8 Hz, 1H), 2.25-2.02 (m, 2H), 1.78- 1.60 (m, 2H). |
| | (6S,9R)-N-(5-Chloro-2-fluoro- | | |
| | 4-(6-(2,2,2- | | |
| | trifluoroethyl)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 41^{(d)} | 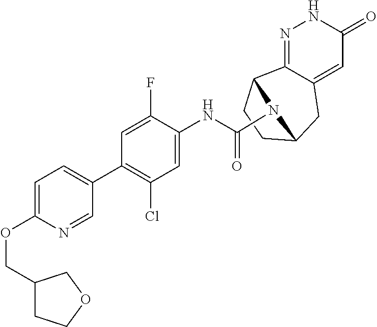 [SMILES: O=C(Nc1cc(Cl)c(-c2ccc(OCC3CCOC3)nc2)cc1F)N1[C@H]2CC[C@@H]1c1n[nH]c(=O)cc1C2] | Method 1 526.2, 528.4 at 1.14 min | ¹H NMR (400 MHz, DMSO- d6) δ 12.72 (s, 1H), 8.88 (s, 1H), 8.20 (dd, J = 2.5, 0.8 Hz, 1H), 7.81 (dd, J = 8.6, 2.5 Hz, 1H), 7.76 (d, J = 7.3 Hz, 1H), 7.37 (d, J = 11.1 Hz, 1H), 6.90 (dd, J = 8.6, 0.8 Hz, 1H), 6.69 (s, 1H), 5.08 (d, J = 5.6 Hz, 1H), 4.64 (t, J = 6.1 Hz, 1H), 4.32-4.15 (m, 2H), 3.83-3.72 (m, 2H), 3.70-3.62 (m, 1H), 3.54 (dd, J = 8.6, 5.5 Hz, 1H), 3.20 (dd, J = 18.2, 5.2 Hz, 1H), 2.76-2.61 (m, 2H), 2.28-2.11 (m, 2H), 2.09- 1.96 (m, 1H), 1.87-1.75 (m, 1H), 1.73-1.60 (m, 2H). |
| | (6S,9R)-N-(5-Chloro-2-fluoro- | | |
| | 4-(6-((tetrahydrofuran-3- | | |
| | yl)methoxy)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 42^{(c)} | 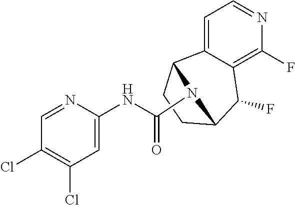 [SMILES: O=C(Nc1cc(Cl)c(Cl)cn1)N1[C@@H]2CC[C@H]1[C@@H](F)c1c2ccnc1F] | Method 1 385.0, 387.0 at 1.51 min | ¹H NMR (400 MHz, DMSO- d6) δ 10.15 (s, 1H), 8.47 (s, 1H), 8.19 (d, J = 5.0 Hz, 1H), 8.10 (s, 1H), 7.33 (dt, J = 5.2, 1.6 Hz, 1H), 6.23 (dd, J = 50.9, 5.8 Hz, 1H), 5.44 (d, J = 6.0 Hz, 1H), 5.05 (q, J = 4.9 Hz, 1H), 2.27-2.05 (m, 3H), 1.86-1.76 (m, 1H). |
| | (5R,8S,9S)-N-(4,5- | | |
| | Dichloropyridin-2-yl)-1,9- | | |
| | difluoro-6,7,8,9-tetrahydro-5H- | | |
| | 5,8- | | |
| | epiminocyclohepta[c]pyridine- | | |
| | 10-carboxamide | | |
| 43^{(c)} | 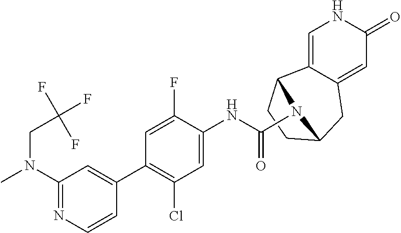 [SMILES: CN(CC(F)(F)F)c1cc(-c2cc(F)c(NC(=O)N3[C@H]4CC[C@@H]3c3c[nH]c(=O)cc3C4)cc2Cl)ccn1] | Method 1 536.0, 538.2 at 1.37 min | ¹H NMR (400 MHz, DMSO- d6) δ 11.28 (s, 1H), 8.66 (s, 1H), 8.18 (d, J = 5.6 Hz, 1H), 7.79 (d, J = 7.3 Hz, 1H), 7.39 (d, J = 11.0 Hz, 1H), 7.21 (s, 1H), 6.78 (dd, J = 3.4, 1.7 Hz, 2H), 6.11 (s, 1H), 5.07 (d, J = 5.8 Hz, 1H), 4.60 (t, J = 6.4 Hz, 1H), 4.52 (q, J = 9.6 Hz, 2H), 3.19-3.13 (m, 1H), 3.11 (s, 3H), 2.63-2.52 (m, 1H), 2.18 (q, J = 10.9 Hz, 1H), 2.09 (q, J = 6.1 Hz, 1H), 1.69 (ddd, J = 22.3, 11.6, 7.2 Hz, 2H). |
| | (6S,9R)-N-(5-Chloro-2-fluoro- | | |
| | 4-(2-(methyl(2,2,2- | | |
| | trifluoroethyl)amino)pyridin-4- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide | | |
| 44^{(d)} | 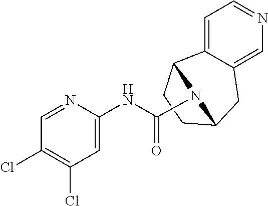 [SMILES: O=C(Nc1cc(Cl)c(Cl)cn1)N1[C@H]2CC[C@@H]1c1ccncc1C2] | Method 1 349.2, 351.0 at 1.55 min | ¹H NMR (400 MHz, DMSO- d6) δ 9.85 (s, 1H), 8.44 (s, 1H), 8.33-8.29 (m, 2H), 8.12 (s, 1H), 7.17 (d, J = 5.0 Hz, 1H), 5.31-5.25 (m, 1H), 4.89-4.82 (m, 1H), 3.33- 3.23 (m, 1H), 2.65 (d, J = 17.0 Hz, 1H), 2.25-2.09 (m, 2H), 1.87-1.75 (m, 1H), 1.73-1.60 (m, 1H). |
| | (5R,8S)-N-(4,5-Dichloropyridin- | | |
| | 2-yl)-6,7,8,9-tetrahydro-5H-5,8- | | |
| | epiminocyclohepta[c]pyridine- | | |
| | 10-carboxamide | | |
| 45^{(d)} | 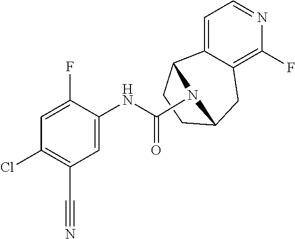 [SMILES: N#Cc1cc(NC(=O)N2[C@H]3CC[C@@H]2c2ccnc(F)c2C3)c(F)cc1Cl] | Method 1 373.0, 375.0^{(f)} at 1.57 min | ¹H NMR (400 MHz, DMSO- d6) δ 8.96 (bs, 1H), 8.12 (d, J = 7.9 Hz, 1H), 8.00 (d, J = 5.0 Hz, 1H), 7.82 (d, J = 10.4 Hz, 1H), 7.20 (dd, J = 5.0, 1.7 Hz, 1H), 5.21 (d, J = 5.9 Hz, 1H), 4.78 (t, J = 6.1 Hz, 1H), 3.19 (dd, J = 17.4, 5.0 Hz, 1H), 2.58 (d, J = 17.3 Hz, 1H), 2.32-2.12 (m, 2H), 1.89-1.81 (m, 1H), 1.79- 1.70 (m, 1H). |
| | (5R,8S)-N-(4-Chloro-5-cyano- | | |
| | 2-fluorophenyl)-1-fluoro- | | |
| | 6,7,8,9-tetrahydro-5H-5,8- | | |
| | epiminocyclohepta[c]pyridine- | | |
| | 10-carboxamide | | |
| 46^{(d)} | 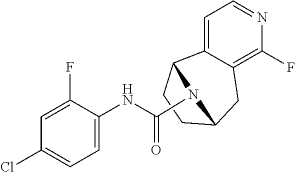 [SMILES: O=C(Nc1ccc(Cl)cc1F)N1[C@H]2CC[C@@H]1c1ccnc(F)c1C2] | Method 2 350.3, 352.2 at 1.75 min | ¹H NMR (400 MHz, DMSO- d6) δ 8.73 (s, 1H), 8.00 (d, J = 5.0 Hz, 1H), 7.59 (dd, J = 6.9, 2.7 Hz, 1H), 7.28-7.10 (m, 3H), 5.20 (d, J = 6.0 Hz, 1H), 4.77 (t, J = 6.2 Hz, 1H), 3.18 (dd, J = 17.3, 5.0 Hz, 1H), 2.57 (d, J = 17.4 Hz, 1H), 2.31-2.12 (m, 2H), 1.90-1.80 (m, 1H), 1.80- 1.69 (m, 1H). |
| | (5R,8S)-N-(4-Chloro-2- | | |
| | fluorophenyl)-1-fluoro-6,7,8,9- | | |
| | tetrahydro-5H-5,8- | | |
| | epiminocyclohepta[c]pyridine- | | |
| | 10-carboxamide | | |
| 47^{(d)} | 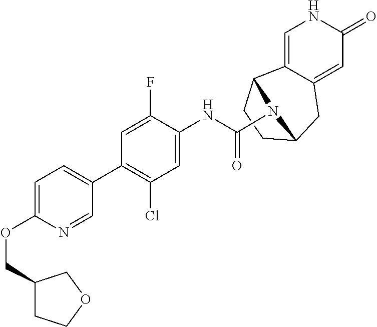 [SMILES: O=C(Nc1cc(Cl)c(-c2ccc(OC[C@@H]3CCOC3)nc2)cc1F)N1[C@H]2CC[C@@H]1c1c[nH]c(=O)cc1C2] | Method 1 525.2, 527.2 at 1.43 min | ¹H NMR (400 MHz, DMSO- d6) δ 11.27 (s, 1H), 8.63 (s, 1H), 8.20 (dd, J = 2.5, 0.7 Hz, 1H), 7.80 (dd, J = 8.6, 2.5 Hz, 1H), 7.77 (d, J = 7.4 Hz, 1H), 7.37 (d, J = 11.1 Hz, 1H), 7.20 (s, 1H), 6.90 (dd, J = 8.6, 0.8 Hz, 1H), 6.11 (s, 1H), 5.06 (d, J = 5.8 Hz, 1H), 4.59 (t, J = 6.4 Hz, 1H), 4.28 (dd, J = 10.5, 6.7 Hz, 1H), 4.20 (dd, J = 10.5, 8.0 Hz, 1H), 3.83-3.72 (m, 2H), 3.71-3.61 (m, 1H), 3.54 (dd, J = 8.7, 5.5 Hz, 1H), 3.15 (dd, J = 18.7, 5.0 Hz, 1H), 2.74-2.63 (m, 1H), 2.56 (d, J = 17.9 Hz, 1H), 2.24-1.96 (m, 3H), 1.77- 1.61 (m, 3H). |
| | (6S,9R)-N-(5-Chloro-2-fluoro- | | |
| | 4-(6-(((tetrahydrofuran-3- | | |
| | yl)methoxy)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide Isomer 1 | | |
| 48^{(d)} | 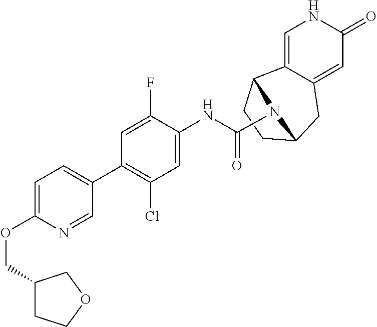 [SMILES: O=C(Nc1cc(Cl)c(-c2ccc(OC[C@H]3CCOC3)nc2)cc1F)N1[C@H]2CC[C@@H]1c1c[nH]c(=O)cc1C2] | Method 1 525.0, 527.0 at 1.43 min | ¹H NMR (400 MHz, DMSO- d6) δ 11.27 (s, 1H), 8.63 (s, 1H), 8.20 (dd, J = 2.5, 0.8 Hz, 1H), 7.80 (dd, J = 8.6, 2.5 Hz, 1H), 7.77 (d, J = 7.4 Hz, 1H), 7.37 (d, J = 11.1 Hz, 1H), 7.20 (s, 1H), 6.90 (dd, J = 8.6, 0.8 Hz, 1H), 6.11 (s, 1H), 5.06 (d, J = 5.8 Hz, 1H), 4.59 (t, J = 6.4 Hz, 1H), 4.28 (dd, J = 10.5, 6.7 Hz, 1H), 4.20 (dd, J = 10.5, 7.9 Hz, 1H), 3.83-3.72 (m, 2H), 3.71-3.61 (m, 1H), 3.54 (dd, J = 8.6, 5.5 Hz, 1H), 3.15 (dd, J = 18.6, 4.9 Hz, 1H), 2.74-2.63 (m, 1H), 2.56 (d, J = 17.9 Hz, 1H), 2.25-1.95 (m, 3H), 1.77- 1.60 (m, 3H). |
| | (6S,9R)-N-(5-Chloro-2-fluoro- | | |
| | 4-(6-(((tetrahydrofuran-3- | | |
| | yl)methoxy)pyridin-3- | | |
| | yl)phenyl)-3-oxo-3,5,6,7,8,9- | | |
| | hexahydro-2H-6,9- | | |
| | epiminocyclohepta[c]pyridine- | | |
| | 10-carboxamide Isomer 2 | | |
| 49^{(d)} | 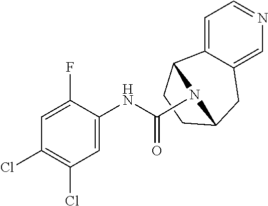 [SMILES: O=C(Nc1cc(Cl)c(Cl)cc1F)N1[C@H]2CC[C@@H]1c1ccncc1C2] | Method 1 366.0, 368.0 at 1.61 min | ¹H NMR (400 MHz, DMSO- d6) δ 8.76 (s, 1H), 8.34- 8.28 (m, 2H), 7.81 (d, J = 7.6 Hz, 1H), 7.66 (d, J = 10.3 Hz, 1H), 7.16 (d, J = 4.9 Hz, 1H), 5.13 (d, J = 6.0 Hz, 1H), 4.74 (t, J = 6.1 Hz, 1H), 3.31 (dd, J = 17.3, 4.7 Hz, 1H), 2.66 (d, J = 17.0 Hz, 1H), 2.30- 2.10 (m, 2H), 1.84-1.75 (m, 1H), 1.74-1.63 (m, 1H). |
| | (5R,8S)-N-(4,5-Dichloro-2- | | |
| | fluorophenyl)-6,7,8,9- | | |
| | tetrahydro-5H-5,8- | | |
| | epiminocyclohepta[c]pyridine- | | |
| | 10-carboxamide | | |
| 50^{(d)} | 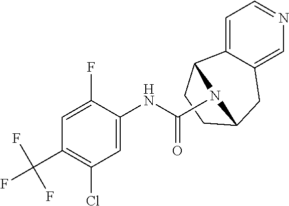 [SMILES: O=C(Nc1cc(Cl)c(C(F)(F)F)cc1F)N1[C@H]2CC[C@@H]1c1ccncc1C2] | Method 1 400.2, 402.0 at 1.75 min | ¹H NMR (400 MHz, DMSO- d6) δ 9.02 (s, 1H), 8.31 (d, J = 5.1 Hz, 2H), 8.01 (d, J = 6.9 Hz, 1H), 7.78 (d, J = 11.0 Hz, 1H), 7.18 (d, J = 4.9 Hz, 1H), 5.17 (d, J = 5.9 Hz, 1H), 4.79 (t, J = 6.0 Hz, 1H), 3.32 (dd, J = 17.0, 5.0 Hz, 1H), 2.68 (d, J = 17.0 Hz, 1H), 2.31- 2.11 (m, 2H), 1.86-1.76 (m, 1H), 1.74-1.65 (m, 1H). |
| | (5R,8S)-N-(5-Chloro-2-fluoro- | | |
| | 4-(trifluoromethyl)phenyl)- | | |
| | 6,7,8,9-tetrahydro-5H-5,8- | | |
| | epiminocyclohepta[c]pyridine- | | |
| | 10-carboxamide | | |
| 53^{(c)} | 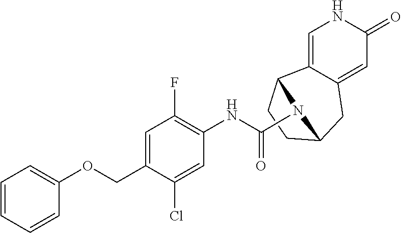 [SMILES: O=C(Nc1cc(Cl)c(COc2ccccc2)cc1F)N1[C@H]2CC[C@@H]1c1c[nH]c(=O)cc1C2] | Method 3 454.2 at 1.41 min | ¹H NMR (400 MHz, DMSO- d6) δ 12.25 (s, 1H), 8.57 (s, 1H), 7.72 (d, J = 7.2 Hz, 1H), 7.44 (d, J = 11.2 Hz, 1H), 7.33-7.29 (m, 2H), 7.19 (s, 1H), 7.02-6.95 (m, 3H), 6.10 (s, 1H), 5.08-5.04 (m, 3H), 4.58 (t, J = 6.4 Hz, 1H), 3.16-3.10 (m, 1H), 2.55 (d, J = 18 Hz, 1H), 2.12-2.03 (m, 2H), 1.74-1.61 (m, 2H). |
| | (6S,9R)-N-(5-Chloro-2-fluoro- | | |
| | 4-(phenoxymethyl)phenyl)-3- | | |
| | oxo-3,5,6,7,8,9-hexahydro-2H- | | |
| | 6,9- | | |
| | epiminocyclohepta[c]pyridine- | | |
| | 10-carboxamide | | |
| 70^{(e)} | 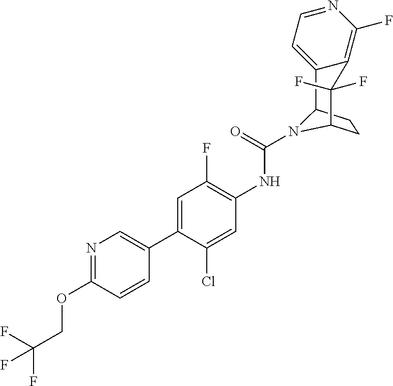 [SMILES: O=C(Nc1cc(Cl)c(-c2ccc(OCC(F)(F)F)nc2)cc1F)N1C2CCC1C(F)(F)c1c2ccnc1F] | Method 3 560.9 at 2.81 min | ¹H NMR (400 MHz, DMSO- d6) δ 9.14 (s, 1H), 8.38 (d, J = 5.2 Hz, 1H), 8.27 (d, J = 2.4 Hz, 1H), 7.93 (dd, J = 8.8, 2.8 Hz, 1H), 7.76 (d, J = 7.2 Hz, 1H), 7.50 (d, J = 5.2 Hz, 1H), 7.43 (d, J = 11.2 Hz, 1H), 7.08 (d, J = 8.4 Hz, 1H), 5.46 (d, J = 6.4 Hz, 1H), 5.27- 5.22 (m, 1H), 5.05 (q, J = 9.2 Hz, 2H), 2.38-2.29 (m, 1H), 2.24-2.14 (m, 1H), 1.93-1.88 (m, 1H), 1.75- 1.69 (m, 1H). |
| | (+)-N-(5-Chloro-2-fluoro-4-(6- | | |
| | (2,2,2-trifluoroethoxy)pyridin-3- | | |
| | yl)phenyl)-1,9,9-trifluoro- | | |
| | 6,7,8,9-tetrahydro-5H-5,8- | | |
| | epiminocyclohepta[c]pyridine- | | |
| | 10-carboxamide | | |
| 79^{(d)} | 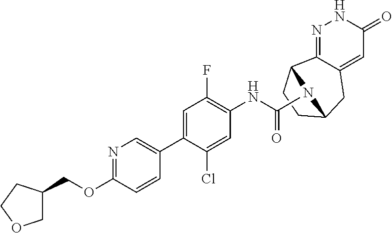 [SMILES: O=C(Nc1cc(Cl)c(-c2ccc(OC[C@@H]3CCOC3)nc2)cc1F)N1[C@H]2CC[C@@H]1c1n[nH]c(=O)cc1C2] | Method 8 526.1 at 1.38 min | ¹H NMR (500 MHz, DMSO- d6) δ 12.73 (s, 1H), 8.88 (s, 1H), 8.21 (d, J = 2.5 Hz, 1H), 7.81 (dd, J = 8.6, 2.6 Hz, 1H), 7.77 (d, J = 7.3 Hz, 1H), 7.38 (d, J = 11.1 Hz, 1H), 6.91 (d, J = 8.6 Hz, 1H), 6.70 (s, 1H), 5.08 (d, J = 5.8 Hz, 1H), 4.65 (d, J = 6.3 Hz, 1H), 4.29 (dd, J = 10.5, 6.7 Hz, 1H), 4.21 (dd, J = 10.5, 8.0 Hz, 1H), 3.78 (ddd, J = 13.3, 8.3, 6.4 Hz, 2H), 3.67 (q, J = 7.7 Hz, 1H), 3.55 (dd, J = 8.7, 5.5 Hz, 1H), 3.20 (dd, J = |
| | (6S,9R)-N-(5-Chloro-2-fluoro- | | 8.3, 5.3 Hz, 1H), 2.75- |
| | 4-(6-(((R)-tetrahydrofuran-3- | | 2.61 (m, 2H), 2.28-2.14 (m, |
| | yl)methoxy)pyridin-3- | | 2H), 2.03 (dtd, J = 13.7, 8.1, |
| | yl)phenyl)-3-oxo-3,5,6,7,8,9- | | 5.7 Hz, 1H), 1.85-1.77 (m, |
| | hexahydro-2H-6,9- | | 1H), 1.68 (dt, J = 11.8, 6.9 |
| | epiminocyclohepta[c]pyridazine- | | Hz, 2H). |
| | 10-carboxamide | | |
| 80^{(d)} | 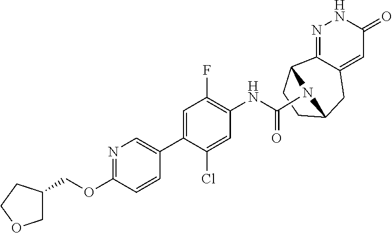 [SMILES: O=C(Nc1cc(Cl)c(-c2ccc(OC[C@H]3CCOC3)nc2)cc1F)N1[C@H]2CC[C@@H]1c1n[nH]c(=O)cc1C2] | Method 8 526.1 at 1.41 min | ¹H NMR (500 MHz, DMSO- d6) δ 12.73 (s, 1H), 8.88 (s, 1H), 8.21 (d, J = 2.6 Hz, 1H), 7.81 (dd, J = 8.6, 2.6 Hz, 1H), 7.77 (d, J = 7.3 Hz, 1H), 7.38 (d, J = 11.1 Hz, 1H), 6.91 (d, J = 8.6 Hz, 1H), 6.70 (s, 1H), 5.08 (d, J = 5.7 Hz, 1H), 4.65 (d, J = 6.3 Hz, 1H), 4.29 (dd, J = 10.5, 6.7 Hz, 1H), 4.21 (dd, J = 10.5, 8.0 Hz, 1H), 3.78 (ddd, J = 13.4, 8.4, 6.4 Hz, 2H), 3.67 (q, J = 7.6 Hz, 1H), 3.55 (dd, J = 8.6, 5.5 Hz, 1H), 3.20 (dd, J = |
| | (6S,9R)-N-(5-Chloro-2-fluoro- | | 18.3, 5.2 Hz, 1H), 2.78- |
| | 4-(6-(((S)-tetrahydrofuran-3- | | 2.57 (m, 2H), 2.29-2.09 (m, |
| | yl)methoxy)pyridin-3- | | 2H), 2.03 (dtd, J = 13.5, 8.2, |
| | yl)phenyl)-3-oxo-3,5,6,7,8,9- | | 5.6 Hz, 1H), 1.81 (t, J = 9.7 |
| | hexahydro-2H-6,9- | | Hz, 1H), 1.67 (dq, J = 13.2, |
| | epiminocyclohepta[c]pyridazine- | | 6.7 Hz, 2H). |
| | 10-carboxamide | | |
| 81^{(d)} | 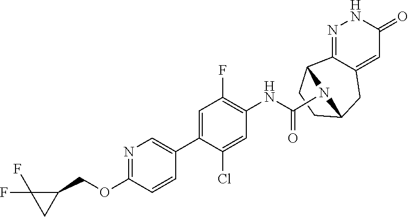 [SMILES: O=C(Nc1cc(Cl)c(-c2ccc(OC[C@H]3CC3(F)F)nc2)cc1F)N1[C@H]2CC[C@@H]1c1n[nH]c(=O)cc1C2] | Method 8 532.1, 534.0 at 1.54 min | ¹H NMR (500 MHz, DMSO- d6) δ 12.72 (s, 1H), 8.88 (s, 1H), 8.22 (d, J = 2.5 Hz, 1H), 7.83 (dd, J = 8.6, 2.5 Hz, 1H), 7.76 (d, J = 7.3 Hz, 1H), 7.38 (d, J = 11.1 Hz, 1H), 6.95 (d, J = 8.6 Hz, 1H), 6.69 (s, 1H), 5.08 (d, J = 5.8 Hz, 1H), 4.65 (d, J = 6.4 Hz, 1H), 4.56-4.46 (m, 1H), 4.29- 4.18 (m, 1H), 3.20 (dd, J = 18.3, 5.2 Hz, 1H), 2.65 (d, J = 18.2 Hz, 1H), 2.34-2.12 (m, 3H), 1.86-1.77 (m, 1H), |
| | (6S,9R)-N-(5-Chloro-4-(6-(((R)- | | 1.77-1.61 (m, 2H), 1.59- |
| | 2,2-difluorocyclopropyl)methoxy)py | | 1.46 (m, 1H). |
| | ridin-3-yl)-2-fluorophenyl)-3- | | |
| | oxo-3,5,6,7,8,9-hexahydro-2H- | | |
| | 6,9-epiminocyclohepta[c]pyridazine- | | |
| | 10-carboxamide | | |
| 82^{(d)} | 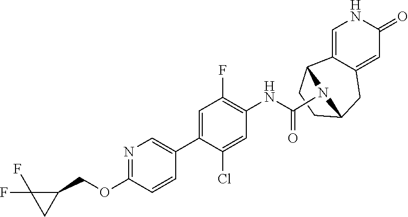 [SMILES: O=C(Nc1cc(Cl)c(-c2ccc(OC[C@H]3CC3(F)F)nc2)cc1F)N1[C@H]2CC[C@@H]1c1c[nH]c(=O)cc1C2] | Method 8 531.0, 533.0 at 1.54 min | ¹H NMR (500 MHz, DMSO- d6) δ 11.27 (s, 1H), 8.63 (s, 1H), 8.22 (d, J = 2.5 Hz, 1H), 7.83 (dd, J = 8.6, 2.5 Hz, 1H), 7.77 (d, J = 7.3 Hz, 1H), 7.37 (d, J = 11.1 Hz, 1H), 7.20 (s, 1H), 6.95 (d, J = 8.6 Hz, 1H), 6.11 (s, 1H), 5.06 (d, J = 6.0 Hz, 1H), 4.63- 4.56 (m, 1H), 4.54-4.46 (m, 1H), 4.28-4.20 (m, 1H), 3.15 (dd, J = 17.8, 5.0 Hz, 1H), 2.56 (d, J = 17.9 Hz, 1H), 2.33-2.21 (m, 1H), |
| | (6S,9R)-N-(5-Chloro-4-(6-(((R)- | | 2.17 (t, J = 10.5 Hz, 1H), |
| | 2,2-difluorocyclopropyl)methoxy)py | | 2.13-2.03 (m, 1H), 1.77- |
| | ridin-3-yl)-2-fluorophenyl)-3- | | 1.60 (m, 3H), 1.57-1.47 (m, |
| | oxo-3,5,6,7,8,9-hexahydro-2H- | | 1H). |
| | 6,9-epiminocyclohepta[c]pyridine- | | |
| | 10-carboxamide | | |
| 83^{(d)} | 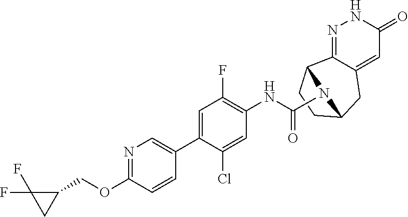 [SMILES: O=C(Nc1cc(Cl)c(-c2ccc(OC[C@@H]3CC3(F)F)nc2)cc1F)N1[C@H]2CC[C@@H]1c1n[nH]c(=O)cc1C2] | Method 8 532.0, 534.0 at 1.58 min | ¹H NMR (500 MHz, DMSO- d6) δ 12.72 (s, 1H), 8.88 (s, 1H), 8.22 (d, J = 2.5 Hz, 1H), 7.83 (dd, J = 8.6, 2.5 Hz, 1H), 7.76 (d, J = 7.3 Hz, 1H), 7.38 (d, J = 11.0 Hz, 1H), 6.95 (d, J = 8.6 Hz, 1H), 6.69 (s, 1H), 5.08 (d, J = 5.8 Hz, 1H), 4.65 (d, J = 6.4 Hz, 1H), 4.50 (ddd, J = 10.2, 6.8, 2.9 Hz, 1H), 4.24 (ddd, J = 11.4, 8.4, 1.7 Hz, 1H), 3.20 (dd, J = 18.2, 5.2 Hz, 1H), 2.65 (d, J = 18.1 Hz, 1H), 2.33-2.22 |
| | (6S,9R)-N-(5-Chloro-4-(6-(((S)- | | (m, 1H), 2.22-2.13 (m, 2H), |
| | 2,2- | | 1.86-1.68 (m, 3H), 1.57- |
| | difluorocyclopropyl)methoxy) | | 1.47 (m, 1H). |
| | pyridin-3-yl)-2-fluorophenyl)-3- | | |
| | ox0-3,5,6,7,8,9-hexahydro-2H- | | |
| | 6,9- | | |
| | epiminocyclohepta[c]pyridazine- | | |
| | 10-carboxamide | | |
| 84 | 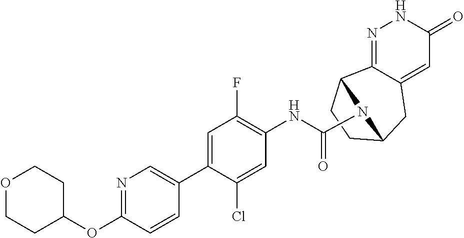 [SMILES: O=C(Nc1cc(Cl)c(-c2ccc(OC3CCOCC3)nc2)cc1F)N1[C@H]2CC[C@@H]1c1n[nH]c(=O)cc1C2] | Method 8 526.1 at 1.44 min | ¹H NMR (500 MHz, DMSO- d6) δ 12.76 (s, 1H), 8.91 (s, 1H), 8.21 (d, J = 2.6 Hz, 1H), 7.81 (dd, J = 8.6, 2.6 Hz, 1H), 7.76 (d, J = 7.3 Hz, 1H), 7.38 (d, J = 11.1 Hz, 1H), 6.89 (d, J = 8.6 Hz, 1H), 6.70 (s, 1H), 5.23 (tt, J = 8.9, 4.2 Hz, 1H), 5.08 (d, J = 5.7 Hz, 1H), 4.64 (t, J = 6.1 Hz, 1H), 3.88 (dt, J = 11.7, 4.3 Hz, 2H), 3.50 (ddd, J = 12.0, 9.9, 2.7 Hz, 2H), 3.20 (dd, J = 18.3, 5.2 Hz, 1H), 2.66 (d, J = 18.2 Hz, 1H), 2.20 (td, J = 12.9, 8.3 Hz, 2H), 2.03 (dq, J = |
| | (6S,9R)-N-(5-Chloro-2-fluoro- | | 12.5, 4.0 Hz, 2H), 1.81 (dd, |
| | 4-(6-((tetrahydro-2H-pyran-4- | | J = 11.1, 8.5 Hz, 1H), 1.66 |
| | yl)oxy)pyridin-3-yl)phenyl)-3- | | (dtd, J = 17.2, 10.5, 4.6 Hz, |
| | oxo-3,5,6,7,8,9-hexahydro-2H- | | 3H). |
| | 6,9- | | |
| | epiminocyclohepta[c]pyridazine- | | |
| | 10-carboxamide | | |
| 85^{(d)} | 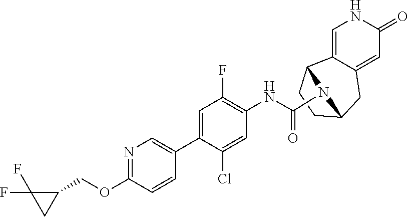 [SMILES: O=C(Nc1cc(Cl)c(-c2ccc(OC[C@@H]3CC3(F)F)nc2)cc1F)N1[C@H]2CC[C@@H]1c1c[nH]c(=O)cc1C2] | Method 8 531.1, 533.0 at 1.55 min | ¹H NMR (500 MHz, DMSO- d6) δ 12.76 (s, 1H), 8.91 (s, 1H), 8.21 (d, J = 2.6 Hz, 1H), 7.81 (dd, J = 8.6, 2.6 Hz, 1H), 7.76 (d, J = 7.3 Hz, 1H), 7.38 (d, J = 11.1 Hz, 1H), 6.89 (d, J = 8.6 Hz, 1H), 6.70 (s, 1H), 5.23 (tt, J = 8.9, 4.2 Hz, 1H), 5.08 (d, J = 5.7 Hz, 1H), 4.64 (t, J = 6.1 Hz, 1H), 3.88 (dt, J = 11.7, 4.3 Hz, 2H), 3.50 (ddd, J = 12.0, 9.9, 2.7 Hz, 2H), 3.20 (dd, J = 18.3, 5.2 Hz, 1H), 2.66 (d, J = |
| | (6S,9R)-N-(5-Chloro-4-(6-(((S)- | | 18.2 Hz, 1H), 2.20 (td, J = |
| | 2,2- | | 12.9, 8.3 Hz, 2H), 2.03 (dq, J = |
| | difluorocyclopropyl)methoxy) | | 12.5, 4.0 Hz, 2H), 1.81 (dd, |
| | pyridin-3-yl)-2-fluorophenyl)-3- | | J = 11.1, 8.5 Hz, 1H), 1.66 |
| | oxo-3,5,6,7,8,9-hexahydro-2H- | | (dtd, J = 17.2, 10.5, 4.6 Hz, |
| | 6,9- | | 3H). |
| | epiminocyclohepta[c]pyridine- | | |
| | 10-carboxamide | | |
| 86^{(c)} | 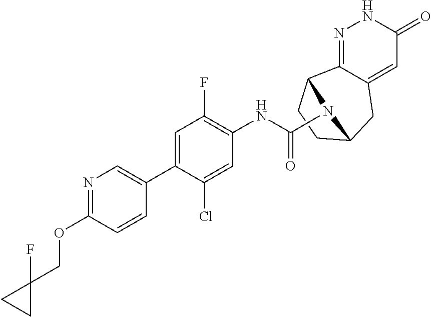 [SMILES: O=C(Nc1cc(Cl)c(-c2ccc(OCC3(F)CC3)nc2)cc1F)N1[C@H]2CC[C@@H]1c1n[nH]c(=O)cc1C2] | Method 8 514.2 at 1.41 min | ¹H NMR (500 MHz, DMSO- d6) δ 12.73 (s, 1H), 8.89 (s, 1H), 8.21 (d, J = 2.6 Hz, 1H), 7.84 (dd, J = 8.6, 2.6 Hz, 1H), 7.77 (d, J = 7.3 Hz, 1H), 7.39 (d, J = 11.0 Hz, 1H), 7.00 (d, J = 8.6 Hz, 1H), 6.70 (s, 1H), 5.09 (d, J = 5.7 Hz, 1H), 4.65 (s, 2H), 4.60 (s, 1H), 2.66 (d, J = 18.2 Hz, 1H), 2.29-2.12 (m, 2H), 1.86-1.76 (m, 1H), 1.69 (dt, J = 9.5, 4.9 Hz, 1H), 1.13 (dd, J = 18.5, 6.7 Hz, 2H), 0.91 (td, J = 8.2, 6.1 Hz, 2H). (1 CH not observed) |
| | (6S,9R)-N-(5-Chloro-2-fluoro- | | |
| | 4-(6-((1- | | |
| | fluorocyclopropyl)methoxy) | | |
| | pyridin-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 87(c) | 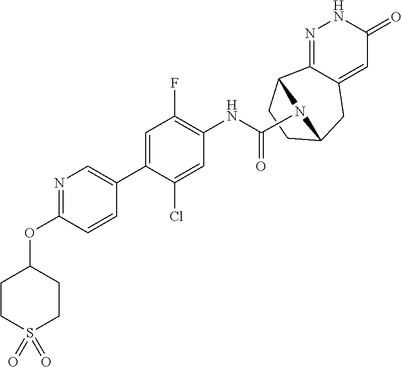 [SMILES: O=C(Nc1cc(Cl)c(-c2ccc(OC3CCS(=O)(=O)CC3)nc2)cc1F)N1[C@H]2CC[C@@H]1c1n[nH]c(=O)cc1C2] | Method 2 574.3, 576.4 at 1.17 min | ¹H NMR (500 MHz, DMSO- d6) δ 12.73 (d, J = 2.1 Hz, 1H), 8.89 (s, 1H), 8.23 (d, J = 2.6 Hz, 1H), 7.85 (dd, J = 8.6, 2.6 Hz, 1H), 7.77 (d, J = 7.3 Hz, 1H), 7.38 (d, J = 11.0 Hz, 1H), 6.97 (d, J = 8.5 Hz, 1H), 6.70 (s, 1H), 5.37 (tt, J = 6.8, 3.3 Hz, 1H), 5.08 (d, J = 5.7 Hz, 1H), 4.65 (t, J = 6.2 Hz, 1H), 3.28-3.15 (m, 5H), 2.66 (d, J = 18.2 Hz, 1H), 2.39-2.13 (m, 6H), 1.84- 1.77 (m, 1H), 1.70 (d, J = 13.1 Hz, 1H) |
| | 5 (6S,9R)-N-(5-Chloro-4-(6-((1,1- | | |
| | dioxidotetrahydro-2H- | | |
| | thiopyran-4-yl)oxy)pyridine-3- | | |
| | yl)-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 88^{(c)} | 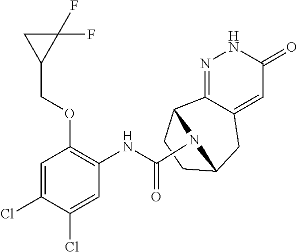 [SMILES: O=C(Nc1cc(Cl)c(Cl)cc1OCC1CC1(F)F)N1[C@H]2CC[C@@H]1c1n[nH]c(=O)cc1C2] | Method 2 471.2, 473.2 at 1.43 min | ¹H NMR (500 MHz, DMSO- d6) δ 12.72 (s, 1H), 8.15 (d, J = 4.0 Hz, 1H), 7.90-7.80 (m, 1H), 7.35 (d, J = 6.7 Hz, 1H), 6.66 (s, 1H), 4.99 (d, J = 5.9 Hz, 1H), 4.60 (p, J = 4.7 Hz, 1H), 4.26-4.04 (m, 2H), 3.25-3.12 (m, 1H), 2.65 (d, J = 18.0 Hz, 1H), 2.30-2.11 (m, 3H), 1.88-1.59 (m, 3H), 1.59-1.38 (m, 1H). |
| | (6S,9R)-N-(4,5-Dichloro-2- | | |
| | ((2,2- | | |
| | difluorocyclopropyl)methoxy) | | |
| | phenyl)-3-oxo-3,5,6,7,8,9- | | |
| | hexahydro-2H-6,9- | | |
| | epiminocyclohepta[c]pyridazine- | | |
| | 10-carboxamide | | |
| 89^{(c)} | 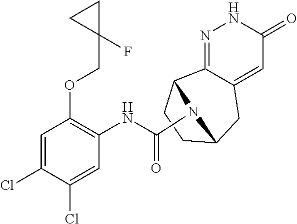 [SMILES: O=C(Nc1cc(Cl)c(Cl)cc1OCC1(F)CC1)N1[C@H]2CC[C@@H]1c1n[nH]c(=O)cc1C2] | Method 2 453.2, 455.2 at 1.40 min | 1H NMR (400 MHz, DMSO- d6) δ 12.70 (d, J = 2.0 Hz, 1H), 8.12 (s, 1H), 7.81 (s, 1H), 7.35 (s, 1H), 6.65 (d, J = 2.3 Hz, 1H), 4.95 (d, J = 5.9 Hz, 1H), 4.57 (t, J = 6.0 Hz, 1H), 4.37 (dd, J = 22.5, 1.7 Hz, 2H), 3.19 (dd, J = 18.4, 5.2 Hz, 1H), 2.65 (d, J = 18.1 Hz, 1H), 2.29-2.12 (m, 2H), 1.85-1.74 (m, 1H), 1.68 (t, J = 8.4 Hz, 1H), 1.17-1.01 (m, 2H), 0.91-0.77 (m, 2H). |
| | (6S,9R)-N-(4,5-Dichloro-2-((1- | | |
| | fluorocyclopropyl)methoxy) | | |
| | phenyl)-3-oxo-3,5,6,7,8,9- | | |
| | hexahydro-2H-6,9- | | |
| | epiminocyclohepta[c]pyridazine- | | |
| 91^{(d)} | 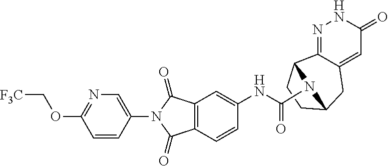 [SMILES: O=C1c2ccc(NC(=O)N3[C@H]4CC[C@@H]3c3n[nH]c(=O)cc3C4)cc2C(=O)N1c1ccc(OCC(F)(F)F)nc1] | Method 8 540.6 at 1.50 min | ¹H NMR (400 MHz, DMSO- d6) δ 12.75 (d, J = 2.0 Hz, 1H), 9.47 (s, 1H), 8.28 (dd, J = 2.6, 0.7 Hz, 1H), 8.18 (t, J = 1.8 Hz, 1H), 7.95-7.88 (m, 2H), 7.86 (d, J = 8.3 Hz, 1H), 7.16 (dd, J = 8.8, 0.7 Hz, 1H), 6.69 (s, 1H), 5.19 (d, J = 5.8 Hz, 1H), 5.05 (q, J = 9.1 Hz, 2H), 4.72 (t, J = 6.3 Hz, 1H), 3.19 (dd, J = 18.3, |
| | (6S,9R)-N-(1,3-Dioxo-2-(6- | | 5.3 Hz, 1H), 2.69 (d, J = 18.2 |
| | (2,2,2-trifluoroethoxy)pyridin-3- | | Hz, 1H), 2.30-2.13 (m, 2H), |
| | yl)isoindolin-5-yl)-3-oxo- | | 1.88-1.77 (m, 1H), 1.70 (t, |
| | 3,5,6,7,8,9-hexahydro-2H-6,9- | | J = 8.8 Hz, 1H). |
| | epiminocyclohepta[c]pyridazine- | | |
| | 10-carboxamide | | |
| 92^{(d)} | 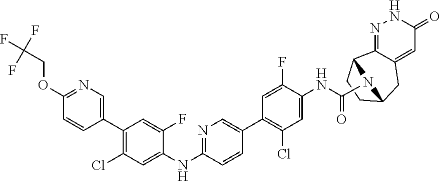 [SMILES: O=C(Nc1cc(Cl)c(-c2ccc(Nc3cc(Cl)c(-c4ccc(OCC(F)(F)F)nc4)cc3F)nc2)cc1F)N1[C@H]2CC[C@@H]1c1n[nH]c(=O)cc1C2] | Method 2 744.0, 746.2, 748.7 at 2.27 min | ¹H NMR (400 MHz, DMSO- d6) δ 12.72 (d, J = 2.3 Hz, 1H), 9.27 (d, J = 2.1 Hz, 1H), 8.87 (s, 1H), 8.71 (d, J = 7.8 Hz, 1H), 8.33-8.27 (m, 2H), 7.95 (dd, J = 8.6, 2.5 Hz, 1H), 7.81-7.73 (m, 2H), 7.45 (d, J = 11.9 Hz, 1H), 7.40 (d, J = 11.1 Hz, 1H), 7.21 (d, J = 8.7 Hz, 1H), 7.09 (d, J = 8.3 Hz, 1H), 6.69 (s, 1H), 5.11-5.00 (m, 3H), |
| | (6S,9R)-N-(5-Chloro-4-(6-((5- | | 4.64 (t, J = 5.7 Hz, 1H), 3.20 |
| | chloro-2-fluoro-4-(6-(2,2,2- | | (dd, J = 18.8, 4.0 Hz, 1H), |
| | trifluoroethoxy)pyridin-3- | | 2.65 (d, J = 18.2 Hz, 1H), |
| | yl)phenyl)amino)pyridin-3-yl)-2- | | 2.29-2.12 (m, 2H), 1.85- |
| | fluorophenyl)-3-oxo- | | 1.76 (m, 1H), 1.73-1.61 (m, |
| | 3,5,6,7,8,9-hexahydro-2H-6,9- | | 1H). = 24 H |
| | epiminocyclohepta[c]pyridazine- | | |
| | 10-carboxamide | | |

Key: ^{(a)}was purified by prep-TLC (MeOH/DCM),
^{(b)}Et₃N and DMAP were added to reactuion mixture,
^{(c)}was purified by prep HPLC,
^{(d)} was purified by chromatography on silica gel (MeOH/DCM),
(e)was purified by prep-TLC (EtOAc/petroleum ether),
^{(f)}no mass ion observed in ES⁺, ES⁻ ions reported instead.

### Intermediate 1 (1-1) route 1

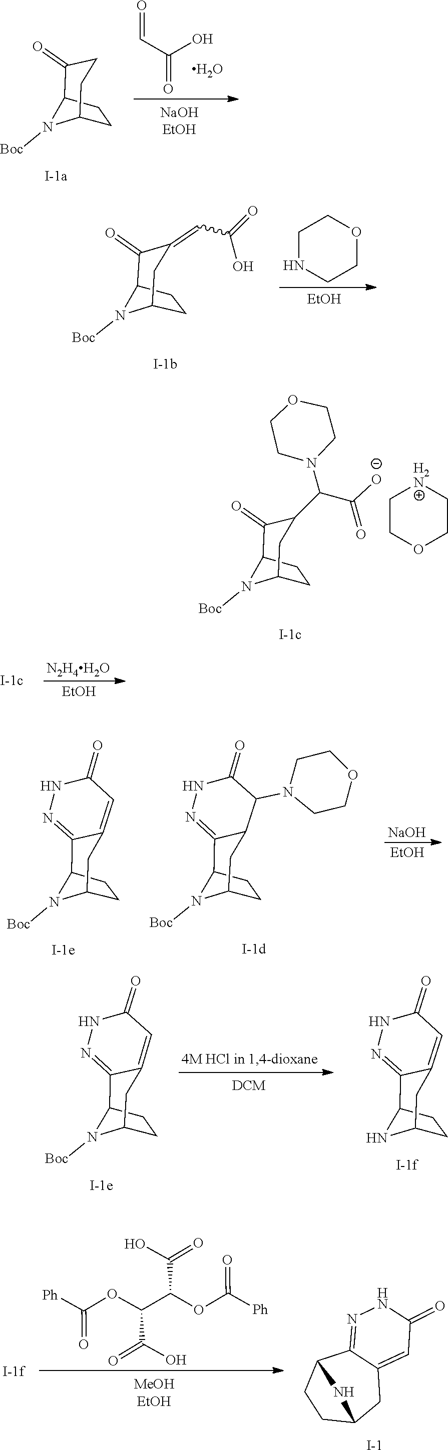

Step 1: To a solution of tert-butyl (±)-2-oxo-8-azabicyclo[3.2.1]octane-8-carboxylate I-1a (2.00 g, 8.88 mmol) and glyoxylic acid monohydrate (1.14 g, 12.4 mmol) in EtOH (20 ml) was added an aqueous 2 M sodium hydroxide solution (7.10 ml, 14.2 mmol). The resultant mixture was stirred at RT for 1 h and the reaction mixture was concentrated in vacuo. The aqueous was washed once with DCM (5 ml) before 1 M aq. HCl was carefully added to the remaining aqueous solution until ~pH 6 was reached. The product was extracted with DCM (100 ml) and the aqueous layer was further extracted with 10% MeOH/DCM (3×50 ml) and the combined organic layers were dried over MgSO₄, filtered and concentrated to provide 2-((±)-8-(tert-butoxycarbonyl)-2-oxo-8-azabicyclo[3.2.1]octa n-3-ylidene)acetic acid I-1b as an off white solid. ¹H NMR (400 MHz, DMSO-d6) δ 6.64-6.54 (m, 1H), 5.75 (s, 1H), 4.35 (d, J=8.1 Hz, 1H), 3.32 (s, 1H), 3.06 (t, J=2.6 Hz, 1H), 2.32-2.16 (m, 1H), 2.16-1.96 (m, 1H), 1.79-1.51 (m, 2H), 1.37 (s, 9H). (Exchangeable —OH not observed)

Step 2: To a solution of 2-((±)-8-(tert-butoxycarbonyl)-2-oxo-8-azabicyclo[3.2.1]octan-3-ylidene)acetic acid I-1b (4.1 g, 13 mmol) in EtOH (30 ml) at 0° C. was added morpholine (2.3 ml, 27 mmol) dropwise. The reaction was stirred at this temperature for 1 h, then allowed to warm to RT and stirred for 72 h. The mixture was concentrated in vacuo to give 2-((±)-8-(tert-butoxycarbonyl)-2-oxo-8-azabicyclo[3.2.1]octan-3-yl)-2-morpholinoacetic acid, morpholine salt I-1c as a sticky yellow oil that was used in the next step without any further purification or analysis.

Step 3: To a solution of 2-((±)-8-(tert-butoxycarbonyl)-2-oxo-8-azabicyclo[3.2.1]octan-3-yl)-2-morpholinoacetic acid, morpholine salt I-1c (17.02 g, 37.37 mmol) in EtOH (120 ml) was added hydrazine monohydrate in water (11.3 ml, 149.5 mmol). The resultant mixture was heated to 78° C. for 2.5 h, cooled to RT and then the mixture was concentrated in vacuo. The resultant yellow residue was dissolved in DCM (500 ml) and water (150 ml). The layers were separated, and the aqueous layer was extracted with 10% MeOH in DCM (3×200 ml). The combined organic layers were dried over MgSO₄ and concentrated in vacuo to provide a pale yellow solid. The solid was dissolved in the minimum amount of EtOH and allowed to sit at RT for 16 h and the resulting precipitate was isolated by filtration to provide tert-butyl (±)-3-oxo-3,5,6,7,8,9-hexahydro-2H-6,9-epiminocyclohepta[c]pyridazine-10-carboxylate I-1e as a white powder. The filtrate was concentrated to provide a mixture of tert-butyl (±)-3-oxo-3,5,6,7,8,9-hexahydro-2H-6,9-epiminocyclohepta[c]pyridazine-10-carboxylate I-1e and tert-butyl (±)-4-morpholino-3-oxo-3,4,4a,5,6,7,8,9-octahydro-2H-6,9-epiminocyclohepta[c]pyridazine-10-carboxylate I-1d as a sticky brown oil.

1-1e: ¹H NMR (400 MHz, DMSO-d6) δ 6.67 (s, 1H), 4.71 (d, J=6.2 Hz, 1H), 4.31 (br s, 1H), 3.03 (d, J=17.9 Hz, 1H), 2.61 (d, J=18.1 Hz, 1H), 2.22-2.07 (m, 2H), 1.73 (t, J=9.6 Hz, 1H), 1.60 (t, J=8.1 Hz, 1H), 1.35 (s, 9H). (Exchangeable proton not visible in spectrum)

I-1d: ¹H NMR (400 MHz, DMSO-d6) δ 10.48 (s, 1H), 4.42 (d, J=6.9 Hz, 1H), 4.22 (d, J=6.7 Hz, 1H), 3.55-3.48 (m, 4H), 3.09-2.92 (m, 4H), 2.69 (dd, J=12.1, 6.0 Hz, 2H), 2.06-2.00 (m, 2H), 1.92 (d, J=10.1 Hz, 1H), 1.69-1.65 (m, 2H), 1.56 (d, J=12.7 Hz, 1H), 1.38 (s, 9H).

Step 4: To a solution of tert-butyl (±)-3-oxo-3,5,6,7,8,9-hexahydro-2H-6,9-epiminocyclohepta[c]pyridazine-10-carboxylate I-1e and tert-butyl (±)-4-morpholino-3-oxo-3,4,4a,5,6,7,8,9-octahydro-2H-6,9-epiminocyclohepta[c]pyridazine-10-carboxylate I-1d (3.2 g, 66% Mol. Wt I-1d) in EtOH (40 ml) was added a 2 M NaOH solution (10 ml, 20 mmol) and the resultant mixture was stirred at RT for 16 h. The reaction mixture was concentrated in vacuo and the aqueous was neutralised to pH 7 using 1 M HCl. The product was extracted with 10% MeOH in DCM (3×150 ml) and the combined organics were dried with MgSO₄. The solvent was removed in vacuo to give tert-butyl (±)-3-oxo-3,5,6,7,8,9-hexahydro-2H-6,9-epiminocyclohepta[c]pyridazine-10-carboxylate I-1e as a white solid.

Step 5: To a solution of tert-butyl (±)-3-oxo-3,5,6,7,8,9-hexahydro-2H-6,9-epiminocyclohepta[c]pyridazine-10-carboxylate (2.6 g, 9.4 mmol) in DCM (20 ml) was added a solution of HCl in 1,4-dioxane (23 ml, 4 M, 94 mmol). The reaction was stirred at RT for 72 h and then concentrated in vacuo to give the HCl salt of I-1f. The HCl salt was dissolved in MeOH (150 ml), AcOH was added and the solution was loaded onto SCX resin (50 g). The cartridge was washed with MeOH and the product was eluted with 0.7 M NH₃ in MeOH solution. To give (±)-2,5,6,7,8,9-hexahydro-3H-6,9-epiminocyclohepta[c]pyridazin-3-one I-1f as an off white solid.

Alternatively the HCl was dissolved in MeOH and MP-carbonate (3 eq.) was added and the mixture was left for 1 h. The mixture was filtered. The resin was washed with MeOH. The filtrate was concentrated in vacuo to give (±)-2,5,6,7,8,9-hexahydro-3H-6,9-epiminocyclohepta[c]pyridazin-3-one I-1f as an off white solid.

### Step 6

To a solution of (±)-2,5,6,7,8,9-hexahydro-3H-6,9-epiminocyclohepta[c]pyridazin-3-one I-1f (200 mg, 1.13 mmol) in MeOH (10 ml) was added a solution of dibenzoyl-L-tartaric acid in MeOH (5 ml). The solvent was removed in vacuo and the residue was redissolved in EtOH (40 ml), heated to 60° C. for 1 h and allowed to cool and left standing for 2 h. The solid was filtered off and dried in vacuo and then dissolved in MeOH (10 ml) and MP-carbonate (600 mg, 1.8 mmol) was added and the mixture was left to stand for 1 h. The solid was filtered and the filtrate concentrated in vacuo to give (5R,8S)-3,5,6,7,8,9-hexahydro-2H-5,8-epiminocyclohepta[d]pyrimidin-2-one 1-1 as an off white solid. ¹H NMR (400 MHz, DMSO— d6) δ 12.48 (s, 1H), 6.56 (s, 1H), 4.05-3.99 (m, 1H), 3.63 (t, J=5.9 Hz, 1H), 2.90-2.78 (m, 1H), 2.45 (t, J=1.4 Hz, 1H), 1.98-1.81 (m, 2H), 1.65 (t, J=9.3 Hz, 1H), 1.53-1.39 (m, 1H). (NH not observed).

Intermediate 2 (1-2)

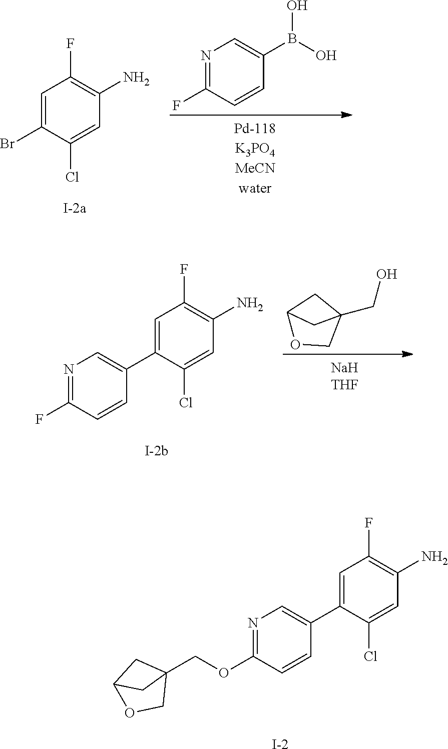

Step 1: A flask containing 4-bromo-5-chloro-2-fluoroaniline I-2a (50.0 g, 222.8 mmol), (6-fluoropyridin-3-yl)boronic acid (31.4 g, 222.8 mmol) was purged with N₂ for 5 min before MeCN (500 mL) was added and N₂ was bubbled through the reaction mixture. Pd-118 (3.63 g, 5.57 mmol) was added followed by an aqueous solution of tripotassium phosphate (334 mL, 2 M, 668.3 mmol). The reaction mixture was purged with N₂ for 5 min, heated to 90° C., stirred for 2 h and then allowed to cool to RT before being left to stir for 16 h, at which time a precipitate had formed. This was filtered off and washed with acetonitrile (50 mL), water (50 mL) and DCM (200 mL) and dried in vacuo to give 5-chloro-2-fluoro-4-(6-fluoropyridin-3-yl)aniline I-2b as a brown solid. ¹H NMR (400 MHz, DMSO-d6) δ 8.23 (d, J=2.6 Hz, 1H), 8.02 (td, J=8.2, 2.6 Hz, 1H), 7.27-7.16 (m, 2H), 6.93 (d, J=8.3 Hz, 1H), 5.69 (s, 2H).

Step 2: To a suspension of (2-oxabicyclo[2.1.1]hexan-4-yl)methanol (69 mg, 607 μmol) in THF (2 mL) at 0° C. was added sodium hydride (16 mg, 658 μmol) and the resulting suspension was stirred at RT for 30 min before 5-chloro-2-fluoro-4-(6-fluoropyridin-3-yl)aniline I-2b (123 mg, 506 μmol) in THF (2 mL) was added. The reaction was stirred at RT for 2 h. The reaction mixture was warmed to 60° C. for 1 h. The reaction was cooled to RT and a further portion of sodium hydride (16 mg, 658 μmol) was added and the resulting mixture was stirred at 60° C. for 72 h. The reaction was cooled to RT and a further portion of sodium hydride (16 mg, 658 μmol) was added and stirring was continued at 60° C. for 1 h. The mixture was cooled and a final portion of sodium hydride (16 mg, 658 μmol) was added and stirring at 60° C. was continued for 1 h. The reaction was cooled to RT, water (0.1 mL) was added and the product was partitioned between brine (25 mL) and DCM (25 mL). The aqueous was extracted with DCM (2×25 mL) and the combined organics were dried over MgSO₄, filtered and concentrated in vacuo. The product was purified by chromatography on silica gel (0-40% EtOAc/isohexane) to afford 4-(6-((2-oxabicyclo[2.1.1]hexan-4-yl)methoxy)pyridin-3-yl)-5-chloro-2-fluoroaniline 1-2 as a sticky yellow oil. ¹H NMR (400 MHz, DMSO-d6) δ 8.13 (dd, J=2.6, 0.8 Hz, 1H), 7.73 (dd, J=8.6, 2.5 Hz, 1H), 7.12 (d, J=11.8 Hz, 1H), 7.00-6.78 (m, 2H), 5.59 (s, 2H), 4.60 (s, 2H), 4.50 (d, J=1.1 Hz, 1H), 3.59 (s, 2H), 1.88-1.78 (m, 2H), 1.48 (dd, J=4.5, 1.8 Hz, 2H).

### Intermediate 3 (1-3)

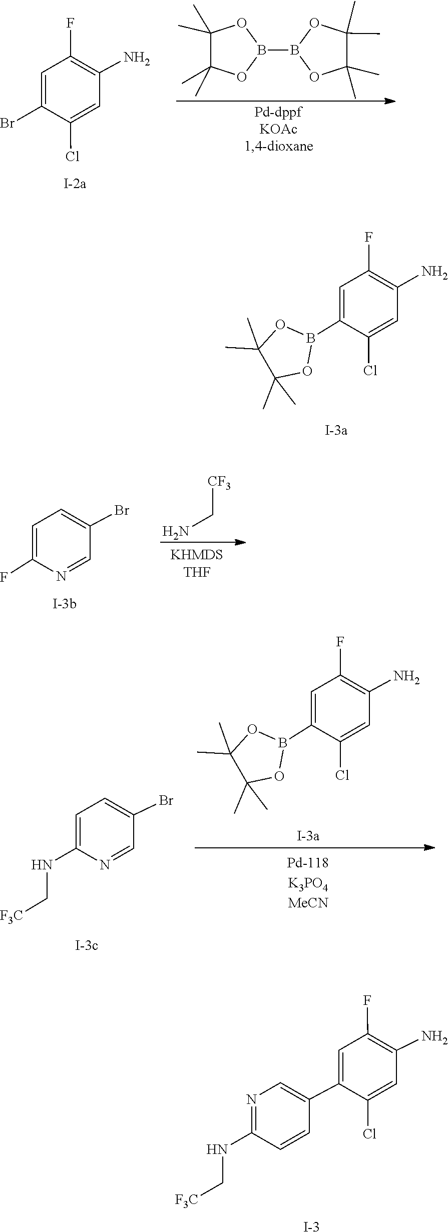

Step 1: To a solution of 4-bromo-5-chloro-2-fluoroaniline I-2a (10.0 g, 44.6 mmol), potassium acetate (13.1 g, 134 mmol) and bis(pinacolato)diboron (17.0 g, 66.8 mmol) in 1,4-dioxane (100 mL) was added Pd(dppf)Cl₂ (2.73 g, 3.34 mmol). The reaction was heated to 100° C. for 4 h, before cooling to RT and stirring for 20 h. The resulting mixture was diluted with EtOAc (200 mL) before being filtered. The precipitate was washed with additional EtOAc (200 mL) and the filtrate was concentrated in vacuo. The product was partitioned between DCM (150 mL) and saturated aqueous NaHCO₃ (150 mL) and the organics were washed with saturated aqueous NaHCO₃ (150 mL) and brine (150 mL) and concentrated in vacuo. The residue was suspended in pentane (300 mL), stirred for 45 minutes and then cooled to 0° C. before being filtered. The precipitate was washed with additional pentane (2×50 mL) and dried in vacuo to afford 5-chloro-2-fluoro-4-(4,4,5,5-tetramethyl-1,3,2-dioxaborolan-2-yl)-benzenamine I-3a as a black powder. ¹H NMR (400 MHz, DMSO-d6) δ 7.14 (d, J=11.9 Hz, 1H), 6.73 (d, J=7.7 Hz, 1H), 5.89 (s, 2H), 1.16 (s, 12H)

Step 2: To a solution of 2,2,2-trifluoroethylamine (674 μL, 8.52 mmol) and 5-bromo-2-fluoro-pyridine I-3b (585 μL, 5.68 mmol) in THF (3 mL) was slowly added a solution of KHMDS in THF (8.5 mL, 1 M, 8.52 mmol). The reaction was stirred at RT for 1 h then quenched with MeOH (10 mL) and the mixture was concentrated in vacuo. The product was purified by chromatography on silica gel (0-30% EtOAc/isohexane) to afford 5-bromo-N-(2,2,2-trifluoroethyl)pyridin-2-amine I-3c as a sticky white solid. ¹H NMR (400 MHz, DMSO-d6) δ 8.09 (d, J=2.5 Hz, 1H), 7.62 (dd, J=8.9, 2.5 Hz, 1H), 7.38 (t, J=6.6 Hz, 1H), 6.62 (d, J=8.8 Hz, 1H), 4.13 (qd, J=9.8, 6.6 Hz, 2H).

Step 3: To a suspension of 5-bromo-N-(2,2,2-trifluoroethyl)pyridin-2-amine I-3c (250 mg, 980 μmol), 5-chloro-2-fluoro-4-(4,4,5,5-tetramethyl-1,3,2-dioxaborolan-2-yl)aniline I-3a (505 mg, 1.08 mmol) and Pd-118 (32 mg, 49.0 μmol) in MeCN (4 mL) under N₂ atmosphere was added a pre-degassed aqueous solution of potassium phosphate tribasic (1.47 mL, 2 M, 2.94 mmol). The reaction was heated to 77° C. for 2 h and then cooled to RT before being poured into brine (40 mL), followed by extraction with EtOAc (2×40 mL). The combined organics were washed with brine (30 mL), dried over magnesium sulphate, filtered and concentrated in vacuo. The material was purified by chromatography on silica gel (0-30% EtOAc/isohexane) to afford 5-(4-amino-2-chloro-5-fluorophenyl)-N-(2,2,2-trifluoroethyl)pyridin-2-amine 1-3 as a tan solid. ¹H NMR (400 MHz, DMSO-d6) δ 8.01 (dd, J=2.4, 0.8 Hz, 1H), 7.49 (dd, J=8.6, 2.4 Hz, 1H), 7.25 (t, J=6.6 Hz, 1H), 7.05 (d, J=12.0 Hz, 1H), 6.88 (d, J=8.4 Hz, 1H), 6.65 (dd, J=8.7, 0.8 Hz, 1H), 5.50 (s, 2H), 4.18 (qd, J=9.8, 6.5 Hz, 2H).

### Intermediate 4 (1-4)

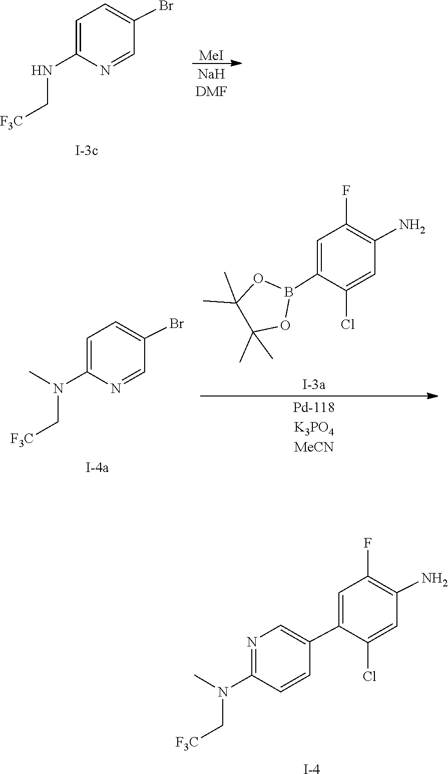

Step 1: To a solution of 5-bromo-N-(2,2,2-trifluoroethyl)pyridin-2-amine I-3c (200 mg, 784 μmol) in DMF (1.00 mL) at 0° C. was added NaH (38 mg, 60% Wt, 941 μmol) in a single portion. Mel (98 μL, 1.57 mmol) was added and stirring was continued at 0° C. for 30 min. The reaction was quenched with water (1 mL) and the product was extracted with EtOAc (3×5 mL). The combined organics were dried over MgSO₄, filtered and concentrated in vacuo. The product was purified by chromatography on silica gel (0-15% EtOAc/isohexane) to afford 5-bromo-N-methyl-N-(2,2,2-trifluoroethyl)pyridin-2-amine I-4a as a colourless oil. ¹H NMR (400 MHz, DMSO-d6) δ 8.20 (dd, J=2.6, 0.7 Hz, 1H), 7.76 (dd, J=9.0, 2.6 Hz, 1H), 6.79 (dd, J=9.1, 0.7 Hz, 1H), 4.45 (q, J=9.6 Hz, 2H), 3.07 (d, J=1.1 Hz, 3H).

Step 2: 5-(4-Amino-2-chloro-5-fluorophenyl)-N-methyl-N-(2,2,2-trifluoroethyl)pyridin-2-amine 1-4 was synthesised from 5-bromo-N-methyl-N-(2,2,2-trifluoroethyl)pyridin-2-amine I-4a and 5-chloro-2-fluoro-4-(4,4,5,5-tetramethyl-1,3,2-dioxaborolan-2-yl)aniline I-3a using a procedure essentially the same as for 1-3. ¹H NMR (400 MHz, DMSO-d6) δ 8.13 (dd, J=2.4, 0.8 Hz, 1H), 7.63 (dd, J=8.8, 2.5 Hz, 1H), 7.08 (d, J=12.0 Hz, 1H), 6.89 (d, J=8.4 Hz, 1H), 6.82 (dd, J=8.9, 0.9 Hz, 1H), 5.52 (d, J=3.3 Hz, 2H), 4.50 (q, J=9.6 Hz, 2H), 3.11 (s, 3H).

### Intermediate 5 (1-5)

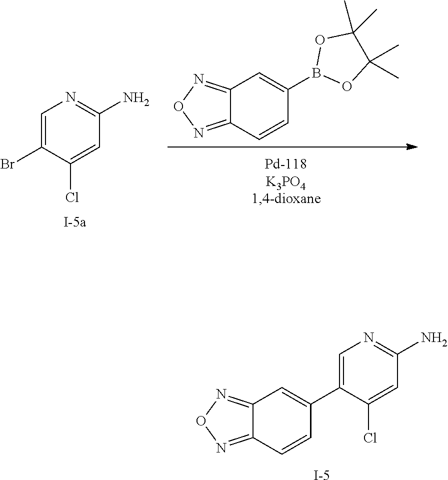

To a mixture of 5-(4,4,5,5-tetramethyl-1,3,2-dioxaborolan-2-yl)benzo[c][1,2,5]oxadiazole (297 mg, 1.21 mmol), 5-bromo-4-chloropyridin-2-amine I-5a (250 mg, 1.21 mmol) and Pd-118 (20 mg, 30.1 μmol) in pre-degassed 1,4-dioxane (10 mL) was added a pre-degassed aqueous solution of potassium phosphate tribasic (1.8 mL, 2 molar, 3.62 mmol). The resulting suspension was heated to 95° C. for 1 h before the reaction was allowed to cool to RT and partitioned between DCM (25 mL) and brine (25 mL). The aqueous phase was extracted with additional DCM (25 mL) and the combined organics were concentrated in vacuo. The product was purified by chromatography on silica gel (0-10% (0.7 M ammonia/MeOH/DCM) to afford 5-(benzo[c][1,2,5]oxadiazol-5-yl)-4-chloropyridin-2-amine I-5 as a brown solid. ¹H NMR (400 MHz, DMSO-d6) δ 8.11-8.06 (m, 2H), 8.04 (t, J=1.2 Hz 1H), 7.68 (dd, J=9.3, 1.4 Hz, 1H), 6.65 (s, 1H), 6.60 (s, 2H).

### Intermediate 6 (1-6)

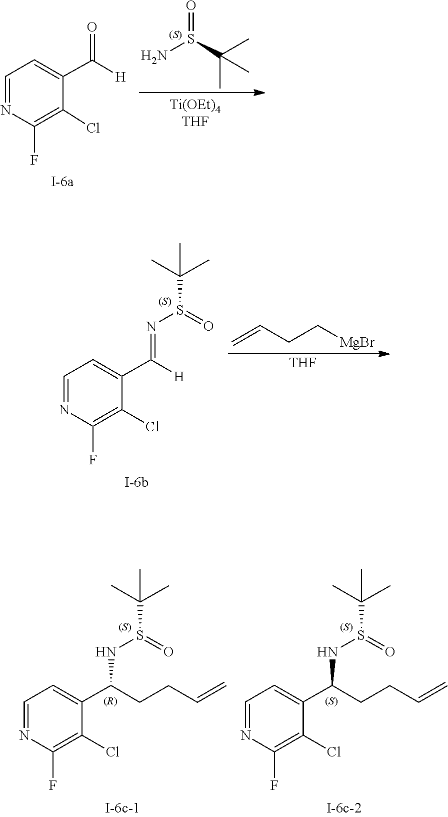

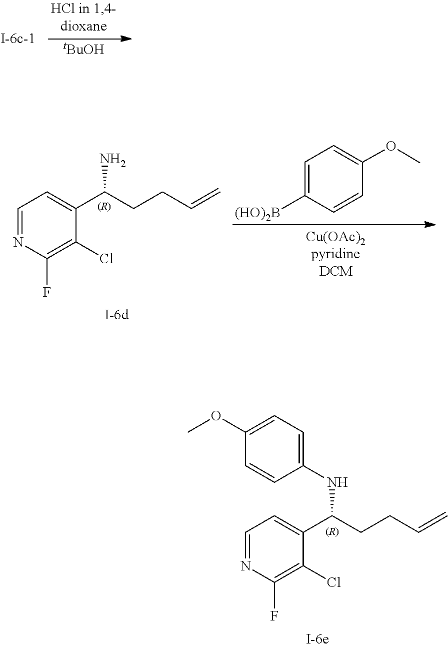

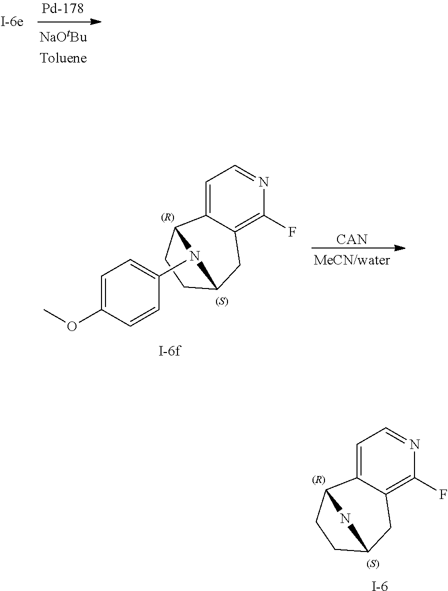

Step 1: To a solution of 3-chloro-2-fluoroisonicotinaldehyde I-6a (1.00 g, 6.27 mmol) in THF (17 ml) was added titanium (IV) ethoxide (2.63 ml, 12.5 mmol) in one portion at RT. The reaction was stirred at RT for 5 min before (S)-tert-butylsulfinamide (760 mg, 6.27 mmol) was added in one portion. The mixture was stirred at RT for 16 h before brine (30 ml) was added, and the mixture was stirred for 10 min then filtered through celite. The filtrate was extracted with EtOAc (2×20 ml) and the combined organics were dried over MgSO₄ and concentrated in vacuo to give (S)-N-((3-chloro-2-fluoropyridin-4-yl)methylene)-2-methylpropane-2-sulfinamide (1-6b) as a pale yellow solid. LCMS (Method 1) m/z 227.5 (M-CI)+(ES+), at 1.36 min. ¹H NMR (500 MHz, DMSO-d6) δ 8.81 (s, 1H), 8.33 (dt, J=5.1, 0.8 Hz, 1H), 7.90 (d, J=5.1 Hz, 1H), 1.22 (s, 9H).

Step 2: To a solution of (S)-N-((3-chloro-2-fluoropyridin-4-yl)methylene)-2-methylpropane-2-sulfinamide (1.38 g, 5.26 mmol) (1-6b) in THF (22 ml) was added but-3-en-1-yl magnesium bromide (31.6 ml, 0.5 M, 15.8 mmol) dropwise at −78° C. The mixture was allowed to warm to RT slowly and stirred for 72 h. Saturated NH₄Cl solution (10 ml) was added and the product was extracted with EtOAc (3×10 ml). The combined organics were dried with MgSO₄ and concentrated in vacuo. The product was purified by silica gel chromatography (0-100% EtOAc/isohexane) to give (S)-N-(l-1-(3-chloro-2-fluoropyridin-4-yl)pent-4-en-1-yl)-2-methylpropane-2-sulfinamide (1-6c-1) as a pale yellow solid. LCMS (Method 1) m/z 316.7, 319.1 (M−H)⁻ (ES-), at 1.39 min. ¹H NMR (500 MHz, DMSO-d6) δ 8.21 (dd, J=5.2, 0.8 Hz, 1H), 7.53 (d, J=5.2 Hz, 1H), 5.90 (d, J=7.3 Hz, 1H), 5.81 (dddd, J=17.3, 10.2, 7.1, 6.1 Hz, 1H), 5.10 (dq, J=17.2, 1.7 Hz, 1H), 5.02 (ddt, J=10.2, 2.3, 1.2 Hz, 1H), 4.66 (ddd, J=8.6, 7.3, 5.3 Hz, 1H), 2.27-2.08 (m, 2H), 1.-7-1.87 (m, 1H), 1.-0-1.70 (m, 1H), 1.07 (s, 9H).

(S)-N-((S)-1-(3-chloro-2-fluoropyridin-4-yl)pent-4-en-1-yl)-2-methylpropane-2-sulfinamide (I-6c-2) was also obtained from the purification

Step 3: To a solution of (S)N(R)-1-(3-chloro-2-fluoropyridin-4-yl)pent-4-en-1-yl)-2-methylpropane-2-sulfinamide (1-6c) (714 mg, 2.015 mmol) in ^{t}BuOH (7.2 ml) was added a solution of HCl in 1,4-dioxane (3.0 ml, 4 M, 12.09 mmol) at RT and the resulting mixture was stirred for 2.5 h. The reaction was then quenched with saturated aqueous NaHCO₃ solution (100 ml) and extracted with DCM (3×30 ml). The combined organics were dried over MgSO₄ and concentrated in vacuo to afford (R)-1-(3-chloro-2-fluoropyridin-4-yl)pent-4-en-1-amine (1-6d) as a pale yellow liquid. LCMS (Method 1): m/z 215.4, 217.3 (M+H)+(ES+); at 1.17 min, ¹H NMR (500 MHz, DMSO-d6) δ 8.16 (dd, J=5.1, 0.9 Hz, 1H), 7.62 (d, J=5.1 Hz, 1H), 5.80 (ddt, J=16.9, 10.2, 6.6 Hz, 1H), 5.02 (dq, J=17.4, 1.8 Hz, 1H), 4.96 (ddt, J=10.2, 2.3, 1.3 Hz, 1H), 4.22 (dd, J=8.1, 5.1 Hz, 1H), 2.23-2.01 (m, 2H), 1.76-1.50 (m, 2H), 2 NH protons not observed.

Step 4: To a solution of (R)-1-(3-chloro-2-fluoropyridin-4-yl)pent-4-en-1-amine (1-6d) (644.3 mg, 3.001 mmol), (4-methoxyphenyl)boronic acid (1.37 g, 9.0 mmol), and Cu(OAc)₂ (820 mg, 4.50 mmol) in DCM (100 ml) was added pyridine (1.2 ml, 15.0 mmol) dropwise. The mixture was stirred at RT for 16 h, open to air before 2M NaOH aqueous solution (20 ml) was added followed by water (20 ml) and the mixture extracted with DCM (3×20 ml). The combined organics were dried over MgSO₄ and concentrated in vacuo. The product was purified by silica gel chromatography (0%-100% DCM/isohexane to give (R)-N-(1-(3-chloro-2-fluoropyridin-4-yl)pent-4-en-1-yl)-4-methoxyaniline (1-6e) as a yellow oil. LCMS (Method 1): m/z 321.1, 323.4 (M+H)+(ES+); at 1.73 min, ¹H NMR (500 MHz, DMSO-d6) δ 8.10 (d, J=5.1 Hz, 1H), 7.41 (d, J=5.1 Hz, 1H), 6.65 (d, J=8.9 Hz, 2H), 6.38 (d, J=8.9 Hz, 2H), 6.09 (d, J=8.3 Hz, 1H), 5.84 (ddt, J=17.0, 10.2, 6.6 Hz, 1H), 5.10-4.88 (m, 2H), 4.68 (td, J=8.5, 4.7 Hz, 1H), 3.57 (s, 3H), 2.34-2.24 (m, 1H), 2.23-2.12 (m, 1H), 1.87-1.69 (m, 2H).

Step 5: A three-neck flask was charged with Pd-178 (7.4 mg, 15.6 μmol) and sodium tert-butoxide (22.5 mg, 234 μmol) and purged with N₂ before a solution of (R)-N-(1-(3-chloro-2-fluoropyridin-4-yl)pent-4-en-1-yl)-4-methoxyaniline (1-6e) (50.0 mg, 156 μmol) in toluene (1 ml) was added dropwise. The resulting mixture was heated to 95° C. for 1.5 h before being allowed to cool to RT and then filtered through celite, washing with EtOAc (3×20 ml). The filtrate was concentrated in vacuo and the product was purified by silica gel chromatography (0%-50% EtOAc/heptane) to give (5R,8S)-1-fluoro-10-(4-methoxyphenyl)-6,7,8,9-tetrahydro-5H-5,8-epiminocyclohepta[c]pyridine (1-6f) as a white solid. LCMS (Method 1) m/z 285.3 (M+H)+(ES+), at 1.35 min. ¹H NMR (500 MHz, DMSO-d6) δ 7.95 (d, J=4.9 Hz, 1H), 7.29 (dd, J=5.0, 1.7 Hz, 1H), 6.80 (d, J=9.1 Hz, 2H), 6.71 (d, J=9.1 Hz, 2H), 4.92 (d, J=5.6 Hz, 1H), 4.56 (t, J=5.9 Hz, 1H), 3.61 (s, 3H), 2.92 (dd, J=17.6, 4.9 Hz, 1H), 2.35 (d, J=17.5 Hz, 1H), 2.32-2.19 (m, 2H), 1.91-1.82 (m, 1H), 1.81-1.74 (m, 1H).

Step 6: A solution of (5R,8S)-1-fluoro-10-(4-methoxyphenyl)-6,7,8,9-tetrahydro-5H-5,8-epiminocyclohepta[c]pyridine (1-6f) (69 mg, 232 μmol) in MeCN (3.2 ml) was cooled to 0° C. before a solution of CAN (381 mg, 696 μmol) in water (3.2 ml) was added dropwise. After the addition was complete, the reaction was stirred for 1 h at 0° C. before a 2 M aqueous solution of NaOH (5 ml) and water (5 ml) were added and the mixture extracted with DCM (3×10 ml). The combined organics were dried over MgSO₄ and concentrated in vacuo to give (5R,8S)-1-fluoro-6,7,8,9-tetrahydro-5H-5,8-epiminocyclohepta[c]pyridine (1-6) as a pale pink solid. LCMS (Method 1): m/z 179.2 (M+H)+(ES+); at 0.69 min, ¹H NMR (500 MHz, DMSO-d6) δ 7.91 (dd, J=5.0, 0.9 Hz, 1H), 7.04 (dd, J=5.0, 1.9 Hz, 1H), 4.17 (d, J=5.3 Hz, 1H), 3.78 (t, J=6.0 Hz, 1H), 2.85 (dd, J=17.1, 5.2 Hz, 1H), 2.74 (s, 1H), 2.38 (d, J=17.0 Hz, 1H), 2.01-1.85 (m, 2H), 1.73 (t, J=9.1 Hz, 1H), 1.51 (dt, J=9.4, 5.4 Hz, 1H).

### Intermediate 7 (1-7)

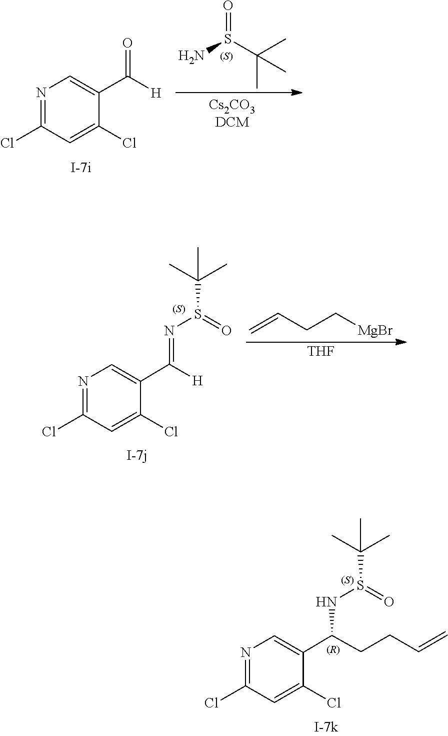

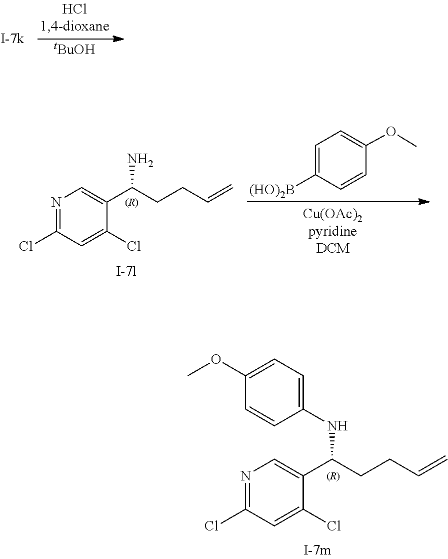

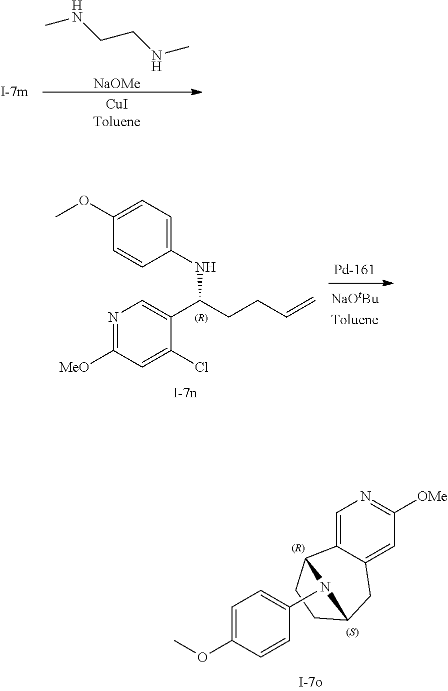

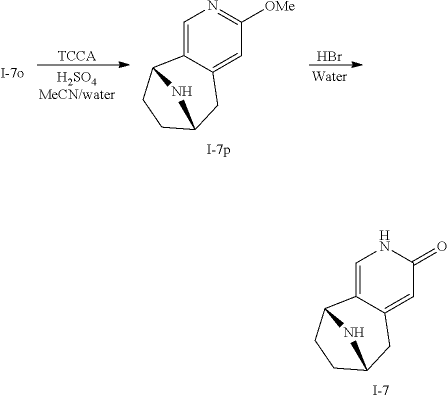

Step 1: To a mixture of 4,6-dichloronicotinaldehyde-1-7i (17.3 g, 88.5 mmol) and (S)-2-methylpropane-2-sulfinamide (10.7 g, 88.5 mmol) in iCM (150 ml) was added cesium carbonate (28.8 g, 88.5 mmol). The reaction was stirred at RT for 16 h before the solid residue was isolated by filtration and washed with CM (150 ml). The solvent was evaporated to give (S)-N-((4,6-dichloropyridin-3-yl)methylene)-2-methylpropane-2-sulfinamide 1-7j as an off-white solid. ¹H NMR (400 MHz, DMSO-d6) 8.97 (s, 1H), 8.76 (s, 1H), 8.04 (s, 1H), 1.21 (s, 9H).

Step 2: A solution of but-3-en-1-ylmagnesium bromide (0.5 M in THF) (60.9 ml, 30.4 mmol) was slowly added to a solution of (S)-N-((4,6-dichloropyridin-3-yl)methylene)-2-methylpropane-2-sulfinamide 1-7j (5.00 g, 1, 17.9 mmol) in THF (100 ml-) at −78° C. The reaction was allowed to slowly warm up to RT over 16 h and then cooled to 0-10° C. before saturated aqueous NH₄Cl (100 ml) was added and the resulting mixture was stirred for 5 min. The aqueous phase was extracted with EtOAc (2×250 ml) and the combined organics were washed with brine (50 ml), dried with Na₂SO₄ and concentrated in vacuo. The product was purified by chromatography on silica gel (0-30% MTBE in DCM) to afford (S)-N-((R)-1-(4,6-dichloropyridin-3-yl)pent-4-en-1-yl)-2-methylpropane-2-sulfinamide 1-7k as a clear yellow oil. ¹H NMR (400 MHz, DMSO-d6) δ 8.53 (s, 1H), 7.77 (s, 1H), 5.85-5.75 (m, 2H), 5.07 (dq, J=17.2, 1.6 Hz, 1H), 5.00 (ddt, J=10.3, 2.3, 1.3 Hz, 1H), 4.66-4.56 (m, 1H), 2.20-2.05 (m, 2H), 1.98 (dtd, J=13.9, 8.0, 5.7 Hz, 1H), 1.83 (ddt, J=13.3, 8.7, 6.5 Hz, 1H), 1.07 (s, 9H).

Step 3: A solution of HCl (4 M in dioxane) (41 ml, 0.16 mol) was added to a solution of (S)-N-((R)-1-(4,6-dichloropyridin-3-yl)pent-4-en-1-yl)-2-methylpropane-2-sulfinamide 1-7k (11 g, 33 mmol) in ^{t}BuOH (50 ml) and the resulting mixture was stirred at RT for 90 min. The reaction was cooled in an ice bath and water (220 ml) was added and the mixture was stirred for 10 min. The aqueous portion was extracted with MTBE (3×30 ml) and the organic layer was extracted with water (2×30 ml). The combined aqueous portions were basified using saturated aqueous NaHCO₃ solution and more solid NaHCO₃ and the mixture was stirred for 15 min before the product was extracted with MTBE (3×100 ml). The combined oragnics were washed with water (500 ml), dried with Na₂SO₄ and concentrated in vacuo to to give (R)-1-(4,6-dichloropyridin-3-yl)pent-4-en-1-amine 1-71 as an orange oil. ¹H NMR (400 MHz, DMSO-d6) δ 8.60 (s, 1H), 7.69 (s, 1H), 5.80 (ddt, J=16.9, 10.1, 6.6 Hz, 1H), 5.01 (dq, J=17.2, 1.7 Hz, 1H), 4.94 (ddt, J=10.2, 2.3, 1.3 Hz, 1H), 4.15 (dd, J=8.0, 5.2 Hz, 1H), 2.17-2.01 (m, 4H), 1.74-1.54 (m, 2H).

Step 4: To a solution of (R)-1-(4,6-dichloropyridin-3-yl)pent-4-en-1-amine 1-71 (7.04 g, 30.5 mmol) in DCM (70 ml) was added (4-methoxyphenyl)boronic acid (13.9 g, 91.4 mmol), copper (II) acetate (6.09 g, 33.5 mmol) and Et₃N (21.2 ml, 152 mmol). The resultant mixture was stirred at RT for 20 h and then further portions of copper (II) acetate (2.21 g, 12.2 mmol), (4-methoxyphenyl)boronic acid (5.55 g, 36.6 mmol) and Et₃N (5.09 ml, 36.6 mmol) were added and the mixture was stirred for a further 20 h. Aqueous HCl (1 M, 200 ml) was added and the layers were separated before ammonium hydroxide solution (28% w/v, 100 and 200 ml) was added to the organic and aqueous layers respectively. The layers were separated and the aqueous was extracted with DCM (100 ml). The combined organics were washed with water (100 ml) and dried with MgSO₄. The product was purified by chromatography on silica gel (0-50% EtOAc/iso-hexane) to afford (R)-N-(1-(4,6-dichloropyridin-3-yl)pent-4-en-1-yl)-4-methoxyaniline 1-7 m as a thick colourless oil. ¹H NMR (400 MHz, DMSO-d6) δ 8.42 (s, 1H), 7.75 (s, 1H), 6.70-6.59 (m, 2H), 6.46-6.34 (m, 2H), 6.01 (d, J=8.5 Hz, 1H), 5.90-5.77 (m, 1H), 5.08-4.94 (m, 2H), 4.64 (td, J=8.6, 4.9 Hz, 1H), 3.58 (s, 3H), 2.27 (ddd, J=12.7, 9.3, 6.1 Hz, 1H), 2.22-2.08 (m, 1H), 1.92-1.72 (m, 2H).

Step 5: To a solution of (R)-N-(1-(4,6-dichloropyridin-3-yl)pent-4-en-1-yl)-4-methoxyaniline I-7m (2.73 g, 8.10 mmol) in toluene (20 ml) was added N,N-dimethylethane-1,2-diamine (86.8 pl, 810 μmol), copper(I) iodide (30.8 mg, 162 μmol) and sodium methoxide (656 mg, 243 μmol). The resultant mixture was heated at 100° C. for 96 h before being allowed to cool to RT and then filtered through a pad of celite. The filtrate was concentrated in vacuo and the product was purified by chromatography on silica gel (0-30% MTBE in iso-hexane) to afford (R)-N-(1-(4-chloro-6-methoxypyridin-3-yl)pent-4-en-1-yl)-4-methoxyaniline I-7n as a thick yellow oil. ¹H NMR (400 MHz, DMSO-d6) δ 8.17 (s, 1H), 6.94 (s, 1H), 6.66-6.62 (m, 2H), 6.43-6.37 (m, 2H), 5.91 (d, J=8.5 Hz, 1H), 5.89-5.78 (m, 1H), 5.07-4.93 (m, 2H), 4.57 (td, J=8.5, 5.1 Hz, 1H), 3.79 (s, 3H), 3.58 (s, 3H), 2.27 (dt, J=14.3, 7.3 Hz, 1H), 2.22-2.07 (m, 1H), 1.80 (dddd, J=20.8, 13.8, 10.1, 5.1 Hz, 2H).

Step 6: A 3-necked RB flask was charged with Pd-161 (763.6 mg, 1.65 mmol) and NaO^{t}Bu (2.38 g, 24.8 mmol) and the system was purged under vacuum and backfilled with N₂ (3 times). A second flask containing (R)-N-(1-(4-chloro-6-methoxypyridin-3-yl)pent-4-en-1-yl)-4-methoxyaniline I-7n (5.79 g, 16.52 mmol) was purged under vacuum and backfilled with N₂ (3 times). Toluene (180 ml) was added to amine and the resultant solution was transferred to the 3-necked RB flask which was then was purged under vacuum and backfilled with N₂ (3 times). The resultant mixture was heated at 95° C. for 2 h, allowed to cool to RT and filtered through a pad of celite. The filter cake was washed with EtOAc and the filtrate was concentrated in vacuo. The product was purified by chromatography on silica gel (0-50% EtOAc/iso-hexane) to afford (6S,9R)-3-methoxy-10-(4-methoxyphenyl)-6,7,8,9-tetrahydro-5H-6,9-epiminocyclohepta[c]pyridine 1-70 as a light orange solid. ¹H NMR (500 MHz, DMSO-d6) δ 8.01 (s, 1H), 6.82-6.73 (m, 2H), 6.73-6.64 (m, 2H), 6.39 (s, 1H), 4.83 (d, J=5.5 Hz, 1H), 4.43 (m, 1H), 3.74 (s, 3H), 3.61 (s, 3H), 3.04 (dd, J=18.0, 4.9 Hz, 1H), 2.41 (d, J=17.9 Hz, 1H), 2.32-2.14 (m, 2H), 1.84-1.63 (m, 2H).

Step 7: To a solution of (6S,9R)-3-methoxy-10-(4-methoxyphenyl)-6,7,8,9-tetrahydro-5H-6,9-epiminocyclohepta[c]pyridine 1-70 (2.00 g, 6.61 mmol) in MeCN (75 ml) and water (75 ml) was added sulfuric acid (6.6 ml, 1 M, 6.61 mmol) followed by trichloroisocyanuric acid (769 mg, 3.31 mmol). The reaction mixture was stirred at RT for 16 h and then extracted with DCM (3×200 ml). The combined organics were extracted with water (50 ml) and the aqueous layer was basified with KOH (3.6 ml, 5 M) and extracted with 10% MeOH in DCM (300 ml). Further KOH (1.8 ml, 5 M) was added and the aqueous layer was extracted with 10% MeOH in DCM (100 ml). A further portion of KOH (1.8 ml, 5 M) was added and the aqueous layer was extracted with 10% MeOH in DCM (150 ml). The combined organics were dried over Na₂SO₄ and concentrated in vacuo to give (6S,9R)-3-methoxy-6,7,8,9-tetrahydro-5H-6,9-epiminocyclohepta[c]pyridine I-7p was isolated as a brown oil. ¹H NMR (400 MHz, DMSO-d6) δ 7.78 (s, 1H), 6.47 (s, 1H), 4.17-4.11 (m, 1H), 3.76 (s, 3H), 3.66 (td, J=5.2, 2.7 Hz, 1H), 2.95 (ddd, J=17.4, 3.9, 2.4 Hz, 1H), 2.57 (s, 1H), 2.46 (dt, J=17.3, 1.2 Hz, 1H), 1.96-1.81 (m, 2H), 1.72-1.60 (m, 1H), 1.50-1.36 (m, 1H).

Step 8: A solution of (6S,9R)-3-methoxy-6,7,8,9-tetrahydro-5H-6,9-epiminocyclohepta[c]pyridine I-7p (0.99 g, 5.2 mmol) in HBr (48% in water) (8.8 ml, 78 mmol) was heated at reflux for 16 h. The mixture was concentrated in vacuo and the concentrate was diluted with MeOH, loaded onto a SCX cartridge (80 g), the cartridge was 15 washed with MeOH and the product was eluted with NH₃ in MeOH solution (0.7M) to afford (6S,9R)-2,5,6,7,8,9-hexahydro-3H-6,9-epiminocyclohepta[c]pyridin-3-one 1-7 as a brown solid. ¹H NMR (400 MHz, DMSO-d6) δ 11.11 (s, 1H), 7.03 (s, 1H), 6.01 (s, 1H), 4.04 (d, J=5.4 Hz, 1H), 3.62 (t, J=5.9 Hz, 1H), 2.83 (dd, J=17.7, 5.1 Hz, 1H), 2.40 (dt, J=17.7, 1.3 Hz, 1H), 1.92-1.78 (m, 2H), 1.68-1.56 (m, 1H), 1.45 (dt, J=9.3, 4.8 Hz, 1H), Exchangeable NH not observed.

### Intermediate 8 (1-8)

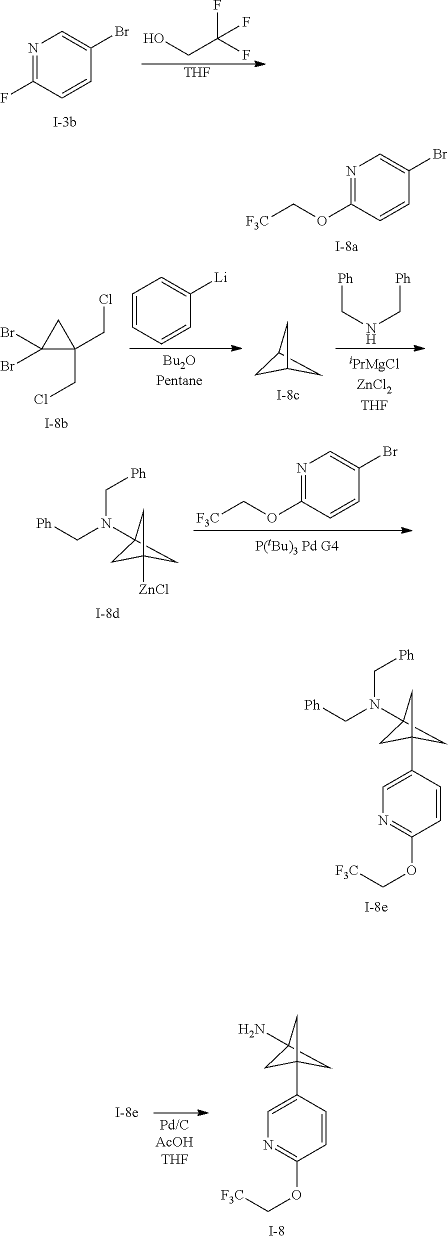

Step 1: To a solution of 4-bromo-2-fluoropyridine I-3b (2.00 g, 10.8 mmol) and trifluoroethanol (3.14 mL, 43.2 mmol) in THF (10 mL) at 0° C. was added a solution of KO^{t}Bu in THF (26.1 mL, 20% Wt, 43.2 mmol). The reaction was allowed to warm to RT and stirred for 18 h before DCM (100 mL) and water (150 mL) were added and the layers separated.

The aqueous phase was extracted with additional DCM (3×50 mL) and the combined organics were washed with water (2×50 mL) and brine (100 mL), dried over sodium sulfate and concentrated in vacuo to give 4-bromo-2-(2,2,2-trifluoroethoxy)pyridine I-8a as a colourless oil that solidified over standing. ¹H NMR (400 MHz, DMSO-d6) δ 8.34 (dd, J=2.6, 0.7 Hz, 1H), 8.01 (dd, J=8.8, 2.6 Hz, 1H), 7.01 (dd, J=8.8, 0.7 Hz, 1H), 4.97 (q, J=9.0 Hz, 2H).

Step 2: To a cooled suspension of 1,1′-bis(chloromethyl)-2,2-dibromocyclopropane (10.0 g, 33.7 mmol) in dibutyl ether (10 mL) at −20° C. was slowly added a solution of phenyllithium in dibutyl ether (37.4 mL, 1.8 M, 67.4 mmol) over 20 min. The reaction mixture was then stirred at 0° C. for 2 h before pentane (50 mL) was added, and the mixture was distilled to yield a 0.31M stock solution of tricyclo[1.1.1.01,3]pentane I-8c. ¹H NMR (400 MHz, CDCl₃) δ 1.93 (s, 6H).

Step 3: In a vial, isopropylmagnesium chloride lithium chloride complex solution 1.3M in THF (2.62 mL, 1.3 M, 3.41 mmol) was added dropwise to a solution of dibenzylamine (596.7 μL, 3.1 mmol) in THF (5 mL) at 0° C. The resulting solution was then stirred at RT for 1 h before a solution of tricyclo[1.1.1.01,3]pentane I-8c (10 mL, 0.31 M, 3.100 mmol) was added slowly under an N₂ atmosphere. The resulting mixture was sealed and heated to 50° C. for 2 h. The reaction was then cooled to 0° C. and degassed with vaccum/nitrogen cycles before a solution of zinc chloride in 2MeTHF (3.26 mL, 1.9 M, 6.2 mmol) was added dropwise, and the mixture was warmed to RT and stirred for 1 h. The resulting mixture of (3-(dibenzylamino)bicyclo[1.1.1]pentan-1-yl)zinc(II) chloride I-8d was purged with nitrogen and used directly in the next step.

Step 4: To the solution of (3-(dibenzylamino)bicyclo[1.1.1]pentan-1-yl)zinc(II) chloride I-8d (563 mg, 1.55 mmol) in THF was added 5-bromo-2-(2,2,2-trifluoroethoxy)pyridine I-8a (397 mg, 1.55 mmol) followed by a nitrogen purged solution of P(tBu)₃ Pd G4 (45.5 mg, 77.5 μmol) in THF (2 mL). The reaction mixture was heated to 50° C. for 16 h before a saturated aqueous ammonium chloride solution (5 mL) and EtOAc (30 mL) were slowly added and the resulting suspension was passed through a pad of diatomaceous earth that was washed EtOAc. The aqueous phase was separated and further extracted with EtOAc (2×15 mL).

The combined organics were washed with brine, dried over magnesium sulphate, and concentrated in vacuo. The product was purified on silica gel (0-30% ethyl acetate/iso-hexane) to give N,N-dibenzyl-3-(6-(2,2,2-trifluoroethoxy)pyridin-3-yl)bicyclo[1.1.1]pentan-1-amine I-8e as a yellowish oil. 1H NMR (400 MHz, DMSO-d6) δ 7.95 (dd, J=2.5, 0.8 Hz, 1H), 7.59 (dd, J=8.5, 2.3 Hz, 1H), 7.42-7.25 (m, 8H), 7.24-7.17 (m, 2H), 6.87 (dd, J=8.4, 0.8 Hz, 1H), 4.93 (q, J=9.1 Hz, 2H), 3.65 (s, 4H), 1.92 (s, 6H).

Step 5: A solution of N,N-dibenzyl-3-(6-(2,2,2-trifluoroethoxy)pyridin-3-yl)bicyclo[1.1.1]pentan-1-amine I-8e (336 mg, 383 μmol) in THF (2 mL) was purged with nitrogen followed by addition of palladium on carbon (35.0 mg, 10% Wt, 329 μmol). The reaction was stirred at RT under H₂ atm. (5 bars) over 64 h. THF (1 mL) was added followed by acetic acid (50 μL, 2.3 Eq, 873 μmol) and the resulting mixture was stirred at RT under a H₂ atm. (5 bars) for 24 h. The reaction mixture was filtered over celite and washed with EtOH (ca. 20 mL) and the filtrate was concentrated in vacuo. The product was purified by chromatography on silica gel (0-100% EtOAc:EtOH(7:3)/iso-hexane) to afford 3-(6-(2,2,2-trifluoroethoxy)pyridin-3-yl)bicyclo[1.1.1]pentan-1-amine 1-8 as a sticky colourless glass. ¹H NMR (400 MHz, DMSO-d6) δ 7.99 (d, J=2.4 Hz, 1H), 7.63 (dd, J=8.4, 2.4 Hz, 1H), 6.90 (d, J=8.5 Hz, 1H), 4.95 (q, J=9.2 Hz, 2H), 1.96 (s, 6H) 2 exchangeable protons not observed.

### Intermediate 9

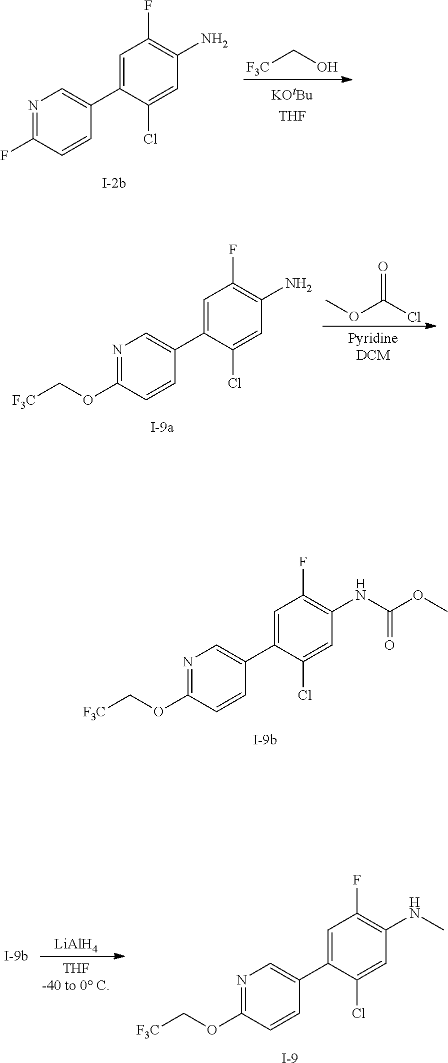

Step 1: To a solution of 5-chloro-2-fluoro-4-(6-fluoropyridin-3-yl)aniline I-2b (18.00 g, 71.06 mmol) and trifluoroethanol (20.7 mL, 284.2 mmol) in THF (160 mL) at 0° C. was added a solution of KO^{t}Bu in THF (172 mL, 20% Wt, 284.2 mmol) dropwise. The reaction was allowed to warm to RT and stirred for 72 h before water (400 mL) was added and the layers were separated. The organic layer was diluted with DCM (400 mL) and then washed with water (400 mL). The combined aqueous layers were extracted with additional DCM (3×200 mL) before the combined organics were dried over sodium sulfate and concentrated in vacuo. The product was purified by chromatography on silica gel (0-30% EtOAc/iso-hexane) to afford 5-chloro-2-fluoro-4-(6-(2,2,2-trifluoroethoxy)pyridin-3-yl)aniline I-9a as a dark orange oil that solidified upon standing. ¹H NMR (400 MHz, DMSO-d6) δ 8.19 (dd, J=2.5, 0.8 Hz, 1H), 7.83 (dd, J=8.5, 2.5 Hz, 1H), 7.15 (d, J=11.9 Hz, 1H), 7.03 (dd, J=8.6, 0.8 Hz, 1H), 6.92 (d, J=8.3 Hz, 1H), 5.63 (s, 2H), 5.02 (q, J=9.1 Hz, 2H).

Step 2: To a solution of 5-chloro-2-fluoro-4-(6-(2,2,2-trifluoroethoxy)pyridin-3-yl)aniline I-9a (900 mg, 2.81 mmol) in DCM (15 mL) was added pyridine (226 μL, 2.81 mmol) and the resultant solution was cooled to 0° C. before methyl chloroformate (239 μL, 3.09 mmol) was added. The reaction mixture was stirred at 0° C. for 45 min before warming to RT for 1 h and then the mixture was washed with a 1:1 mixture of water and brine (20 mL). The aqueous was extracted with additional DCM (50 mL) and the combined organics were concentrated in vacuo to give methyl (5-chloro-2-fluoro-4-(6-(2,2,2-trifluoroethoxy)pyridin-3-yl)phenyl)carbamate I-9b as a yellow solid. ¹H NMR (400 MHz, DMSO-d6) δ 9.77 (s, 1H), 8.28 (dd, J=2.5, 0.8 Hz, 1H), 7.96 (d, J=7.4 Hz, 1H), 7.93 (dd, J=8.6, 2.5 Hz, 1H), 7.45 (d, J=11.2 Hz, 1H), 7.09 (dd, J=8.6, 0.8 Hz, 1H), 5.05 (q, J=9.1 Hz, 2H), 3.70 (s, 3H).

Step 3: A solution of methyl (5-chloro-2-fluoro-4-(6-(2,2,2-trifluoroethoxy)pyridin-3-yl)phenyl)carbamate I-9b (850 mg, 2.24 mmol) in THF (25 mL) was cooled to −40° C. A solution of lithium aluminium hydride in THF (1.00 mL, 2.4 M, 2.40 mmol) was added and the resulting solution was stirred at −40° C. for 30 min before warming to 0° C. The reaction mixture was then slowly warmed to RT for 20 h before being cooled to 0° C. and a solution of lithium aluminum hydride in THF (1.50 mL, 2.4 M, 3.6 mmol) was added. The resulting mixture was allowed to slowly warm to RT and then stirred for 24 h. It was then cooled to 0° C. and a solution of lithium aluminum hydride (1.50 mL, 2.4 M, 3.6 mmol) was added and the reaction mixture was warmed to RT for 4 h, cooled to 0° C. and Na₂SO₄.10H₂0 (17.4 g, 53.9 mmol) was added. The resulting mixture was diluted with water (121 μL, 6.73 mmol) and the resulting suspension was warmed to RT. The reaction mixture was filtered, and the solid was washed with THF (25 mL). The filtrate was concentrated in vacuo. The product was purified by chromatography on silica gel (0-10% EtOAc/iso-hexane) to afford 5-chloro-2-fluoro-N-methyl-4-(6-(2,2,2-trifluoroethoxy)pyridin-3-yl)aniline as a yellow solid. ¹H NMR (400 MHz, DMSO-d6) δ 8.20 (d, J=2.4 Hz, 1H), 7.85 (dd, J=8.6, 2.4 Hz, 1H), 7.18 (d, J=12.2 Hz, 1H), 7.04 (d, J=8.6 Hz, 1H), 6.76 (d, J=8.2 Hz, 1H), 6.17-5.98 (m, 1H), 5.03 (q, J=9.1 Hz, 2H), 2.75 (d, J=4.9 Hz, 3H).

### Intermediate 10

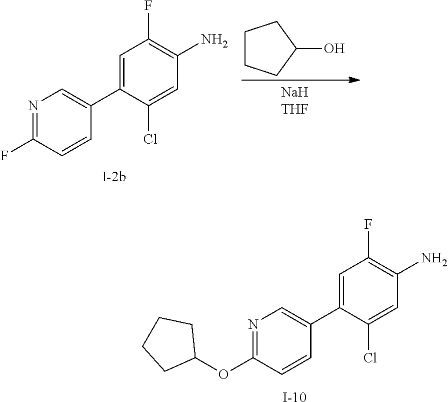

5-Chloro-4-(6-(cyclopentyloxy)pyridin-3-yl)-2-fluoroaniline 1-10 was synthesied from 5-chloro-2-fluoro-4-(6-fluoropyridin-3-yl)aniline I-2b using a procedure essentially the same as for 1-2. ¹H NMR (400 MHz, DMSO-d6) δ 8.13 (dd, J=2.6, 0.7 Hz, 1H), 7.69 (dd, J=8.6, 2.5 Hz, 1H), 7.11 (d, J=11.9 Hz, 1H), 6.90 (d, J=8.3 Hz, 1H), 6.77 (dd, J=8.5, 0.7 Hz, 1H), 5.58 (s, 2H), 5.48-5.31 (m, 1H), 1.99-1.86 (m, 2H), 1.82-1.66 (m, 4H), 1.66-1.45 (m, 2H).

### Intermediate 11 (1-11)

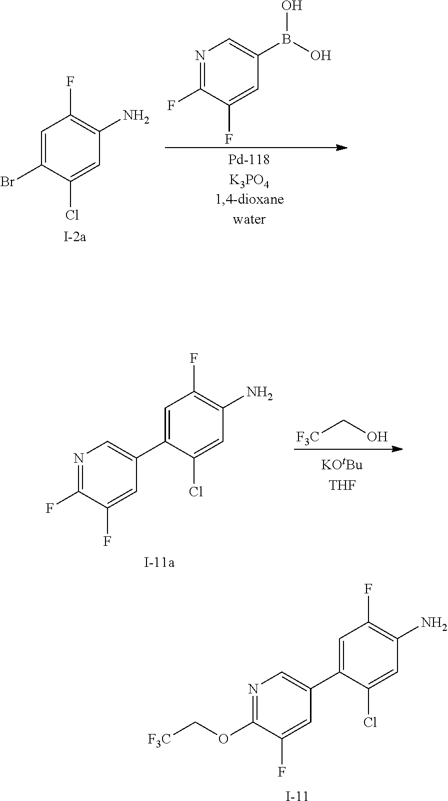

Step 1: A vial containing 4-bromo-5-chloro-2-fluoroaniline I-2a (424 mg, 1.89 mmol), (5,6-difluoropyridin-3-yl)boronic acid (250 mg, 1.57 mmol) and Pd-118 (29 mg, 44.9 μmol) was flushed with N₂, before 1,4-dioxane (3 mL) was added, followed by a degassed aqueous solution of potassium phosphate (2.36 mL, 2 M, 4.72 mmol). The reaction mixture was heated to 95° C. for 24 h, allowed to cool to RT and water (15 mL) was added and the product was extracted with DCM (3×10 mL). The combined organics were passed through a hydrophobic frit and the filtrate was concentrated in vacuo. The product was purified by chromatography on silica gel (30-100% DCM/Heptane), and a subsequent trituration of material from n-pentane gave 5-chloro-4-(5,6-difluoropyridin-3-yl)-2-fluoroaniline I-11a as a light yellow solid. ¹H NMR (400 MHz, DMSO-d6) δ 8.14 (ddd, J=10.8, 9.3, 2.1 Hz, 1H), 8.08 (t, J=1.9 Hz, 1H), 7.24 (d, J=11.9 Hz, 1H), 6.93 (d, J=8.3 Hz, 1H), 5.76 (s, 2H) Step 2: To a solution of 5-chloro-4-(5,6-difluoropyridin-3-yl)-2-fluoroaniline 1-11a (300 mg, 1.10 mmol) and trifluoroethanol (320 μL, 4.41 mmol) in THF (10 mL) at 0° C., was added a solution of KO^{t}Bu in THF (2.66 mL, 20% Wt, 4.41 mmol) and the resulting solution was allowed to warm to RT for 20 h. The reaction mixture was partitioned between brine (20 mL) and DCM (20 mL) and the layers were separated. The aqueous was extracted with DCM (20 mL), the organics were combined and concentrated in vacuo. The product was purified by chromatography on silica gel (0-20% (0.7 M ammonia/MeOH)/DCM) to afford 5-chloro-2-fluoro-4-(5-fluoro-6-(2,2,2-trifluoroethoxy)pyridin-3-yl)aniline 1-11 as a pale yellow oil. 1H NMR (400 MHz, DMSO-d6) δ 8.04 (d, J=1.9 Hz, 1H), 7.89 (dd, J=11.3, 2.0 Hz, 1H), 7.20 (d, J=11.9 Hz, 1H), 6.92 (d, J=8.3 Hz, 1H), 5.69 (s, 2H), 5.12 (q, J=9.0 Hz, 2H).

### Intermediate 12 (1-12)

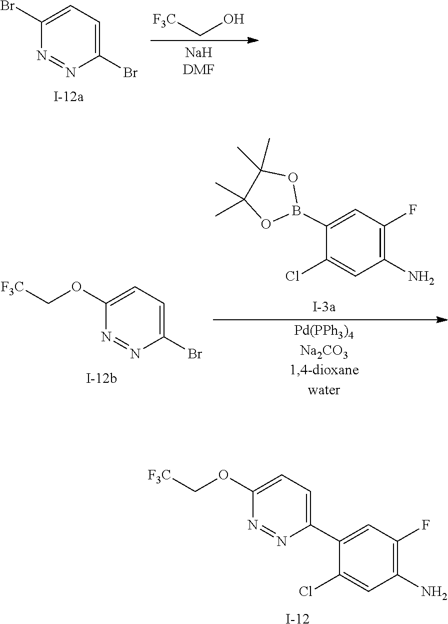

Step 1: To a solution of trifluoroethanol (305 μL, 4.24 mmol) in DMF (10 mL) was added NaH (339 mg, 8.48 mmol) at 0° C. After stirring at 0° C. for 30 min 3,6-dibromopyridazine I-12a (1 g, 4.24 mmol) was added and the reaction was stirred at RT for 16 h. The mixture was poured into NH₄Cl aqueous solution (5 mL) and extracted with EtOAc (3×30 mL). The combined organics were washed with brine (80 mL), dried over Na₂SO₄ and concentrated in vacuo. The product was purified by chromatography on silica gel (10% EtOAc/petroleum ether) to give 3-bromo-6-(2,2,2-trifluoroethoxy)pyridazine I-12b as a light yellow solid. ¹H NMR (400 MHz, DMSO-d6) δ 8.00 (d, J=9.2 Hz, 1H), 7.45 (d, J=9.2 Hz, 1H), 5.15 (q, J=8.8 Hz, 2H).

Step 2: A mixture of 5-chloro-2-fluoro-4-(4,4,5,5-tetramethyl-1,3,2-dioxaborolan-2-yl) aniline I-3a (300 mg, 1.10 mmol), 3-bromo-6-(2,2,2-trifluoroethoxy)pyridazine I-12b (284 mg, 1.1 mmol), Na₂CO₃ (352 mg, 3.30 mmol) and Pd(PPh₃)₄(128 mg, 0.11 mmol) in 1,4-dioxane (10 mL) and water (2 mL) was heated to 100° C. for 16 h. The reaction was allowed to cool to RT and concentrated in vacuo. The product was purified by chromatography on silica gel (10% EtOAc/petroleum ether) to give 5-chloro-2-fluoro-4-(6-(2,2,2-trifluoroethoxy)pyridazin-3-yl)aniline 1-12 as a white solid. LCMS (Method 5) m/z 322.0 (M+H)⁺ (ES⁺), at 1.39 min

### Intermediate 13 (1-13)

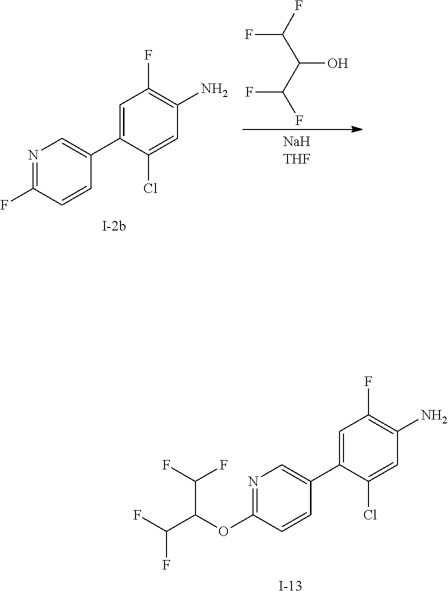

5-Chloro-2-fluoro-4-(6-((1,1,3,3-tetrafluoropropan-2-yl)oxy)pyridin-3-yl)aniline 1-13 was synthesied from 5-chloro-2-fluoro-4-(6-fluoropyridin-3-yl)aniline I-2b and 1,1,3,3-tetrafluoropropan-2-ol using a procedure essentially the same as for 1-2. ¹H NMR (400 MHz, DMSO-d6) δ 8.19 (dd, J=2.5, 0.7 Hz, 1H), 7.86 (dd, J=8.6, 2.5 Hz, 1H), 7.17 (d, J=11.9 Hz, 1H), 7.10-7.02 (m, 1H), 6.92 (d, J=8.4 Hz, 1H), 6.71-6.32 (m, 2H), 6.07 (tt, J=12.3, 3.2 Hz, 1H), 5.63 (s, 2H).

### Intermediate 14 (1-14)

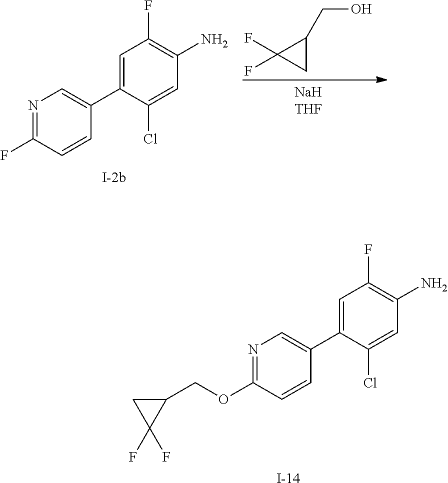

5-Chloro-4-(6-((2,2-difluorocyclopropyl)methoxy)pyridin-3-yl)-2-fluoroaniline 1-14 was synthesised from 5-chloro-2-fluoro-4-(6-fluoropyridin-3-yl)aniline I-2b and (2,2-difluorocyclopropyl)methanol using a procedure essentially the same as for 1-2. ¹H NMR (400 MHz, DMSO-d6) δ 8.14 (dd, J=2.6, 0.8 Hz, 1H), 7.75 (dd, J=8.6, 2.5 Hz, 1H), 7.12 (d, J=11.9 Hz, 1H), 6.99-6.78 (m, 2H), 5.59 (s, 2H), 4.61-4.39 (m, 1H), 4.22 (ddd, J=11.7, 8.6, 1.8 Hz, 1H), 2.36-2.14 (m, 1H), 1.79-1.71 (m, 1H), 1.53-1.46 (m, 1H).

### Intermediate 15 (1-15)

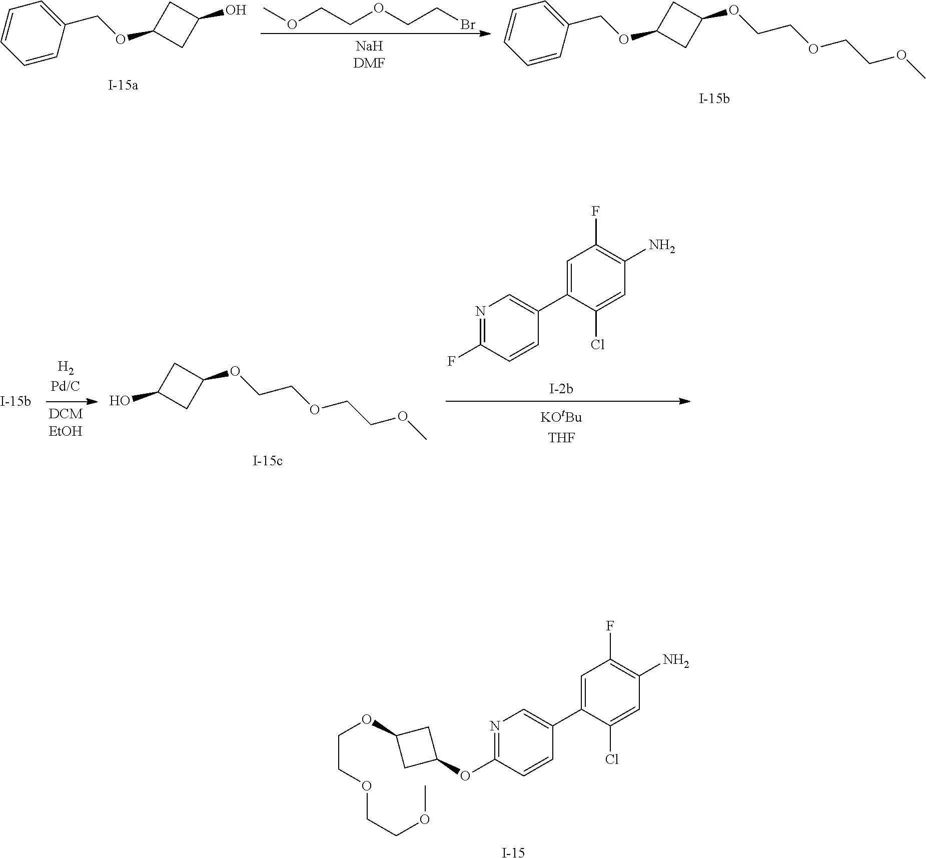

Step 1: To a solution of (1s,3s)-3-(benzyloxy)cyclobutan-1-ol I-15a (500 mg, 2.81 mmol) in DMF (11 mL) at 5° C. was added sodium hydride (168 mg, 60% Wt, 4.21 mmol) and the reaction mixture was stirred at RT for 1 h. 2-(2-methoxyethoxy)ethylbromide (418 μL, 3.09 mmol) was added and the reaction was stirred at RT for 3 days. The resulting mixture was cooled down to 10° C. and sodium hydride (111 mg, 60% Wt, 2.78 mmol) was added. After 30 min, 2-(2-methoxyethoxy)ethylbromide (154 μL, 1.14 mmol) was added and the reaction was warmed to RT for 2 h before being poured into an aqueous LiCl solution (70 mL, 1 M).

The product was extracted with EtOAc (3×50 mL) and the combined organics were dried over magnesium sulfate and concentrated in vacuo. The product was purified by chromatography on silica gel (0-50% EtOAc/iso-hexane) to afford (((1s,3s)-3-(2-(2-methoxyethoxy)ethoxy)cyclobutoxy)methyl)benzene I-15b as a clear yellow oil. 1H NMR (400 MHz, DMSO-d6) δ 7.38-7.23 (m, 5H), 4.35 (s, 2H), 3.72-3.53 (m, 2H), 3.52-3.45 (m, 4H), 3.45-3.40 (m, 2H), 3.40-3.35 (m, 2H), 3.23 (s, 3H), 2.63-2.52 (m, 2H), 1.80-1.66 (m, 2H).

Step 2: A solution of (((1s,3s)-3-(2-(2-methoxyethoxy)ethoxy)cyclobutoxy)methyl)benzene I-15b (470 mg, 1.51 mmol) in DCM (3 mL) and EtOH (1 mL) was purged with nitrogen followed by addition of 10% Pd/C (100.0 mg, 94 μmol). The reaction was stirred at RT under a H₂ atmosphere (4 Bar) for 16 h. The reaction mixture was filtered through a GF/F filter paper. The filter paper was washed with MeOH (10 mL) and the filtrate was concentrated in vacuo to give (1s,3s)-3-(2-(2-methoxyethoxy)ethoxy)cyclobutan-1-ol I-15c as a colourless oil. 1H NMR (400 MHz, DMSO-d6) b 3.70-3.61 (m, 1H), 3.51-3.44 (m, 5H), 3.44-3.40 (m, 2H), 3.35 (dd, J=5.8, 3.9 Hz, 2H), 3.23 (s, 3H), 2.56-2.47 (m, 2H over DMSO peak), 1.74-1.60 (m, 2H). 1 exchangeable proton not observed.

Step 3: 5-Chloro-2-fluoro-4-(6-((1s,3s)-3-(2-(2-methoxyethoxy)ethoxy)cyclobutoxy)pyridin-3-yl)aniline 1-15 was synthesised from (1s,3s)-3-(2-(2-methoxyethoxy)ethoxy)cyclobutan-1-ol I-15c and 5-chloro-2-fluoro-4-(6-fluoropyridin-3-yl)aniline I-2b using a procedure essentially the same as for 1-11. ¹H NMR (400 MHz, DMSO-d6) δ 8.11 (d, J=2.6 Hz, 1H), 7.72 (dd, J=8.6, 2.5 Hz, 1H), 7.12 (d, J=11.9 Hz, 1H), 6.89 (d, J=8.4 Hz, 1H), 6.83 (d, J=8.5 Hz, 1H), 5.61 (s, 2H), 4.81 (p, J=7.1 Hz, 1H), 3.75 (p, J=7.0 Hz, 1H), 3.54-3.47 (m, 4H), 3.47-3.40 (m, 4H), 3.23 (s, 3H), 2.88-2.77 (m, 2H), 2.01-1.88 (m, 2H).

### Intermediate 16 (1-16)

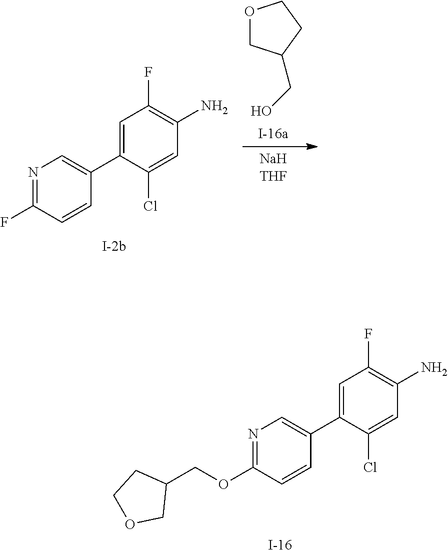

5-Chloro-2-fluoro-4-(6-((tetrahydrofuran-3-yl)methoxy)pyridin-3-yl)aniline 1-16 was synthesised from 5-chloro-2-fluoro-4-(6-fluoropyridin-3-yl)aniline I-2b and (tetrahydrofuran-3-yl)methanol I-16a using a procedure essentially the same as for 1-16. ¹H NMR (400 MHz, DMSO-d6) δ 8.13 (dd, J=2.6, 0.7 Hz, 1H), 7.73 (dd, J=8.5, 2.5 Hz, 1H), 7.11 (d, J=11.9 Hz, 1H), 6.91 (d, J=8.4 Hz, 1H), 6.85 (dd, J=8.5, 0.8 Hz, 1H), 5.59 (s, 2H), 4.26 (dd, J=10.5, 6.7 Hz, 1H), 4.17 (dd, J=10.5, 8.0 Hz, 1H), 3.83-3.71 (m, 2H), 3.65 (td, J=8.0, 6.9 Hz, 1H), 3.54 (dd, J=8.6, 5.5 Hz, 1H), 2.74-2.61 (m, J=7.1, 6.4 Hz, 1H), 2.08-1.96 (m, 1H), 1.74-1.57 (m, 1H).

### Intermediate 17 (1-17)

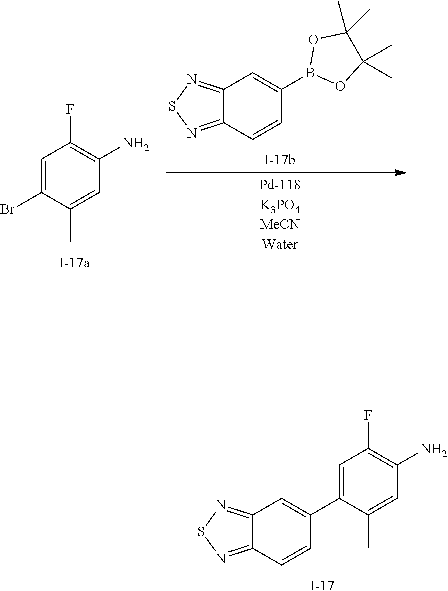

4-(Benzo[c][1,2,5]thiadiazol-5-yl)-2-fluoro-5-methylaniline 1-17 was synthesised from 4-bromo-2-fluoro-5-methylaniline I-17a and 5-(4,4,5,5-tetramethyl-1,3,2-dioxaborolan-2-yl)benzo[c][1,2,5]thiadiazole I-17b using a procedure essentially the same as for I-2b. ¹H NMR (400 MHz, DMSO-d6) δ 8.07 (dd, J=9.1, 0.8 Hz, 1H), 7.93 (dd, J=1.7, 0.8 Hz, 1H), 7.71 (dd, J=9.1, 1.7 Hz, 1H), 7.03 (d, J=12.2 Hz, 1H), 6.72 (d, J=9.3 Hz, 1H), 5.29 (s, 2H), 2.18 (s, 3H).

### Intermediate 18 (1-18)

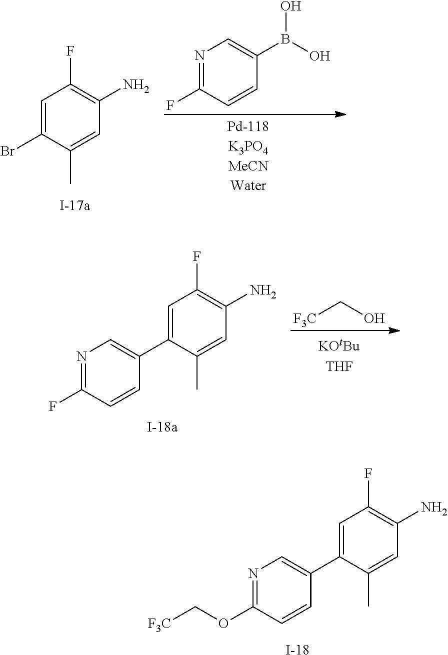

Step 1: 2-Fluoro-4-(6-fluoropyridin-3-yl)-5-methylaniline I-18a was synthesised from 4-bromo-2-fluoro-5-methylaniline I-17a and (6-fluoropyridin-3-yl)boronic acid using a procedure essentially the same as for I-2b. ¹H NMR (400 MHz, DMSO-d6) δ 8.15 (dt, J=2.6, 0.9 Hz, 1H), 7.92 (td, J=8.2, 2.6 Hz, 1H), 7.19 (ddd, J=8.5, 2.9, 0.7 Hz, 1H), 6.92 (d, J=12.1 Hz, 1H), 6.74-6.64 (m, 1H), 5.24 (s, 2H), 2.09 (s, 3H).

Step 2: 2-Fluoro-5-methyl-4-(6-(2,2,2-trifluoroethoxy)pyridin-3-yl)aniline 1-18 was synthesied from 2-fluoro-4-(6-fluoropyridin-3-yl)-5-methylaniline I-18a and trifluoroethanol using a procedure essentially the same as for 1-11. ¹H NMR (500 MHz, DMSO-d6) δ 8.11 (d, J=2.4 Hz, 1H), 7.75 (dd, J=8.5, 2.5 Hz, 1H), 7.00 (d, J=8.5 Hz, 1H), 6.88 (d, J=12.1 Hz, 1H), 6.68 (d, J=9.3 Hz, 1H), 5.18 (s, 2H), 5.01 (q, J=9.1 Hz, 2H), 2.09 (s, 3H)

### Intermediate 19 (1-19)

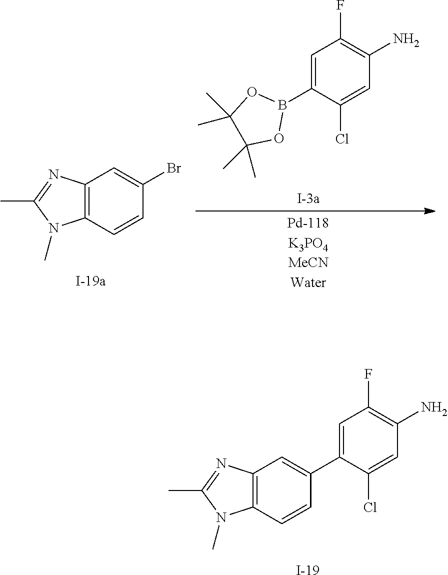

5-Chloro-4-(1,2-dimethyl-1H-benzo[d]imidazol-5-yl)-2-fluoroaniline 1-19 was synthesised from 5-bromo-1,2-dimethyl-1H-benzo[d]imidazole I-19a and 5-chloro-2-fluoro-4-(4,4,5,5-tetramethyl-1,3,2-dioxaborolan-2-yl)aniline I-3a using a procedure essentially the same as for 1-4. ¹H NMR (400 MHz, DMSO-d6) δ 7.50-7.44 (m, 2H), 7.17 (dd, J=8.2, 1.8 Hz, 1H), 7.06 (d, J=12.0 Hz, 1H), 6.90 (d, J=8.4 Hz, 1H), 5.48 (s, 2H), 3.74 (s, 3H), 2.53 (s, 3H).

### Intermediate 20 (1-20)

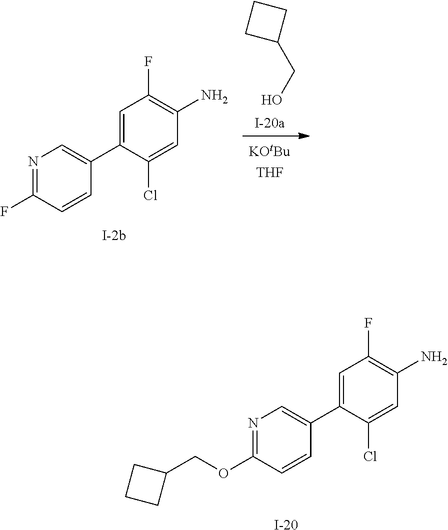

5-Chloro-4-(6-(cyclobutylmethoxy)pyridin-3-yl)-2-fluoroaniline 1-20 was synthesised from 5-chloro-2-fluoro-4-(6-fluoropyridin-3-yl)aniline I-2b and cyclobutylmethanol I-20a using a procedure essentially the same as for 1-11. ¹H NMR (400 MHz, DMSO-d6) δ 8.12 (dd, J=2.5, 0.7 Hz, 1H), 7.71 (dd, J=8.6, 2.5 Hz, 1H), 7.12 (d, J=11.9 Hz, 1H), 6.90 (d, J=8.4 Hz, 1H), 6.83 (dd, J=8.6, 0.7 Hz, 1H), 5.63-5.57 (m, 2H), 4.25 (d, J=7.0 Hz, 2H), 2.81-2.67 (m, 1H), 2.13-2.01 (m, 2H), 1.96-1.79 (m, 4H).

### Intermediate 21 (1-21)

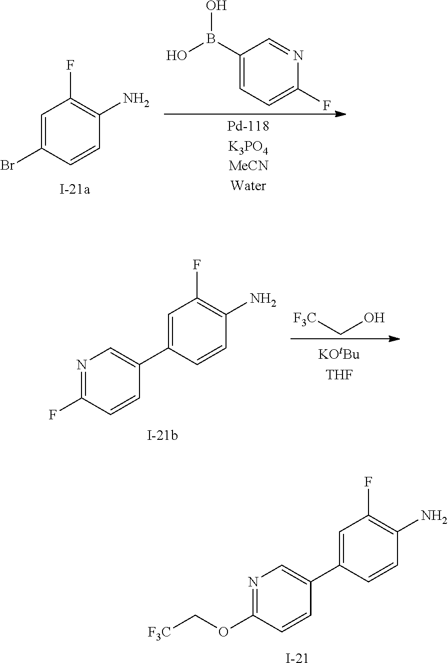

Step 1: 2-Fluoro-4-(6-fluoropyridin-3-yl)aniline 1-21b was synthesised from 4-bromo-2-fluoroaniline I-21a and (6-fluoropyridin-3-yl)boronic acid using a procedure essentially the same as for I-2b. ¹H NMR (400 MHz, DMSO-d6) δ 8.45 (d, J=2.6 Hz, 1H), 8.18 (td, J=8.2, 2.7 Hz, 1H), 7.44 (dd, J=13.0, 2.1 Hz, 1H), 7.28 (dd, J=8.3, 2.1 Hz, 1H), 7.19 (dd, J=8.6, 3.0 Hz, 1H), 6.84 (dd, J=9.5, 8.3 Hz, 1H), 5.42 (s, 2H).

Step 2. 2-Fluoro-4-(2-(2,2,2-trifluoroethoxy)pyridin-4-yl)aniline 1-21 was synthesised from 2-fluoro-4-(6-fluoropyridin-3-yl)aniline I-21b and trifluorethanol using a procedure essentially the same as for 1-11. ¹H NMR (400 MHz, DMSO-d6) δ 8.41 (dd, J=2.6, 0.8 Hz, 1H), 8.01 (dd, J=8.6, 2.6 Hz, 1H), 7.38 (dd, J=13.0, 2.1 Hz, 1H), 7.24 (dd, J=8.3, 2.1 Hz, 1H), 7.00 (dd, J=8.6, 0.7 Hz, 1H), 6.84 (dd, J=9.5, 8.3 Hz, 1H), 5.31 (s, 2H), 5.00 (q, J=9.2 Hz, 2H).

### Intermediate 22 (1-22)

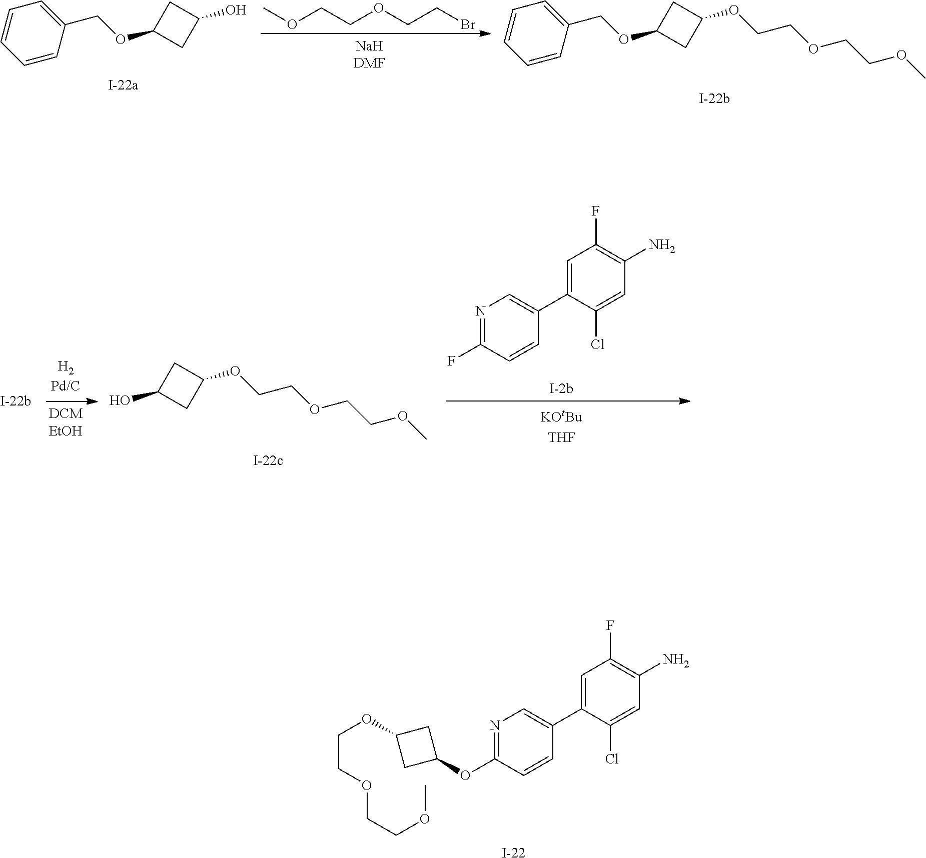

Step 1: (((1r,3r)-3-(2-(2-Methoxyethoxy)ethoxy)cyclobutoxy)methyl)benzene I-22b was synthesised from (1r,3r)-3-(benzyloxy)cyclobutan-1-ol I-22a and 2-(2-methoxyethoxy)ethylbromide using a procedure essentially the same as for I-15b. ¹H NMR (400 MHz, DMSO-d6) δ 7.39-7.23 (m, 5H), 4.35 (s, 2H), 4.19-4.04 (m, 2H), 3.54-3.46 (m, 4H), 3.46-3.36 (m, 4H), 3.23 (s, 3H), 2.23-2.06 (m, 4H).

Step 2: (1r,3r)-3-(2-(2-Methoxyethoxy)ethoxy)cyclobutan-1-ol I-22c was synthesised from (((1r,3r)-3-(2-(2-methoxyethoxy)ethoxy)cyclobutoxy)methyl)benzene I-22b using a procedure essentially the same as for I-15b. ¹H NMR (400 MHz, DMSO-d6) δ 4.29-4.17 (m, 1H), 4.09-4.00 (m, 1H), 3.52-3.45 (m, 4H), 3.44-3.40 (m, 2H), 3.38-3.32 (m, 2H), 3.23 (s, 3H), 2.11 (ddt, J=12.6, 7.1, 3.5 Hz, 2H), 2.02-1.94 (m, 2H). 1 exchangeable proton not observed.

Step 3: 5-Chloro-2-fluoro-4-(6-((1r,3r)-3-(2-(2-methoxyethoxy)ethoxy)cyclobutoxy)pyridin-3-yl)aniline 1-22 was synthesised from (1r,3r)-3-(2-(2-Methoxyethoxy)ethoxy)cyclobutan-1-ol I-22c and 5-chloro-2-fluoro-4-(6-fluoropyridin-3-yl)aniline I-2b using a proceure essentially the same as for 1-11. ¹H NMR (400 MHz, DMSO-d6) δ 8.14-8.09 (m, 1H), 7.73 (dd, J=8.6, 2.5 Hz, 1H), 7.12 (dd, J=11.8, 2.4 Hz, 1H), 6.90 (d, J=8.4 Hz, 1H), 6.83 (d, J=7.8 Hz, 1H), 5.61 (s, 2H), 5.31-5.21 (m, 1H), 4.25-4.14 (m, 1H), 3.54-3.49 (m, 4H), 3.46-3.41 (m, 4H), 3.24 (s, 3H), 2.46-2.36 (m, 2H), 2.36-2.27 (m, 2H).

### Intermediate 23 (1-23)

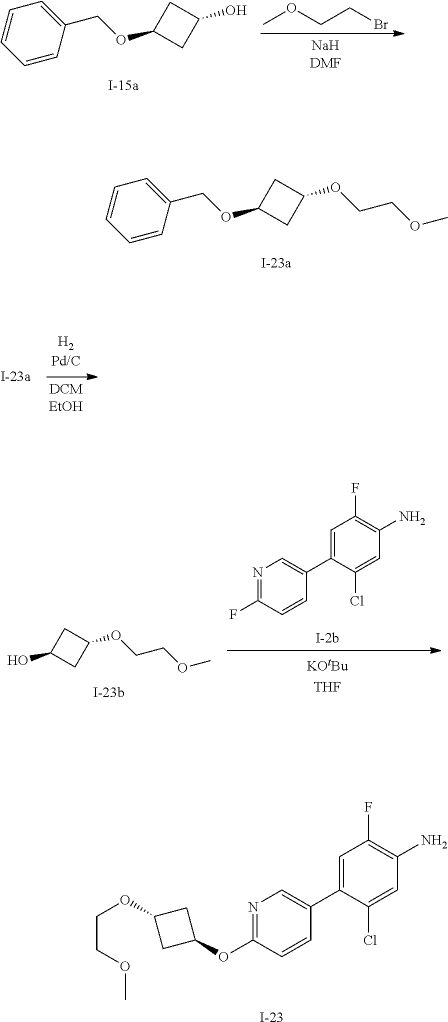

Step 1: (((1s,3s)-3-(2-Methoxyethoxy)cyclobutoxy)methyl)benzene I-23a was synthesised from (1s,3s)-3-(benzyloxy)cyclobutan-1-ol I-15a and 1-bromo-2-methoxyethane using a procedure essentially the same as for I-15b. ¹H NMR (400 MHz, DMSO-d6) δ 7.38-7.21 (m, 5H), 4.35 (s, 2H), 3.64 (p, J=7.1 Hz, 1H), 3.57 (p, J=6.9 Hz, 1H), 3.44-3.33 (m, 4H), 3.23 (s, 3H), 2.63-2.52 (m, 2H), 1.79-1.67 (m, 2H).

Step 2: (1s,3s)-3-(2-methoxyethoxy)cyclobutan-1-ol I-23b was synthesised from (((1s,3s)-3-(2-Methoxyethoxy)cyclobutoxy)methyl)benzene I-23a using a procedure essentially the same as for 1-15c. ¹H NMR (400 MHz, DMSO-d6) b 3.71-3.60 (m, 1H), 3.51-3.39 (m, 1H), 3.41-3.37 (m, 2H), 3.37-3.32 (m, 2H), 3.22 (s, 3H), 2.55-2.46 (m, 2H), 1.72-1.60 (m, 2H). 1 exchangeable proton not observed.

Step 3: 5-Chloro-2-fluoro-4-(6-((1s,3s)-3-(2-methoxyethoxy)cyclobutoxy)pyridin-3-yl)aniline 1-23 was synthesised from (1s,3s)-3-(2-methoxyethoxy)cyclobutan-1-ol I-23b and 5-chloro-2-fluoro-4-(6-fluoropyridin-3-yl)aniline I-2b using a procedure essentially the same as for I-11. ¹H NMR (400 MHz, DMSO-d6) δ 8.11 (dd, J=2.5, 0.7 Hz, 1H), 7.72 (dd, J=8.6, 2.6 Hz, 1H), 7.12 (d, J=11.9 Hz, 1H), 6.89 (d, J=8.4 Hz, 1H), 6.83 (dd, J=8.6, 0.8 Hz, 1H), 5.61 (s, 2H), 4.81 (p, J=7.3 Hz, 1H), 3.74 (p, J=6.9 Hz, 1H), 3.43 (s, 4H), 3.24 (s, 3H), 2.88-2.77 (m, 2H), 1.99-1.87 (m, 2H).

### Intermediate 24 (1-24)

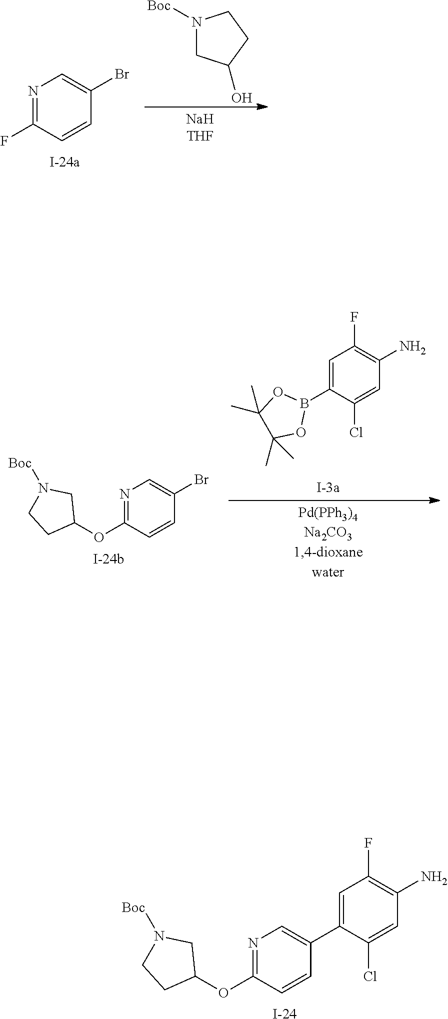

Step 1: To a solution of (±)-tert-butyl 3-hydroxypyrrolidine-1-carboxylate (535 mg, 2.85 mmol) in THF (8 mL) was added NaH (229 mg, 60% Wt, 5.70 mmol) at 0° C. After stirring at 0° C. for 30 min, 5-bromo-2-fluoropyridine I-24a (500 mg, 2.85 mmol) was added and the resulting mixture was warmed to RT for 16 h. The reaction was then poured into a saturated aqueous NH₄Cl solution (5 mL) and the product was extracted with EtOAc (3×30 mL). The combined organics were washed with brine (80 mL), dried over Na₂SO₄ and concentrated in vacuo. The product was purified by chromatography on silica gel (20% EtOAc/petroleum ether) to give (±)-tert-butyl 3-((5-bromopyridin-2-yl)oxy)pyrrolidine-1-carboxylate I-24b as a light yellow solid. ¹H NMR (400 MHz, DMSO-d6) δ 8.31 (d, J=2.4 Hz, 1H), 7.93 (dd, J=2.4, 8.8 Hz, 1H), 6.86 (d, J=8.8 Hz, 1H), 5.48-5.41 (m, 1H), 3.63-3.45 (m, 4H), 2.21-2.08 (m, 2H), 1.42 (d, J=7.6 Hz, 9H).

Step 2: (±)-tert-butyl 3-((5-(4-amino-2-chloro-5-fluorophenyl)pyridin-2-yl)oxy)pyrolidine-1-carboxylate 1-24 was synthesised from (±)-tert-butyl 3-((5-bromopyridin-2-yl)oxy)pyrrolidine-1-carboxylate I-24b and 5-chloro-2-fluoro-4-(4,4,5,5-tetramethyl-1,3,2-dioxaborolan-2-yl)aniline I-3a using a procedure essentially the same as for 1-12. LCMS (Method 5) m/z 408.1, 410.0 (M+H)⁺ (ES⁺), at 2.41 min

### Intermediate 25 (1-25)

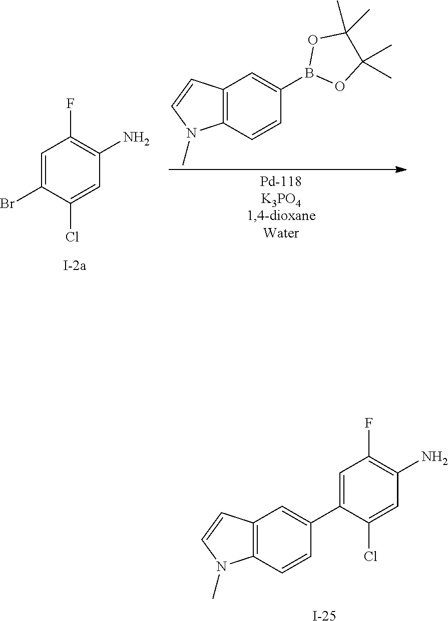

5-Chloro-2-fluoro-4-(1-methyl-1 H-indol-5-yl)aniline 1-25 was synthesised from 4-bromo-5-chloro-2-fluoroaniline I-2a and 1-methyl-5-(4,4,5,5-tetramethyl-1,3,2-dioxaborolan-2-yl)-1H-indole using a procedure essentially the same as for I-11a. ¹H NMR (400 MHz, DMSO-d6) δ 7.51 (s, 1H), 7.44 (d, J=8.4 Hz, 1H), 7.34 (d, J=2.8 Hz, 1H), 7.15 (dd, J=1.6, 8.4 Hz, 1H), 7.06 (d, J=12 Hz, 1H), 6.92 (d, J=8.4 Hz, 1H), 6.43 (d, J=2.8 Hz, 1H), 6.44 (s, 2H), 3.80 (s, 3H).

### Intermediate 26 (1-26)

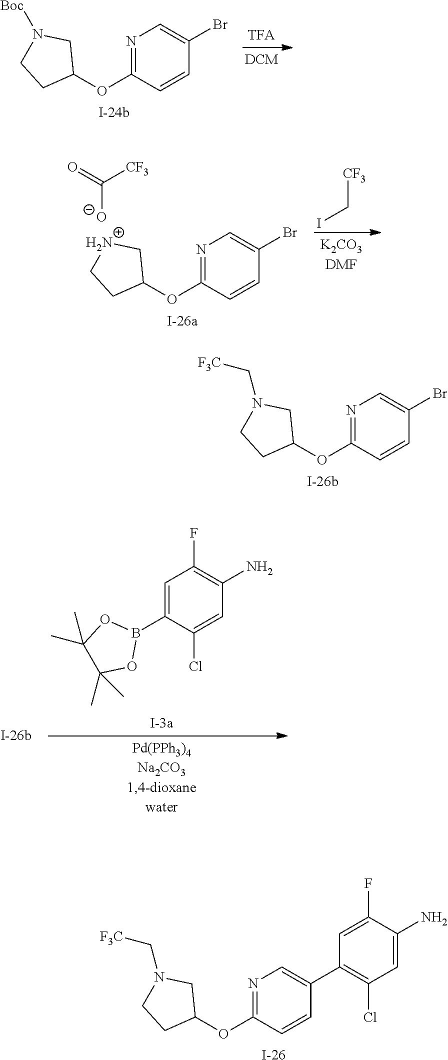

Step 1: To a solution of (±)-tert-butyl 3-((5-bromopyridin-2-yl)oxy)pyrrolidine-1-carboxylate I-24b (500 mg, 1.47 mmol) in DCM (5 mL) was added TFA (3 ml) at RT. The reaction mixture was stirred at RT for 16 h before the mixture was concentrated in vacuo to give (±)-5-bromo-2-(pyrrolidin-3-yloxy)pyridine trifluoracetate salt I-26a as a yellow oil. LCMS (Method 7) m/z 243.0, 245.0 (M+H)⁺ (ES⁺), at 0.31 min Step 2: To a solution of (±)-5-bromo-2-(pyrrolidin-3-yloxy)pyridine trifluoracetate salt I-26a (542 mg, 1.52 mmol) in DMF (5 mL) was added K₂CO₃ (629 mg, 4.55 mmol) and 1,1,1-trifluoro-2-iodoethane (956 mg, 4.55 mmol). The reaction mixture was heated at 130° C. for 1.5 h in a microwave, allowed to cool to RT, poured into water (20 mL) and the product was extracted with EtOAc (3×20 mL). The combined organics were washed with brine (20 mL), dried over Na₂SO₄ and concentrated in vacuo. The product was purified by TLC (20% EtOAc/petroleum ether) to give (±)-5-bromo-2-((1-(2,2,2-trifluoroethyl)pyrrolidin-3-yl)oxy)pyridine I-26b as a white solid. LCMS (Method 7) m/z 325.0, 327.0 (M+H)⁺ (ES⁺), at 0.31 min

Step 3: (±)-5-Chloro-2-fluoro-4-(6-((1-(2,2,2-trifluoroethyl)pyrrolidin-3-yl)oxy) pyridin-3-yl)aniline 1-26 was synthesised from (±)-5-bromo-2-((1-(2,2,2-trifluoroethyl)pyrrolidin-3-yl)oxy)pyridine I-26b and 5-chloro-2-fluoro-4-(4,4,5,5-tetramethyl-1,3,2-dioxaborolan-2-yl)aniline I-3a using a procedure essentially the same as for 1-12. LCMS (Method 7) m/z 390.1 (M+H)⁺ (ES⁺), at 1.85 min Intermediate 27 (1-27)

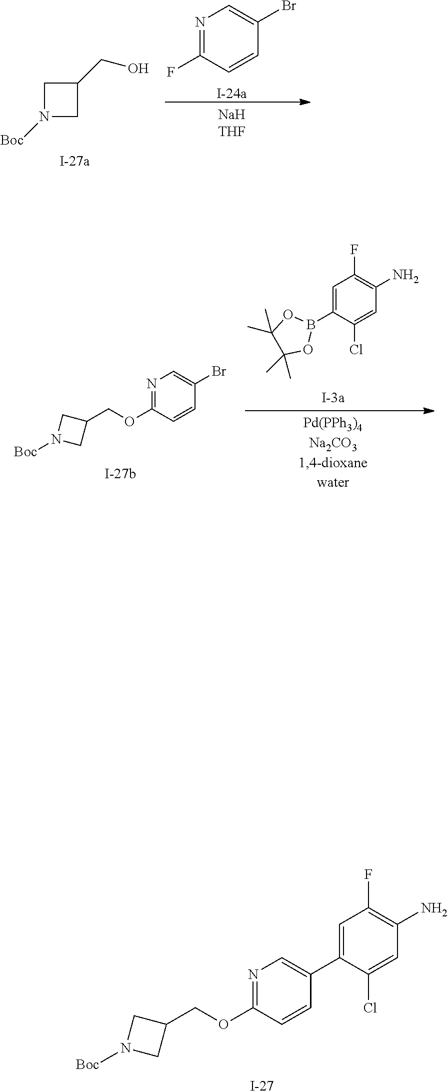

Step 1: To a solution of tert-butyl 3-(hydroxymethyl)azetidine-1-carboxylate I-27a (5.34 g, 28.55 mmol) in dry DMF (50 mL) was added NaH (1.71 g, 60% wt., 42.83 mmol) slowly at 0° C. The reaction was warmed to RT for 1 h before 5-bromo-2-fluoropyridine I-24a (5 g, 28.55 mmol) was added and the mixture was stirred at RT for 16 h. The reaction was quenched by pouring onto ice water (50 mL) and the product was extracted with EtOAc (2×100 mL). The combined organics were combined and concentrated in vacuo. The product was purified by chromatography on silica gel (10% EtOAc/petroleum ether) to give tert-butyl 3-(((5-bromopyridin-2-yl)oxy)methyl)azetidine-1-carboxylate I-27b as a yellow oil. LCMS (Method 5) m/z 342.9 (M+H)⁺ (ES⁺), at 2.01 min.

Step 2: tert-Butyl 3-(((5-(4-amino-2-chloro-5-fluorophenyl)pyridin-2-yl)oxy)methyl)azetidine-1-carboxylate 1-27 was synthesised from tert-butyl 3-(((5-bromopyridin-2-yl)oxy)methyl)azetidine-1-carboxylate I-27b and 5-chloro-2-fluoro-4-(4,4,5,5-tetramethyl-1,3,2-dioxaborolan-2-yl) aniline I-3a using a procedure essentially the same as for 1-12. LCMS (Method 5) m/z 407.9 (M+H)⁺ (ES⁺), at 1.96 min.

### Intermediate 28 (1-28)

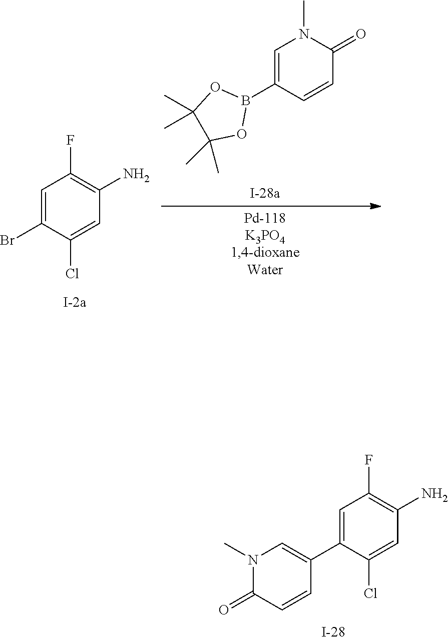

5-(4-Amino-2-chloro-5-fluorophenyl)-1-methylpyridin-2(1H)-one 1-28 was synthesised from 4-bromo-5-chloro-2-fluoroaniline I-2a and 1-methyl-5-(4,4,5,5-tetramethyl-1,3,2-dioxaborolan-2-yl)pyridin-2(1 H)-one I-28a using a procedure essentially the same as for I-11a. ¹H NMR (400 MHz, DMSO-d6) δ 7.74 (d, J=2.4 Hz, 1H), 7.48 (dd, J=2.4, 9.2 Hz, 1H), 7.09 (d, J=12.0 Hz, 1H), 6.88 (d, J=8.4 Hz, 1H), 6.39 (d, J=9.6 Hz, 1H), 5.54 (s, 2H), 3.45 (s, 3H).

### Intermediate 29 (1-29)

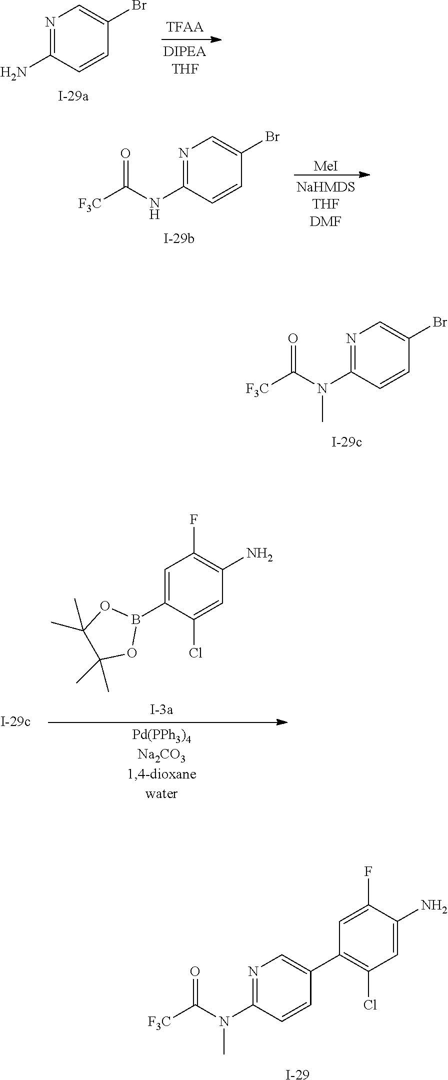

Step 1: To a solution of 5-bromopyridin-2-amine I-29a (1.0 g, 5.78 mmol) in THF (10 mL) was added TFAA (0.93 μL, 6.93 mmol) and DIPEA(2.02 mL, 11.56 mmol). The reaction was stirred at RT for 16 h before water (15 mL) was added and the product was extracted with EtOAc (3×15 mL). The combined organics were washed with brine (15 mL), dried over Na₂SO₄ and concentrated in vacuo to give N-(5-bromopyridin-2-yl)-2,2,2-trifluoroacetamide I-29b as a white solid. LCMS (Method 5) m/z 270 (M+H)⁺ (ES⁺), at 1.82 min.

Step 2: To a solution of N-(5-bromopyridin-2-yl)-2,2,2-trifluoroacetamide (1.2 g, 4.46 mmol) in THF (9 mL) was added a solution of NaHMDS in THF (2.3 mL, 2 M, 4.68 mmol) at −60° C. and the resulting mixture was stirred at −60° C. for 15 min before Mel (310 μL, 5.35 mmol) in DMF (9 mL) was added. The reaction was warmed to RT for 16 h before water (10 mL) was added and the product was extracted with EtOAc (3×20 mL). The combined organics were washed with brine (20 mL), dried over Na₂SO₄ and concentrated in vacuo. The product was purified by chromatography on silica gel (10% EtOAc/petroleum ether) to give the N-(5-bromopyridin-2-yl)-2,2,2-trifluoro-N-methylacetamide I-29c as a yellow oil. ¹H NMR (400 MHz, DMSO-d6) δ 8.77 (d, J=2.0 Hz, 1H), 8.25-8.23 (m, 1H), 8.19-8.16 (m, 1H), 3.85 (s, 3H).

Step 3: N-(5-(4-amino-2-chloro-5-fluorophenyl)pyridin-2-yl)-2,2,2-trifluoro-N-methylacetamide 1-29 was synthesised from N-(5-bromopyridin-2-yl)-2,2,2-trifluoro-N-methylacetamide I-29c and 5-chloro-2-fluoro-4-(4,4,5,5-tetramethyl-1,3,2-dioxaborolan-2-yl)aniline I-3a using a procedure essentially the same as for 1-12. LCMS (Method 5) m/z 348 (M+H)⁺ (ES⁺), at 1.77 min.

### Intermediate 30 (1-30)

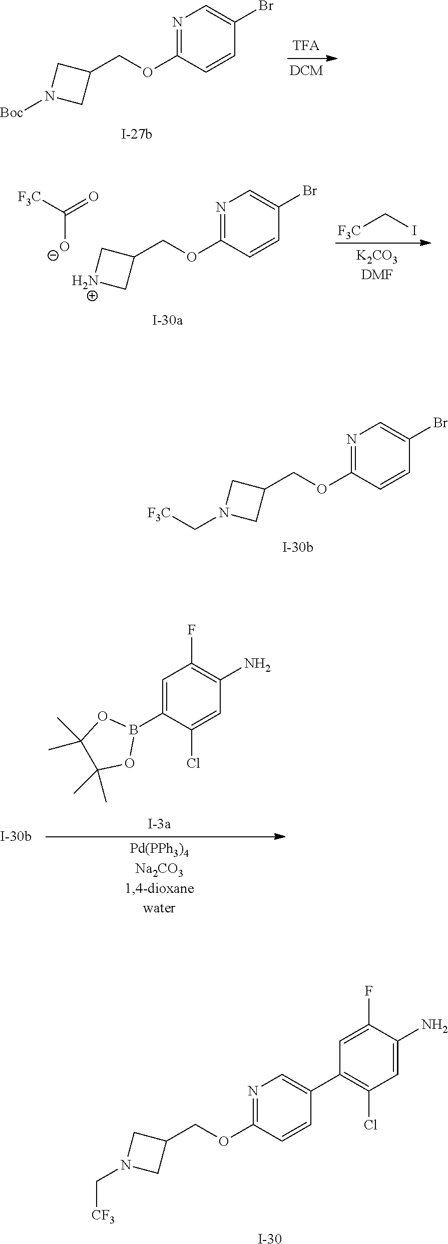

Step 1: 2-(Azetidin-3-ylmethoxy)-5-bromopyridine tiflouroacetic acid salt I-30a was synthesied from 3-(((5-bromopyridin-2-yl)oxy)methyl) azetidine-1-carboxylate I-27b using a procedure essentially the same as for 1-26a. LCMS (Method 5) m/z 242.9 (M+H)⁺ (ES⁺), at 0.94 min.

Step 2: 5-Bromo-2-((1-(2,2,2-trifluoroethyl)azetidin-3-yl)methoxy)pyridine I-30b was synthesised from 2-(azetidin-3-ylmethoxy)-5-bromopyridine tiflouroacetic acid salt I-30a and 1,1,1-trifluoro-2-iodoethane using a procedure essentially the same as for I-26b. LCMS (Method 5) m/z 324.9 (M+H)⁺ (ES⁺), at 1.03 min.

Step 3: 5-Chloro-2-fluoro-4-(6-((1-(2,2,2-trifluoroethyl)azetidin-3-yl)methoxy)pyridin-3-yl)aniline 1-30 was synthesised from 5-bromo-2-((1-(2,2,2-trifluoroethyl)azetidin-3-yl)methoxy)pyridine I-30b and 5-chloro-2-fluoro-4-(4,4,5,5-tetramethyl-1,3,2-dioxaborolan-2-yl)aniline I-3a using a procedure essentially the same as for 1-12. LCMS (Method 5) m/z 389.9 (M+H)⁺ (ES⁺), at 1.18 min.

### Intermediate 31 (1-31)

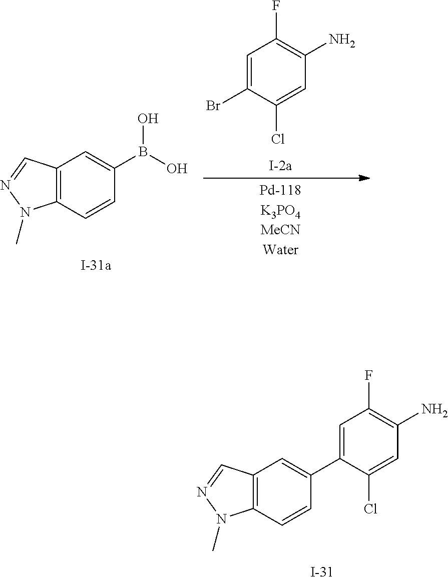

5-Chloro-2-fluoro-4-(1-methyl-1H-indazol-5-yl)aniline 1-31 was synthesised from (1-methyl-1H-indazol-5-yl)boronic acid I-31a and 4-bromo-5-chloro-2-fluoroaniline I-2a using a procedure essentially the same as for I-2b. LCMS (Method 2) m/z 276.2, 278.2 (M+H)⁺(ES⁺), at 1.75 min.

### Intermediate 32 (1-32)

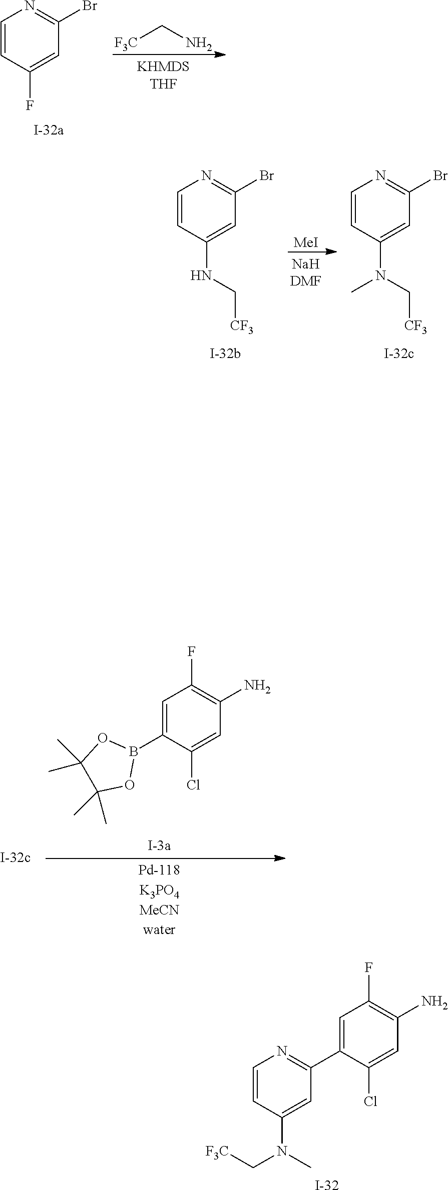

Step 1: 2-Bromo-N-(2,2,2-trifluoroethyl)pyridin-4-amine I-32b was synthesised from 2-bromo-4-fluoro-pyridine I-32a and trifluoroethylamine using a procedure essentially the same as for I-3c. ¹H NMR (400 MHz, DMSO-d6) δ 7.86 (d, J=5.8 Hz, 1H), 7.39 (t, J=6.9 Hz, 1H), 6.92 (d, J=2.2 Hz, 1H), 6.73 (dd, J=5.8, 2.2 Hz, 1H), 4.09 (qd, J=9.6, 6.9 Hz, 2H).

Step 2: 2-Bromo-N-methyl-N-(2,2,2-trifluoroethyl)pyridin-4-amine I-32c was synthesised from 2-bromo-N-(2,2,2-trifluoroethyl)pyridin-4-amine I-32b using a procedure essentially the same as for 1-4a. ¹H NMR (400 MHz, DMSO-d6) δ 7.95 (d, J=6.1 Hz, 1H), 6.99 (d, J=2.4 Hz, 1H), 6.87 (dd, J=6.0, 2.5 Hz, 1H), 4.39 (q, J=9.4 Hz, 2H), 3.04 (s, 3H).

Step 3: 2-(4-Amino-2-chloro-5-fluorophenyl)-N-methyl-N-(2,2,2-trifluoroethyl)pyridin-4-amine 1-32 was synthesised from 2-bromo-N-methyl-N-(2,2,2-trifluoroethyl)pyridin-4-amine I-32c and 5-chloro-2-fluoro-4-(4,4,5,5-tetramethyl-1,3,2-dioxaborolan-2-yl)aniline I-3a using a procedure essentially the same as for 1-3. ¹H NMR (400 MHz, DMSO-d6) δ 8.22 (d, J=6.0 Hz, 1H), 7.22 (d, J=12.2 Hz, 1H), 6.97 (d, J=2.6 Hz, 1H), 6.86 (d, J=8.3 Hz, 1H), 6.76 (dd, J=6.0, 2.6 Hz, 1H), 5.62 (s, 2H), 4.36 (q, J=9.5 Hz, 2H), 3.07 (s, 3H).

### Intermediate 33 (1-33)

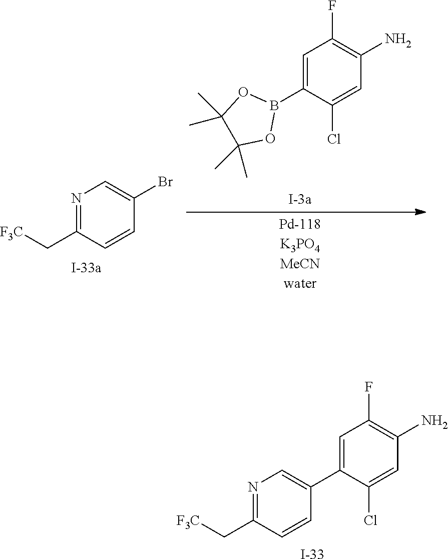

5-Chloro-2-fluoro-4-(6-(2,2,2-trifluoroethyl)pyridin-3-yl)aniline 1-33 was synthesised from 5-bromo-2-(2,2,2-trifluoroethyl)pyridine I-33a and 5-chloro-2-fluoro-4-(4,4,5,5-tetramethyl-1,3,2-dioxaborolan-2-yl)aniline I-3a using a procedure essentially the same as for 1-3. ¹H NMR (400 MHz, DMSO-d6) δ 8.58 (dd, J=2.4, 0.8 Hz, 1H), 7.86 (dd, J=8.0, 2.4 Hz, 1H), 7.50 (d, J=8.0 Hz, 1H), 7.21 (d, J=11.9 Hz, 1H), 6.93 (d, J=8.3 Hz, 1H), 5.69 (s, 2H), 3.85 (q, J=11.5 Hz, 2H).

### Intermediate 34 (1-34)

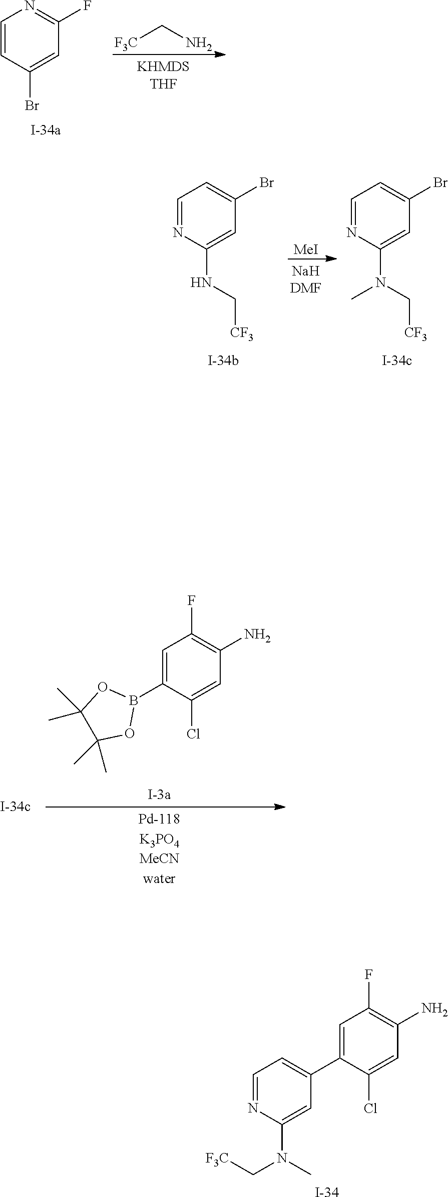

Step 1: 4-Bromo-N-(2,2,2-trifluoroethyl)pyridin-2-amine I-34b was synthesised from 4-bromo-2-fluoropyridine I-34a and trifluoroethylamine using a procedure essentially the same as for I-3c. ¹H NMR (400 MHz, DMSO-d6) δ 7.92 (d, J=5.4 Hz, 1H), 7.38 (t, J=6.6 Hz, 1H), 6.86 (d, J=1.6 Hz, 1H), 6.82 (dd, J=5.4, 1.7 Hz, 1H), 4.16 (qd, J=9.8, 6.6 Hz, 2H).

Step 2: 4-Bromo-N-methyl-N-(2,2,2-trifluoroethyl)pyridin-2-amine I-34c was synthesised from 4-bromo-N-(2,2,2-trifluoroethyl)pyridin-2-amine I-34b using a procedure essentially the same as for 1-4a. ¹H NMR (400 MHz, DMSO-d6) δ 7.15 (dd, J=5.4, 1.6 Hz, 1H), 6.12 (d, J=1.6 Hz, 1H), 6.09-5.98 (m, 1H), 3.58 (q, J=9.1 Hz, 2H), 2.30 (s, 3H).

Step 3: 4-(4-Amino-2-chloro-5-fluorophenyl)-N-methyl-N-(2,2,2-trifluoroethyl)pyridin-2-amine 1-34 was synthesised from 4-bromo-N-methyl-N-(2,2,2-trifluoroethyl)pyridin-2-amine I-34c and 5-chloro-2-fluoro-4-(4,4,5,5-tetramethyl-1,3,2-dioxaborolan-2-yl)aniline I-3a using a procedure essentially the same as for 1-3. ¹H NMR (500 MHz, DMSO-d6) δ 8.12 (dd, J=5.2, 0.8 Hz, 1H), 7.18 (d, J=11.9 Hz, 1H), 6.90 (d, J=8.2 Hz, 1H), 6.75 (dd, J=5.2, 1.4 Hz, 1H), 6.72 (s, 1H), 5.69 (s, 2H), 4.51 (q, J=9.6 Hz, 2H), 3.11 (s, 3H).

### Intermediate 35 (1-35) and Intermediate 36 (1-36)

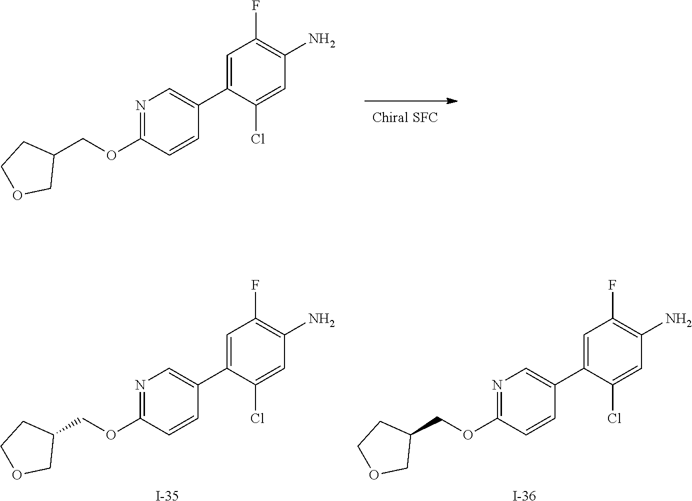

(±)-5-chloro-2-fluoro-4-(6-((tetrahydrofuran-3-yl)methoxy)pyridin-3-yl) 1-16 was dissolved to 32 mg/mL in MeOH, filtered and was then separated by chiral SFC on a Sepiatec with UV detection by DAD at 220 nm, 40° C., 120 bar using a Chiralpak IH 10×150 mm column, 5 um, flow rate 20 mL/min at 50% MeOH, 50% C02. The clean fractions were pooled, rinsed with methanol and dried in vacuo to give 5-chloro-2-fluoro-4-(6-fluoropyridin-3-yl)aniline enantiomer 1 1-35 as a light brown oil and 5-chloro-2-fluoro-4-(6-fluoropyridin-3-yl)aniline enantiomer 2 1-36 as a light brown oil.

5-chloro-2-fluoro-4-(6-fluoropyridin-3-yl)aniline enantiomer 1 1-35 ¹H NMR (400 MHz, DMSO-d6) δ 8.13 (dd, J=2.5, 0.7 Hz, 1H), 7.73 (dd, J=8.6, 2.5 Hz, 1H), 7.11 (d, J=11.9 Hz, 1H), 6.91 (d, J=8.4 Hz, 1H), 6.85 (dd, J=8.5, 0.8 Hz, 1H), 5.59 (s, 2H), 4.26 (dd, J=10.5, 6.7 Hz, 1H), 4.18 (dd, J=10.5, 8.0 Hz, 1H), 3.83-3.72 (m, 2H), 3.54 (dd, J=8.6, 5.5 Hz, 1H), 2.06-1.97 (m, 1H), 1.72-1.60 (m, 1H).

5-chloro-2-fluoro-4-(6-fluoropyridin-3-yl)aniline enantiomer 1 1-36 1H NMR (400 MHz, DMSO-d6) δ 8.13 (dd, J=2.6, 0.8 Hz, 1H), 7.73 (dd, J=8.5, 2.5 Hz, 1H), 7.11 (d, J=11.9 Hz, 1H), 6.91 (d, J=8.4 Hz, 1H), 6.85 (dd, J=8.6, 0.8 Hz, 1H), 5.59 (s, 2H), 4.26 (dd, J=10.5, 6.7 Hz, 1H), 4.18 (dd, J=10.5, 8.0 Hz, 1H), 3.83-3.72 (m, 2H), 3.71-3.60 (m, 1H), 3.54 (dd, J=8.6, 5.5 Hz, 1H), 2.75-2.60 (m, 1H), 2.08-1.95 (m, 1H), 1.72-1.60 (m, 1H).

### Intermediate 37 (1-37)

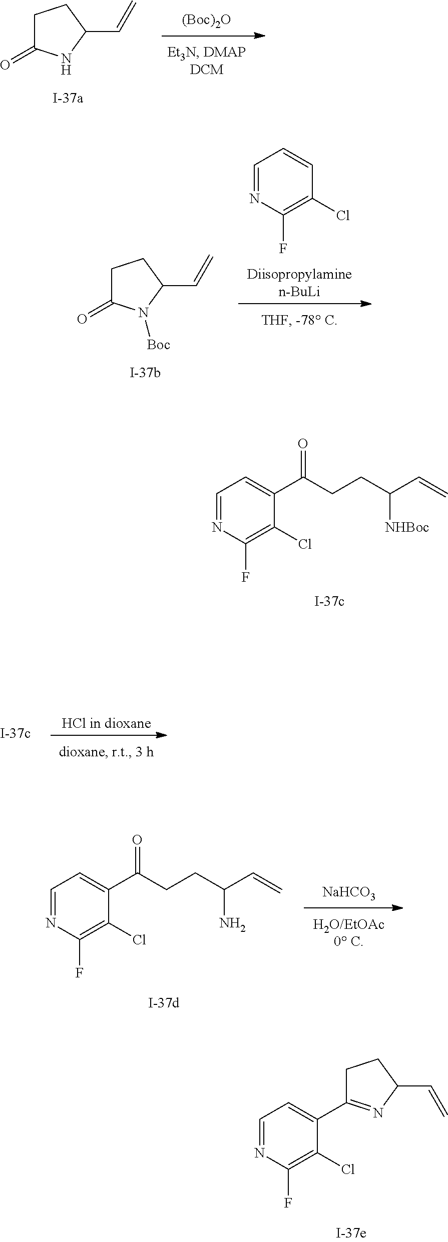

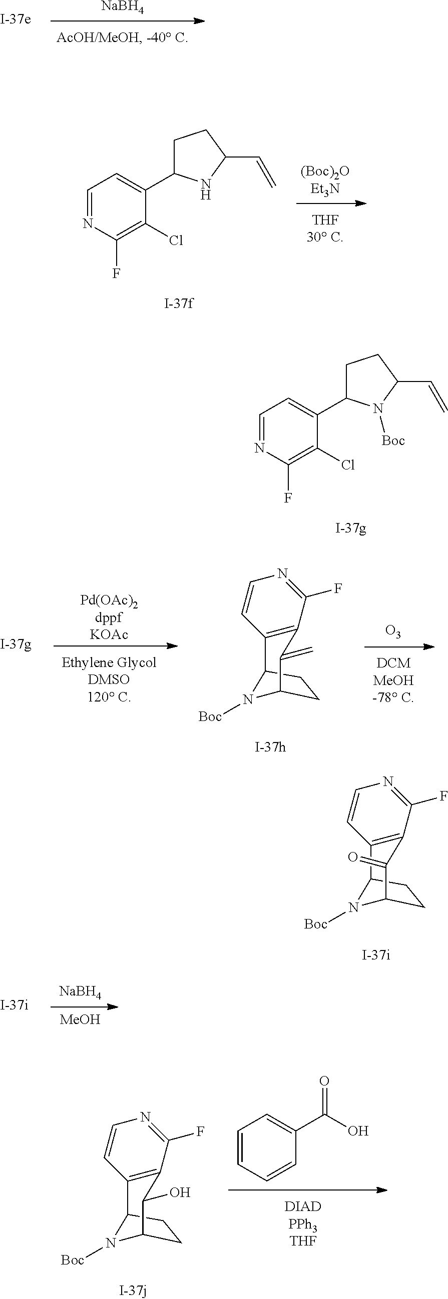

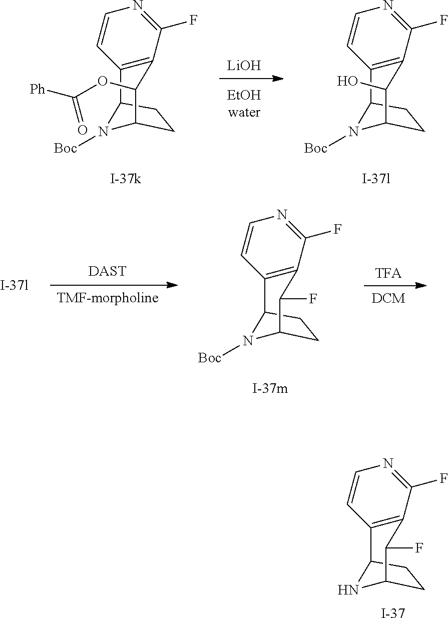

Step 1: To a solution of 5-vinylpyrrolidin-2-one I-37a (1.1 g, 10 mmol) in DCM (25 ml) was added di-tert-butyl dicarbonate (3.3 g, 15 mmol), DMAP (122 mg, 1 mmol) and Et₃N (4.12 ml, 30 mmol). The mixture was stirred at RT for 16 h and then concentrated in vacuo. The product was purified by chromatography on silica gel (5% EtOAc/petroleum ether) to give tert-butyl 2-oxo-5-vinylpyrrolidine-1-carboxylate I-37b as a yellow oil. ¹H NMR (400 MHz, DMSO-d6) δ 5.96 (m, 1H), 5.14-5.07 (m, 2H), 4.57-5.54 (m, 1H), 2.52-2.43 (m, 2H), 2.34-2.13 (m, 2H), 1.72-1.41 (m, 1H), 1.41 (s, 9H).

Step 2: To a solution of diisopropylamine (27 ml, 191.7 mmol) in THF (120 ml) was added ⁿBuLi (76.7 ml, 2.5 M in hexane, 191.75 mmol) drop-wise at −78° C. After stirring at −78° C. for 1 h, a solution of 3-chloro-2-fluoropyridine (16.8 g, 127.8 mmol) in THF (100 ml) was added. The reaction mixture was then stirred at −78° C. for another 1 h before a solution of tert-butyl 2-oxo-5-vinylpyrrolidine-1-carboxylate I-37b (27 g, 127.8 mmol, 1.0 eq) in THF (200 ml) was added and the mixture was stirred at −78° C. for 1 h. The mixture was poured into saturated NH₄Cl solution (800 ml) and extracted with EtOAc (3×200 ml). The combined organics were washed with brine (500 ml), dried over Na₂SO₄ and concentrated in vacuo. The product was purified by chromatography on silica gel column (3%-33% EtOAc/petroleum ether) to give tert-butyl (6-(3-chloro-2-fluoropyridin-4-yl)-6-oxohex-1-en-3-yl)carbamate I-37c as a yellow oil. LCMS (Method 5) m/z 343.1 (M+H)⁺ (ES)⁺at 2.25 min.

Step 3: To a solution of tert-butyl (6-(3-chloro-2-fluoropyridin-4-yl)-6-oxohex-1-en-3-yl) carbamate I-37c (16.7 g, 48.7 mmol) in 1,4-dioxane (80 ml) was added a solution of HCl in 1,4-dioxane (120 ml, 4 M, 480 mmol) at RT under N₂. After stirring for 3 h, the reaction mixture was concentrated in vacuo to give 4-amino-1-(3-chloro-2-fluoropyridin-4-yl)hex-5-en-1-one I-37d as a brown oil. LCMS (Method 5) m/z 243.1 (M+H)⁺ (ES)⁺at 1.88 min.

Step 4: To a solution of NaHCO₃ (10.2 g, 121.5 mmol) in water (50 ml) was added a solution of 4-amino-1-(3-chloro-2-fluoropyridin-4-yl) hex-5-en-1-one I-37d (11.8 g, 48.6 mmol) in EtOAc (200 ml) at 0° C. under N₂. After stirring for 3 h, the reaction mixture was diluted with water (300 ml) and extracted with EtOAc (2×100 ml). The combined organics were washed with brine (100 ml), dried over Na₂SO₄ and concentrated in vacuo. The product was purified by chromatography on silica gel (20% EtOAc/petroleum ether) to give 3-chloro-2-fluoro-4-(2-vinyl-3,4-dihydro-2H-pyrrol-5-yl)pyridine I-37e as a yellow oil. LCMS (Method 5) m/z 224.7 (M+H)⁺ (ES)⁺at 1.24 min.

Step 5: To a solution of AcOH (1.6 ml) in MeOH (20 ml) was added a solution of 3-chloro-2-fluoro-4-(2-vinyl-3,4-dihydro-2H-pyrrol-5-yl)pyridine I-37e (9.4 g, 41.8 mmol) in MeOH (60 ml) drop-wise at −40° C. After stirring at −40° C. for 1 h, NaBH₄ (3.6 g, 96.1 mmol) was added. The progress of reaction was monitored by TLC. After completion, the mixture was poured into saturated NH₄Cl solution (200 ml) and extracted with EtOAc (3×100 ml). The combined organics were washed with brine (100 ml), dried over Na₂SO₄ and concentrated in vacuo. The residue was purified by chromatography on silica gel column (3% EtOAc/petroleum ether) to give 3-chloro-2-fluoro-4-(5-vinylpyrrolidin-2-yl)pyridine I-37f as a yellow oil. LCMS (Method 5) m/z 227.1 (M+H)⁺ (ES)⁺at 0.56 min.

Step 6: To a mixture of 3-chloro-2-fluoro-4-(5-vinylpyrrolidin-2-yl)pyridine I-37f (1.2 g, 5.29 mmol) and Et₃N (1.47 ml, 10.58 mmol) in THF (15 ml) was added (Boc)₂0 (1.73 g, 7.94 mmol). The reaction mixture was stirred at 30° C. for 16 h and then concentrated in vacuo.

The product was purified by chromatography on silica gel column (10% EtOAc/petroleum ether) to give tert-butyl 2-(3-chloro-2-fluoropyridin-4-yl)-5-vinylpyrrolidine-1-carboxylate I-37g as a yellow oil. LCMS (Method 5) m/z 327.1 (M+H)⁺ (ES)⁺at 2.58 min.

Step 7: A mixture of tert-butyl 2-(3-chloro-2-fluoropyridin-4-yl)-5-vinylpyrrolidine-1-carboxylate I-37g (1.7 g, 5.2 mmol), Pd(OAc)₂ (58.4 mg, 0.26 mmol), dppf (216.2 mg, 0.39 mmol) and KOAc (766 mg, 7.8 mmol) in ethylene glycol (1 ml) and DMSO (1.8 mL) was stirred at 120° C. under N₂ for 3 days. The reaction mixture was cooled to RT, diluted with water (100 ml) and the product was extracted with EtOAc (3×100 ml). The combined organics were washed with brine (100 ml), dried over Na₂SO₄ and concentrated in vacuo.

The product was purified by chromatography on silica gel column (20% EtOAc/petroleum ether) to give tert-butyl (±)-1-fluoro-9-methylene-6,7,8,9-tetrahydro-5H-5,8-epiminocyclohepta[c]pyridine-10-carboxylate I-37 h as a yellow oil. ¹H NMR (400 MHz, DMSO-d6) δ 8.06 (d, J=5.2 Hz, 1H), 7.33 (d, J=6.8 Hz, 1H), 5.39-5.38 (m, 1H), 4.99 (d, J=6.4 Hz, 1H), 4.71-4.69 (m, 1H), 3.43-3.41 (m, 1H), 2.32-2.21 (m, 2H), 1.80-1.74 (m, 1H), 1.65-1.62 (m, 1H), 1.33 (s, 9H).

Step 8: Into a mixture of tert-butyl (±)-1-fluoro-9-methylene-6,7,8,9-tetrahydro-5H-5,8-epiminocyclohepta[c]pyridine-10-carboxylate I-37h (400 mg, 1.38 mmol) in DCM/MeOH (10/1, 10 mL) was bubbled O₃ at −78° C. for 10 min. A single drop of dimethyl sulfuide was added into the reaction. The mixture was concentrated in vacuo and the residue was purified by prep-TLC (33% EtOAc/petroleum ether) to give (±)-tert-butyl 1-fluoro-9-oxo-6,7,8,9-tetrahydro-5H-5,8-epiminocyclohepta[c]pyridine-10-carboxylate I-37i as a white solid.

Step 9: To a solution of tert-butyl (±)-1-fluoro-9-oxo-6,7,8,9-tetrahydro-5H-5,8-epiminocyclohepta[c]pyridine-10-carboxylate I-37i (110 mg, 0.38 mmol) in MeOH (5 mL) was added NaBH₄ (29 mg, 0.76 mol) at RT and the resulting mixture was stirred at RT for 1 h. The reaction was concentrated in vacuo, and the product was purified by prep-TLC (50% EtOAc/petroleum ether) to give tert-butyl-(±)-(9S)-1-fluoro-9-hydroxy-6,7,8,9-tetrahydro-5H-5,8-epiminocyclohepta[c]pyridine-10-carboxylate I-37j as a colorless oil. ¹H NMR (400 MHz, DMSO-d6) δ 8.04 (d, J=5.2 Hz, 1H), 7.20 (d, J=6.4 Hz, 1H), 5.11 (d, J=5.6 Hz, 1H), 4.90 (d, J=6.4 Hz, 1H), 4.30 (t, J=14 Hz, 1H), 2.29-2.21 (m, 1H), 2.15-2.05 (m, 1H), 1.93-1.90 (m, 1H), 1.72-1.66 (m, 1H), 1.35 (s, 9H).

Step 9: To a solution of tert-butyl-(±)-(9S)-1-fluoro-9-hydroxy-6,7,8,9-tetrahydro-5H-5,8-epiminocyclohepta[c]pyridine-10-carboxylate I-37j (420 mg, 1.43 mmol), benzoic acid (210 mg, 1.72 mmol) and PPh₃ (750 mg, 2.86 mmol) in THF (16 mL) was added DIAD (578 mg, 2.86 mmol) dropwise at 0° C., before the reaction was allowed to warm to RT for 16 h. The mixture was diluted with water (50 mL) and the product was extracted with EtOAc (3×30 mL). The combined organics were washed with brine (50 mL), dried over Na₂SO₄ and concentrated in vacuo. The product was purified by chromatography on silica gel 16% EtOAc/petroleum ether) to give tert-butyl(±)-(9R)-9-(benzoyloxy)-1-fluoro-6,7,8,9-tetrahydro-5H-5,8-epiminocyclohepta[c]pyridine-10-carboxylate 1-37k as a white solid. ¹H NMR (400 MHz, DMSO-d6) δ 8.25 (d, J=5.2 Hz, 1H), 7.92 (d, J=7.6 Hz, 2H), 7.67 (t, J=15.2 Hz, 1H), 7.51 (t, J=15.6 Hz, 2H), 7.42 (d, J=4.8 Hz, 1H), 5.76 (s, 1H), 5.24 (d, J=6.0 Hz, 1H), 4.77 (s, 1H), 2.25-2.20 (m, 1H), 2.12-2.05 (m, 1H), 1.77-1.68 (m, 1H), 1.64-1.59 (m, 1H), 1.18 (d, J=6.4 Hz, 9H).

Step 10: A mixture of tert-butyl(±)-(9R)-9-(benzoyloxy)-1-fluoro-6,7,8,9-tetrahydro-5H-5,8-epiminocyclohepta[c]pyridine-10-carboxylate 1-37k (569 mg, 1.43 mmol) and LiOH (120 mg, 2.86 mmol) in EtOH/water (33 mL, 2/1, v/v) was stirred at 60° C. for 1 h. The reaction was diluted with water (20 mL) and extracted with EtOAc (3×50 mL). The combined organics were washed with brine (50 mL), dried over Na₂SO₄ and concentrated. The product was purified by chromatography on silica gel (33% EtOAc/petroleum ether) to give tert-butyl (±)-(9R)-1-fluoro-9-hydroxy-6,7,8,9-tetrahydro-5H-5, 8-epiminocyclohepta[c]pyridine-10-carboxylate 1-371 as a white solid. ¹H NMR (400 MHz, DMSO-d6) δ 8.08 (d, J=5.2 Hz, 1H), 7.25 (dd, J=4.8, 1.6 Hz, 1H), 5.58 (d, J=6.0 Hz, 1H), 5.08 (s, 1H), 4.45 (s, 1H), 4.40 (d, J=5.2 Hz, 1H), 2.02-1.9 (m, 3H), 1.52-1.46 (m, 1H), 1.38 (s, 9H).

Step 11: To a solution of DAST (358 mg, 2.2 mmol) in DCM (1 mL) was added 4-(trimethylsilyl)morpholine (352 mg, 2.2 mmol) at −78° C. The resulting mixture was allowed to warm to RT and stirred for 2 h and then a solution of tert-butyl (±)-(9R)-1-fluoro-9-hydroxy-6,7,8,9-tetrahydro-5H-5,8-epiminocyclohepta[c]pyridine-10-carboxylate 1-371 (100 mg, 0.34 mmol) in DCM (1 mL) was added at −78° C. under N₂. The reaction mixture was allowed to warm to RT and stirred for 16 h and then basified with saturated aqueous NaHCO₃ solution and the product was extracted with DCM (3×10 mL). The combined organics were dried over Na₂SO₄ and concentrated in vacuo. The product was purified by prep-TLC (33% EtOAc/petroleum ether) to give tert-butyl (±)-(9S)-1,9-difluoro-6,7,8,9-tetrahydro-5H-5,8-epiminocy clohepta[c]pyridine-10-carboxylate I-37m as a white solid.

Step 12: To a solution of tert-butyl (±)-(9S)-1,9-difluoro-6,7,8,9-tetrahydro-5H-5,8-epiminocyclohepta[c]pyridine-10-carboxylate I-37m (42.5 mg, 0.14 mmol) in DCM (1.5 mL) was added TFA (0.5 mL). The reaction mixture was stirred at RT for 1 h before being concentrated in vacuo to afford (±)-(9S)-1,9-difluoro-6,7,8,9-tetrahydro-5H-5,8-epiminocyclohepta[c]pyridine)trifluoroacetate salt 1-37 as a yellow oil. LCMS (Method 5) m/z 197.0 (M+H)⁺ (ES⁺), at 0.28 min.

### Intermediate 38

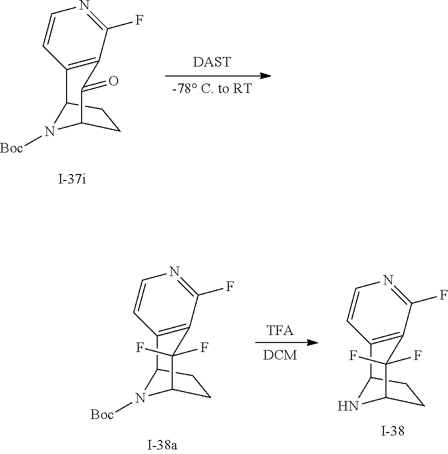

Step 1: To a solution of (±)-tert-butyl 1-fluoro-9-oxo-6,7,8,9-tetrahydro-5H-5,8-epimin ocyclohepta[c]pyridine-10-carboxylate I-37i (998 mg, 3.42 mmol) in DCM (16.5 ml) was added DAST (3.30 g, 20.50 mol) at −78° C. The reaction was allowed to warm to RT and stirred for 16 h and the mixture was basified with saturated aqueous NaHCO₃ solution and extracted with DCM (3×30 ml). The combined organics were dried over Na₂SO₄ and concentrated in vacuo. The product was purified by chromatography on silica gel (33% EtOAc/petroleum ether) to give (±)-tert-butyl 1,9,9-trifluoro-6,7,8,9-tetrahydro-5H-5,8-epiminocyclo hepta[c]pyridine-10-carboxylate I-38a as a light yellow solid. ¹H NMR (400 MHz, DMSO-d6) δ 8.34 (d, J=5.2 Hz, 1H), 7.48 (dd, J=5.2, 1.6 Hz, 1H), 5.26 (d, J=6.8 Hz, 1H), 4.80-4.75 (m, 1H), 2.34-2.10 (m, 2H), 1.89-1.81 (m, 1H), 1.69-1.64 (m, 1H), 1.39 (s, 9H).

Step 2: To a solution of (±)-tert-butyl 1,9,9-trifluoro-6,7,8,9-tetrahydro-5H-5,8-epiminocyclo hepta[c]pyridine-10-carboxylate I-38a (83 mg, 0.26 mmol) in DCM (3 ml) was added TFA (1 ml). The reaction mixture was stirred at RT for 1 h and then concentrated in vacuo to afford (±)-1,9,9-trifluoro-6,7,8,9-tetrahydro-5H-5,8-epiminocyclohepta[c]pyridine 1-38 as a yellow oil. LCMS (Method 5) m/z 215.1 (M+H)⁺ (ES⁺), at 0.38 min.

### Intermediate 39 (1-39)

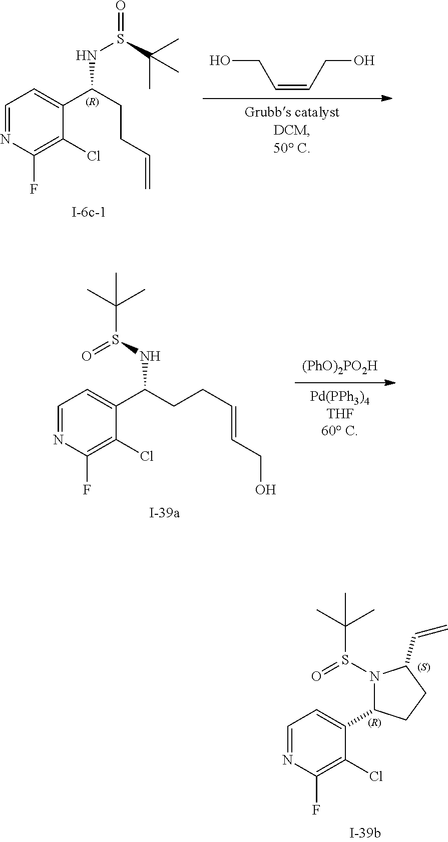

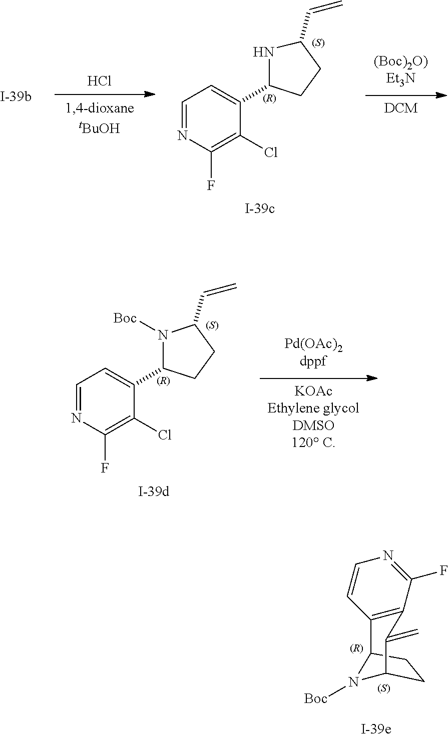

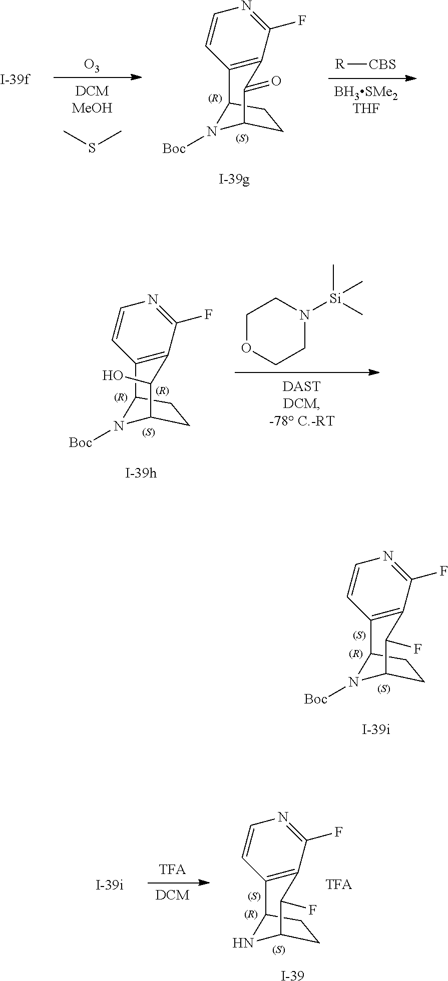

Step 1: To a solution of (S)-N-((R)-1-(3-chloro-2-fluoropyridin-4-yl)pent-4-en-1-yl)-2-methyl propane-2-sulfinamide I-6c-1 (5 g, 15.68 mmol) and (Z)-but-2-ene-1,4-diol (2.76 g, 31.36 mmol) in DCM (30 mL) was added Grubbs catalyst (491 mg, 0.78 mmol, 0.05 eq). The reaction mixture was stirred at 50° C. for 16 h and then concentrated in vacuo. The product was purified by chromatography on silica gel (5-10% EtOAc/petroleum ether) to give (S)-N-((R,E)-1-(3-chloro-2-fluoropyridin-4-yl)-6-hydroxyhex-4-en-1-yl)-2-methylpropane-2-sulfinamide I-39a as a brown oil. LCMS (Method 3) m/z 349.0 (M+H)⁺ (ES⁺), at 1.41 min.

Step 2: To a mixture of (S)-N-((R,E)-1-(3-chloro-2-fluoropyridin-4-yl)-6-hydroxyhex-4-en-1-yl)-2-methylpropane-2-sulfinamide I-39a (4.0 g, 11.46 mmol), (PhO)₂PO₂H (287 mg, 1.15 mmol) in THF (110 mL) was added Pd(PPh₃)₄(1.32 g, 1.15 mmol). The reaction was stirred at 50° C. for 16 h and the resulting mixture was concentrated in vacuo and purified by chromatography on silica gel ((5-10% EtOAc/petroleum ether) to give 4-((2R,5S)-1-(tert-butylsulfinyl)-5-vinylpyrrolidin-2-yl)-3-chloro-2-fluoropyridine I-39b as a brown oil. ¹H NMR (400 MHz, DMSO-d6) δ 8.22 (d, J=5.2 Hz, 1H), 7.54 (d, J=5.2 Hz, 1H), 6.18-6.09 (m, 1H), 5.30-5.19 (m, 2H), 5.07 (dd, J=9.5, 7.0 Hz, 1H), 4.52 (t, J=6.6 Hz, 1H), 2.48-2.43 (m, 1H), 1.93-1.85 (m, 2H), 1.72-1.59 (m, 1H), 1.07 (s, 9H).

Step 3: To a solution of 4-((2R,5S)-1-(tert-butylsulfinyl)-5-vinylpyrrolidin-2-yl)-3-chloro-2-fluoropyridine I-39b (2.2 g, 6.65 mmol) in ^{t}BuOH (10 mL) was added a solution of HCl in 1,4-dioxane (10 mL, 4 M). The reaction was stirred at RT for 2 h, after which the mixture was adjust to pH=9 by the addition of saturated aqueous NaHCO₃. The product was extracted was EtOAc (3×20 mL) and the combined organics were washed with brine (20 mL), dried over Na₂SO₄ and concentrated in vacuo to give 3-chloro-2-fluoro-4-((2R,5S)-5-vinylpyrrolidin-2-yl)pyridine I-39c as a yellow oil. ¹H NMR (400 MHz, DMSO-d6) δ 8.14 (d, J=5.0 Hz, 1H), 7.74 (d, J=5.0 Hz, 1H), 5.96-5.88 (m, 1H), 5.26-5.23 (m, 1H), 5.03-5.00 (m, 1H), 4.54-4.51 (m, 1H), 3.72 (q, J=7.1 Hz, 1H), 2.35-2.30 (m, 1H), 1.96-1.87 (m, 1H), 1.48-1.41 (m, 2H).

Step 4: To a solution of 3-chloro-2-fluoro-4-((2R,5S)-5-vinylpyrrolidin-2-yl)pyridine I-39c (1.1 g, 5.29 mmol) and Et₃N (2.2 mL, 15.87 mmol) in DCM (10 mL) was added Boc₂O (1.7 g, 7.94 mmol). The reaction mixture was stirred at RT for 16 h, after which the mixture was concentrated in vacuo and purified by chromatography on silica gel (2-10% EtOAc/petroleum ether) to give tert-butyl (2R,5S)-2-(3-chloro-2-fluoropyridin-4-yl)-5-vinylpyrrolidine-1-carboxylate I-39d as a light yellow oil. ¹H NMR (400 MHz, DMSO-d6) δ 8.17 (d, J=5.2 Hz, 1H), 7.24 (s, 1H), 6.09-6.00 (m, 1H), 5.27 (d, J=17.2 Hz, 1H), 5.20-5.17 (m, 1H), 5.08 (t, J=7.4 Hz, 1H), 4.40 (s, 1H), 2.44-2.37 (m, 1H), 2.17-2.12 (m, 1H), 1.77-1.63 (m, 2H), 1.40-1.07 (m, 9H).

Step 5: A mixture of tert-butyl (2R,5S)-2-(3-chloro-2-fluoropyridin-4-yl)-5-vinylpyrrolidine-1-carboxylate I-39d (1 g, 3.07 mmol), dppf (128 mg, 0.23 mmol), KOAc (452 mg, 4.6 mmol) and Pd(OAc)₂ (34 mg, 0.15 mmol) in ethylene glycol (2 mL), DMSO (2 mL) and H₂O (2 drops) was stirred at 120° C. for 2 days. The reaction was allowed to cool to RT and then diluted with water (100 mL) and extracted with EtOAc (2×100 mL). The combined organics were dried over Na₂SO₄, concentrated in vacuo and the product was purified by chromatography on silica gel (10% EtOAc/petroleum ether) to give tert-butyl (5R,8S)-1-fluoro-9-methylene-6,7,8,9-tetrahydro-5H-5,8-epiminocyclohepta[c]pyridine-10-carboxylate I-39e as a light yellow solid. ¹H NMR (400 MHz, DMSO-d6) δ 8.05 (d, J=5.2 Hz, 1H), 7.32 (dd, J=1.6, 8.4 Hz, 1H), 5.78 (s, 1H), 5.39 (d, J=4.4 Hz, 1H), 4.99 (d, J=6.4 Hz, 1H), 4.70 (d, J=7.2 Hz, 1H), 2.40-2.15 (m, 2H), 1.80-1.74 (m, 1H), 1.66-1.59 (m, 1H), 1.28 (s, 9H).

Step 6: To a mixture of tert-butyl (5R,8S)-1-fluoro-9-methylene-6,7,8,9-tetrahydro-5H-5,8-epiminocyclohepta[c]pyridine-10-carboxylate I-39e (300 mg, 1.03 mmol) in DCM/MeOH (10/1, 6 mL) was bubbled O₃ at −78° C. for 2 min. Upon completion, one drop of dimethyl sulfide was added into the reaction mixture and it was concentrated in vacuo. The product was purified by chromatography on silica gel (33% EtOAc/petroleum ether) to give tert-butyl(5R,8S)-1-fluoro-9-oxo-6,7,8,9-tetrahydro-5H-5,8-epiminocyclo hepta[c]pyridine-10-carboxylate I-39f as a white solid. ¹H NMR (400 MHz, DMSO-d6) δ 8.44 (d, J=4.8 Hz, 1H), 7.58 (d, J=5.2 Hz, 1H), 5.23 (d, J=6.4 Hz, 1H), 4.50 (d, J=8.4 Hz, 1H), 2.45-2.32 (m, 2H), 1.81-1.74 (m, 2H), 1.25 (s, 9H).

Step 7: To a mixture of tert-butyl (5R,8S)-1-fluoro-9-oxo-6,7,8,9-tetrahydro-5H-5,8-epiminocyclohepta[c]pyridine-10-carboxylate I-39f (1 g, 3.42 mmol) and R-CBS (380 mg, 1.36 mmol) in THF (37 mL) was added BH₃·SMe₂ (0.82 mL, 8.22 mmol) at 0° C. The reaction was stirred at RT for 1 h before water (20 mL) was added and the product was extracted with EtOAc (2×50 mL). The combined organics were dried over Na₂SO₄ and concentrated in vacuo. The product was purified by chromatography on silica gel (20% EtOAc/petroleum ether) to give tert-butyl (5R,8S,9R)-1-fluoro-9-hydroxy-6,7,8,9-tetrahydro-5H-5,8-epiminocyclohepta[c]pyridine-10-carboxylate I-39g as a white solid. ¹H NMR (400 MHz, DMSO-d6) δ 8.03 (d, J=4.8 Hz, 1H), 7.19 (dd, J=1.6, 4.8 Hz, 1H), 5.84 (d, J=6.4 Hz, 1H), 5.10 (t, J=4.0 Hz, 1H), 4.89 (d, J=6.4 Hz, 1H), 4.28 (t, J=6.0 Hz, 1H), 2.28-2.21 (m, 1H), 2.14-2.05 (m, 1H), 1.98-1.85 (m, 1H), 1.71-1.66 (m, 1H), 1.33 (s, 9H).

Step 8: tert-Butyl (5R,8S,9S)-1,9-difluoro-6,7,8,9-tetrahydro-5H-5,8-epiminocyclohepta[c]pyridine-10-carboxylate I-39 h was synthesised from tert-butyl (5R,8S,9R)-1-fluoro-9-hydroxy-6,7,8,9-tetrahydro-5H-5,8-epiminocyclohepta[c]pyridine-10-carboxylate I-39g using a procedures essentially the same as for I-37m. LCMS (method 5) m/z 297.1 (M+H)⁺ (ES⁺), at 2.07 min.

Step 9: (5R,8S,9S)-1,9-difluoro-6,7,8,9-tetrahydro-5H-5,8-epiminocyclohepta[c]pyridine trifluoroacetate salt 1-39 was synthesised from tert-butyl (5R,8S,9S)-1,9-difluoro-6,7,8,9-tetrahydro-5H-5,8-epiminocyclohepta[c]pyridine-10-carboxylate I-39 h using a procedure essentially the same as for 1-37. LCMS (method 5) m/z 197.0 (M+H)⁺ (ES⁺), at 0.28 min.

### Intermediate 40 (1-40)

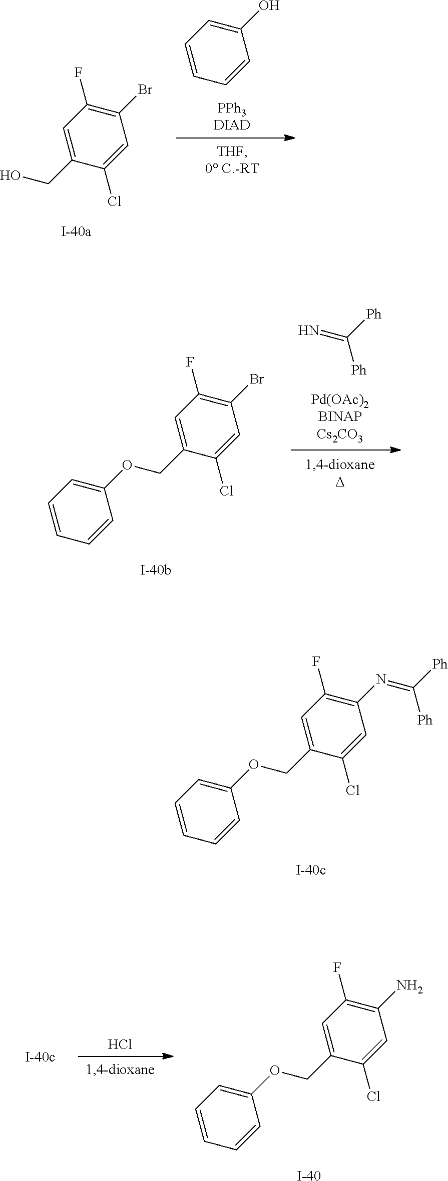

Step 1: To a solution of phenol (157 mg, 1.67 mmol), (4-bromo-2-chloro-5-fluorophenyl)methanol I-40a (400 mg, 1.67 mmol), and PPh₃ (657 mg, 2.5 mmol) in THF (10 mL) at 0° C. was added DIAD (506 mg, 2.5 mmol). The reaction was warmed to RT for 16 h after which, water (40 mL) was added and the product was extracted with EtOAc (3×40 mL). The combined organics were washed with brine (40 mL), dried over Na₂SO₄ and concentrated in vacuo. The product was purified by chromatography on silica gel (5% EtOAc/petroleum ether) to give 1-bromo-5-chloro-2-fluoro-4-(phenoxymethyl)benzene I-40b as a white solid. ¹H NMR (400 MHz, DMSO-d6) δ 6 7.97 (d, J=6.4 Hz, 1H), 7.62 (d, J=9.2 Hz, 1H), 7.32 (t, J=7.6 Hz, 2H), 7.04 (d, J=8.4 Hz, 2H), 6.99 (t, J=7.2 Hz, 1H), 5.11 (s, 2H).

Step 2: To a solution of 1-bromo-5-chloro-2-fluoro-4-(phenoxymethyl)benzene I-40b (350 mg, 1.11 mmol), diphenylmethanimine (202 mg, 1.11 mmol), Pd(OAc)₂ (25 mg, 0.11 mmol) and BINAP (69 mg, 0.11 mmol) in 1,4-dioxane (10 mL) was added Cs2CO₃ (1.09 g, 3.34 mmol). The reaction was heated to 100° C. for 4 h, after which, the reaction was cooled to RT. Water (20 mL) was added and the product was extracted with EtOAc (3×20 mL). The 20 combined organics were washed with brine (40 mL), dried over Na₂SO₄ and concentrated in vacuo. The product was purified by chromatography on silica gel (6% EtOAc/petroleum ether) to give N-(5-chloro-2-fluoro-4-(phenoxymethyl)phenyl)-1, 1-diphenylmethanimine I-40c as a yellow oil. LCMS (Method 5) m/z 416.1 (M+H)⁺ (ES⁺), at 2.28 min.

Step 3: To a solution of N-(5-chloro-2-fluoro-4-(phenoxymethyl)phenyl)-1,1-diphenylmethanimine I-40c (340 mg, 0.82 mmol) in 1,4-dioxane (2.5 mL) was added a 4 M solution of HCl in 1,4-dioxane (2.5 mL, 10 mmol). The reaction was stirred at RT for 2 h, after which the pH was adjusted to pH=8 by addition of a saturated aqueous NaHCO₃ solution and the product was extracted with EtOAc (3×10 mL). The combined organics were washed with brine (20 mL), dried over Na₂SO₄ and concentrated in vacuo. The product was purified by prep-TLC (10% EtOAc/petroleum ether) to give 5-chloro-2-fluoro-4-(phenoxymethyl)aniline 1-40 as a yellow solid. ¹H NMR (400 MHz, DMSO-d6) δ 6 7.29 (t, J=8.4 Hz, 2H), 7.23 (d, J=11.6 Hz, 1H), 6.99 (d, J=8.0 Hz, 2H), 6.94 (t, J=7.2 Hz, 1H), 6.85 (d, J=8.0 Hz, 1H), 5.55 (s, 2H), 4.93 (s, 2H).

### Intermediate 47 (1-47)

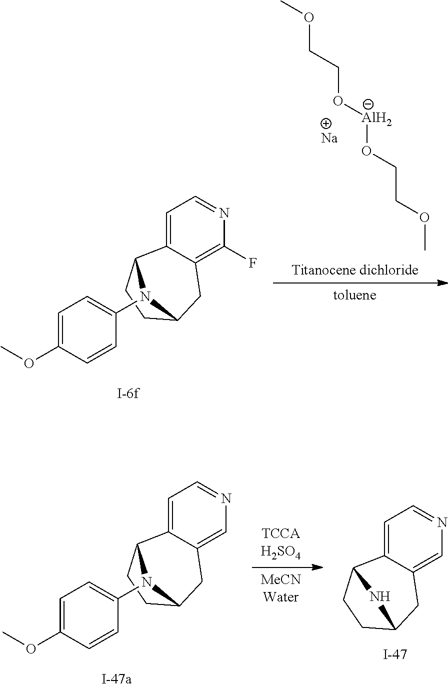

Step 1: To a suspension of titanocene dichloride (219 mg, 879 μmol) in sodium bis(2-methoxyethoxy)aluminiumhydride (6.88 mL, 60% Wt., 21.1 mmol) was added (5R,8S)-1-fluoro-10-(4-methoxyphenyl)-6,7,8,9-tetrahydro-5H-5,8-epiminocyclohepta[c]pyridine I-6f (5.00 g, 17.6 mmol). Toluene (5 mL) was added and the reaction was stirred at RT over 3 days. The mixture was slowly poured into water (200 mL), EtOAc (100 mL) was added and the combined was filtered and the solid was washed with EtOAc (2×50 mL). The filtrate was separated and the aqueous was extracted with EtOAc (2×100 mL). The combined organics were dried over MgSO₄, and concentrated in vacuo. The product was purified by chromatography on silica gel (0-100% EtOAc/iso-hexane) to afford (5R,8S)-10-(4-methoxyphenyl)-6,7,8,9-tetrahydro-5H-5,8-epiminocyclohepta[c]pyridine I-47a as a pale tan oil that solidified upon standing. ¹H NMR (400 MHz, DMSO-d6) δ 8.25 (d, J=4.9 Hz, 1H), 8.12 (s, 1H), 7.25 (d, J=4.9 Hz, 1H), 6.83-6.75 (m, 2H), 6.74-6.65 (m, 2H), 4.80 (d, J=5.7 Hz, 1H), 4.52 (t, J=5.7 Hz, 1H), 3.60 (s, 3H), 3.07 (dd, J=17.1, 4.9 Hz, 1H), 2.43 (d, J=17.2 Hz, 1H), 2.31-2.18 (m, 2H), 1.84-1.69 (m, 2H).

Step 2: (5R,8S)-6,7,8,9-Tetrahydro-5H-5,8-epiminocyclohepta[c]pyridine 1-47 was synthesised from (5R,8S)-10-(4-methoxyphenyl)-6,7,8,9-tetrahydro-5H-5,8-epiminocyclohepta[c]pyridine I-47a using a procedure essentially the same as for I-7p. ¹H NMR (400 MHz, DMSO-d6) δ 8.24-8.18 (m, 2H), 7.01 (d, J=4.8 Hz, 1H), 4.10-4.06 (m, 1H), 3.79-3.71 (m, 1H), 2.99 (dd, J=16.7, 5.1 Hz, 1H), 2.67 (s, 1H), 2.47 (d, J=14.3 Hz, 1H), 1.95-1.88 (m, 2H), 1.74-1.65 (m, 1H), 1.54-1.39 (m, 1H).

### Intermediate 49 (1-49)

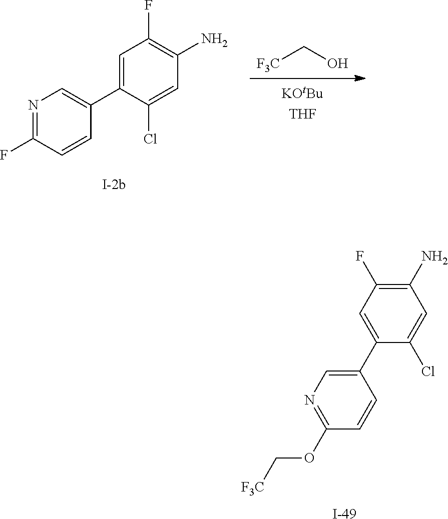

Step 1: 5-Chloro-2-fluoro-4-(6-(2,2,2-trifluoroethoxy)pyridin-3-yl)aniline 1-49 was synthesised from 5-chloro-2-fluoro-4-(6-fluoropyridin-3-yl)aniline I-2b using a procedure essentially the same as for 1-11. ¹H NMR (400 MHz, DMSO-d₆) δ 8.19 (dd, J=2.5, 0.8 Hz, 1H), 7.83 (dd, J=8.6, 2.5 Hz, 1H), 7.15 (d, J=11.9 Hz, 1H), 7.03 (dd, J=8.5, 0.7 Hz, 1H), 6.92 (d, J=8.3 Hz, 1H), 5.63 (s, 2H), 5.02 (q, J=9.1 Hz, 2H).

### Intermediate 50 (1-50)

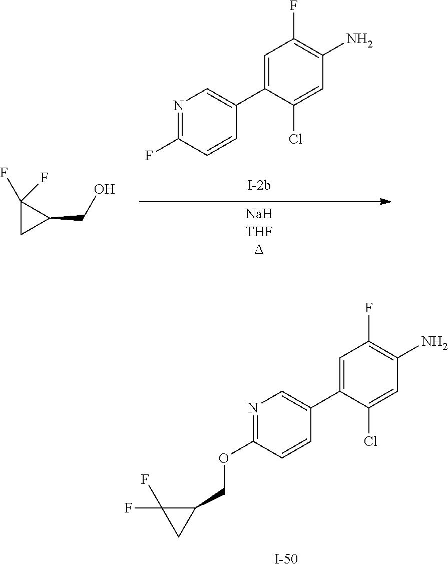

A solution of (R)-(2,2-difluorocyclopropyl)methanol (100 mg, 925 μmol) in THF (3.00 mL) was cooled to 0° C. To this mixture was added sodium hydride (39.5 mg, 60% Wt, 987 μmol) and the resulting suspension stirred at room temperature for 30 minutes before 5-chloro-2-fluoro-4-(6-fluoropyridin-3-yl)aniline I-2b (148 mg, 617 μmol) in THF (3.00 mL) was added. The resulting suspension was heated to 60° C. and stirred for 2.5 h. The mixture was cooled to RT, quenched with water (0.1 mL) and partitioned between brine (25 mL) and DCM (25 mL). The aqueous phase was extracted with DCM (25 mL) and the combined organics were dried over MgSO₄, filtered then concentrated in vacuo.

The product was purified by chromatography on RP Flash C18 (5-100% MeCN/(0.1% ammonium hydroxide in water)) to afford (R)-5-chloro-4-(6-((2,2-difluorocyclopropyl)methoxy)pyridin-3-yl)-2-fluoroaniline as a sticky yellow oil. ¹H NMR (400 MHz, DMSO-d6) δ 8.14 (dd, J=2.6, 0.8 Hz, 1H), 7.75 (dd, J=8.6, 2.5 Hz, 1H), 7.12 (d, J=11.9 Hz, 1H), 6.99-6.78 (m, 2H), 5.59 (s, 2H), 4.61-4.39 (m, 1H), 4.22 (ddd, J=11.7, 8.6, 1.8 Hz, 1H), 2.36-2.14 (m, 1H), 1.79-1.71 (m, 1H), 1.53-1.46 (m, 1H).

### Intermediate 51 (1-51)

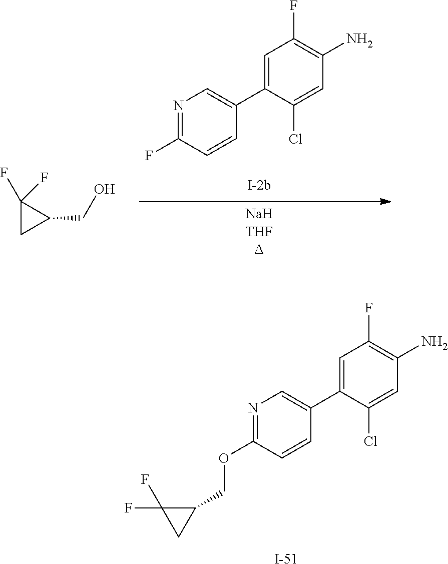

(S)-5-Chloro-4-(6-((2,2-difluorocyclopropyl)methoxy)pyridine-3-yl)-2-fluoroaniline 1-51 was synthesised from 5-chloro-2-fluoro-4-(6-fluoropyridin-3-yl)aniline I-2b and (S)-(2,2-difluorocyclopropyl)methanol using a procedure essentially the same as for 1-50. ¹H NMR (500 MHz, DMSO-d6) δ 8.14 (d, J=2.5 Hz, 1H), 7.75 (dd, J=8.6, 2.5 Hz, 1H), 7.12 (d, J=11.8 Hz, 1H), 6.91 (d, J=4.9 Hz, 1H), 6.89 (d, J=5.2 Hz, 1H), 5.59 (s, 2H), 4.52-4.44 (m, 1H), 4.26-4.18 (m, 1H), 2.32-2.19 (m, 1H), 1.79-1.66 (m, 1H), 1.57-1.46 (m, 1H).

### Intermediate 52 (1-52)

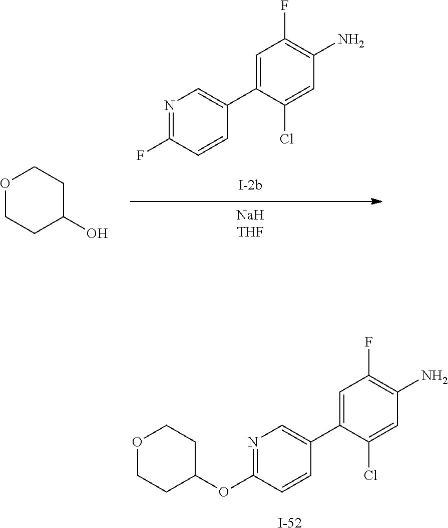

To a solution of tetrahydro-2H-pyran-4-ol (239 μL, 2.49 mmol) in THF (5.00 mL) was added sodium hydride (125 mg, 60% Wt, 3.12 mmol) at 0° C. The resultant mixture was stirred at 0° C. for 15 min before a solution of 5-chloro-2-fluoro-4-(6-fluoropyridin-3-yl)aniline I-2b (0.50 g, 2.08 mmol) in THF (10.0 mL) was added. The mixture was slowly warmed to RT over 16 h. A further portion of sodium hydride (125 mg, 60% Wt, 3.12 mmol) was added and the mixture was stirred for 16 h. Water (20 ml) was added and the product was extracted with DCM (50 ml). The organics were washed with brine (20 ml), dried with sodium sulfate and concentrated in vacuo. The product was purified by chromatography on silica gel (0-100% DCM/EtOAc) to afford 5-chloro-2-fluoro-4-(6-((tetrahydro-2H-pyran-4-yl)oxy)pyridin-3-yl)aniline 1-52 as a colourless solid. ¹H NMR (500 MHz, DMSO-d6) δ 8.13 (d, J=2.5 Hz, 1H), 7.73 (dd, J=8.5, 2.5 Hz, 1H), 7.12 (d, J=11.8 Hz, 1H), 6.91 (d, J=8.4 Hz, 1H), 6.83 (d, J=8.5 Hz, 1H), 5.59 (s, 2H), 5.21 (tt, J=8.9, 4.1 Hz, 1H), 3.88 (dt, J=11.7, 4.3 Hz, 2H), 3.50 (ddd, J=12.0, 9.8, 2.7 Hz, 2H), 2.11-1.94 (m, 2H), 1.65 (dtd, J=13.3, 9.4, 4.1 Hz, 2H).

### Intermediate 53 (1-53)

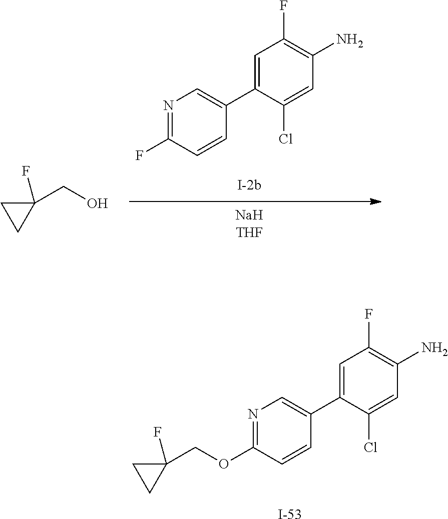

5-Chloro-2-fluoro-4-(6-((1-fluorocyclopropyl)methoxy)pyridine-3-yl)aniline 1-53 was synthesied from (1-fluorocyclopropyl)methanol and 5-chloro-2-fluoro-4-(6-fluoropyridin-3-yl)aniline I-2b using a procedure essentially the same as for 1-52. ¹H NMR (500 MHz, DMSO-d6) δ 8.14 (d, J=2.5 Hz, 1H), 7.77 (dd, J=8.6, 2.6 Hz, 1H), 7.13 (d, J=11.8 Hz, 1H), 6.97-6.87 (m, 2H), 5.60 (s, 2H), 4.60 (d, J=23.2 Hz, 2H), 1.12 (dd, J=18.6, 6.7 Hz, 2H), 0.91 (p, J=7.9 Hz, 2H).

### Intermediate 54 (1-54)

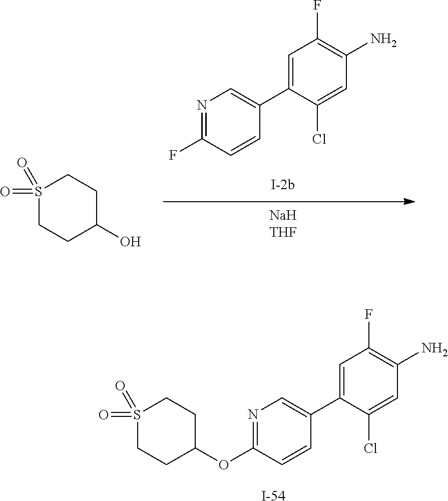

4-((5-(4-Amino-2-chloro-5-fluorophenyl)pyridine-2-yl)oxy)tetrahydro-2H-thiopyran 1,1-dioxide 1-54 was synthesised from 4-hydroxytetrahydro-2H-thiopyran 1,1-dioxide and 5-chloro-2-fluoro-4-(6-fluoropyridin-3-yl)aniline I-2b using a procedure essentially the same as for 1-52. ¹H NMR (500 MHz, DMSO-d6) δ 8.15 (d, J=2.6 Hz, 1H), 7.77 (dd, J=8.5, 2.6 Hz, 1H), 7.13 (d, J=11.8 Hz, 1H), 6.95-6.87 (m, 2H), 5.60 (s, 2H), 5.35 (tt, J=6.8, 3.2 Hz, 1H), 3.22 (p, J=3.6 Hz, 4H), 2.39-2.18 (m, 4H).

### Intermediate 55 (1-55)

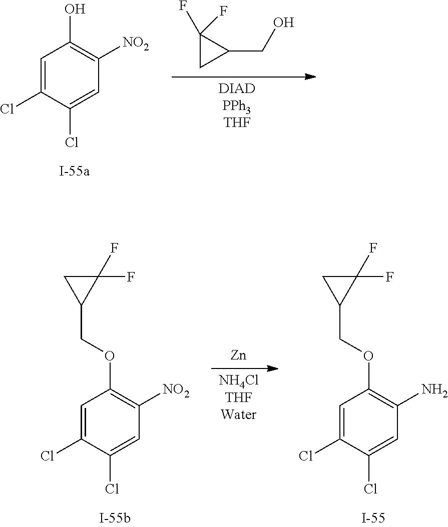

Step 1: To a homogeneous mixture of 4,5-dichloro-2-nitrophenol I-55a (500 mg, 2.40 mmol), triphenylphosphine (946 mg, 3.61 mmol) and (2,2-difluorocyclopropyl)methanol (0.35 mL, 2.88 mmol) in THF (5.00 mL) at 0° C. was added DIAD (701 μL, 3.61 mmol) dropwise. The reaction was slowly warmed to RT over 20 h. Water (10 ml) was added and the product was extracted with DCM (3×20 ml). The combined organics were washed with brine (20 ml) and dried with sodium sulfate and then concentrated in vacuo. The product was purified by chromatography on silica gel (0-50% EtOAc/isohexane) to afford 1,2-dichloro-4-((2,2-difluorocyclopropyl)methoxy)-5-nitrobenzene I-55b as a light yellow solid. ¹H NMR (500 MHz, CDCl₃) δ 7.93 (s, 1H), 7.11 (s, 1H), 4.18 (ddd, J=9.8, 7.3, 1.9 Hz, 1H), 4.06 (dd, J=10.5, 7.4 Hz, 1H), 2.06 (tdd, J=14.7, 9.7, 6.0 Hz, 1H), 1.64-1.53 (m, 1H), 1.31 (dtd, J=12.2, 7.8, 4.0 Hz, 1H).

Step 2: To a solution of 1,2-dichloro-4-((2,2-difluorocyclopropyl)methoxy)-5-nitrobenzene I-55b (216 mg, 724.7 μmol) in THF (4.00 mL) and water (1.00 mL) was added ammonium chloride (232.6 mg, 4.348 mmol) and zinc (284.3 mg, 4.348 mmol). The resultant mixture was stirred at RT for 24 h. Water (10 ml) was added and the product was extracted with DCM (3×20 ml). The combined organics were washed with brine (10 ml) and dried with sodium sulfate. The product was purified by chromatography on silica gel (0-50% EtOAc/isohexane) to afford 4,5-dichloro-2-((2,2-difluorocyclopropyl)methoxy)aniline 1-55 as a colourless solid. ¹H NMR (500 MHz, CDCl₃) δ 6.76 (s, 1H), 6.74 (s, 1H), 3.98 (dtd, J=19.1, 10.8, 5.9 Hz, 2H), 2.08-1.93 (m, 1H), 1.53 (tdd, J=11.9, 8.0, 4.6 Hz, 1H), 1.28-1.14 (m, 1H). 2 exchangeable protons not observed

### Intermediate 56 (1-56)

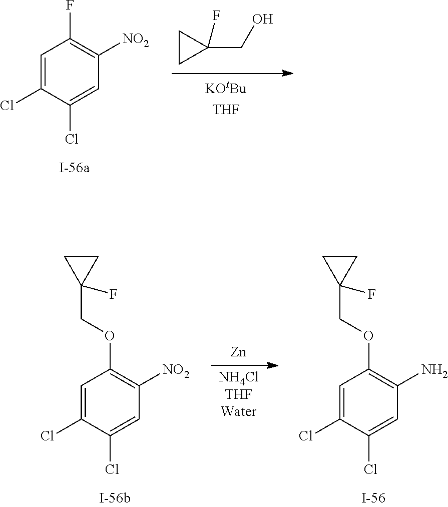

Step 1: To a solution of (1-fluorocyclopropyl)methanol (0.250 g, 2.77 mmol) in THF (5.00 mL) at 0° C. was added potassium tert-butoxide (342 mg, 3.05 mmol). The mixture was stirred at 0° C. for 5 min before a solution of 1,2-dichloro-4-fluoro-5-nitrobenzene I-55a (524 mg, 2.50 mmol) in THF (5.00 mL) was added and the mixture was warmed to RT over 4 days. Water (20 ml) and DCM (20 ml) were added and the layers were separated. The aqueous was extracted with EtOAc (3×30 ml). The combined organics were washed with brine (30 ml), dried with sodium sulfate and concentrated in vacuo. The product was purified by chromatography on silica gel (0-50% EtOAc/isohexane) to afford 1,2-dichloro-4-((1-fluorocyclopropyl)methoxy)-5-nitrobenzene I-56b as a light yellow solid. ¹H NMR (500 MHz, CDCl₃) δ 8.00 (s, 1H), 7.28 (s, 1H), 4.39 (d, J=19.6 Hz, 2H), 1.23 (dd, J=18.5, 7.3 Hz, 2H), 0.89 (q, J=7.6 Hz, 2H).

Step 2: 4,5-Dichloro-2-((1-fluorocyclopropyl)methoxy)aniline 1-56 was synthesised from 1,2-dichloro-4-((1-fluorocyclopropyl)methoxy)-5-nitrobenzene I-55b using a procedure essentially the same as for 1-55. ¹H NMR (500 MHz, DMSO-d6) δ 7.04 (s, 1H), 6.82 (s, 1H), 5.12 (s, 2H), 4.33 (s, 1H), 4.29 (s, 1H), 1.12 (dd, J=18.6, 6.7 Hz, 2H), 0.91-0.82 (m, 2H).

### Intermediate 58 (1-58)

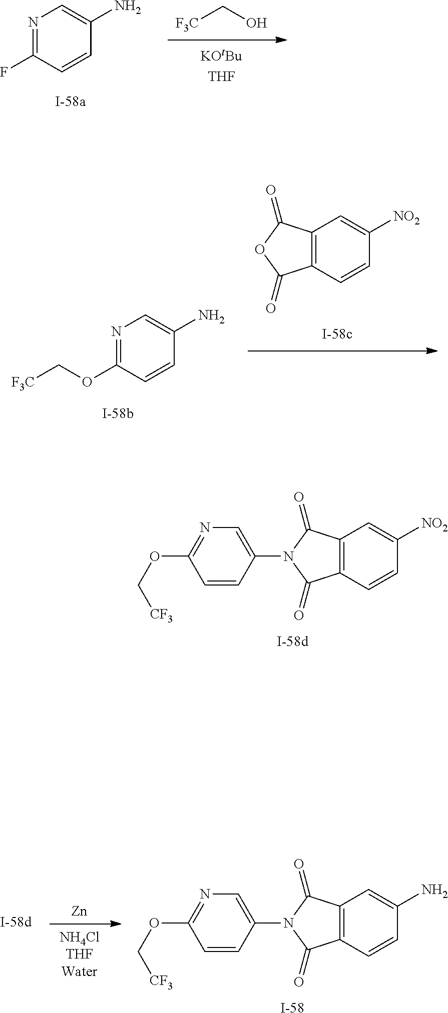

Step 1: To a solution of trifluoroethanol (1.01 mL, 13.4 mmol) in THF (5.00 mL) was added potassium tert-butoxide (1.40 g, 12.5 mmol). The reaction was stirred at RT for 15 min before a solution of 6-fluoropyridin-3-amine I-58a (1.00 g, 8.92 mmol) in THF (5.00 mL) was added and the mixture was stirred at RT for 48 h. A further solution of trifluoroethanol (1.01 mL, 13.4 mmol) in THF (10.0 mL) was cooled to O ° C. and potassium tert-butoxide (1.40 g, 12.5 mmol) was added. The mixture was stirred at 0° C. for 15 min before adding to the previous solution and the combined mixture was warmed to 65° C. for 5 days. The reaction was cooled to RT and water (20 ml) was added. The product was extracted with DCM (3×50 ml). The combined organics were washed with brine (30 ml), dried with sodium sulfate and concentrated in vacuo. The product was purified by chromatography on silica gel (0-100% EtOAc/isohexane) to afford 6-(2,2,2-trifluoroethoxy)pyridin-3-amine I-58b as a thick orange oil. ¹H NMR (500 MHz, CDCl₃) δ 7.63 (d, J=3.0 Hz, 1H), 7.08 (dd, J=8.7, 2.9 Hz, 1H), 6.72 (d, J=8.6 Hz, 1H), 4.69 (q, J=8.7 Hz, 2H), 3.44 (s, 2H).

Step 2: A solution of 6-(2,2,2-trifluoroethoxy)pyridin-3-amine I-58b (570 mg, 2.97 mmol) in acetic acid (10 mL) was added to 5-nitroisobenzofuran-1,3-dione I-58c (630 mg, 3.26 mmol) and the resultant mixture was heated at 120° C. for 24 h. The reaction was cooled to RT. EtOAc (30 ml) and water (30 ml) was added and the mixture was neutralised with sodium bicarbonate until all effervescence stopped. The layers were separated and the aqueous was extracted with EtOAc (2×50 ml). The combined organics were washed with brine (30 ml) and dried with sodium sulfatebefore being concentrated in vacuo.

The product was purified by chromatography on silica gel (0-100% EtOAc/isohexane) to afford 5-nitro-2-(6-(2,2,2-trifluoroethoxy)pyridin-3-yl)isoindoline-1,3-dione I-58d as a thick yellow solid. ¹H NMR (500 MHz, DMSO-d6) δ 8.71 (dd, J=8.2, 2.1 Hz, 1H), 8.63 (d, J=2.1 Hz, 1H), 8.34 (d, J=2.6 Hz, 1H), 8.26 (d, J=8.1 Hz, 1H), 7.94 (dd, J=8.7, 2.6 Hz, 1H), 7.22 (d, J=8.8 Hz, 1H), 5.08 (q, J=9.1 Hz, 2H).

Step 3: 5-Amino-2-(6-(2,2,2-trifluoroethoxy)pyridin-3-yl)isoindoline-1,3-dione 1-58 was synthesised from 5-nitro-2-(6-(2,2,2-trifluoroethoxy)pyridin-3-yl)isoindoline-1,3-dione I-58d using a procedure essentially the same as for 1-55. ¹H NMR (500 MHz, DMSO-d6) δ 8.25 (d, J=2.6 Hz, 1H), 7.87 (dd, J=8.7, 2.7 Hz, 1H), 7.60 (d, J=8.2 Hz, 1H), 7.14 (d, J=8.8 Hz, 1H), 7.02 (d, J=2.1 Hz, 1H), 6.88 (dd, J=8.2, 2.2 Hz, 1H), 6.60 (s, 2H), 5.05 (q, J=9.1 Hz, 2H).

### Intermediate 59 (1-59)

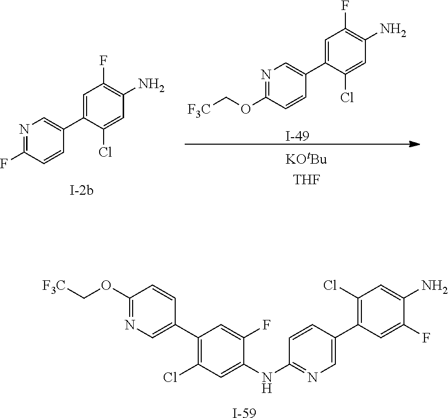

To a solution of 5-chloro-2-fluoro-4-(6-(2,2,2-trifluoroethoxy)pyridin-3-yl)aniline 1-49 (5.00 g, 0.016 mol, in THF (40 mL) was added 5-chloro-2-fluoro-4-(6-fluoropyridin-3-yl)aniline I-2b (4.75 g, 0.02 mol) and the reaction mixture was stirred at RT for 10 min. The reaction mixture was cooled down to 0° C., and then potassium tert-butoxide (8.75 g, 0.078 mol) was added to the mixture and the mixture was stirred at 70° C. for 43 h. The reaction mixture was cooled to RT and an aqueous 2 M HCl solution was added (5 mL). The layers were separated and the aqueous was extracted with EtOAc (35 mL). The combined organic phase was washed with water (10 mL) and concentrated in vacuo. The product was purified by column chromatography (2-7% EtOAc/n-heptane) to give 5-(4-amino-2-chloro-5-fluorophenyl)-N-(5-chloro-2-fluoro-4-(6-(2,2,2-trifluoroethoxy)pyridin-3-yl)phenyl)pyridin-2-amine 1-59 as a yellow solid. ¹H NMR (500 MHz, DMSO-d6) δ 9.20 (s, 1H), 8.74 (d, J=7.8 Hz, 1H), 8.30 (d, J=2.2 Hz, 1H), 8.24 (d, J=2.2 Hz, 1H), 7.95 (dd, J=2.4, 8.5 Hz, 1H), 7.95 (dd, J=2.4, 8.6 Hz, 1H), 7.44 (d, J=11.9 Hz, 1H), 7.14 (m, 3H), 6.92 (d, J=28.4 Hz, 1H), 8.58 (s, 2H), 5.06 (q, J=9.1 Hz, 2H)

### Experimental Scheme 2

### Compound 51 (6S,9R)-N-(5-Chloro-2-fluoro-4-(((5-fluoropyridin-2-yl)oxy)methyl)phenyl)-3-oxo-3,5,6,7,8,9-hexahydro-2H-6,9-epiminocyclohepta[c]pyridazine-10-carboxamide

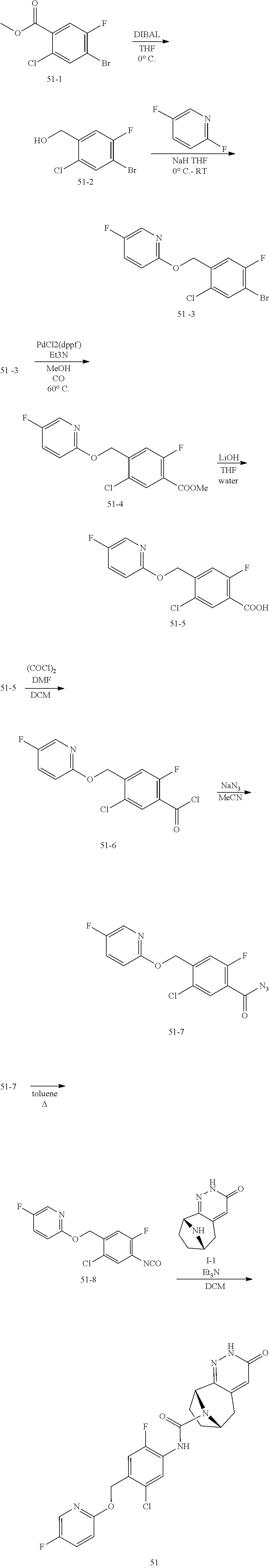

Step 1: To a mixture of methyl 4-bromo-2-chloro-5-fluorobenzoate 51-1 (2.09 g, 7.81 mmol) in THF (30 mL) was added a solution of DIBAL-H in THF (19.5 mL, 1 M, 19.5 mmol) at 0° C. After stirring at 0° C. for 1 h, water (50 mL) was added and the resulting mixture was filtered. The filter cake was washed with EtOAc (20 mL) and the filtrate was separated. The aqueous was extracted with EtOAc (3×20 mL) and the combined organics were dried over Na₂SO₄ and concentrated in vacuo to give (4-bromo-2-chloro-5-fluorophenyl)methanol 51-2 as a white solid. ¹H NMR (400 MHz, DMSO-d6) δ 7.64 (d, J=6.0 Hz, 1H), 7.44 (d, J=9.6 Hz, 1H), 5.63 (t, J=5.6 Hz, 1H), 4.50 (d, J=5.6 Hz, 2H).

Step 2: To a solution of (4-bromo-2-chloro-5-fluorophenyl)methanol 51-2 (890 mg, 3.72 mmol) in THF (10 mL) was added NaH (223 mg, 60% wt., 5.57 mmol) at 0° C. and the mixture was stirred at 0° C. for 30 min, after which 2,5-difluoropyridine (428 mg, 3.72 mmol) was added. The reaction was warmed to RT for 16 h after which the mixture was diluted with water (15 mL) and the product was extracted with EtOAc (3×10 mL). The combined organics were dried over Na₂SO₄ and concentrated in vacuo. The product was purified by chromatography on silica gel (2% EtOAc/petroleum ether) to give 2-((4-bromo-2-chloro-5-fluorobenzyl)oxy)-5-fluoropyridine 51-3 as a white solid. ¹H NMR (400 MHz, DMSO-d6) δ 8.17 (d, J=2.8 Hz, 1H), 7.96 (d, J=6.4 Hz, 1H), 7.78-7.73 (m, 1H), 7.58 (d, J=9.6 Hz, 1H), 7.03 (dd, J=3.6, 8.8 Hz, 1H), 5.34 (s, 2H).

Step 3: To a mixture of 2-((4-bromo-2-chloro-5-fluorobenzyl)oxy)-5-fluoropyridine 51-3 (200 mg, 0.60 mmol) and Et₃N (125 μL, 1.79 mmol) in MeOH (3 mL) was added PdCl₂(dppf) (22 mg, 0.12 mmol). The reaction was warmed to 60° C. under a CO balloon for 16 h after which the mixture was diluted with water (10 mL) and the product was extracted with EtOAc (3×5 mL). The combined organics were dried over Na₂SO₄ and concentrated in vacuo. The product was purified by prep-TLC (10% EtOAc/petroleum ether) to give methyl 5-chloro-2-fluoro-4-(((5-fluoropyridin-2-yl)oxy)methyl)benzoate 51-4 as a white solid. ¹H NMR (400 MHz, DMSO-d6) δ 8.17 (d, J=2.8 Hz, 1H), 7.94 (d, J=6.4 Hz, 1H), 7.79-7.74 (m, 1H), 7.54 (d, J=11.2 Hz, 1H), 7.08 (dd, J=3.6, 9.2 Hz, 1H), 5.42 (s, 2H), 3.87 (s, 3H).

Step 4: To a solution of methyl 5-chloro-2-fluoro-4-(((5-fluoropyridin-2-yl)oxy)methyl)benzoate 51-4 (140 mg, 0.45 mmol) in THF (2 mL) and water (0.5 mL) was added LiOH-monohydrate (37 mg, 0.89 mmol). The reaction mixture was stirred at RT overnight after which the pH was adjusted to pH=3 with 1 M HCl and the product was extracted with EtOAc (3×5 mL). The combined organics were dried over Na₂SO₄ and concentrated in vacuo to give 5-chloro-2-fluoro-4-(((5-fluoropyridin-2-yl)oxy)methyl)benzoic acid 51-5 as a white solid. ¹H NMR (400 MHz, DMSO-d6) δ 8.17 (d, J=2.8 Hz, 1H), 7.97 (d, J=6.4 Hz, 1H), 7.79-7.73 (m, 1H), 7.47 (d, J=10.8 Hz, 1H), 7.07 (dd, J=3.6, 9.2 Hz, 1H), 5.40 (s, 2H). (1 exchangeable proton not observed).

Step 5: To a solution of 5-chloro-2-fluoro-4-(((5-fluoropyridin-2-yl)oxy)methyl)benzoic acid 51-5 (120 mg, 0.40 mmol) in DCM (3 mL) was added (COCI)₂ (51 μL, 0.60 mmol) and a drop of DMF. After stirring at RT for 1 h, the mixture was concentrated in vacuo to give 5-chloro-2-fluoro-4-(((5-fluoropyridin-2-yl)oxy)methyl)benzoyl chloride 51-6, which was used without any further purification in the next step.

Step 6: To a solution of 5-chloro-2-fluoro-4-(((5-fluoropyridin-2-yl)oxy)methyl)benzoyl chloride 51-6 in MeCN (5 mL) was added NaN₃ (104 mg, 1.60 mmol) and the mixture was stirred at RT for 16 h. The reaction was diluted with water (10 mL) and the product was extracted with EtOAc (3×5 mL). The combined organics were dried over Na₂SO₄ and concentrated in vacuo to give 5-chloro-2-fluoro-4-(((5-fluoropyridin-2-yl)oxy)methyl)benzoyl azide 51-7 as a yellow solid. LCMS (Method 7) m/z 325.0, 327.0 (M+H)⁺ (ES⁺), at 2.27 min.

Step 7: A solution of 5-chloro-2-fluoro-4-(((5-fluoropyridin-2-yl)oxy)methyl)benzoyl azide 51-7 (90 mg, 0.28 mmol) in toluene (3 mL) was heated at 120° C. for 30 min. After cooling to RT, the solution of 2-((2-chloro-5-fluoro-4-isocyanatobenzyl)oxy)-5-fluoropyridine 51-8 was used directly in the next step without further purification.

Step 8: To a solution of 2-((2-chloro-5-fluoro-4-isocyanatobenzyl)oxy)-5-fluoropyridine 51-8 in toluene was added a solution of (6S,9R)-2,5,6,7,8,9-hexahydro-3H-6,9-epiminocyclohepta[c]pyridazin-3-one 1-1 (98 mg, 0.28 mmol) and Et₃N (157 μL, 0.55 mmol) 10 in DCM (3 mL). After stirring at RT for 16 h, water (10 mL) was added and the product was extracted with DCM (3×30 mL). The combined organics were dried over Na₂SO₄ and concentrated in vacuo. The product was purified by prep-TLC (5% MeOH/DCM) to give (6S,9R)-N-(5-chloro-2-fluoro-4-(((5-fluoropyridin-2-yl)oxy)methyl)phenyl)-3-oxo-3,5,6,7,8,9-hexahydro-2H-6,9-epiminocyclohepta[c]pyridazine-10-carboxamide 51 as a white solid. LCMS (Method 3) m/z 474.1 (M+H)⁺ (ES⁺), at 2.82 min. ¹H NMR (400 MHz, DMSO-d6) δ 12.71 (s, 1H), 8.81 (s, 1H), 8.16 (d, J=2.4 Hz, 1H), 7.75-7.68 (m, 2H), 7.41 (d, J=11.2 Hz, 1H), 6.99 (dd, J=3.6, 8.8 Hz, 1H), 6.68 (s, 1H), 5.30 (s, 2H), 5.06 (d, J=5.2 Hz, 1H), 4.62 (t, J=5.2 Hz, 1H), 3.18 (dd, J=5.2, 18.0 Hz, 1H), 2.64 (d, J=18.4 Hz, 1H), 2.25-1.12 (m, 2H), 1.81-1.76 (m, 1H), 1.69-1.65 (m, 1H).

The following compounds were prepared using appropriate starting materials in an analogous procedure to that described in Experimental Scheme 2. Where the starting materials are not described in the literature, their synthesis is described below.

| | | Method | |
|---|---|---|---|
| | | LCMS Rt | |
| Compound | Structure | [M + H]⁺ | NMR |
| 52 | 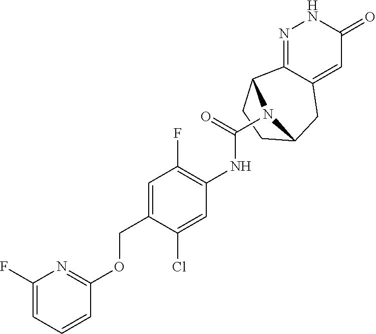 [SMILES: O=C(Nc1cc(Cl)c(COc2cccc(F)n2)cc1F)N1[C@H]2CC[C@@H]1c1n[nH]c(=O)cc1C2] | Method 3 474 at 2.87 min | ¹H NMR (400 MHz, DMSO- d6) δ 12.70 (s, 1H), 8.82 (s, 1H), 7.92 (q, J = 8.0 Hz, 1H), 7.71 (d, J = 7.2 Hz, 1H), 7.58 (d, J = 11.2 Hz, 1H), 6.86- 6.84 (m, 1H), 6.76-6.74 (m, 1H), 6.67 (s, 1H), 5.28 (s, 2H), 5.06 (d, J = 5.6 Hz, 1H), 4.62 (t, J = 5.6 Hz, 1H), 3.18 (dd, J = 4.4, 18 Hz, 1H), 2.64 (d, J = 18 Hz, 1H), 2.25- 2.12 (m, 2H), 1.82-1.62 (m, 2H). |
| | (6S,9R)-N-(5-Chloro-2-fluoro- | | |
| | 4-(((6-fluoropyridin-2- | | |
| | yl)oxy)methyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 54 | 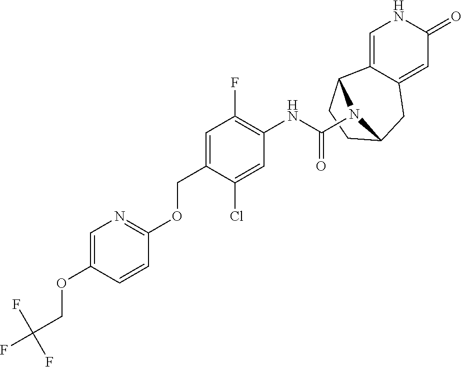 [SMILES: O=C(Nc1cc(Cl)c(COc2ccc(OCC(F)(F)F)cn2)cc1F)N1[C@H]2CC[C@@H]1c1c[nH]c(=O)cc1C2] | Method 7 553.1, 555.1 at 1.53 min | ¹H NMR (400 MHz, DMSO- d6) δ 11.26 (s, 1H), 8.56 (s, 1H), 7.97 (d, J = 3.2 Hz, 1H), 7.68 (d, J = 6.8 Hz, 1H), 7.56 (dd, J = 3.2, 8.8 Hz, 1H), 7.38 (d, J = 10.8 Hz, 1H), 7.19 (s, 1H), 6.93 (d, J = 8.8 Hz, 1H), 6.10 (s, 1H), 5.29 (s, 2H), 5.03 (d, J = 6.8 Hz, 1H), 4.77 (q, J = 8.8 Hz, 2H), 4.58 (t, J = 6.4 Hz, 1H), 3.12 (dd, J = 5.2, 18.0 Hz, 1H), 2.54 (d, J = 18.4 Hz, 1H), 2.21-2.01 (m, 2H), 1.74- 1.61 (m, 2H). |
| | (6S,9R)-N-(5-Chloro-2-fluoro- | | |
| | 4-(((5-(2,2,2- | | |
| | trifluoroethoxy)pyridin-2- | | |
| | yl)oxy)methyl)phenyl)-3-oxo- | | |
| | 3,5,6,7,8,9-hexahydro-2H-6,9- | | |
| | epiminocyclohepta[c]pyridine- | | |
| | 10-carboxamide | | |
| 55 | 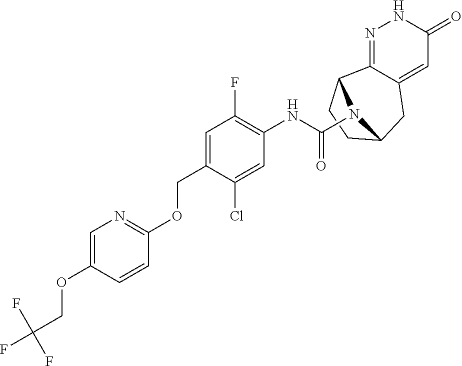 [SMILES: O=C(Nc1cc(Cl)c(COc2ccc(OCC(F)(F)F)cn2)cc1F)N1[C@H]2CC[C@@H]1c1n[nH]c(=O)cc1C2] | Method 7 554.1, 556.1 at 1.61 min | ¹H NMR (400 MHz, DMSO- d6) δ 12.71 (s, 1H), 8.80 (s, 1H), 7.97 (d, J = 2.8 Hz, 1H), 7.68 (d, J = 7.2 Hz, 1H), 7.57 (dd, J = 3.2, 8.8 Hz, 1H), 7.38 (d, J = 10.8 Hz, 1H), 6.93 (d, J = 8.8 Hz, 1H), 6.67 (s, 1H), 5.29 (s, 2H), 5.05 (d, J = 5.6 Hz, 1H), 4.77 (q, J = 8.8 Hz, 2H), 4.62 (t, J = 6.0 Hz, 1H), 3.17 (dd, J = 5.6 Hz, J = 18.4 Hz, 1H), 2.64 (d, J = 18.4 Hz, 1H), 2.24-2.11 (m, 2H), 1.81-1.64 (m, 2H). |
| | (6S,9R)-N-(5-Chloro-2-fluoro- | | |
| | 4-(((5-(2,2,2- | | |
| | trifluoroethoxy)pyridin-2- | | |
| | yl)oxy)methyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 56 | 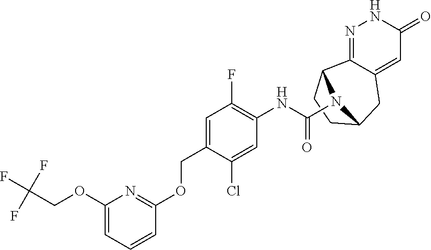 [SMILES: O=C(Nc1cc(Cl)c(COc2cccc(OCC(F)(F)F)n2)cc1F)N1[C@H]2CC[C@@H]1c1n[nH]c(=O)cc1C2] | Method 7 554.1 at 2.07 min | ¹H NMR (400 MHz, DMSO- d6) δ 12.71 (s, 1H), 8.81 (s, 1H), 7.76-7.70 (m, 2H), 7.42 (d, J = 11.2 Hz, 1H), 6.68 (s, 1H), 6.59-6.54 (m, 2H), 5.36 (s, 2H), 5.06 (d, J = 5.2 Hz, 1H), 4.97 (q, J = 8.8 Hz, 2H), 4.62 (s, 1H), 3.17 (dd, J = 4.4, 18.8 Hz, 1H), 2.64 (d, J = 18.4 Hz, 1H), 2.25-2.12 (m, 2H), 1.81-1.76 (m, 1H), 1.69- 1.63 (m, 1H). |
| | (6S,9R)-N-(5-Chloro-2-fluoro- | | |
| | 4-(((6-(2,2,2- | | |
| | trifluoroethoxy)pyridin-2- | | |
| | yl)oxy)methyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 57 | 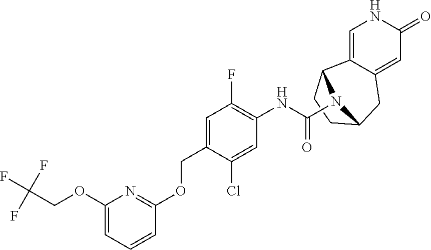 [SMILES: O=C(Nc1cc(Cl)c(COc2cccc(OCC(F)(F)F)n2)cc1F)N1[C@H]2CC[C@@H]1c1c[nH]c(=O)cc1C2] | Method 7 553.1 at 2.01 min | ¹H NMR (400 MHz, DMSO- d6) δ 11.26 (s, 1H), 8.57 (s, 1H), 7.76-7.71 (m, 2H), 7.42 (d, J = 11.2 Hz, 1H), 7.19 (s, 1H), 6.59-6.54 (m, 2H), 6.10 (s, 1H), 5.35 (s, 2H), 5.04 (d, J = 6.0 Hz, 1H), 4.97 (q, J = 9.2 Hz, 2H), 4.58 (t, J = 6.0 Hz, 1H), 3.13 (dd, J = 4.8, 18.0 Hz, 1H), 2.57- 2.50 (m, 1H), 2.21-2.04 (m, 2H), 1.74-1.61 (m, 2H). |
| | (6S,9R)-N-(5-Chloro-2-fluoro- | | |
| | 4-(((6-(2,2,2- | | |
| | trifluoroethoxy)pyridin-2- | | |
| | yl)oxy)methyl)phenyl)-3-oxo- | | |
| | 3,5,6,7,8,9-hexahydro-2H-6,9- | | |
| | epiminocyclohepta[c]pyridine- | | |
| | 10-carboxamide | | |
| 58 | 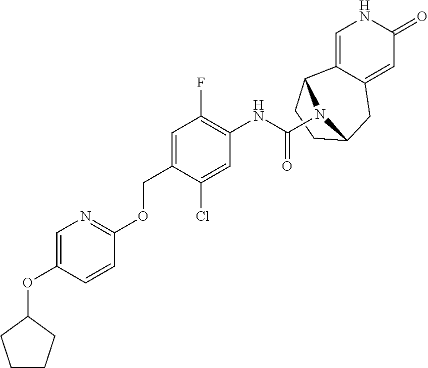 [SMILES: O=C(Nc1cc(Cl)c(COc2ccc(OC3CCCC3)cn2)cc1F)N1[C@H]2CC[C@@H]1c1c[nH]c(=O)cc1C2] | Method 3 539.1 at 2.27 min | ¹H NMR (400 MHz, DMSO- d6) δ 8.55 (s, 1H), 7.81 (d, J = 2.8 Hz, 1H), 7.70-7.67 (m, 1H), 7.40-7.36 (m, 2H), 7.19 (s, 1H), 6.86 (d, J = 8.8 Hz, 1H), 6.10 (s, 1H), 5.26 (s, 2H), 5.04 (d, J = 6.0 Hz, 1H), 4.78 (t, J = 5.6 Hz, 1H), 4.59-4.56 (m ,1H), 3.13 (dd, J = 4.8, 17.6 Hz, 1H), 2.57-2.50 (m, 1H), 2.21- 2.04 (m, 2H), 1.92-1.84 (m, 2H), 1.71-1.57 (m, 8H). (1 exchangeable proton not observed) |
| | (6S,9R)-N-(5-Chloro-4-(((5- | | |
| | (cyclopentyloxy)pyridin-2- | | |
| | yl)oxy)methyl)-2-fluorophenyl)- | | |
| | 3-oxo-3,5,6,7,8,9-hexahydro- | | |
| | 2H-6,9- | | |
| | epiminocyclohepta[c]pyridine- | | |
| | 10-carboxamide | | |
| 59 | 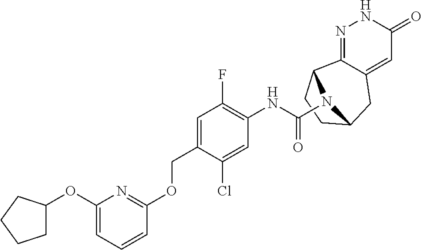 [SMILES: O=C(Nc1cc(Cl)c(COc2cccc(OC3CCCC3)n2)cc1F)N1[C@H]2CC[C@@H]1c1n[nH]c(=O)cc1C2] | Method 7 540.2 at 2.62 min | ¹H NMR (400 MHz, DMSO- d6) δ 12.70 (s, 1H), 8.79 (s, 1H), 7.69 (d, J = 7.2 Hz, 1H), 7.61 (t, J = 8.0 Hz, 1H), 7.34 (d, J = 10.8 Hz, 1H), 6.73 (s, 1H), 6.42 (d, J = 8.0 Hz, 1H), 6.32 (d, J = 8,0 Hz, 1H), 5.33 (s, 2H), 5.22-5.17 (m, 1H), 5.05 (d, J = 5.6 Hz, 1H), 4.63- 4.61 (m, 1H), 3.16 (dd, J = 4.6, 19.6 Hz, 1H), 2.64 (d, J = 18.4 Hz, 1H), 2.43-2.12 (m, 2H), 1.93-1.85 (m, 2H), 1.81-1.53 (m, 8H). |
| | (6S,9R)-N-(5-Chloro-4-(((6- | | |
| | (cyclopentyloxy)pyridin-2- | | |
| | yl)oxy)methyl)-2-fluorophenyl)- | | |
| | 3-oxo-3,5,6,7,8,9-hexahydro- | | |
| | 2H-6,9- | | |
| | epiminocyclohepta[c]pyridazine- | | |
| | 10-carboxamide | | |

### Intermediate 41 (1-41)

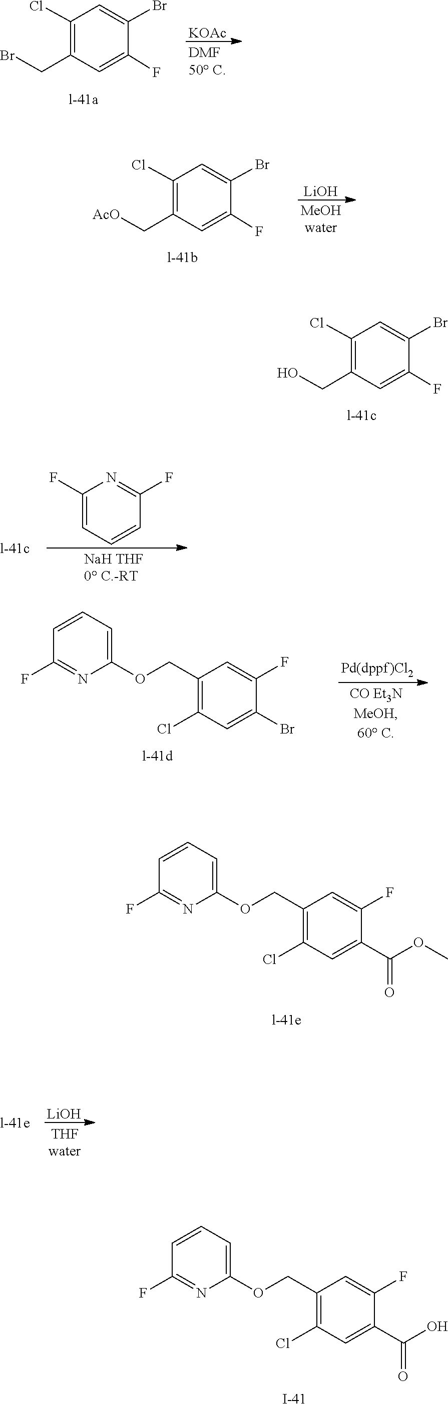

Step 1: To a solution of 1-bromo-4-(bromomethyl)-5-chloro-2-fluorobenzene I-41a (2.0 g, 6.61 mmol) in DMF (20 mL) was added KOAc (1.2 g, 13.22 mmol) and the mixture was warmed at 50° C. for 1 h. Water (15 mL) was added and the product was extracted with EtOAc (3×15 mL). The combined organics were washed with brine (15 mL), dried over Na₂SO₄ and concentrated in vacuo to give 4-bromo-2-chloro-5-fluorobenzyl acetate I-41b as a white solid. ¹H NMR (400 MHz, DMSO-d6) δ 7.95 (d, J=6.4 Hz, 1H), 7.54 (d, J=9.2 Hz, 1H), 5.09 (s, 2H), 2.11 (s, 3H).

Step 2: To a mixture of 4-bromo-2-chloro-5-fluorobenzyl acetate I-41b (795 mg, 2.82 mmol) in MeOH (6 mL) and water (2 mL) was added LiOH (236 mg, 5.64 mmol) and the reaction was stirred at RT for 16 h. The resulting mixture was adjusted to pH=3 with 1 M HCl and the product was extracted with EtOAc (3×5 mL). The combined organics were dried over Na₂SO₄ and concentrated in vacuo to give (4-bromo-2-chloro-5-fluorophenyl)methanol I-41c as a white solid. ¹H NMR (400 MHz, DMSO-d6) δ 7.83 (d, J=6.0 Hz, 1H), 7.44 (d, J=9.2 Hz, 1H), 5.63 (t, J=4.4 Hz, 1H), 4.49 (d, J=4.4 Hz, 2H).

Step 3: To a solution of (4-bromo-2-chloro-5-fluorophenyl)methanol I-41c (847 mg, 3.53 mmol) in THF (10 mL) was added NaH (212 mg, 60% wt., 5.30 mmol) at 0° C., and the reaction was stirred at 0° C. for 30 min. 2,6-Difluoropyridine (407 mg, 3.53 mmol) was added and the mixture was warmed to RT over 16 h before being diluted with water (15 mL) and the product was extracted into EtOAc (3×10 mL). The combined organics were dried over Na₂SO₄ and concentrated in vacuo and the product was purified by chromatography on silica gel (10% EtOAc/petroleum ether) to give 2-((4-bromo-2-chloro-5-fluorobenzyl)oxy)-6-fluoropyridine I-41d as a white solid. ¹H NMR (400 MHz, DMSO-d6) δ 7.98-7.90 (m, 2H), 7.61 (d, J=9.2 Hz, 1H), 6.90-6.88 (m, 1H), 6.78-6.76 (m, 1H), 5.31 (s, 2H).

Step 4: To a mixture of 2-((4-bromo-2-chloro-5-fluorobenzyl)oxy)-6-fluoropyridine I-41d (530 mg, 1.58 mmol) and Et₃N (330 μL, 2.37 mmol) in MeOH (5 mL) was added PdCl₂(dppf) (115 mg, 0.15 mmol) and the reaction was heated at 60° C. under a CO atmosphere for 16 h. The mixture was diluted with water (10 mL) and the product was extracted into EtOAc (3×5 mL). The combined organics were dried over Na₂SO₄ and concentrated in vacuo. The product was purified by chromatography on silica gel (10% EtOAc/petroleum ether) to give methyl 5-chloro-2-fluoro-4-(((6-fluoropyridin-2-yl)oxy)methyl)benzoate 1-41e as a white solid. ¹H NMR (400 MHz, DMSO-d6) δ 7.97-7.91 (m, 2H), 7.56 (d, J=11.2 Hz, 1H), 6.94-6.92 (m, 1H), 6.79-6.77 (m, 1H), 5.39 (s, 2H), 3.86 (s, 3H).

Step 5: To a mixture of methyl 5-chloro-2-fluoro-4-(((6-fluoropyridin-2-yl)oxy)methyl)benzoate I-41e (404 mg, 1.28 mmol) in THF (3 mL) and water (1 mL) was added LiOH monohydrate (108 mg, 2.57 mmol). After stirring at RT for 16 h, the reaction was adjust pH=3 with 1 M HCl and the product was extracted with EtOAc (3×5 mL). The combined organics were dried over Na₂SO₄ and concentrated in vacuo to give 5-chloro-2-fluoro-4-(((6-fluoropyridin-2-yl)oxy)methyl)benzoic acid 1-40 as a white solid. ¹H NMR (400 MHz, DMSO-d6) δ 7.98-7.89 (m, 2H), 7.52 (d, J=11.2 Hz, 1H), 6.94-6.92 (m, 1H), 6.79-6.77 (m, 1H), 5.38 (s, 2H). 1 exchangeable proton not observed.

### Intermediate 42 (1-42)

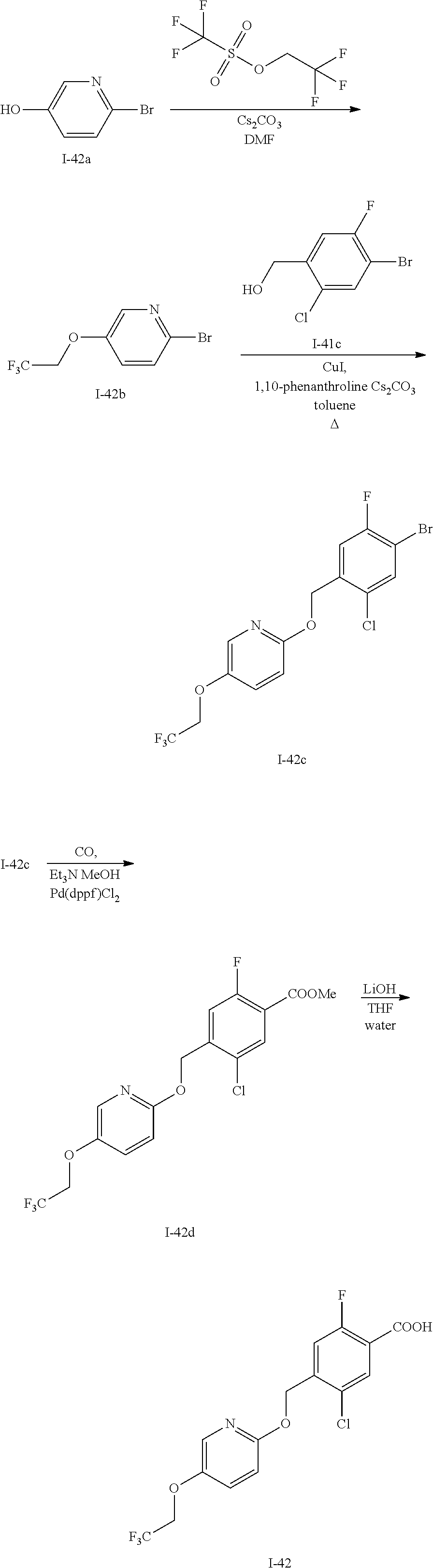

Step 1: To a solution of 6-bromopyridin-3-ol (500 mg, 2.89 mmol) in DMF (5 mL) was added Cs₂CO₃ (1.09 g, 3.34 mmol) and 2,2,2-trifluoroethyl trifluoromethanesulfonate (764 mg, 3.26 mmol) at RT. The reaction was stirred at RT for 1h, before water (50 mL) was added and the product was extracted with EtOAc (3×40 mL). The combined organics were washed with brine (80 mL), dried over Na₂SO₄ and concentrated in vacuo. The product was purified by chromatography on silica gel (10% EtOAc/petroleum ether) to give 2-bromo-5-(2,2,2-trifluoroethoxy)pyridine I-42b as a white solid. ¹H NMR (400 MHz, DMSO-d6) δ 8.25 (d, J=3.2 Hz, 1H), 7.62 (d, J=8.8 Hz, 1H), 7.54 (dd, J=3.2, 8.8 Hz, 1H), 4.89 (q, J=8.8 Hz, 2H).

Step 2: A mixture of 2-bromo-5-(2,2,2-trifluoroethoxy)pyridine I-42b (415 mg, 1.74 mmol), (4-bromo-2-chloro-5-fluorophenyl)methanol I-41c (444 mg, 1.74 mmol), Cs₂CO₃ (851 mg, 2.60 mmol), 1,10-phenanthroline (63 mg, 0.34 mmol) and Cul (34 mg, 0.17 mmol) in toluene (8 mL) was stirred at reflux for 16 h. The reaction was allowed to cool to RT and concentrated in vacuo. The product was purified by chromatography on silica gel (5% EtOAc/petroleum ether) to give 2-((4-bromo-2-chloro-5-fluorobenzyl)oxy)-5-(2,2,2-trifluoroethoxy)pyridine I-42c as a white solid. ¹H NMR (400 MHz, DMSO-d6) δ 7.98 (d, J=3.2 Hz, 1H), 7.97 (d, J=6.4 Hz, 1H), 7.62-7.55 (m, 2H), 6.99 (d, J=8.8 Hz, 1H), 5.34 (s, 2H), 4.80 (q, J=8.8 Hz, 2H).

Step 3: Methyl 5-chloro-2-fluoro-4-(((5-(2,2,2-trifluoroethoxy)pyridin-2-yl)oxy)methyl)benzoate I-42d was synthesised from 2-((4-bromo-2-chloro-5-fluorobenzyl)oxy)-5-(2,2,2-trifluoroethoxy)pyridine I-42c using a procedure essentially the same as for I-41e. ¹H NMR (400 MHz, DMSO-d6) δ 7.98 (d, J=3.2 Hz, 1H), 7.91 (d, J=6.4 Hz, 1H), 7.60 (dd, J=2.8, 8.8 Hz, 1H), 7.50 (d, J=7.2 Hz, 1H), 7.00 (d, J=9.2 Hz, 1H), 5.40 (s, 2H), 4.78 (q, J=8.8 Hz, 2H), 3.86 (s, 3H).

Step 4: 5-chloro-2-fluoro-4-(((5-(2,2,2-trifluoroethoxy)pyridin-2-yl)oxy)methyl)benzoic acid I-42 was synthesised from methyl 5-chloro-2-fluoro-4-(((5-(2,2,2-trifluoroethoxy)pyridin-2-yl)oxy)methyl)benzoate I-42d using a procedure essentially the same as for 1-41. LCMS (Method 5) m/z 379.9, 381.8 (M+H)⁺ (ES⁺), at 1.74 min.

### Intermediate 43 (1-43)

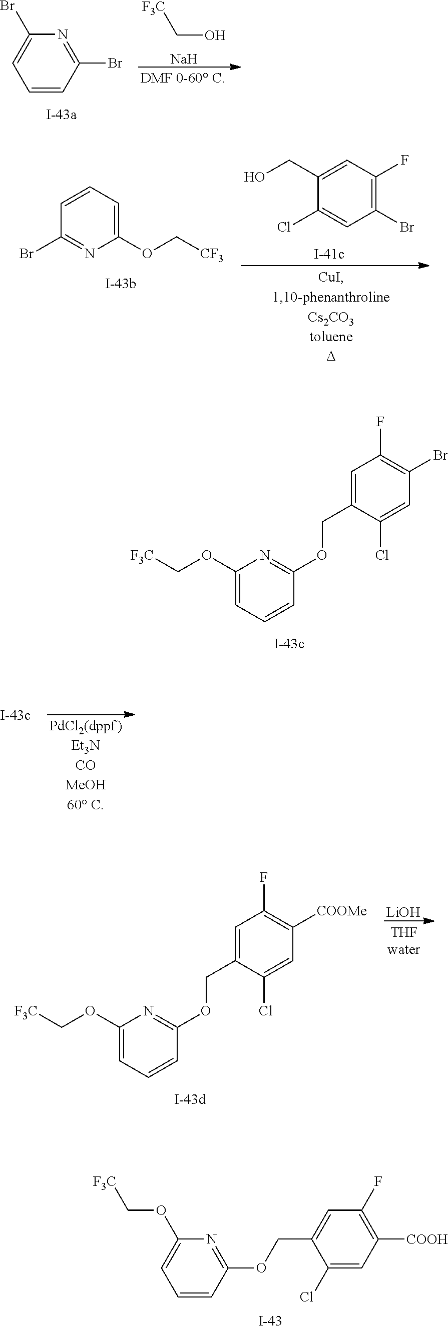

Step 1: To a mixture of 2,6-dibromopyridine I-43a (1 g, 4.22 mmol) and NaH (186 mg, 60% wt., 4.64 mmol) in dry DMF (2 mL) was added 2,2,2-trifluoroethan-1-ol (365 μL, 5.07 mmol) dropwise at 0° C. The reaction was warmed to 60° C. for 16 h and the mixture was diluted with water (10 mL) and the product extracted into EtOAc (3×5 mL). The combined organics were dried over Na₂SO₄, filtered and concentrated in vacuo. The product was purified by chromatography on silica gel (petroleum ether) to give 2-bromo-6-(2,2,2-trifluoroethoxy)pyridine I-43b as a colorless oil. ¹H NMR (400 MHz, DMSO-d6) δ 7.76 (t, J=7.6 Hz, 1H), 7.37 (d, J=7.2 Hz, 1H), 7.05 (d, J=8.4 Hz, 1H), 4.97 (q, J=8.8 Hz, 2H)

Step 2: 2-((4-Bromo-2-chloro-5-fluorobenzyl)oxy)-6-(2,2,2-trifluoroethoxy)pyridine I-43c was synthesised from 2-bromo-6-(2,2,2-trifluoroethoxy)pyridine I-43b and (4-bromo-2-chloro-5-fluorophenyl)methanol I-41c using a procedure essentially the same as for I-42c. ¹H NMR (400 MHz, DMSO-d6) δ 7.96 (d, J=6.4 Hz, 1H), 7.76 (t, J=7.6 Hz, 1H), 7.56 (d, J=9.2 Hz, 1H), 6.62 (d, J=7.6 Hz, 1H), 6.56 (d, J=8.0 Hz, 1H), 5.39 (s, 2H), 4.95 (q, J=8.8 Hz, 2H)

Step 3: Methyl 5-chloro-2-fluoro-4-(((6-(2,2,2-trifluoroethoxy)pyridin-2-yl)oxy)methyl)benzoate I-43d was synthesised from 2-((4-bromo-2-chloro-5-fluorobenzyl)oxy)-6-(2,2,2-trifluoroethoxy)pyridine I-43c using a procedure essentially the same as for I-41e. ¹H NMR (400 MHz, DMSO-d6) δ 7.94 (d, J=6.4 Hz, 1H), 7.77 (t, J=8.0 Hz, 1H), 7.51 (d, J=10.8 Hz, 1H), 6.66 (d, J=8.0 Hz, 1H), 6.57 (d, J=8.0 Hz, 1H), 5.47 (s, 2H), 4.92 (q, J=8.8 Hz, 2H), 3.87 (s, 3H).

Step 4: 5-Chloro-2-fluoro-4-(((6-(2,2,2-trifluoroethoxy)pyridin-2-yl)oxy)methyl)benzoic acid 1-43 was synthesised from methyl 5-chloro-2-fluoro-4-(((6-(2,2,2-trifluoroethoxy)pyridin-2-yl)oxy)methyl)benzoate I-43d using a procedure essentially the same as for 1-41. ¹H NMR (400 MHz, DMSO-d6) δ 7.90 (d, J=6.4 Hz, 1H), 7.77 (t, J=8.0 Hz, 1H), 7.46 (d, J=10.8 Hz, 1H), 6.65 (d, J=8.0 Hz, 1H), 6.57 (d, J=8.0 Hz, 1H), 5.46 (s, 2H), 4.92 (q, J=9.2 Hz, 2H). (1 exchangeable proton not observed)

### Intermediate 44 (1-44)

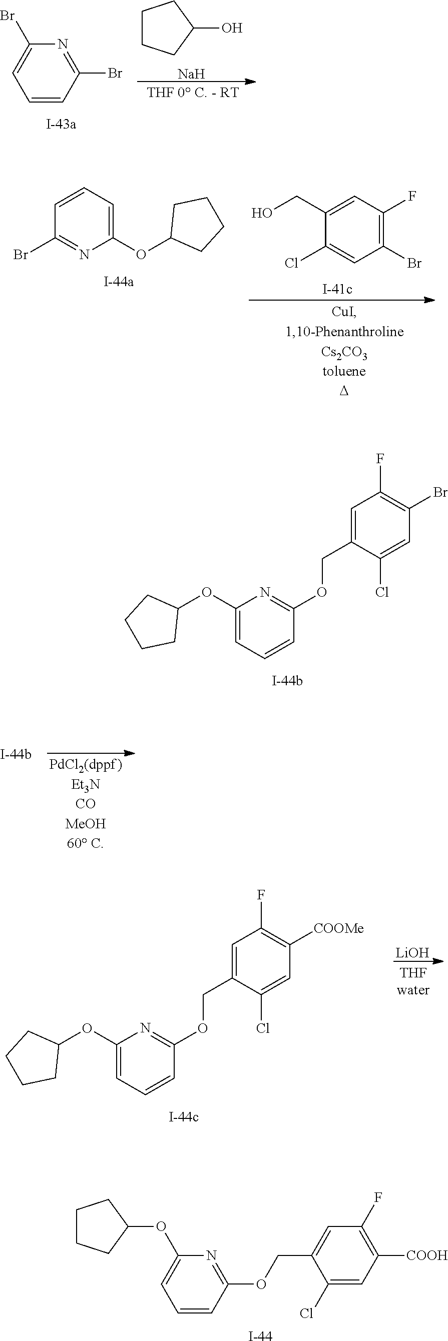

Step 1: To a mixture of 2,6-dibromopyridine I-43a (2.75 g, 11.6 mmol, 2 eq) and NaH (348 mg, 60% wt., 8.7 mmol) in THF (15 mL) was added a solution of cyclopentanol (500 mg, 5.80 mmol) dropwise at 0° C. The reaction was warmed to RT and stirred at RT for 16 h, after which the mixture was diluted with saturated aqueous NH₄Cl (20 ml) and the product was extracted with EtOAc (3×30 mL). The combined organics were dried over Na₂SO₄, filtered and concentrated in vacuo. The product was purified by chromatography on silica gel (petroleum ether) to give 2-bromo-6-(cyclopentyloxy)pyridine I-44a as a white solid. ¹H NMR (400 MHz, DMSO-d6) δ 7.62 (t, J=7.6 Hz, 1H), 7.17 (d, J=7.2 Hz, 1H), 6.79 (d, J=8.4 Hz, 1H), 5.29-5.25 (m, 1H), 1.96-1.90 (m, 2H), 1.72-1.56 (m, 6H)

Step 2: 2-((4-Bromo-2-chloro-5-fluorobenzyl)oxy)-6-(cyclopentyloxy)pyridine I-44b was synthesised from 2-bromo-6-(cyclopentyloxy)pyridine I-44a and (4-bromo-2-chloro-5-fluorophenyl)methanol I-41c using a procedure essentially the same as for I-42c. ¹H NMR (400 MHz, DMSO-d6) δ 7.96 (d, J=6.4 Hz, 1H), 7.63 (t, J=7.6 Hz, 1H), 7.49 (d, J=9.2 Hz, 1H), 6.46 (d, J=7.6 Hz, 1H), 6.34 (d, J=8.0 Hz, 1H), 5.36 (s, 2H), 5.19-5.15 (m, 1H), 1.90-1.79 (m, 2H), 1.73-1.48 (m, 6H).

Step 3: Methyl 5-chloro-4-(((6-(cyclopentyloxy)pyridin-2-yl)oxy)methyl)-2-fluorobenzoate I-44c was synthesised from 2-((4-bromo-2-chloro-5-fluorobenzyl)oxy)-6-(cyclopentyloxy)pyridine I-44b using a procedure essentially the same as for I-41e. ¹H NMR (400 MHz, DMSO-d6) δ 7.94 (d, J=6.4 Hz, 1H), 7.64 (t, J=8.0 Hz, 1H), 7.43 (d, J=10.8 Hz, 1H), 6.50 (d, J=8.0 Hz, 1H), 6.35 (d, J=8.0 Hz, 1H), 5.44 (s, 2H), 5.14-5.11 (m, 1H), 3.86 (s, 3H), 1.87-1.77 (m, 2H), 1.71-1.47 (m, 6H).

Step 4: 5-Chloro-4-(((6-(cyclopentyloxy)pyridin-2-yl)oxy)methyl)-2-fluorobenzoic acid 1-44 was synthesised from methyl 5-chloro-4-(((6-(cyclopentyloxy)pyridin-2-yl)oxy)methyl)-2-fluorobenzoate I-44c using a procedure essentially the same as for 1-41. ¹H NMR (400 MHz, DMSO-d6) δ 13.58 (s, 1H), 7.90 (d, J=6.4 Hz, 1H), 7.64 (t, J=8.0 Hz, 1H), 7.39 (d, J=10.8 Hz, 1H), 6.50 (d, J=8.0 Hz, 1H), 6.35 (d, J=8.0 Hz, 1H), 5.43 (s, 2H), 5.15-5.11 (m, 1H), 1.84-1.77 (m, 2H), 1.67-1.50 (m, 6H).

### Intermediate 45 (1-45)

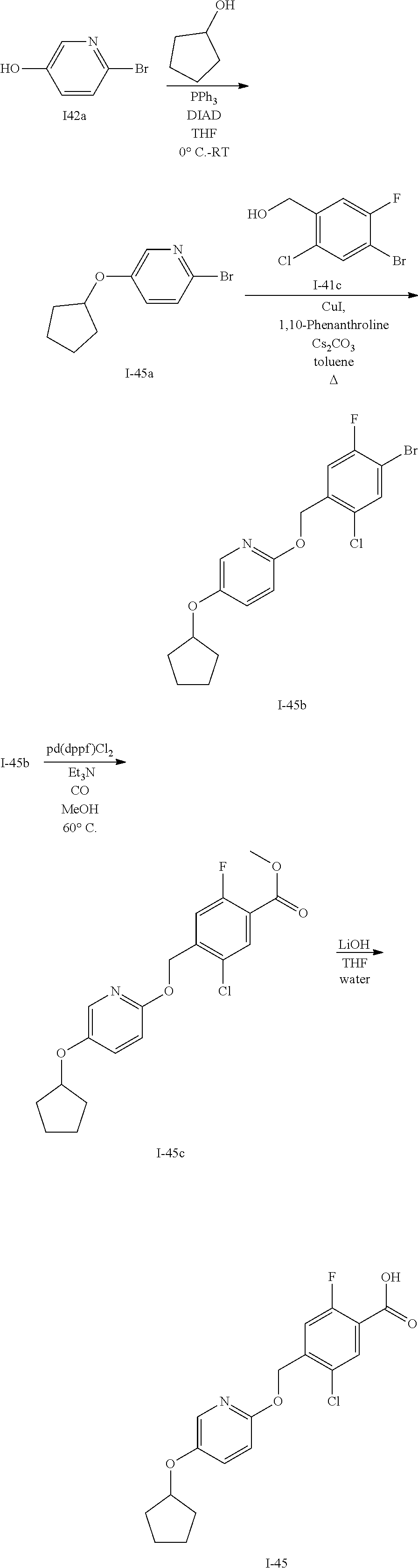

Step 1: To a solution of 6-bromopyridin-3-ol I-42a (2 g, 11.49 mmol), cyclopentanol (0.99 g, 11.49 mmol) and PPh₃ (6.0 g, 22.98 mmol) in THF (20 mL) was added DIAD (4.6 g, 22.98 mmol) at 0° C. The resulting solution was warmed to RT and stirred for 16 h before the reaction mixture was diluted with water (20 mL) and the product was extracted with EtOAc (3×10 mL). The combined organics were dried over Na₂SO₄, filtered and concentrated in vacuo. The product was purified by chromatography on silica gel (10% EtOAc/petroleum ether) to give 2-bromo-5-(cyclopentyloxy)pyridine I-45a as a pink oil. ¹H NMR (400 MHz, DMSO-d6) δ 8.06 (d, J=3.2 Hz, 1H), 7.52-7.50 (m, 1H), 7.36-7.33 (m, 1H), 4.88 (t, J=5.6 Hz, 1H), 1.96-1.89 (m, 2H), 1.71-1.56 (m, 6H).

Step 2: 2-((4-Bromo-2-chloro-5-fluorobenzyl)oxy)-5-(cyclopentyloxy)pyridine I-45b was synthesised from 2-bromo-5-(cyclopentyloxy)pyridine I-45a and (4-bromo-2-chloro-5-fluorophenyl)methanol I-41c using a procedure essentially the same as for I-42c. ¹H NMR (400 MHz, DMSO-d6) δ 7.94 (d, J=6.4 Hz, 1H), 7.80 (d, J=2.8 Hz, 1H), 7.53 (d, J=9.2 Hz, 1H), 7.42-7.39 (m, 1H), 6.89 (d, J=8.8 Hz, 1H), 5.29 (s, 2H), 4.78 (t, J=5.6 Hz, 1H), 1.91-1.84 (m, 2H), 1.69-1.53 (m, 6H)

Step 3: Methyl 5-chloro-4-(((5-(cyclopentyloxy)pyridin-2-yl)oxy)methyl)-2-fluorobenzoate I-45c was synthesised from 2-((4-bromo-2-chloro-5-fluorobenzyl)oxy)-5-(cyclopentyloxy)pyridine I-45b using a procedure essentially the same as for I-41e. ¹H NMR (400 MHz, DMSO-d6) δ 7.92 (d, J=6.4 Hz, 1H), 7.80 (d, J=2.8 Hz, 1H), 7.49 (d, J=11.2 Hz, 1H), 7.43-7.40 (m, 1H), 6.94 (d, J=8.8 Hz, 1H), 5.37 (s, 2H), 4.78 (t, J=5.2 Hz, 1H), 3.86 (s, 3H), 1.91-1.84 (m, 2H), 1.69-1.53 (m, 6H).

Step 4: 5-Chloro-4-(((5-(cyclopentyloxy)pyridin-2-yl)oxy)methyl)-2-fluorobenzoic acid 1-45 was synthesised from methyl 5-chloro-4-(((5-(cyclopentyloxy)pyridin-2-yl)oxy)methyl)-2-fluorobenzoate I-45c using a procedure essentially the same as for 1-41. LCMS (Method 7) m/z 64.1 (M−H)⁻ (ES-), at 1.80 min.

### Experimental Scheme 3

Compound 60 (6S,9R)-N-(4-(6-((2-azabicyclo[2.1.1]hexan-4-yl)methoxy)pyridin-3-yl)-5-chloro-2-fluorophenyl)-3-oxo-3,5,6,7,8,9-hexahydro-2H-6,9-epiminocyclohepta[c]pyridine-10-carboxamide

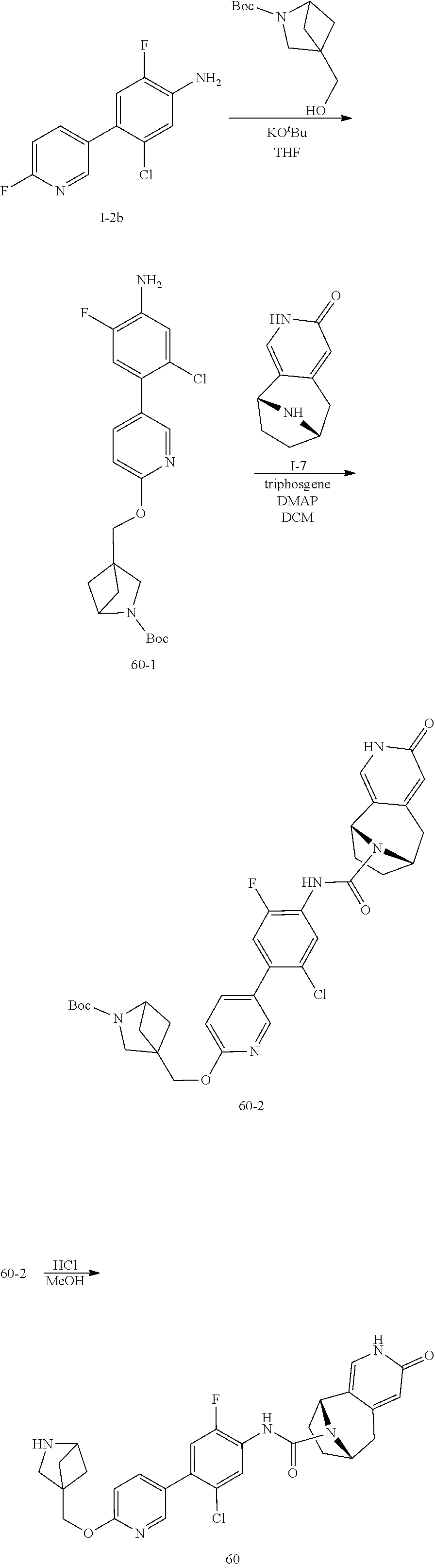

Step 1: To a solution of 5-chloro-2-fluoro-4-(6-fluoropyridin-3-yl)aniline I-2b (113 mg, 469 μmol) and tert-butyl 4-(hydroxymethyl)-2-azabicyclo[2.1.1]hexane-2-carboxylate (100 mg, 469 μmol) in THF (2 mL) at 0° C. was added a solution of potassium tert-butoxide in THF (1.20 mL, 1 M, 1.20 mmol). The reaction was allowed to warm to room temperature and stirred for 2 h before water (50 ml) was added and the product was extracted with DCM (50 ml). The aqueous was extracted with additional DCM (2×50 mL). The combined organics were washed with water (100 mL) and brine (100 mL), dried over sodium sulfate and concentrated in vacuo to give tert-butyl 4-(((5-(4-amino-2-chloro-5-fluorophenyl)pyridin-2-yl)oxy)methyl)-2-azabicyclo[2.1.1]hexane-2-carboxylate 60-1 as a dark red oil. ¹H NMR (400 MHz, DMSO-d6) δ 8.13 (d, J=2.5 Hz, 1H), 7.74 (dd, J=8.5, 2.5 Hz, 1H), 7.12 (d, J=11.8 Hz, 1H), 6.89 (dd, J=10.0, 8.5 Hz, 2H), 5.61 (s, 2H), 4.54 (s, 1H), 4.51 (s, 2H), 4.23 (s, 2H), 3.23 (s, 4H), 1.40 (s, 9H).

Step 2: A solution of tert-butyl 4-(((5-(4-amino-2-chloro-5-fluorophenyl)pyridin-2-yl)oxy)methyl)-2-azabicyclo[2.1.1]hexane-2-carboxylate 60-1 (164 mg, 321 μmol) and DMAP (118 mg, 964 μmol) in DCM (2 mL) was added to a solution of triphosgene (38.1 mg, 129 μmol) in DCM (2 mL) and the mixture was stirred at RT for 15 min. This mixture was added to a suspension of (6S,9R)-2,5,6,7,8,9-hexahydro-3H-6,9-epiminocyclohepta[c]pyridin-3-one 1-7 (56.6 mg, 321 μmol) in DCM (2 mL) and the resulting reaction was stirred at RT for 2 h. After this, 1 M aq. soln HCl (15 mL) and extracted with DCM (3×15 mL). The combined organic layer was washed with brine (50 mL), dried with sodium sulfate and concentrated in vacuo. The product was purified by chromatography on silica gel (0-10% MeOH/DCM) to afford tert-butyl 4-(((5-(2-chloro-5-fluoro-4-((6S,9R)-3-oxo-3,5,6,7,8,9-hexahydro-2H-6,9-epiminocyclohepta[c]pyridine-10-carboxamido)phenyl)pyridin-2-yl)oxy)methyl)-2-azabicyclo[2.1.1]hexane-2-carboxylate 60-2 as a white solid. LCMS (Method 2) m/z 636.5, 638.5 (M+H)⁺ (ES⁺), at 1.98 min. ¹H NMR (400 MHz, DMSO-d6) δ 11.27 (s, 1H), 8.63 (s, 1H), 8.20 (dd, J=2.6, 0.8 Hz, 1H), 7.81 (dd, J=8.6, 2.5 Hz, 1H), 7.77 (d, J=7.4 Hz, 1H), 7.37 (d, J=11.1 Hz, 1H), 7.20 (s, 1H), 6.93 (dd, J=8.6, 0.7 Hz, 1H), 6.11 (s, 1H), 5.06 (d, J=5.8 Hz, 1H), 2.26-2.02 (m, 2H), 4.60 (t, J=6.5 Hz, 1H), 4.54 (s, 2H), 4.23 (d, J=1.8 Hz, 1H), 3.24 (s, 3H), 3.20-3.09 (m, 1H), 2.56 (d, J=17.9 Hz, 1H), 1.95-1.83 (m, 2H), 1.77-1.60 (m, 2H), 1.43-1.42 (m, 1H), 1.41 (s, 9H).

Step 3: To a solution of tert-butyl 4-(((5-(2-chloro-5-fluoro-4-((6S,9R)-3-oxo-3,5,6,7,8,9-hexahydro-2H-6,9-epiminocyclohepta[c]pyridine-10-carboxamido)phenyl)pyridin-2-yl)oxy)methyl)-2-azabicyclo[2.1.1]hexane-2-carboxylate 60-2 (69.0 mg, 108 μmol) in MeOH (5 mL) was added a solution of HCl in MeOH (362 μL, 3 M, 1.08 mmol) and the reaction was stirred at RT for 24 h. A further portion of HCl in MeOH (362 μL, 3 M, 1.08 mmol) was added and the reaction was stirred at RT for 24 h. The resulting mixture was concentrated in vacuo and the residue was redissolved in MeOH (3 mL) followed by addition of MP-carbonate (111 mg) and the suspension was stirred for 4 h. The mixture was filtered and the filtrate was concentrated in vacuo. The material was stirred with SCX resin (500 mg) and MeOH (5 mL), with 2 drops of acetic acid, for 1 h. The resin was then washed with MeOH (15 mL). The product was eluted with 0.35 M ammonia in MeOH and the eluant was concentrated in vacuo to give (6S,9R)-N-(4-(6-((2-azabicyclo[2.1.1]hexan-4-yl)methoxy)pyridin-3-yl)-5-chloro-2-fluorophenyl)-3-oxo-3,5,6,7,8,9-hexahydro-2H-6,9-epiminocyclohepta[c]pyridine-10-carboxamide 60 as an off-white solid. LCMS (Method 1) m/z 536.3, 538.8 (M+H)⁺ (ES⁺), at 1.23 min. ¹H NMR (400 MHz, DMSO-d6) δ 8.66 (s, 1H), 8.19 (d, J=2.5 Hz, 1H), 7.80 (dd, J=8.6, 2.5 Hz, 1H), 7.76 (d, J=7.4 Hz, 1H), 7.38 (d, J=11.1 Hz, 1H), 7.21 (s, 1H), 6.91 (d, J=8.6 Hz, 1H), 6.11 (s, 1H), 5.06 (d, J=5.8 Hz, 1H), 4.59 (t, J=6.4 Hz, 1H), 4.53 (s, 2H), 3.58 (s, 1H), 3.14 (dd, J=18.7, 4.6 Hz, 1H), 2.79 (s, 2H), 2.58 (s, 1H), 2.24-2.03 (m, 2H), 1.80-1.60 (m, 4H), 1.28 (dd, J=4.4, 1.7 Hz, 2H), 1.23 (s, 1H).

### Experimental Scheme 4

### Compound 61 (6S,9R)-N-(5-Chloro-2-fluoro-4-(6-((2-methyl-2-azabicyclo[2.1.1]hexan-4-yl)methoxy)pyridin-3-yl)phenyl)-3-oxo-3,5,6,7,8,9-hexahydro-2H-6,9-epiminocyclohepta[c]pyridine-10-carboxamide

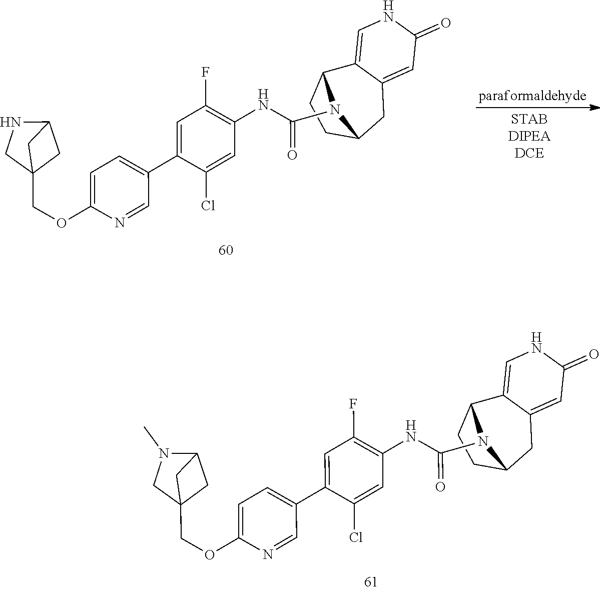 [SMILES: O=C(Nc1cc(Cl)c(-c2ccc(OCC34CNC(C3)C4)nc2)cc1F)N1[C@H]2CC[C@@H]1c1c[nH]c(=O)cc1C2 ; CN1CC2(COc3ccc(-c4cc(F)c(NC(=O)N5[C@H]6CC[C@@H]5c5c[nH]c(=O)cc5C6)cc4Cl)cn3)CC1C2]

To a solution of (6S,9R)-N-(4-(6-((2-azabicyclo[2.1.1]hexan-4-yl)methoxy)pyridin-3-yl)-5-chloro-2-fluorophenyl)-3-oxo-3,5,6,7,8,9-hexahydro-2H-6,9-epiminocyclohepta[c]pyridine-10-carboxamide 60 (20.0 mg, 37.3 μmol) in DCE (1 mL) was added paraformaldehyde (7.40 mg, 246 μmol) and DIPEA (44 μL, 254 μmol) followed by addition of 4 A molecular sieves. The mixture was stirred at RT for 1 h after which STAB (35 mg, 164 μmol) was added and the reaction was stirred at RT for 18 h. A saturated aqueous NaHCO₃ solution (10 mL) was added and the product was extracted with DCM:IPA 7:3 (4×10 mL). The combined organics was passed through a hydrophobic frit and the filtrate was concentrated in vacuo. The product was purified by prep HPLC (25-100% MeCN/0.1% formic acid in water) to give (6S,9R)-N-(5-chloro-2-fluoro-4-(6-((2-methyl-2-azabicyclo[2.1.1]hexan-4-yl)methoxy)pyridin-3-yl)phenyl)-3-oxo-3,5,6,7,8,9-hexahydro-2H-6,9-epiminocyclohepta[c]pyridine-10-carboxamide 61 as a light yellow solid. LCMS (Method 1) m/z 550.3, 552.3 (M+H)⁺ (ES⁺), at 1.23 min. ¹H NMR (400 MHz, DMSO-d6) δ 11.25 (s, 1H), 8.64 (s, 1H), 8.19 (dd, J=2.5, 0.8 Hz, 1H), 7.80 (dd, J=8.6, 2.6 Hz, 1H), 7.77 (d, J=7.4 Hz, 1H), 7.37 (d, J=11.1 Hz, 1H), 7.20 (s, 1H), 6.90 (dd, J=8.6, 0.7 Hz, 1H), 6.11 (s, 1H), 5.06 (d, J=5.8 Hz, 1H), 4.63-4.56 (m, 1H), 4.46 (s, 2H), 3.24 (t, J=1.8 Hz, 1H), 3.15 (dd, J=17.9, 4.9 Hz, 1H), 2.60-2.53 (m, 3H), 2.34 (s, 3H), 2.24-2.02 (m, 2H), 1.78-1.59 (m, 4H), 1.56-1.52 (m, 2H).

### Experimental Scheme 5

### Compound 62 (6S,9R)-N-(5-Chloro-2-fluoro-4-(6-((2-methyl-2-azabicyclo[2.1.1]hexan-4-yl)methoxy)pyridin-3-yl)phenyl)-3-oxo-3,5,6,7,8,9-hexahydro-2H-6,9-epiminocyclohepta[c]pyridine-10-carboxamide

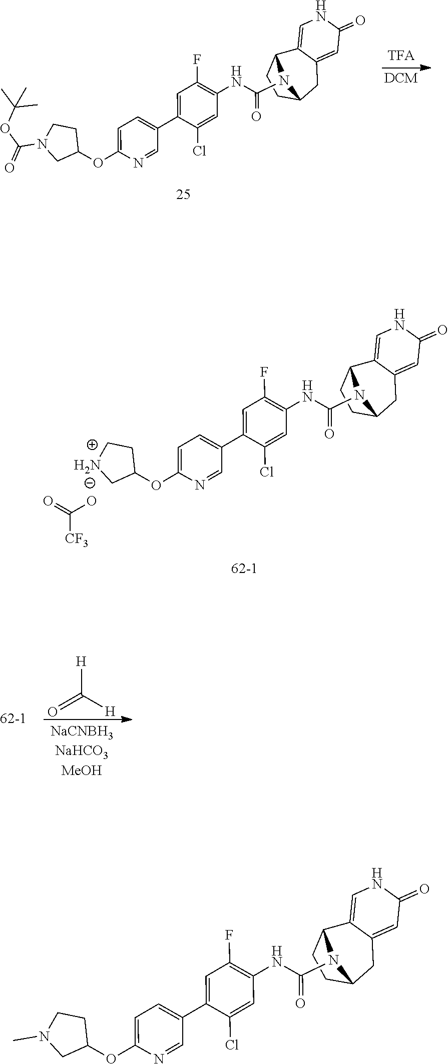

Step 1: To a solution of tert-butyl 3-((5-(2-chloro-5-fluoro-4-((6S,9R)-3-oxo-3,5,6,7,8,9-hexahydro-2H-6,9-epiminocyclohepta[c]pyridine-10-carboxamido)phenyl)pyridin-2-yl)oxy)pyrrolidine-1-carboxylate 25 (240 mg, 0.39 mmol) in DCM (3 mL) was added TFA (1 mL) at RT and the mixture was stirred for 1 h. The reaction was concentrated in vacuo and the product was purified by prep-HPLC to give (6S,9R)-N-(5-chloro-2-fluoro-4-(6-(pyrrolidin-3-yloxy)pyridin-3-yl)phenyl)-3-oxo-3,5,6,7,8,9-hexahydro-2H-6,9-epiminocyclohepta[c]pyridine-10-carboxamide trifluoroacetate salt 62-1 as a white solid. ¹H NMR (400 MHz, DMSO-d6) δ 9.11-8.98 (m, 1H), 8.66 (s, 1H), 8.24 (d, J=2.4 Hz, 1H), 7.86 (dd, J=2.8 Hz, J=8.8 Hz, 1H), 7.80-7.77 (m, 1H), 7.36 (d, J=11.2 Hz, 1H), 7.23 (s, 1H), 6.92 (d, J=8.4 Hz, 1H), 6.14 (s, 1H), 5.63-5.61 (m, 1H), 5.07 (d, J=5.6 Hz, 1H), 4.60 (t, J=6.0 Hz, 1H), 3.55-3.34 (m, 4H), 3.14 (dd, J=4.8 Hz, J=18.8 Hz, 1H), 2.67-2.55 (m, 1H), 2.33-2.07 (m, 4H), 1.75-1.62 (m, 2H). (1 exchangeable proton not observed) Step 2: To a mixture of (6S,9R)-N-(5-chloro-2-fluoro-4-(6-(pyrrolidin-3-yloxy)pyridin-3-yl)phenyl)-3-oxo-3,5,6,7,8,9-hexahydro-2H-6,9-epiminocyclohepta[c]pyridine-10-carboxamide trifluoroacetate salt 62-1 (160 mg, 0.26 mmol) and NaHCO₃ (44 mg, 0.52 mmol) in MeOH (3 mL) was added a solution of formaldehyde in water (44 pl, 37% wt., 0.52 mmol) at RT. After stirring at RT for 30 min, sodium cyanoborohydride (32 mg, 0.52 mmol) was added and the mixture was stirred at RT for a further 16 h. The reaction was diluted with water (10 mL) and the product was extracted into DCM (3×10 mL). The combined organics were dried over Na₂SO₄ and concentrated in vacuo and the product was purified by chromatography on silica gel (10% (10% NH₄0H in MeOH) in DCM) to give (6S,9R)-N-(5-chloro-2-fluoro-4-(6-((2-methyl-2-azabicyclo[2.1.1]hexan-4-yl)methoxy)pyridin-3-yl)phenyl)-3-oxo-3,5,6,7,8,9-hexahydro-2H-6,9-epiminocyclohepta[c]pyridine-10-carboxamide 62 as a white solid. LCMS (Method 7) m/z 524.1, 526.2 (M+H)⁺ (ES⁺), at 1.44 min. ¹H NMR (400 MHz, DMSO-d6) δ 8.65 (s, 1H), 8.19 (d, J=2.4 Hz, 1H), 7.81-7.76 (m, 2H), 7.36 (d, J=10.8 Hz, 1H), 7.23 (s, 1H), 6.87 (d, J=8.8 Hz, 1H), 6.11 (s, 1H), 5.42-5.37 (m, 1H), 5.06 (d, J=5.6 Hz, 1H), 4.59 (t, J=6.0 Hz, 1H), 3.14 (dd, J=4.4, 18.0 Hz, 1H), 2.86-2.82 (m, 1H), 2.77-2.67 (m, 2H), 2.58-2.50 (m, 1H), 2.44-2.39 (m, 1H), 2.33-2.26 (m, 4H), 2.19-2.03 (m, 2H), 1.87-1.64 (m, 3H). (1 Exchangeable proton not observed)

### Experimental Scheme 6

### Compound 63 (6S,9R)-N-(4-(6-(Azetidin-3-ylmethoxy)pyridin-3-yl)-5-chloro-2-fluorophenyl)-3-oxo-3,5,6,7,8,9-hexahydro-2H-6,9-epiminocyclohepta[c]pyridine-10-carboxamide trifluoroacetate salt

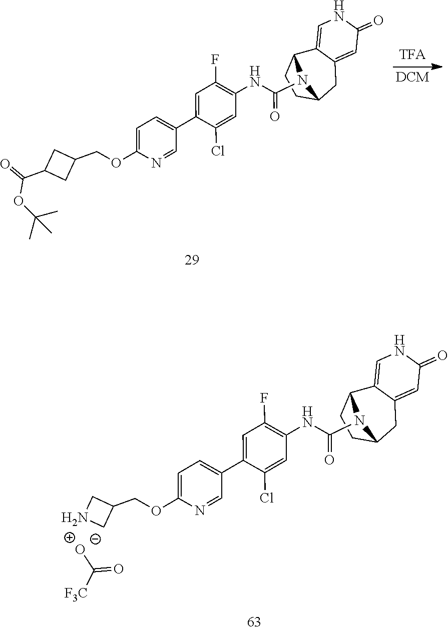 [SMILES: CC(C)(C)OC(=O)C1CC(COc2ccc(-c3cc(F)c(NC(=O)N4[C@H]5CC[C@@H]4c4c[nH]c(=O)cc4C5)cc3Cl)cn2)C1 ; O=C(Nc1cc(Cl)c(-c2ccc(OCC3C[NH2+]C3)nc2)cc1F)N1[C@H]2CC[C@@H]1c1c[nH]c(=O)cc1C2 ; O=C(O)C(F)(F)F]

(6S,9R)-N-(4-(6-(Azetidin-3-ylmethoxy)pyridin-3-yl)-5-chloro-2-fluorophenyl)-3-oxo-3,5,6,7,8,9-hexahydro-2H-6,9-epiminocyclohepta[c]pyridine-10-carboxamide trifluoroacetate salt 63 was synthesised from tert-butyl 3-(((5-(2-chloro-5-fluoro-4-((6S,9R)-3-oxo-3,5,6,7,8,9-hexahydro-2H-6,9-epiminocyclohepta[c]pyridine-10-carboxamido)phenyl)pyridin-2-yl)oxy)methyl)azetidine-1-carboxylate 29 using a procedure essentially the same as for 62-1. LCMS (Method 3) m/z 510.2 (M+H)⁺ (ES⁺), at 0.69 min. ¹H NMR (400 MHz, DMSO— d6) δ 8.66 (s, 1H), 8.38 (s, 1H), 8.22 (d, J=2.4 Hz, 1H), 7.84-7.81 (m, 1H), 7.77 (d, J=7.2 Hz, 1H), 7.36 (d, J=11.2 Hz, 1H), 7.21 (s, 1H), 6.93 (d, J=8.4 Hz, 1H), 6.11 (s, 1H), 5.06 (d, J=4.4 Hz, 1H), 4.60 (t, J=5.6 Hz, 1H), 4.46 (d, J=6.4 Hz, 2H), 3.96 (t, J=8.0 Hz, 2H), 3.75 (t, J=6.0 Hz, 2H), 3.21-3.12 (m, 2H), 2.58-2.53 (m, 1H), 2.22-2.07 (m, 2H), 1.75-1.64 (m, 2H). (1 exchangeable proton not observed)

### Experimental Scheme 7

### Compound 64 (6S,9R)-N-(5-Chloro-2-fluoro-4-(((6-(2,2,2-trifluoroethoxy)pyridin-2-yl)methyl)amino)phenyl)-3-oxo-3,5,6,7,8,9-hexahydro-2H-6,9-epiminocyclohepta[c]pyridazine-10-carboxamide

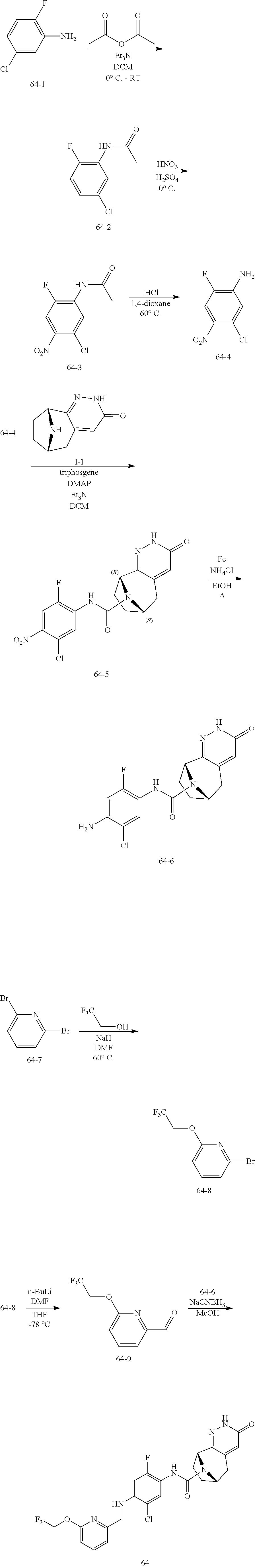

Step 1: To a solution of 5-chloro-2-fluoroaniline 64-1 (300 mg, 2.07 mmol) in DCM (5 mL) at 0° C. was added Et₃N (0.7 mL, 5.17 mmol) and acetic anhydride (0.23 mL, 2.48 mmol). The mixture was stirred at RT for 16 h and after this time water (10 mL) was added and the product was extracted with EtOAc (2×10 mL). The combined organics were dried over Na₂SO₄ and concentrated in vacuo. The product was purified by chromatography on silica gel (20% EtOAc/petroleum ether) to give N-(5-chloro-2-fluorophenyl)acetamide 64-2 as a white solid. ¹H NMR (400 MHz, DMSO-d6) δ 9.89 (s, 1H), 8.08-8.06 (m, 1H), 7.32-7.19 (m, 1H), 7.19-7.15 (m, 1H), 2.10 (s, 3H).

Step 2: To a solution of HNO₃ in H₂SO₄ (0.1 mL/2 mL) was added N-(5-chloro-2-fluorophenyl) acetamide 64-2 (370 mg, 1.98 mmol) at 0° C. and the reaction was stirred at 0° C. for 30 min. The pH of the mixture was adjusted to pH 7-8 by addition of saturated aqueous NaHCO₃. The product was extracted with EtOAc (10 mL), the organics were washed with brine (10 mL), dried over Na₂SO₄ and concentrated in vacuo. The product was purified by chromatography on silica gel (20% EtOAc/petroleum ether) to give N-(5-chloro-2-fluoro-4-nitrophenyl)acetamide 64-3 as a yellow solid. ¹H NMR (400 MHz, DMSO-d6) δ 10.37 (s, 1H), 8.53 (d, J=7.2 Hz, 1H), 8.22 (d, J=10.8 Hz, 1H), 2.18 (s, 3H).

Step 3: To a solution of N-(5-chloro-2-fluoro-4-nitrophenyl)acetamide 64-3 (350 mg, 1.5 mmol) in 1,4-dioxane (5 mL) was added concentrated HCl (5 mL) in a sealed tube and the reaction was warmed to 60° C. for 1 h. The pH of the reaction mixture was adjusted to pH to 7-8 by addition of saturated aqueous NaHCO₃ and the aqueous layer was extracted with DCM (2×10 mL). The combined organics were dried over Na₂SO₄ and concentrated in vacuo to give 5-chloro-2-fluoro-4-nitroaniline 64-4 as a yellow solid. ¹H NMR (400 MHz, DMSO-d6) δ 7.98 (d, J=11.6 Hz, 1H), 6.90-6.87 (m, 3H).

Step 4: To a mixture of 5-chloro-2-fluoro-4-nitroaniline 64-4 (290 mg, 1.53 mmol) and triphosgene (227 mg, 0.76 mmol) in DCM (5 mL) was added a solution of DMAP (596 mg, 4.88 mmol) in DCM (3 mL) at 0° C. The resulting mixture was stirred at 0° C. for 30 mins, before being added to a mixture of (6S,9R)-2,5,6,7,8,9-hexahydro-3H-6,9-epiminocyclohe pta[c]pyridazin-3-one 1-1 (268 mg, 1.51 mmol) and Et₃N (840 μL) in DCM (5 mL) and the mixture was stirred at RT for 16 h. The reaction mixture was diluted with water (10 mL) and the product was extracted with DCM (2×10 mL). The combined organics were dried with Na₂SO₄, and concentrated in vacuo. The product was purified by chromatography on silica gel (2% MeOH/DCM) to give (6S,9R)-N-(5-chloro-2-fluoro-4-nitrophenyl)-3-oxo-3,5,6,7,8,9-hexahydro-2H-6,9-epiminocyclohepta[c]pyridazine-10-carboxamide 64-5 as a yellow solid. ¹H NMR (400 MHz, DMSO-d6) δ 12.74 (s, 1H), 9.26 (s, 1H), 8.17-8.07 (m, 2H), 6.68 (s, 1H), 5.11-5.10 (m, 1H), 4.66 (t, J=5.6 Hz, 1H), 3.21-3.16 (m, 1H), 2.66 (d, J=18.4 Hz, 1H), 2.26-2.12 (m, 2H), 1.83-1.78 (m, 1H), 1.71-1.66 (m, 1H).

Step 5: To a solution of (6S,9R)-N-(5-chloro-2-fluoro-4-nitrophenyl)-3-oxo-3,5,6,7,8,9-hexahydro-2H-6,9-epiminocyclohepta[c]pyridazine-10-carboxamide 64-5 (240 mg. 0.61 mmol) in EtOH (2 mL) was added saturated aqueous NH₄Cl (1 mL) and Fe (170 mg, 3.04 mmol). The reaction was stirred at 80° C. for 1 h before the resulting mixture was filtered and diluted with H₂O (5 mL) and the product was extracted with DCM (10 mL). The organics were dried with sodium sulfate and concentrated in vacuo to give (6S,9R)-N-(4-amino-5-chloro-2-fluorophenyl)-3-oxo-3,5,6,7,8,9-hexahydro-2H-6,9-epiminocyclohepta[c]pyridazine-10-carboxamide 64-6 as a yellow solid. LCMS (Method 3) m/z 363.9 (M+H)⁺ (ES⁺), at 0.60 min.

Step 6: To a solution of 2,6-dibromopyridine 64-7 (1.95 g, 8.30 mmol) in DMF (10 mL) was added NaH (365 mg, 60% w.w, 9.13 mmol), and 2,2,2-trifluoroethan-1-ol (1 g, 9.96 mmol, 1.2 eq) dropwise. After the addition, the reaction was stirred at 60° C. for 16 h and then diluted with saturated aqueous NH₄Cl (20 mL) before the product was extracted with EtOAc (20 mL). The organics were washed with brine (2×20 mL), dried over Na₂SO₄ and concentrated in vacuo. The product was purified by chromatography on silica gel column (100% petroleum ether) to give 2-bromo-6-(2,2,2-trifluoroethoxy)pyridine 64-8 as a colorless oil. ¹H NMR (400 MHz, DMSO-d6) δ 7.76 (t, J=7.6 Hz, 1H), 7.37 (d, J=7.6 Hz, 1H), 7.04 (d, J=8.4 Hz, 1H), 5.00-4.93 (m, 2H).

Step 7: To a solution of 2-bromo-6-(2,2,2-trifluoroethoxy)pyridine 64-8 (500 mg, 1.96 mmol) in THF (5 mL) was added a solution of n-BuLi in hexanes (344 μL, 2.5 M, 0.86 mmol) at −78° C. The reaction was stirred at −78° C. for 1.5 h before DMF (0.3 mL, 3.92 mmol) was added and then the mixture was stirred for another 1 h. The reaction was diluted with water (10 mL) and the product was extracted into DCM (10 mL). The organics were dried with Na₂SO₄ and concentrated in vacuo and the product was purified by chromatography on silica gel (2 % EtOAc/petroleum ether) to give 6-(2,2,2-trifluoroethoxy)picolinaldehyde 64-9 as a white solid. ¹H NMR (400 MHz, DMSO-d6) δ 9.90 (s, 1H), 8.06 (t, J=7.6 Hz, 1H), 7.70 (d, J=7.2 Hz, 1H), 7.33 (d, J=8.0 Hz, 1H), 5.15-5.08 (m, 2H).

Step 8: To a solution of (6S,9R)-N-(4-amino-5-chloro-2-fluorophenyl)-3-oxo-3,5,6,7,8,9-hexahydro-2H-6,9-epiminocyclohepta[c]pyridazine-10-carboxamide 1-1 (140 mg, 0.38 mmol) in MeOH (3 mL) was added 6-(2,2,2-trifluoroethoxy)picolinaldehyde 64-9 (80 mg, 0.38 mmol). The reaction was stirred at RT for 1 h before NaCNBH₃ (50 mg, 0.79 mmol) was added and the mixture was stirred at RT for 16 h. The solvent was removed in vacuo and the product was purified by prep-HPLC to give (6S,9R)-N-(5-chloro-2-fluoro-4-(((6-(2,2,2-trifluoroethoxy)pyridin-2-yl)methyl)amino)phenyl)-3-oxo-3,5,6,7,8,9-hexahydro-2H-6,9-epiminocyclohepta[c]pyridazine-10-carboxamide 64 as a colourless solid. LCMS (Method 7) m/z 553.1 (M+H)⁺ (ES⁺), at 1.94 min. ¹H NMR (400 MHz, DMSO-d6) δ 8.33 (s, 1H), 7.75 (t, J=7.6, 1.0 Hz, 1H), 7.24 (d, J=8.0 Hz, 1H), 7.00 (d, J=7.6 Hz, 1H), 6.83 (d, J=8.4 Hz, 1H), 6.66 (s, 1H), 6.42 (d, J=12.8 Hz, 1H), 5.04-4.95 (m, 3H), 4.53 (s, 1H), 4.39 (s, 2H), 3.16-3.12 (m, 1H), 2.67-2.57 (m, 1H), 2.15 (s, 2H), 1.75-1.64 (m, 2H), 1 exchangeable proton not observed.

The following compounds were prepared using appropriate starting materials in an analogous procedure to that described in Experimental Scheme 7 Where the starting materials are not described in the literature, their synthesis is described below.

| | | Method | |
|---|---|---|---|
| | | LCMS Rt | |
| Compound | Structure | [M + H]⁺ | NMR |
| 65 | 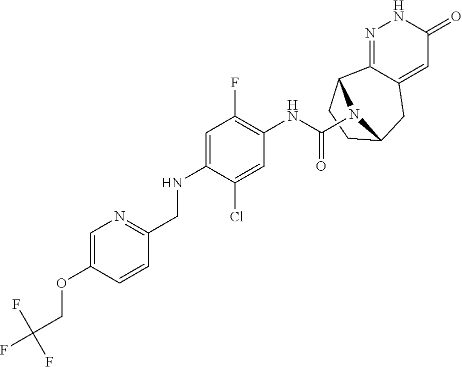 [SMILES: O=C(Nc1cc(Cl)c(NCc2ccc(OCC(F)(F)F)cn2)cc1F)N1[C@H]2CC[C@@H]1c1n[nH]c(=O)cc1C2] | Method 7 553.1 at 1.61 min | ¹H NMR (400 MHz, DMSO-d6) δ 12.67 (s, 1H), 8.36-8.15 (m, 2H), 7.50 (dd, J = 8.8, 3.2 Hz, 1H), 7.32 (d, J = 8.8 Hz, 1H), 7.22 (d, J = 8.0 Hz, 1H), 6.66 (s, 1H), 6.41 (d, J = 12.8 Hz, 1H), 6.29-6.28 (m, 1H), 4.95 (d, J = 5.6 Hz, 1H), 4.84 (q, J = 8.8 Hz, J = 17.6 Hz, 2H), 4.54- 4.51 (m, 1H), 4.40 (d, J = 6.0 Hz, 2H), 3.15 (dd, J = 5.2, 18.8 Hz, 1H), 2.59 (d, J = 18.4 Hz, 1H), 2.22-2.09 (m, 2H), 1.77- 1.72 (m, 1H), 1.67-1.62 (m, 1H). |
| | (6S,9R)-N-(5-Chloro-2-fluoro- | | |
| | 4-(((5-(2,2,2- | | |
| | trifluoroethoxy)pyridin-2- | | |
| | yl)methyl)amino)phenyl)-3-oxo- | | |
| | 3,5,6,7,8,9-hexahydro-2H-6,9- | | |
| | epiminocyclohepta[c]pyridazine- | | |
| | 10-carboxamide | | |

### Intermediate 46 (I-46)

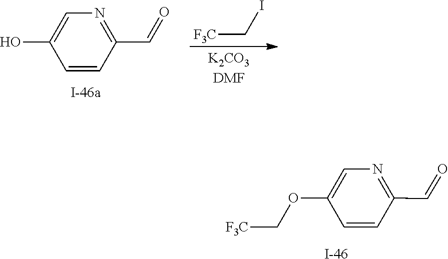

To a mixture of 5-hydroxypicolinaldehyde I-46a (200 mg, 1.62 mmol) and 1,1,1-trifluoro-2-iodoethane (512 mg, 2.43 mmol) in DMF (4 mL) was added K₂CO₃ (673 mg, 4.87 mmol). The reaction was warmed to 100° C. for 16 h and the resulting mixture was poured into water (20 mL) and the product was extracted into EtOAc (3×20 mL). The organics were washed with brine (20 mL), dried over Na₂SO₄ and concentrated in vacuo. The product was purified by TLC (20% EtOAc/petroleum ether) to give 5-(2,2,2-trifluoroethoxy)picolinaldehyde 1-46 as a yellow oil. ¹H NMR (400 MHz, DMSO-d6) δ 9.46 (s, 1H), 8.17 (d, J=2.8 Hz, 1H), 7.53 (d, J=8.8 Hz, 1H), 7.29 (dd, J=2.8, 8.4 Hz, 1H), 4.59 (q, J=8.8 Hz, 2H).

### Experimental Scheme 8

### Compound 66 (5R,8S)-10-((4,5-Dichloro-2-fluorophenyl)carbamoyl)-6,7,8,9-tetrahydro-5H-5,8-epiminocyclohepta[c]pyridine 2-oxide

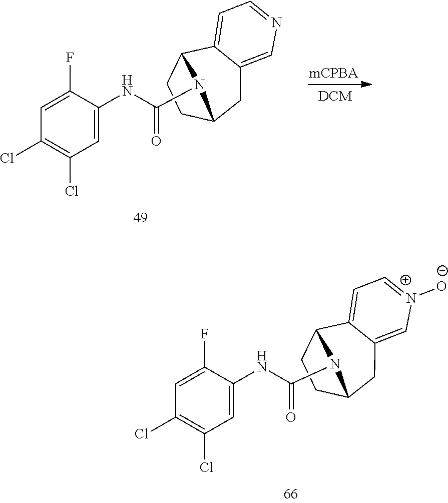 [SMILES: O=C(Nc1cc(Cl)c(Cl)cc1F)N1[C@H]2CC[C@@H]1c1ccncc1C2 ; O=C(Nc1cc(Cl)c(Cl)cc1F)N1[C@H]2CC[C@@H]1c1cc[n+]([O-])cc1C2]

To a solution of (5R,8S)-N-(4,5-dichloro-2-fluorophenyl)-6,7,8,9-tetrahydro-5H-5,8-epiminocyclohepta[c]pyridine-10-carboxamide 49 (50 mg, 137 μmol) in DCM (2 mL) was added mCPBA (37 mg, 70% Wt., 150 μmol) and the reaction was stirred at RT for 3 h. The resulting mixture was diluted with DCM (10 mL) and the organics were washed with a 1M aqueous sodium metabisulfite solution (10 mL) before being passed through a hydrophobic frit. The filtrate was concentrated in vacuo. The product was purified by chromatography on silica gel (0-100% MeOH/DCM) to afford (5R,8S)-10-((4,5-dichloro-2-fluorophenyl)carbamoyl)-6,7,8,9-tetrahydro-5H-5,8-epiminocyclohepta[c]pyridine 2-oxide 66 as a pale purple solid. LCMS (Method 1) m/z 382.0, 384.0 (M+H)⁺ (ES⁺), at 1.30 min. ¹H NMR (400 MHz, DMSO-d6) δ 8.77 (s, 1H), 8.03 (s, 1H), 7.99 (dd, J=6.5, 1.9 Hz, 1H), 7.81 (d, J=7.6 Hz, 1H), 7.67 (d, J=10.3 Hz, 1H), 7.22 (d, J=6.5 Hz, 1H), 5.14 (d, J=6.0 Hz, 1H), 4.72-4.65 (m, 1H), 3.23 (dd, J=17.5, 4.9 Hz, 1H), 2.60 (d, J=17.4 Hz, 1H), 2.28-2.05 (m, 2H), 1.85-1.75 (m, 1H), 1.74-1.64 (m, 1H).

The following compounds were prepared using appropriate starting materials in an analogous procedure to that described in Experimental Scheme 8

| | | Method | |
|---|---|---|---|
| | | LCMS Rt | |
| Compound | Structure | [M + H]⁺ | NMR |
| 67 | 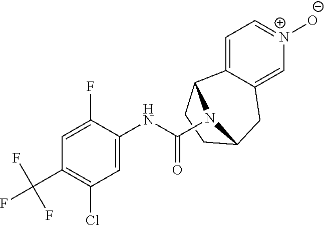 [SMILES: O=C(Nc1cc(Cl)c(C(F)(F)F)cc1F)N1[C@H]2CC[C@@H]1c1cc[n+]([O-])cc1C2] | Method 1 416.0, 418.0 at 1.44 min | ¹H NMR (400 MHz, DMSO-d6) δ 9.04 (s, 1H), 8.04-7.97 (m, 3H), 7.79 (d, J = 11.0 Hz, 1H), 7.24 (d, J = 6.5 Hz, 1H), 5.19 (d, J = 5.9 Hz, 1H), 4.73 (t, J = 6.2 Hz, 1H), 3.24 (dd, J = 17.4, 5.0 Hz, 1H), 2.62 (d, J = 17.4 Hz, 1H), 2.29-2.07 (m, 2H), 1.86-1.76 (m, 1H), 1.75- 1.64 (m, 1H) |
| | (5R,8S)-10-((5-Chloro-2-fluoro-4- | | |
| | (trifluoromethyl)phenyl)carbamoyl)- | | |
| | 6,7,8,9-tetrahydro-5H-5,8- | | |
| | epiminocyclohepta[c]pyridine | | |
| | 2-oxide | | |

### Experimental Scheme 9

### Compound 68 (6S,9R)-N-(5-Chloro-2-fluoro-4-(2-methyl-1 H-benzo[d]imidazol-5-yl)phenyl)-3-oxo-3,5,6,7,8,9-hexahydro-2H-6,9-epiminocyclohepta[c]pyridine-10-carboxamide

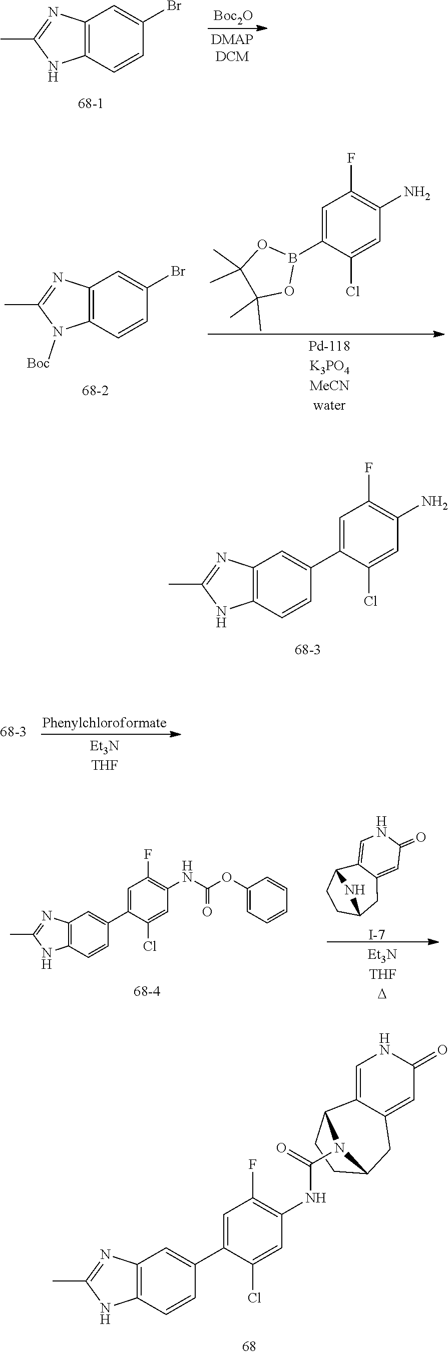

Step 1: To a solution of 5-bromo-2-methyl-1 H-benzo[d]imidazole 68-1 (1.00 g, 4.74 mmol) in DCM (80 mL) was added di-tert-butyl dicarbonate (1.96 g, 9.00 mmol) and DMAP (1.10 g, 9.00 mmol) and the reaction was stirred at RT for 16 h. The resulting mixture was diluted with water (50 ml) and the layers were separated. The aqueous was extracted with DCM (2×100 mL) and the combined organics were dried over Na₂SO₄, filtered and concentrated in vacuo. The product was purified by chromatography on silica gel (0-40% MTBE/heptane) to afford tert-butyl 5-bromo-2-methyl-1H-benzo[d]imidazole-1-carboxylate 68-2 as a pale yellow solid.

Step 2: 5-Chloro-2-fluoro-4-(2-methyl-1H-benzo[d]imidazol-5-yl)aniline 68-3 was synthesised from tert-butyl 5-bromo-2-methyl-1 H-benzo[d]imidazole-1-carboxylate 68-2 and 5-chloro-2-fluoro-4-(4,4,5,5-tetramethyl-1,3,2-dioxaborolan-2-yl)aniline I-3a using a 10 procedure essentially the same as for 1-3. ¹H NMR (400 MHz, DMSO-d6) δ 12.22 (s, 1H), 7.40 (d, J=8.5 Hz, 2H), 7.14-7.02 (m, 2H), 6.90 (d, J=8.4 Hz, 1H), 5.48 (s, 2H), 2.48 (d, J=5.0 Hz, 3H).

Step 3: A solution of 5-chloro-2-fluoro-4-(2-methyl-1H-benzo[d]imidazol-5-yl)aniline 68-3 (84.0 mg, 247 μmol) in THF (1 mL) was cooled to 0° C. and phenyl chloroformate (31 μL, 247 μmol) was added and the resulting mixture was warmed to RT for 2 h. The reaction was cooled to 0° C. and a further portion of phenyl chloroformate (31 μL, 247 μmol) was added dropwise before the reaction mixture was warmed to RT for 1 h. Et₃N (34.4 μL, 247 μmol) was added dropwise and the mixture was stirred at RT for 30 min. The reaction was concentrated in vacuo and the resultant off-white solid was dissolved in DCM (20 mL) and the organics were washed with water (2×1 mL), dried over Na₂SO₄ and concentrated in vacuo to provide phenyl (5-chloro-2-fluoro-4-(2-methyl-1H-benzo[d]imidazol-5-yl)phenyl)carbamate 68-4 as a waxy yellow solid that was used in the next step without any further purification.

Step 4: To a solution of phenyl (5-chloro-2-fluoro-4-(2-methyl-1 H-benzo[d]imidazol-5-yl)phenyl)carbamate 68-4 and (6S,9R)-2,5,6,7,8,9-hexahydro-3H-6,9-epiminocyclohepta[c]pyridin-3-one 1-7 (48 mg, 247 μmol) in THF (1 mL) was added Et₃N (103 μL, 740 μmol) and the reaction was heated to 65° C. for 18 h. The resulting mixture was cooled to RT and diluted with 10% MeOH/DCM (10 mL). The organics were separated, washed with water (2×2 mL),dried over Na₂SO₄ and concentrated in vacuo. The product 30 was purified by chromatography on silica gel (0-35% (0.7M NH₃ in MeOH)/DCM) to afford (6S,9R)-N-(5-chloro-2-fluoro-4-(2-methyl-1 H-benzo[d]imidazol-5-yl)phenyl)-3-oxo-3,5,6,7,8,9-hexahydro-2H-6,9-epiminocyclohepta[c]pyridine-10-carboxamide 68 as a fine tan powder. LCMS (Method 1) m/z 478.2, 480.3 (M+H)⁺ (ES⁺), at 0.95 min. ¹H NMR (400 MHz, DMSO-d6) δ 12.34 (s, 1H), 11.28 (s, 1H), 8.61 (s, 1H), 7.73 (d, J=7.4 Hz, 1H), 7.55-7.43 (m, 2H), 7.30 (d, J=11.2 Hz, 1H), 7.21 (s, 1H), 7.15 (dd, J=8.3, 1.7 Hz, 1H), 6.11 (s, 1H), 5.07 (d, J=5.8 Hz, 1H), 4.60 (t, J=6.4 Hz, 1H), 3.32 (s, 3H), 3.23-3.08 (m, 1H), 2.56 (d, J=17.9 Hz, 1H), 2.28-1.97 (m, 2H), 1.80-1.54 (m, 2H).

### Separation of Enantiomers by Chiral Chromatography

It will be appreciated that the enantiomers of the compounds described above can be isolated using techniques well known in the art, including, but not limited to, chiral chromatography. For example, a racemic mixture can be dissolved in a solvent, for example, methanol, followed by separation by chiral SFC on a Waters prep 15 with UV detection by DAD at 210-400 nm, 40° C., 120 bar on a Chiralpak® IG (Daicel Ltd.) column (1×25 cm, 5 μm particle size), flow rate 15 ml/min-1 using 50% ethanol in 0.1% DEA to afford both enantiomers as the separated pure compounds.

For example, (±)-N-(5-chloro-2-fluoro-4-(6-(2,2,2-trifluoroethoxy)pyridin-3-yl)phenyl)-1,9,9-trifluoro-6,7,8,9-tetrahydro-5H-5,8-epiminocyclohepta[c]pyridine-10-carboxamide (compound 70 was dissolved in MeOH:DCM (2:1) mixture (22 mg/mL) with sonication, filtered and was then purified by chiral SFC on a Waters prep 100 with a PDA and a QDa detectors, 40° C., 120 bar. The column was a Lux Amylose-1 5 μm, 21 mm×250 mm; flow rate 65 mL/min of 55% MeOH (no additive), 45% C02.to afford (5R,8S)-N-(5-chloro-2-fluoro-4-(6-(2,2,2-trifluoroethoxy)pyridin-3-yl)phenyl)-1,9,9-trifluoro-6,7,8,9-tetrahydro-5H-5,8-epiminocyclohepta[c]pyridine-10-carboxamide (compound 71) and (5S,8R)-N-(5-chloro-2-fluoro-4-(6-(2,2,2-trifluoroethoxy)pyridin-3-yl)phenyl)-1,9,9-trifluoro-6,7,8,9-tetrahydro-5H-5,8-epiminocyclohepta[c]pyridine-10-carboxamide (compound 72) both as colourless solids. LCMS and NMR data for compounds 71 and 72 are found in the table below.

The following compounds were prepared using appropriate starting materials in an analogous procedure to that described for 71 and 72.

| | | Method | | |
|---|---|---|---|---|
| | | LCMS | | |
| | | Rt | Analytical | |
| Compound | Structure | [M + H]⁺ | SFC Rt | NMR |
| 71 | 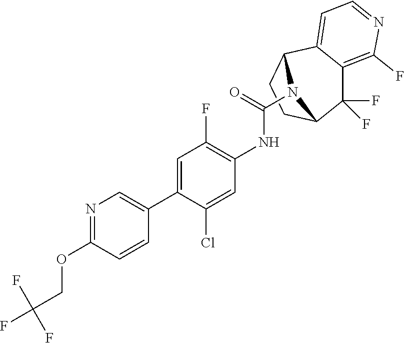 [SMILES: O=C(Nc1cc(Cl)c(-c2ccc(OCC(F)(F)F)nc2)cc1F)N1[C@@H]2CC[C@H]1C(F)(F)c1c2ccnc1F] | Method 1 561.1, 563.2, at 1.81 min | SFC method 1 1.2 min 40% MeOH (0.1% ammonia), 60% CO₂ | ¹H NMR (400 MHz, DMSO- d6) δ 9.15 (s, 1H), 8.38 (d, J = 5.1 Hz, 1H), 8.27 (d, J = 2.5 Hz, 1H), 7.92 (dd, J = 8.6, 2.5 Hz, 1H), 7.77 (d, J = 7.4 Hz, 1H), 7.51 (d, J = 5.1 Hz, 1H), 7.44 (d, J = 11.1 Hz, 1H), 7.09 (d, J = 8.6 Hz, 1H), 5.47 (d, J = 6.5 Hz, 1H), 5.33-5.18 (m, 1H), 5.05 (q, J = 9.1 Hz, 2H), 2.40- 2.25 (m, 1H), 2.27-2.10 (m, 1H), 1.99-1.85 (m, 1H), 1.80-1.63 (m, 1H). |
| | (5R,8S)-N-(5-Chloro-2-fluoro-4-(6- | | | |
| | (2,2,2-trifluoroethoxy)pyridin-3- | | | |
| | yl)phenyl)-1,9,9-trifluoro-6,7,8,9- | | | |
| | tetrahydro-5H-5,8- | | | |
| | epiminocyclohepta[c]pyridine-10- | | | |
| | carboxamide | | | |
| 72 | 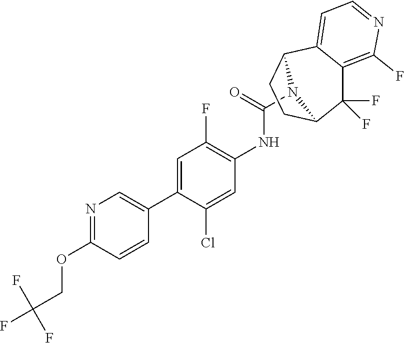 [SMILES: O=C(Nc1cc(Cl)c(-c2ccc(OCC(F)(F)F)nc2)cc1F)N1[C@H]2CC[C@@H]1C(F)(F)c1c2ccnc1F] | Method 1 561.3, 563.3, at 1.81 min | SFC method 1 3.14 min 40% MeOH (0.1% ammonia), 60% CO₂ | ¹H NMR (400 MHz, DMSO- d6) δ 9.15 (s, 1H), 8.38 (d, J = 5.1 Hz, 1H), 8.27 (d, J = 2.5 Hz, 1H), 7.92 (dd, J = 8.6, 2.5 Hz, 1H), 7.77 (d, J = 7.4 Hz, 1H), 7.51 (d, J = 5.1 Hz, 1H), 7.44 (d, J = 11.1 Hz, 1H), 7.09 (d, J = 8.6 Hz, 1H), 5.47 (d, J = 6.5 Hz, 1H), 5.33- 5.18 (m, 1H), 5.05 (q, J = 9.1 Hz, 2H), 2.40-2.25 (m, 1H), 2.27-2.10 (m, 1H), 1.99- 1.85 (m, 1H), 1.80-1.63 (m, 1H). |
| | (5S,8R)-N-(5-Chloro-2-fluoro-4-(6- | | | |
| | (2,2,2-trifluoroethoxy)pyridin-3- | | | |
| | yl)phenyl)-1,9,9-trifluoro-6,7,8,9- | | | |
| | tetrahydro-5H-5,8- | | | |
| | epiminocyclohepta[c]pyridine-10- | | | |
| | carboxamide | | | |
| 73^{(d)} | 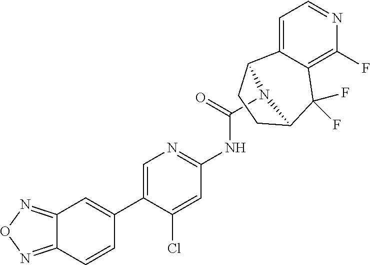 [SMILES: O=C(Nc1cc(Cl)c(-c2ccc3nonc3c2)cn1)N1[C@H]2CC[C@@H]1C(F)(F)c1c2ccnc1F] | Method 1 487.4, 489.0 at 1.62 min | SFC method 2 2.25 min 40% MeOH (0.1% ammonia), 60% CO₂ | ¹H NMR (400 MHz, DMSO- d6) δ 10.29 (s, 1H), 8.47 (s, 1H), 8.39 (d, J = 5.1 Hz, 1H), 8.21 (t, J = 1.2 Hz, 1H), 8.15 (dd, J = 9.3, 1.0 Hz, 1H), 8.08 (s, 1H), 7.74 (dd, J = 9.3, 1.4 Hz, 1H), 7.51 (d, J = 5.0 Hz, 1H), 5.56 (d, J = 6.4 Hz, 1H), 5.49-5.37 (m, 1H), 2.42- 2.26 (m, 1H), 2.27-2.13 (m, 1H), 2.01-1.86 (m, 1H), 1.78-1.68 (m,1H). |
| | (5S,8R)-N-(5- | | | |
| | (Benzo[c][1,2,5]oxadiazol-5-yl)-4- | | | |
| | chloropyridin-2-yl)-1,9,9-trifluoro- | | | |
| | 6,7,8,9-tetrahydro-5H-5,8- | | | |
| | epiminocyclohepta[c]pyridine-10- | | | |
| | carboxamide | | | |
| 74^{(d)} | 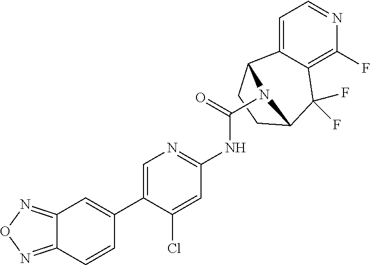 [SMILES: O=C(Nc1cc(Cl)c(-c2ccc3nonc3c2)cn1)N1[C@@H]2CC[C@H]1C(F)(F)c1c2ccnc1F] | Method 1 487.1, 489.3 at 1.62 min | SFC method 2 3.19 min 40% MeOH (0.1% ammonia), 60% CO₂ | ¹H NMR (400 MHz, DMSO- d6) δ 10.29 (s, 1H), 8.47 (s, 1H), 8.39 (d, J = 5.1 Hz, 1H), 8.21 (t, J = 1.2 Hz, 1H), 8.15 (dd, J = 9.3, 1.0 Hz, 1H), 8.08 (s, 1H), 7.74 (dd, J = 9.3, 1.4 Hz, 1H), 7.51 (d, J = 5.0 Hz, 1H), 5.56 (d, J = 6.4 Hz, 1H), 5.49-5.37 (m, 1H), 2.42- 2.26 (m, 1H), 2.27-2.13 (m, 1H), 2.01-1.86 (m, 1H), 1.78-1.68 (m,1H). |
| | (5R,8S)-N-(5- | | | |
| | (Benzo[c][1,2,5]oxadiazol-5-yl)-4- | | | |
| | chloropyridin-2-yl)-1,9,9-trifluoro- | | | |
| | 6,7,8,9-tetrahydro-5H-5,8- | | | |
| | epiminocyclohepta[c]pyridine-10- | | | |
| | carboxamide | | | |
| 75^{(b)} | 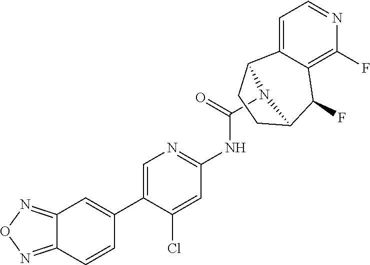 [SMILES: O=C(Nc1cc(Cl)c(-c2ccc3nonc3c2)cn1)N1[C@@H]2CC[C@H]1c1ccnc(F)c1[C@H]2F] | Method 1 469.1, 471.1 at 1.59 min | SFC method 3 2.72 min 40% MeOH (0.1% ammonia), 60% CO₂ | ¹H NMR (400 MHz, DMSO- d6) δ 10.25 (s, 1H), 8.47 (s, 1H), 8.22-8.19 (m, 2H), 8.15 (dd, J = 9.4, 1.1 Hz, 1H), 8.11 (s, 1H), 7.73 (dd, J = 9.3, 1.5 Hz, 1H), 7.35 (d, J= 5.1 Hz, 1H), 6.27 (dd, J = 51.1, 5.9 Hz, 1H), 5.48 (d, J = 6.1 Hz, 1H), 5.15-5.03 (m, 1H), 2.27-2.07 (m, 3H), 1.89-1.78 (m, 1H). |
| | (5S,8R,9R)-N-(5- | | | |
| | (Benzo[c][1,2,5]oxadiazol-5-yl)-4- | | | |
| | chloropyridin-2-yl)-1,9-difluoro- | | | |
| | 6,7,8,9-tetrahydro-5H-5,8- | | | |
| | epiminocyclohepta[c]pyridine-10- | | | |
| | carboxamide | | | |
| 76^{(b)} | 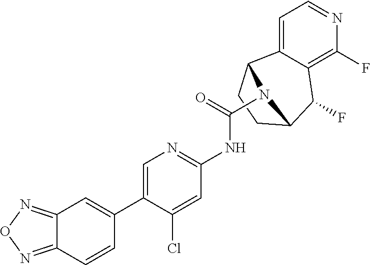 [SMILES: O=C(Nc1cc(Cl)c(-c2ccc3nonc3c2)cn1)N1[C@@H]2CC[C@H]1[C@@H](F)c1c2ccnc1F] | Method 1 469.1, 471.1 at 1.59 min | SFC method 3 3.12 min 40% MeOH (0.1% ammonia), 60% CO₂ | ¹H NMR (400 MHz, DMSO- d6) δ 10.25 (s, 1H), 8.47 (s, 1H), 8.22-8.1 8(m, 2H), 8.15 (dd, J = 9.3, 1.1 Hz, 1H), 8.11 (s, 1H), 7.73 (dd, J = 9.3, 1.4 Hz, 1H), 7.40- 7.29 (m, 1H), 6.27 (dd, J = 51.0, 5.8 Hz, 1H), 5.48 (d, J= 6.1 Hz, 1H), 5.14-4.95 (m, 1H), 2.26-2.04 (m, 3H), 1.89-1.76 (m, 1H). |
| | (5R,8S,9S)-N-(5- | | | |
| | (Benzo[c][1,2,5]oxadiazol-5-yl)-4- | | | |
| | chloropyridin-2-yl)-1,9-difluoro- | | | |
| | 6,7,8,9-tetrahydro-5H-5,8- | | | |
| | epiminocyclohepta[c]pyridine-10- | | | |
| | carboxamide | | | |
| 77^{(a)(c)} | 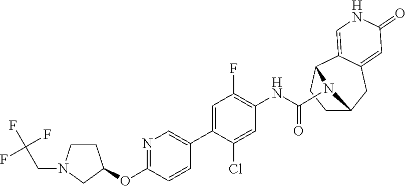 [SMILES: O=C(Nc1cc(Cl)c(-c2ccc(O[C@@H]3CCN(CC(F)(F)F)C3)nc2)cc1F)N1[C@H]2CC[C@@H]1c1c[nH]c(=O)cc1C2] | Method 1 592.4 at 1.42 min | SFC method 1 4.00 min 35% MeOH (0.1% ammonia), 65% CO₂ | ¹H NMR (400 MHz, DMSO- d6) δ 11.28 (s, 1H), 8.64 (s, 1H), 8.21 (dd, J = 2.5, 0.8 Hz, 1H), 7.89-7.71 (m, 2H), 7.37 (d, J = 11.1 Hz, 1H), 7.20 (s, 1H), 6.89 (dd, J = 8.6, 0.7 Hz, 1H), 6.11 (s, 1H), 5.41 (td, J = 6.3, 2.9 Hz, 1H), 5.06 (d, J = 5.8 Hz, 1H), 4.59 (t, J = 6.3 Hz, 1H), 3.28 (d, J = 10.2 Hz, 1H), 3.21- 3.09 (m, 2H), 3.02-2.80 (m, |
| | (6S,9R)-N-(5-Chloro-2-fluoro-4-(6- | | | 2H), 2.68 (q, J = 7.6 Hz, 1H), |
| | ((1-(2,2,2-trifluoroethyl)pyrrolidin- | | | 2.54 (d, J = 17.7 Hz, 1H), |
| | 3-yl)oxy)pyridin-3-yl)phenyl)-3- | | | 2.27 (dt, J = 13.8, 6.8 Hz, |
| | oxo-3,5,6,7,8,9-hexahydro-2H-6,9- | | | 1H), 2.23-2.13 (m, 1H), |
| | epiminocyclohepta[c]pyridine-10- | | | 2.13-2.01 (m, 1H), 1.96- |
| | carboxamide Isomer 1 | | | 1.82 (m, 1H), 1.79-1.56 (m, |
| | | | | 2H). 1 CH under water |
| | | | | peak |
| 78^{(a)(c)} | 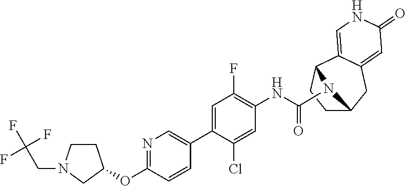 [SMILES: O=C(Nc1cc(Cl)c(-c2ccc(O[C@H]3CCN(CC(F)(F)F)C3)nc2)cc1F)N1[C@H]2CC[C@@H]1c1c[nH]c(=O)cc1C2] | Method 2 592.3 at 1.38 min | SFC method 1 5.24 min 35% MeOH (0.1% ammonia), 65% CO₂ | ¹H NMR (400 MHz, DMSO- d6) δ 11.27 (s, 1H), 8.63 (s, 1H), 8.21 (d, J = 2.5 Hz, 1H), 7.92-7.71 (m, 2H), 7.37 (d, J = 11.1 Hz, 1H), 7.20 (s, 1H), 6.89 (d, J = 8.6 Hz, 1H), 6.11 (s, 1H), 5.41 (ddt, J = 8.9, 6.1, 2.9 Hz, 1H), 5.06 (d, J = 5.8 Hz, 1H), 4.59 (t, J = 6.4 Hz, 1H), 3.28 (d, J = 10.2 Hz, 1H), 3.20- 3.06 (m, 2H), 2.94 (q, J = 7.5 Hz, 1H), 2.86 |
| | (6S,9R)-N-(5-Chloro-2-fluoro-4-(6- | | | (dd, J = 10.7, 2.7 Hz, 1H), |
| | ((1-(2,2,2-trifluoroethyl)pyrrolidin- | | | 2.75-2.63 (m, 1H), 2.55 (d, J = |
| | 3-yl)oxy)pyridin-3-yl)phenyl)-3- | | | 17.9 Hz, 1H), 2.35-2.22 (m, |
| | oxo-3,5,6,7,8,9-hexahydro-2H-6,9- | | | 1H), 2.18 (q, J = 10.8 Hz, 1H), |
| | epiminocyclohepta[c]pyridine-10- | | | 2.08 (tt, J = 11.1, 5.6 Hz, |
| | carboxamide Isomer 2 | | | 1H), 1.93-1.81 (m, 1H), |
| | | | | 1.69 (ddd, J = 21.9, 11.4, 7.0 |
| | | | | Hz, 2H), 0.92-0.80 (m, 2H). |
| | | | | 1 CH under water peak |

^{(a)}purified on Sepiatec prep SFC 50,
^{(b)}purified with a Phenomenex Lux ® 5 μm i-Cellulose-4, LC Column 250 × 21 mm,
^{(c)}purified with a Phenomenex Lux ® A1 5 μm, LC Column 250 × 10 mm,
^{(d)}purified with a Chiralpak IH 10 × 250 mm, 5 μm particle size column, SFC method 1 Phenomenex Lux ® A1 4.6 × 250 mm, 5 μm particle size, flow rate 4 ml/min SFC method 2-Chiralpak IH 4.6 × 250 mm, 5 um particle size, flow rate 4 ml/min SFC method 3-Phenomenex Lux® Cellulose-4 4.6 × 250 mm, 5 μm particle size, flow rate 4 ml/min

Human GPR65 Cyclic Adenosine Monophosphate (cAMP) Homogeneous Time Resolved Fluorescence (HTRF) Antagonist Assay Procedure

IC₅₀data was obtained by the following procedure:

1321N1 human astrocytoma cells stably expressing human recombinant GPR65 (1321N1-hrGPR65 cells, EuroscreenFast) were cultured according to the vendor's instructions.

Compounds were tested for their ability to antagonise GPR65, through measuring the concentration of cytoplasmic cAMP following treatment of the cells at a pH of 7.2 to activate GPR65 signalling and addition of the compound to be tested. The extent to which the expected rise in cAMP concentration upon GPR65 activation was suppressed by the added compound is indicative of its potency. The assay was carried out according to EuroscreenFast assay Methodology as follows.

On the day of the assay, test compounds were added to 384-well, low volume, white microtiter plates by acoustic dispensing. KRH buffer (5 mM KCl, 1.25 mM MgSO₄, 124 mM NaCl, 25 mM HEPES, 13.3 mM Glucose, 1.25 mM KH₂PO₄ and 1.45 mM CaCl₂) was adjusted to pH 6.5, pH 7.6 and pH 8.4 by adding NaOH. 1321N1-hGPR65 cells were rapidly thawed and diluted in KRH, pH 7.6 prior to centrifugation at 300×g for 5 min and resuspension in assay buffer (KRH, pH 7.6, supplemented with 1 mM 3-isobutyl-1-methylxanthine (IBMX) and 200 μM ethylenediaminetetraacetic acid (EDTA)). Cells were added to assay plates at a density of 2,000 cells per well in a volume of 5 pl. Assay plates were briefly centrifuged at 100×g and then incubated at room temperature for 30 min. Cells were stimulated by the addition of 5 μL KRH, pH 6.5, to achieve an assay pH of 7.2, while control wells received 5 pl KRH, pH 8.4 to achieve an assay pH of 7.9. Assay plates were briefly centrifuged at 100×g and then incubated at room temperature for 30 min.

Accumulation of cAMP was detected by cAMP HTRF kit (Cisbio). d2-labeled cAMP and cryptate-labeled anti-cAMP antibody in Lysis and Detection Buffer (Cisbio) were added to assay plates, and the plates were incubated at room temperature for 1 h. HTRF measurements were performed using a Pherastar FSX instrument. Acceptor and donor emission signals were measured at 665 nm and 620 nm, respectively, and HTRF ratios were calculated as signal_{665 nm}/signal_{620 nm}×104. Data were normalised to high and low control values and fitted with 4-parameter logistic regression to determine hGPR65 IC₅₀ values for the test compounds, which are shown in Table 1.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Activity of selected compounds according to the invention

| | |
|---|---|
| 1 | High |
| 2 | High |
| 3 | High |
| 4 | High |
| 5 | High |
| 6 | High |
| 7 | Medium |
| 8 | High |
| 9 | Medium |
| 10 | High |
| 11 | High |
| 12 | High |
| 13 | High |
| 14 | High |
| 15 | High |
| 16 | High |
| 17 | High |
| 18 | High |
| 19 | Medium |
| 20 | High |
| 21 | High |
| 22 | High |
| 23 | High |
| 24 | High |
| 25 | High |
| 26 | High |
| 27 | High |
| 28 | High |
| 29 | High |
| 30 | Medium |
| 31 | Medium |
| 32 | High |
| 33 | High |
| 34 | High |
| 35 | High |
| 36 | High |
| 37 | High |
| 38 | High |
| 39 | High |
| 40 | High |
| 41 | High |
| 42 | High |
| 43 | High |
| 44 | High |
| 45 | High |
| 46 | High |
| 47 | High |
| 48 | High |
| 49 | High |
| 50 | High |
| 51 | High |
| 52 | High |
| 53 | High |
| 54 | High |
| 55 | High |
| 56 | High |
| 57 | High |
| 58 | High |
| 59 | High |
| 60 | Medium |
| 61 | Medium |
| 62 | Medium |
| 63 | Medium |
| 64 | High |
| 65 | Medium |
| 66 | High |
| 67 | High |
| 68 | Medium |
| 70 | High |
| 71 | High |
| 72 | High |
| 73 | Medium |
| 74 | High |
| 75 | High |
| 76 | High |
| 77 | High |
| 78 | High |
| 79 | High |
| 80 | High |
| 81 | High |
| 82 | High |
| 83 | High |
| 84 | High |
| 85 | High |
| 86 | High |
| 87 | High |
| 88 | Low |
| 89 | Low |
| 91 | Medium |
| 92 | High |

High = IC50 < 500 nM;
Medium = IC50 > 500 nM and <5 μM;
Low > 5 μM

### REFERENCES

Bohn, T. et al. (2018). Tumor immunoevasion via acidosis-dependent induction of regulatory tumor-associated macrophages. Nature Immunology, 1319-1326.
Damaghi, M. et al. (2013). pH Sensing and Regulation in Cancer. Frontiers in Physiology.
Gaublomme, J. et al. (2015). Single-Cell Genomics Unveils Critical Regulators of Th17 Cell Pathogenicity. Cell, 1400-1412.
Hernandez, J. (2018). GPR65, a critical regulator of Th17 cell pathogenicity, is regulated by the CRTC2/CREB pathway. The Journal of Immunology, 200 (Supplement).
Korn, T. et al. (2009). IL-17 and Th17 Cells. Annual Reviews in Immunology, 485-517.
Wang, J. et al. (2004). TDAG8 is a proton-sensing and psychosine-sensitive G-protein-coupled receptor. Journal of Biological Chemistry, 45626-45633.
Yoshida, N. et al. (2016). ICER is requisite for Th17 differentiation. Nature Communications, 12993.
Hardin, M. et al. (2014). The clinical and genetic features of COPD-asthma overlap syndrome. Eur Respir J. 2014 August; 44(2):341-50.
Kottyan, L. et al. (2009). Eosinophil viability is increased by acidic pH in a cAMP- and GPR65-dependent manner. Blood. 2009 Sep. 24; 114(13):2774-82.
Tsurumaki, H. et al (2015). Int J Mol Sci. Protective Role of Proton-Sensing TDAG8 in Lipopolysaccharide-Induced Acute Lung Injury. Dec 4;16(12):28931-42.
Schultz and Wolfe (2011), Organic Letters, Intramolecular Alkene Carboamination Reactions for the Synthesis of Enantiomerically Enriched Tropane Derivatives, 13 (11), 2962-2965

1. A compound of formula (1), or a pharmaceutically acceptable salt or solvate thereof, 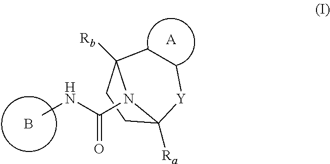
wherein:
ring A is selected from: 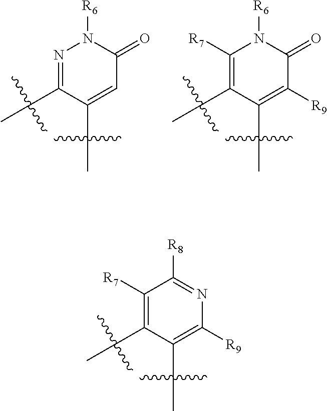
Y is CR₁₀R_{10′}, wherein R₁₀ and R_{10′} are each independently selected from H, F, alkyl, and haloalkyl;
Rₐ and R_{b} are each independently selected from H and alkyl;
ring B is:
a monocyclic aryl group; or
a monocyclic heteroaryl group,
each of which is substituted by:
(i) a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
wherein said —(CH₂)_{q}-heterocycloalkyl group is further substituted by one or more substituents selected from CO₂R₁₄, halo, haloalkyl, haloalkoxy and alkyl;
(ii) optionally one or more substituents selected from halo, haloalkyl, haloalkoxy, alkyl and alkoxy;
q is an integer from 0 to 3;
R₆ is selected from H and alkyl, more preferably H;
R₇, R₈ and R₉ are each independently selected from H, halo and alkyl;
each R₁₁, R_{11′}, R₁₂, R_{12′}, R₁₃ and R_{13′} is independently selected from H, alkyl, and alkoxyalkyl;
R₁₄ is selected from alkyl and haloalkyl;
R₁₅ is selected from H and alkyl; and
R₁₆ is selected from alkyl, haloalkyl and CO-haloalkyl.

2. A compound according to claim 1 which is of formula (Ia) or a pharmaceutically acceptable salt or solvate thereof, 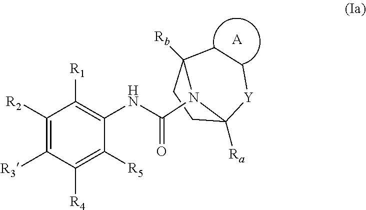
wherein:
R₁, R₂, R₄ and R₅ are each independently selected from H, halo, haloalkyl, haloalkoxy, alkyl and alkoxy; and
R_{3′} is a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
wherein said —(CH₂)_{q}-heterocycloalkyl group is further substituted by one or more substituents selected from CO₂R₁₄, halo, haloalkyl, haloalkoxy and alkyl.

3. A compound according to claim 1 which is of formula (Ib) or a pharmaceutically acceptable salt or solvate thereof, 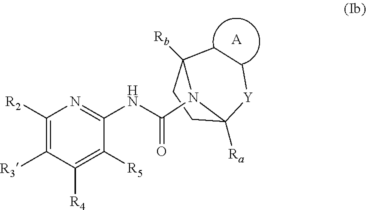
wherein:
R₂, R₄ and R₅ are each independently selected from H, halo, haloalkyl, haloalkoxy, alkyl and alkoxy; and
R_{3′} is a heteroaryl group substituted by at least one group selected from —NR₁₅R₁₆, and —O—(CH₂)_{q}-heterocycloalkyl, and optionally further substituted by one or more substituents selected from halo, alkyl, alkoxy, haloalkyl, haloalkoxy, CN, OH, SO₂-alkyl, hydroxyalkyl, CO₂R₁₄, NR₁₁R_{11′}, CONR₁₂R_{12′}, alkyl-NR₁₃R_{13′}, alkoxy-alkyl, alkoxy-alkoxy and NHSO₂-alkyl;
wherein said —(CH₂)_{q}-heterocycloalkyl group is further substituted by one or more substituents selected from CO₂R₁₄, halo, haloalkyl, haloalkoxy and alkyl.

4. A compound according to claim 2 or claim 3 wherein R_{3′} is a pyridinyl group substituted by at least one group selected from —NR₁₅R₁₆, and —O—(CH₂)_{q}-heterocycloalkyl, wherein said —(CH₂)_{q}-heterocycloalkyl group is further substituted by one or more substituents selected from CO₂R₁₄, halo, haloalkyl, haloalkoxy and alkyl.

5. A compound according to any one of claims 2 to 4 wherein R_{3′} is a pyridinyl group substituted by a group —NR₁₅R₁₆, wherein R₁₅ is H or alkyl, and R₁₆ is a group selected from alkyl, haloalkyl and CO-haloalkyl, more preferably selected from haloalkyl and CO-haloalkyl.

6. A compound according to any one of claims 2 to 4 wherein R_{3′} is a pyridinyl group substituted by a group —O—(CH₂)_{q}-heterocycloalkyl, wherein said heterocycloalkyl group is selected from pyrrolidinyl, azetidinyl, tetrahydrofuranyl, 2-azabicyclo[2.1.1]hexanyl and 2-oxabicyclo[2.1.1]hexanyl, each of which is further substituted by one or more substituents selected from CO₂-alkyl, haloalkyl and alkyl.

7. A compound according to any one of claims 1 to 4 or 6, wherein q is 0 or 1.

8. A compound according to any one of claims 2 to 7, wherein R₂ is H.

9. A compound according to any one of claims 2 to 8, wherein R₅ is H.

10. A compound according to any one of claims 4 to 9 wherein said compound is of formula (Ia) and R₁ is selected from H, F, Me, MeO and Cl, and is preferably H or F.

11. A compound according to any one of claims 2 to 10, wherein R₄ is selected from C₁, Br, and CF₃, more preferably Cl.

12. A compound according to any one of claims 2 to 11 wherein R₂ is H, R₄ is Cl and R₅ is H.

13. A compound according to any one of claims 1 to 12, wherein ring A is of formula: 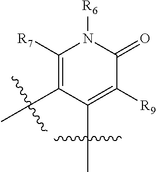
wherein R₆, R₇ and R₉ are all H.

14. A compound according to any one of claims 1 to 12, wherein ring A is of formula: 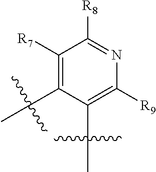
wherein R₇ and R₃ are H, and R₉ is F.

15. A compound according to any one of claims 1 to 12, wherein ring A is of formula: 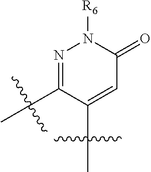
wherein R₆ is H.

16. A compound according to any one of claims 1 to 15, wherein Rₐ and R_{b} are both H.

17. A compound according to any one of claims 1 to 16, wherein Y is selected from CH₂, CHF and CF₂, more preferably CH₂.

18. A compound according to any one of claims 1 to 17 which is selected from the following: 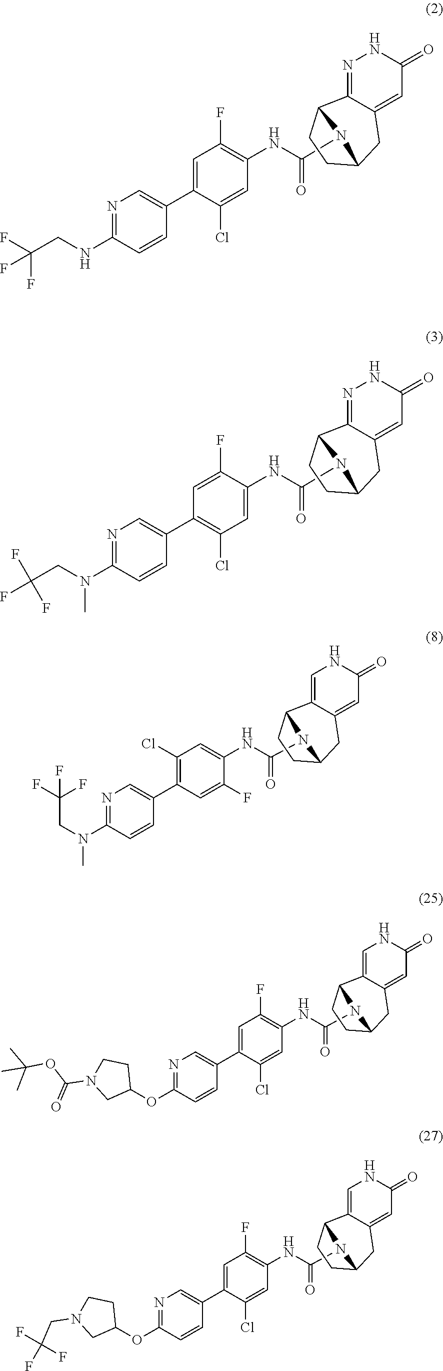 [SMILES: O=C(Nc1cc(Cl)c(-c2ccc(NCC(F)(F)F)nc2)cc1F)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3n[nH]c(=O)cc3C4)cc2Cl)cn1 ; CN(CC(F)(F)F)c1ccc(-c2cc(F)c(NC(=O)N3[C@H]4CC[C@@H]3c3c[nH]c(=O)cc3C4)cc2Cl)cn1 ; CC(C)(C)OC(=O)N1CCC(Oc2ccc(-c3cc(F)c(NC(=O)N4[C@H]5CC[C@@H]4c4c[nH]c(=O)cc4C5)cc3Cl)cn2)C1 ; O=C(Nc1cc(Cl)c(-c2ccc(OC3CCN(CC(F)(F)F)C3)nc2)cc1F)N1[C@H]2CC[C@@H]1c1c[nH]c(=O)cc1C2] 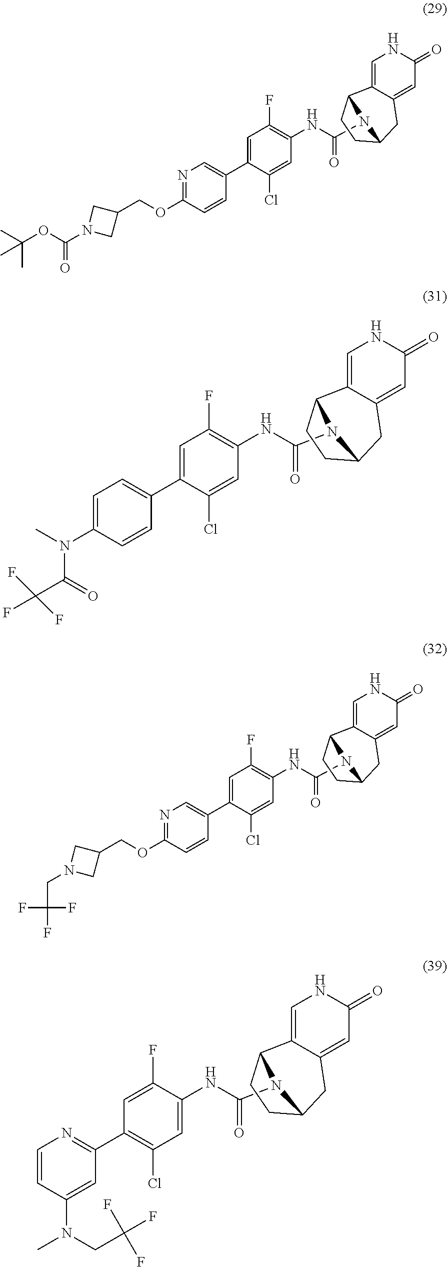 [SMILES: CC(C)(C)OC(=O)N1CC(COc2ccc(-c3cc(F)c(NC(=O)N4[C@H]5CC[C@@H]4c4c[nH]c(=O)cc4C5)cc3Cl)cn2)C1 ; CN(C(=O)C(F)(F)F)c1ccc(-c2cc(F)c(NC(=O)N3[C@H]4CC[C@@H]3c3c[nH]c(=O)cc3C4)cc2Cl)cc1 ; O=C(Nc1cc(Cl)c(-c2ccc(OCC3CN(CC(F)(F)F)C3)nc2)cc1F)N1[C@H]2CC[C@@H]1c1c[nH]c(=O)cc1C2 ; CN(CC(F)(F)F)c1ccnc(-c2cc(F)c(NC(=O)N3[C@H]4CC[C@@H]3c3c[nH]c(=O)cc3C4)cc2Cl)c1] 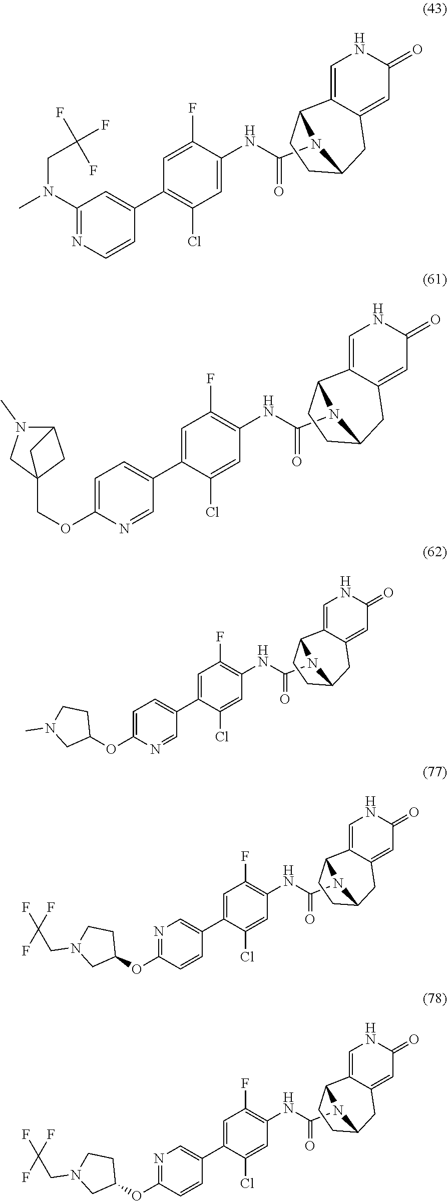 [SMILES: CN(CC(F)(F)F)c1cc(-c2cc(F)c(NC(=O)N3[C@H]4CC[C@@H]3c3c[nH]c(=O)cc3C4)cc2Cl)ccn1 ; CN1CC2(COc3ccc(-c4cc(F)c(NC(=O)N5[C@H]6CC[C@@H]5c5c[nH]c(=O)cc5C6)cc4Cl)cn3)CC1C2 ; CN1CCC(Oc2ccc(-c3cc(F)c(NC(=O)N4[C@H]5CC[C@@H]4c4c[nH]c(=O)cc4C5)cc3Cl)cn2)C1 ; O=C(Nc1cc(Cl)c(-c2ccc(O[C@@H]3CCN(CC(F)(F)F)C3)nc2)cc1F)N1[C@H]2CC[C@@H]1c1c[nH]c(=O)cc1C2 ; O=C(Nc1cc(Cl)c(-c2ccc(O[C@H]3CCN(CC(F)(F)F)C3)nc2)cc1F)N1[C@H]2CC[C@@H]1c1c[nH]c(=O)cc1C2]
and enantiomers thereof, and mixtures of enantiomers thereof, including racemic mixtures, and pharmaceutically acceptable salts and solvates thereof.

19. A compound of formula (II), or a pharmaceutically acceptable salt or solvate thereof, 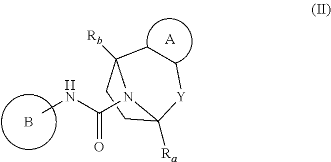
wherein:
ring A is selected from: 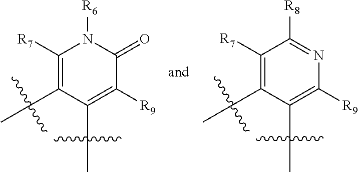
Y is CR₁₀R_{10′}, wherein R₁₀ and R_{10′} are each independently selected from H, F, alkyl, and haloalkyl;
Rₐ and R_{b} are each independently selected from H and alkyl;
ring B is a monocyclic heteroaryl group which is substituted by:
(i) a bicyclic heteroaryl group, wherein said bicyclic heteroaryl group is optionally further substituted by one or more substituents selected from halo, haloalkyl, alkyl, alkoxy, NHCO-alkyl, NR₁₁R_{11′}, SO₂-alkyl, CN, hydroxyalkyl, CONR₁₂R_{12′}, alkyl-NR₁₃R_{13′}, alkylamino-alkoxy, dialkylamino-alkoxy, heterocycloalkyl, alkyl-heterocycloalkyl, alkyl-cycloalkyl, aryl, (CH₂)ₘ—NHSO₂-alkyl, CO₂R₁₄, alkoxy-alkyl, haloalkoxy, heteroaryl, alkoxy-alkoxy, O—(CH₂)ₚ-cycloalkyl, and O—(CH₂)_{q}—heterocycloalkyl,
wherein said cycloalkyl and heterocycloalkyl groups in each of the above substituents are optionally further substituted by one or more halo, haloalkyl, alkyl or alkoxy groups; and
(ii) optionally one or more substituents selected from halo, haloalkyl, haloalkoxy, alkyl, alkoxy, OH and CN;
each of m, p and q is an integer from 0 to 3;
R₆ is selected from H and alkyl, more preferably H;
R₇, R₈ and R₉ are each independently selected from H, halo and alkyl;
R₁₁, R_{11′}, R₁₂, R_{12′}, R₁₃ and R_{13′}, are each independently selected from H, alkyl, and alkoxyalkyl; and
R₁₄ is selected from alkyl and haloalkyl.

20. A compound according to claim 19 wherein the monocyclic heteroaryl group is a pyridinyl group.

21. A compound according to claim 19 or claim 20 which is of formula (IIb), or a pharmaceutically acceptable salt or solvate thereof, 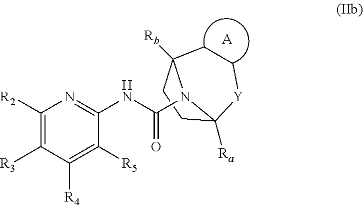
wherein:
R₂, R₄ and R₅ are each independently selected from H, halo, haloalkyl, haloalkoxy, alkyl, alkoxy, OH and CN; and
R₃ is a bicyclic heteroaryl group, wherein said bicyclic heteroaryl group is optionally further substituted by one or more substituents selected from halo, haloalkyl, alkyl, alkoxy, NHCO-alkyl, NR₁₁R_{11′}, SO₂-alkyl, CN, hydroxyalkyl, CONR₁₂R_{12′}, alkyl-NR₁₃R_{13′}, alkylamino-alkoxy, dialkylamino-alkoxy, heterocycloalkyl, alkyl-heterocycloalkyl, alkyl-cycloalkyl, aryl, (CH₂)ₘ NHSO₂-alkyl, CO₂R₁₄, alkoxy-alkyl, haloalkoxy, heteroaryl, alkoxy-alkoxy, O—(CH₂)ₚ-cycloalkyl, and O—(CH₂)_{q}-heterocycloalkyl,
wherein said cycloalkyl and heterocycloalkyl groups in each of the above substituents are optionally further substituted by one or more halo, haloalkyl, alkyl or alkoxy groups.

22. A compound according to claim 21 wherein R₃ is a 9-membered bicyclic heteroaryl group selected from the following: 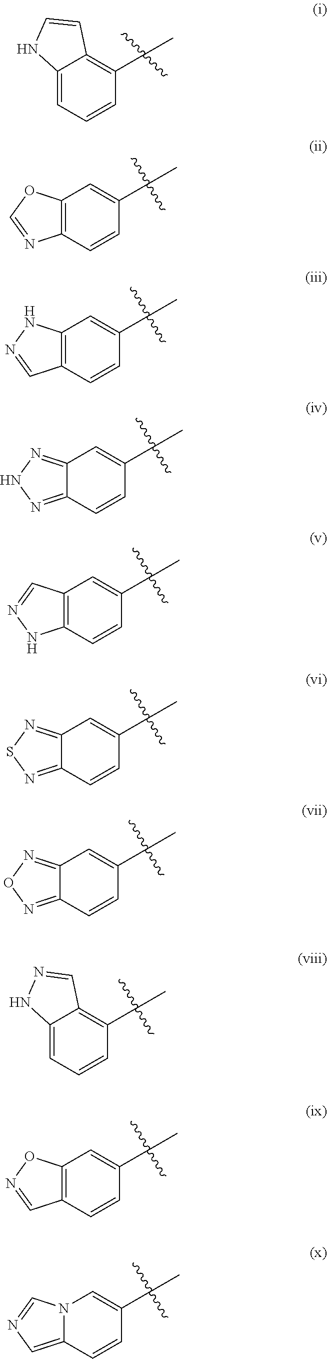 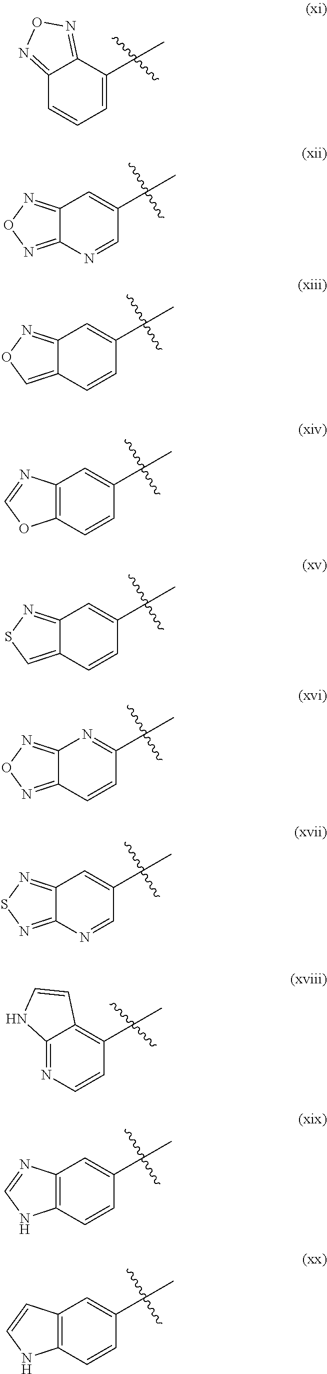
each of which is optionally substituted by one or more substituents selected from alkyl, haloalkyl and halo.

23. A compound according to claim 21 or claim 22 wherein R₃ is selected from the following groups:

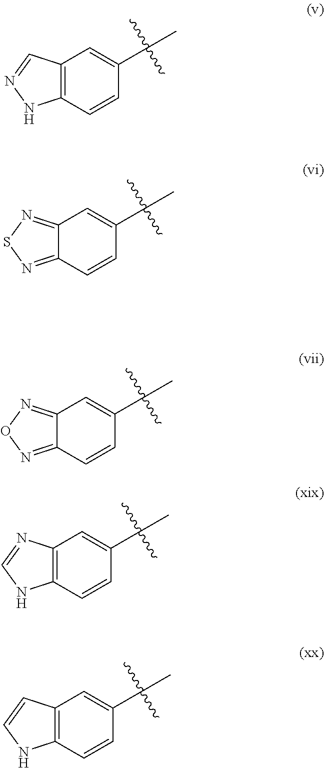 [SMILES: *c1ccc2[nH]ncc2c1 ; *c1ccc2nsnc2c1 ; *c1ccc2nonc2c1 ; *c1ccc2[nH]cnc2c1 ; *c1ccc2[nH]ccc2c1]

24. A compound according to any one of claims 21 to 23, wherein R₂ and R₅ are both H.

25. A compound according to any one of claims 21 to 24, wherein R₄ is selected from C₁, Br, and CF₃, more preferably Cl.

26. A compound according to any one of claims 19 to 25, wherein ring A is of formula: 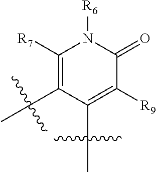
wherein R₆, R₇ and R₉ are all H.

27. A compound according to any one of claims 19 to 25, wherein ring A is of formula: 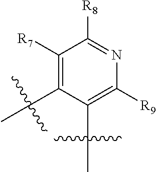
wherein R₇ and R₃ are H, and R₉ is F.

28. A compound according to any one of claims 19 to 27, wherein Rₐ and R_{b} are both H.

29. A compound according to any one of claims 19 to 28, wherein Y is selected from CH₂, CHF and CF₂, more preferably CH₂.

30. A compound according to any one of claims 19 to 29, which is selected from the following: 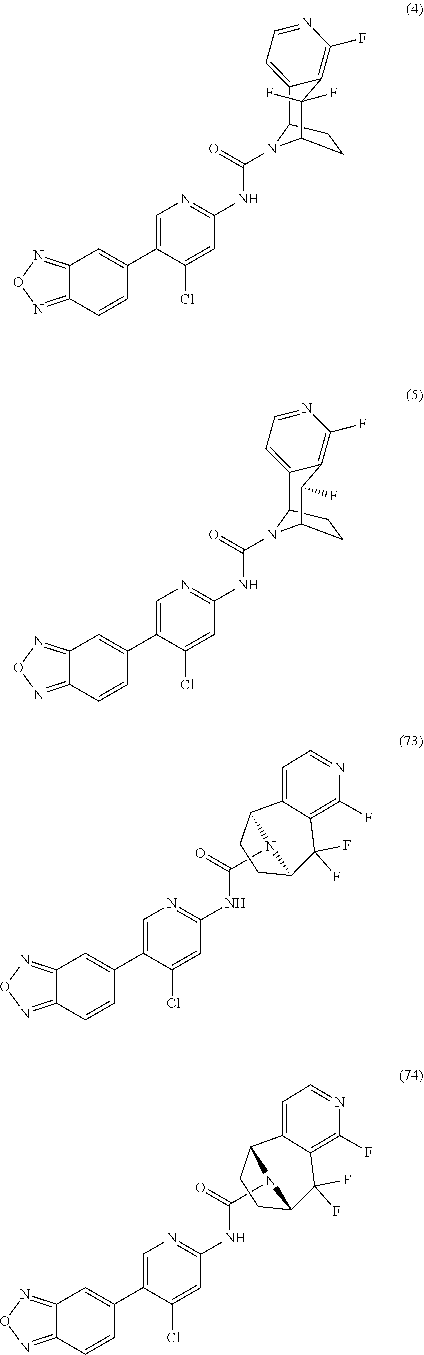 [SMILES: O=C(Nc1cc(Cl)c(-c2ccc3nonc3c2)cn1)N1C2CCC1C(F)(F)c1c2ccnc1F ; O=C(Nc1cc(Cl)c(-c2ccc3nonc3c2)cn1)N1C2CCC1[C@H](F)c1c2ccnc1F ; O=C(Nc1cc(Cl)c(-c2ccc3nonc3c2)cn1)N1[C@H]2CC[C@@H]1C(F)(F)c1c2ccnc1F ; O=C(Nc1cc(Cl)c(-c2ccc3nonc3c2)cn1)N1[C@@H]2CC[C@H]1C(F)(F)c1c2ccnc1F] 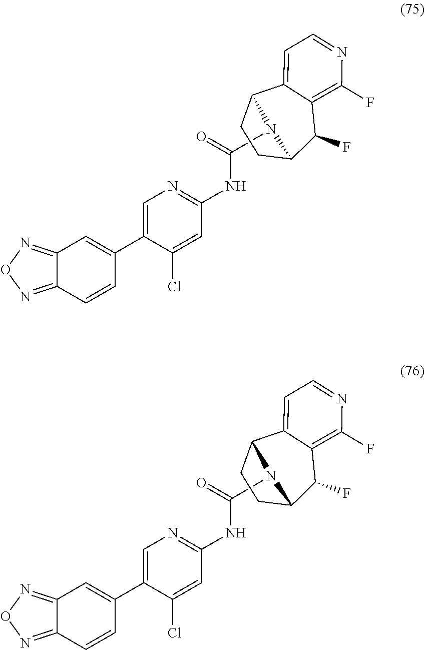 [SMILES: O=C(Nc1cc(Cl)c(-c2ccc3nonc3c2)cn1)N1[C@@H]2CC[C@H]1c1ccnc(F)c1[C@H]2F ; O=C(Nc1cc(Cl)c(-c2ccc3nonc3c2)cn1)N1[C@@H]2CC[C@H]1[C@@H](F)c1c2ccnc1F]
and enantiomers thereof, and mixtures of enantiomers thereof, including racemic mixtures, and pharmaceutically acceptable salts and solvates thereof.

31. A compound which is selected from the following: 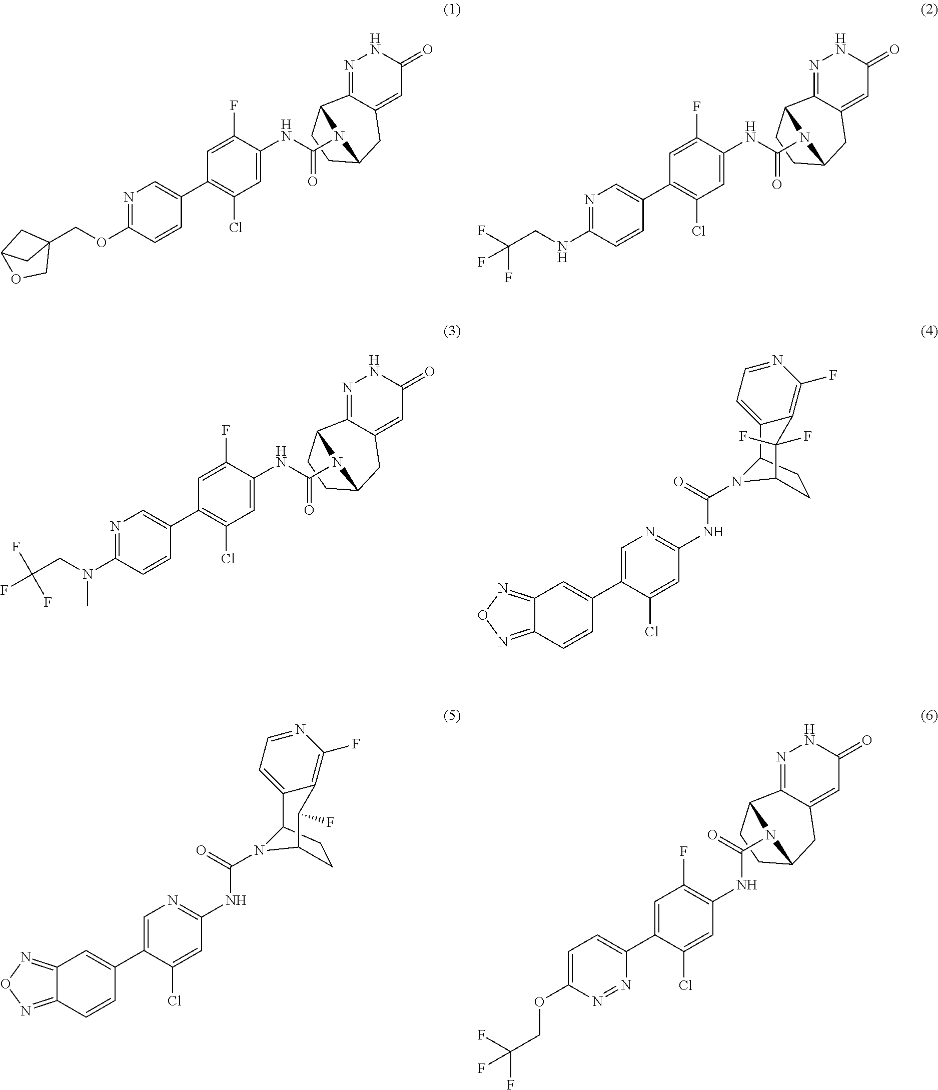 [SMILES: O=C(Nc1cc(Cl)c(-c2ccc(OCC34COC(C3)C4)nc2)cc1F)N1[C@H]2CC[C@@H]1c1n[nH]c(=O)cc1C2 ; O=C(Nc1cc(Cl)c(-c2ccc(NCC(F)(F)F)nc2)cc1F)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccc3nonc3c2)cn1)N1C2CCC1C(F)(F)c1c2ccnc1F ; O=C(Nc1cc(Cl)c(-c2ccc3nonc3c2)cn1)N1C2CCC1[C@H](F)c1c2ccnc1F ; O=C(Nc1cc(Cl)c(-c2ccc(OCC(F)(F)F)nn2)cc1F)N1[C@H]2CC[C@@H]1c1n[nH]c(=O)cc1C2] 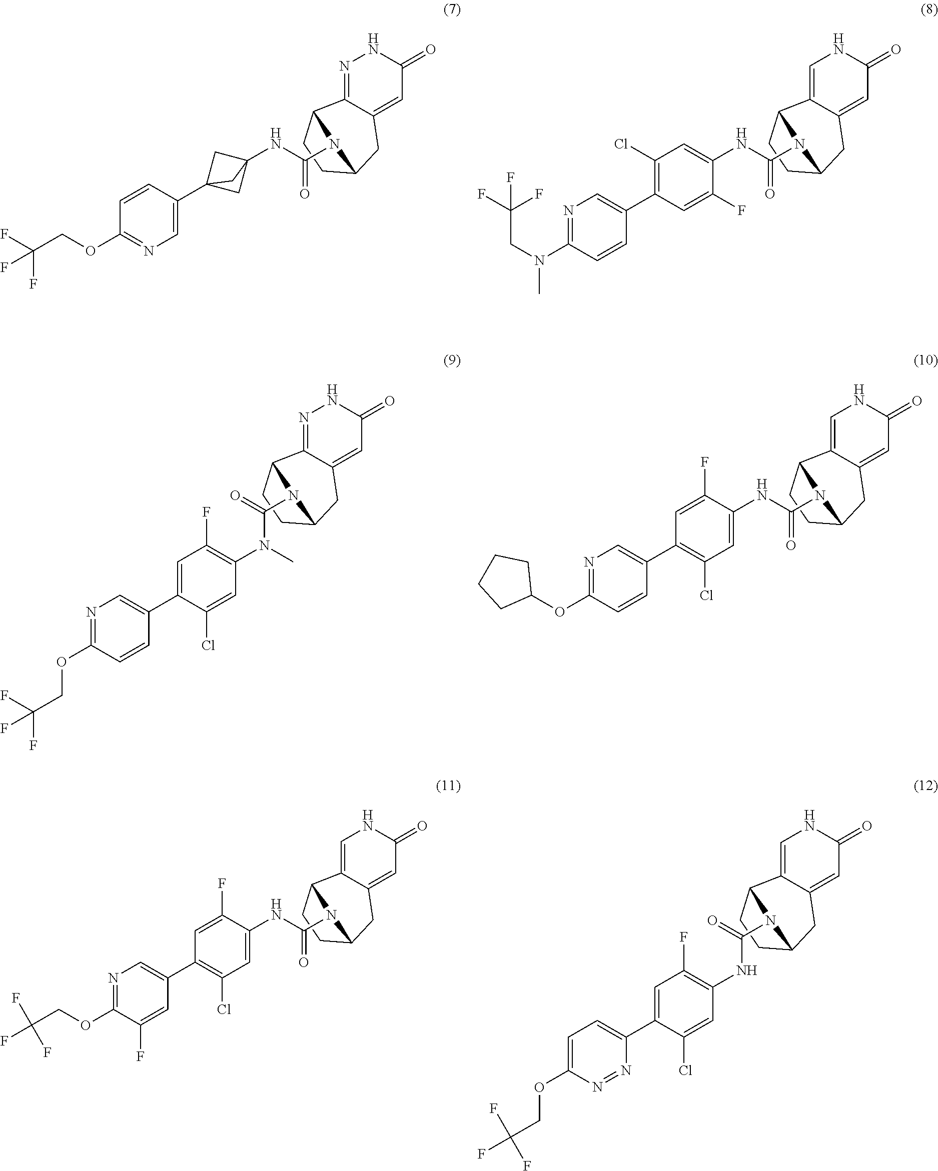 [SMILES: O=C(NC12CC(c3ccc(OCC(F)(F)F)nc3)(C1)C2)N1[C@H]2CC[C@@H]1c1n[nH]c(=O)cc1C2 ; CN(CC(F)(F)F)c1ccc(-c2cc(F)c(NC(=O)N3[C@H]4CC[C@@H]3c3c[nH]c(=O)cc3C4)cc2Cl)cn1 ; CN(C(=O)N1[C@H]2CC[C@@H]1c1n[nH]c(=O)cc1C2)c1cc(Cl)c(-c2ccc(OCC(F)(F)F)nc2)cc1F ; O=C(Nc1cc(Cl)c(-c2ccc(OC3CCCC3)nc2)cc1F)N1[C@H]2CC[C@@H]1c1c[nH]c(=O)cc1C2 ; O=C(Nc1cc(Cl)c(-c2cnc(OCC(F)(F)F)c(F)c2)cc1F)N1[C@H]2CC[C@@H]1c1c[nH]c(=O)cc1C2 ; O=C(Nc1cc(Cl)c(-c2ccc(OCC(F)(F)F)nn2)cc1F)N1[C@H]2CC[C@@H]1c1c[nH]c(=O)cc1C2] 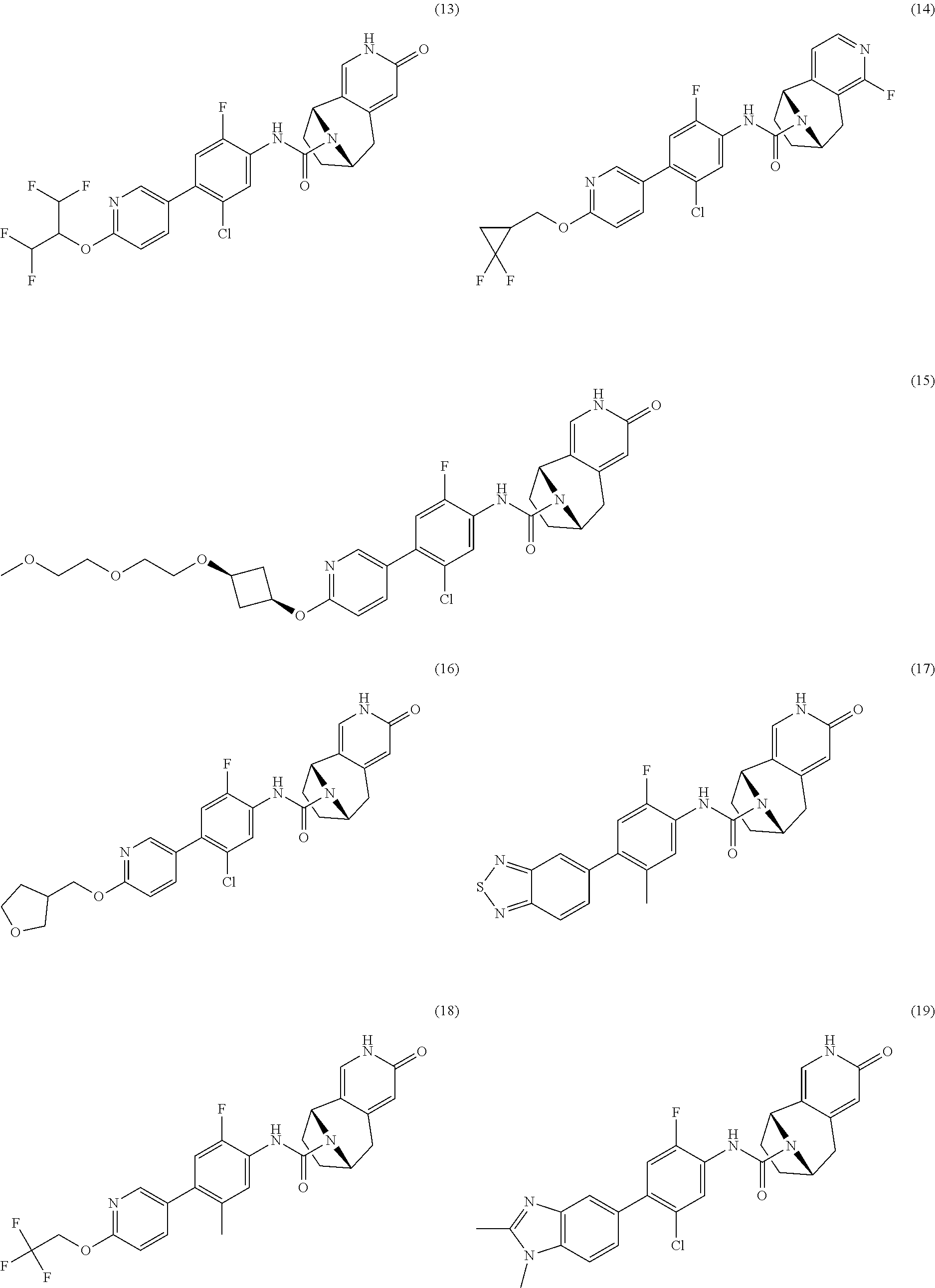 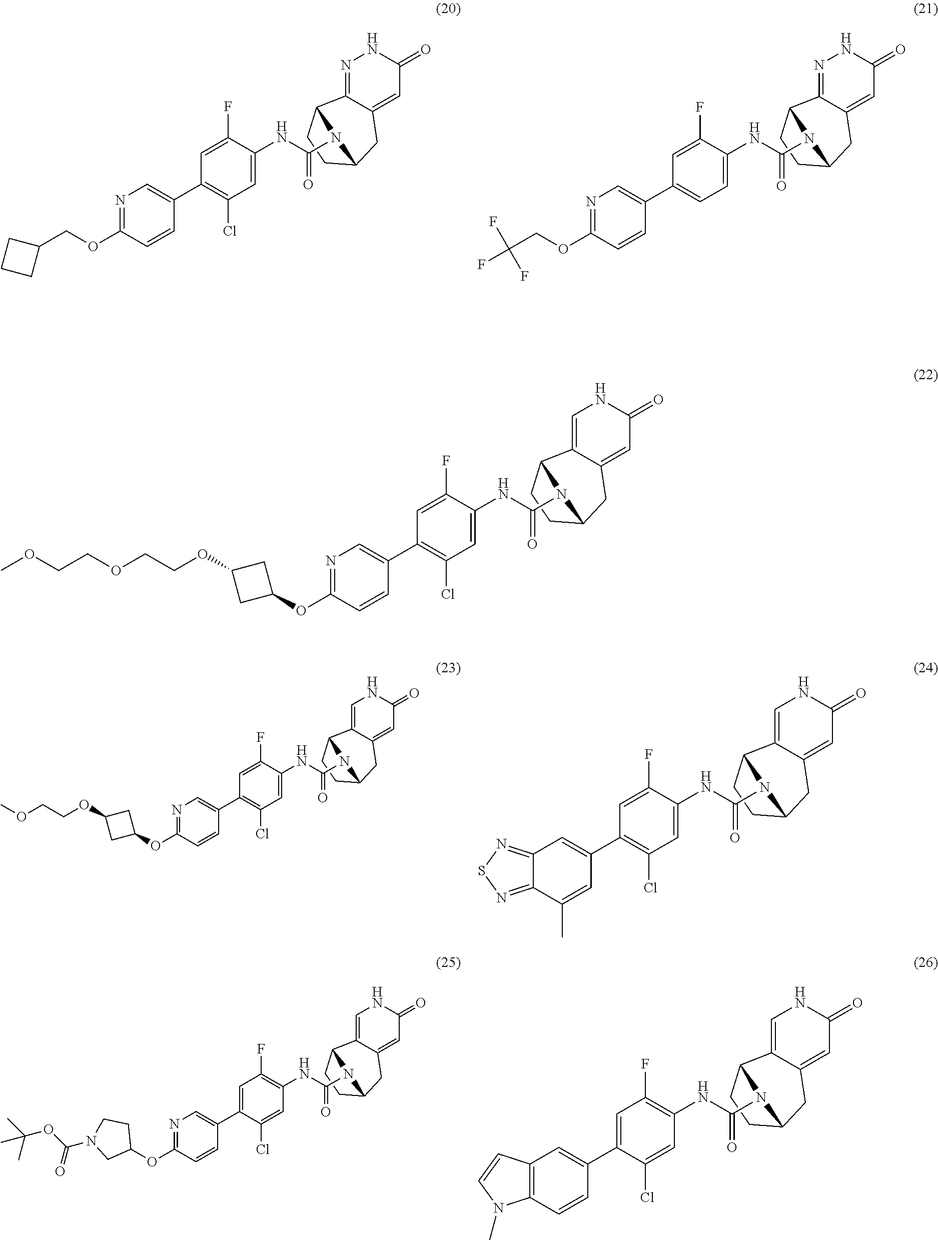 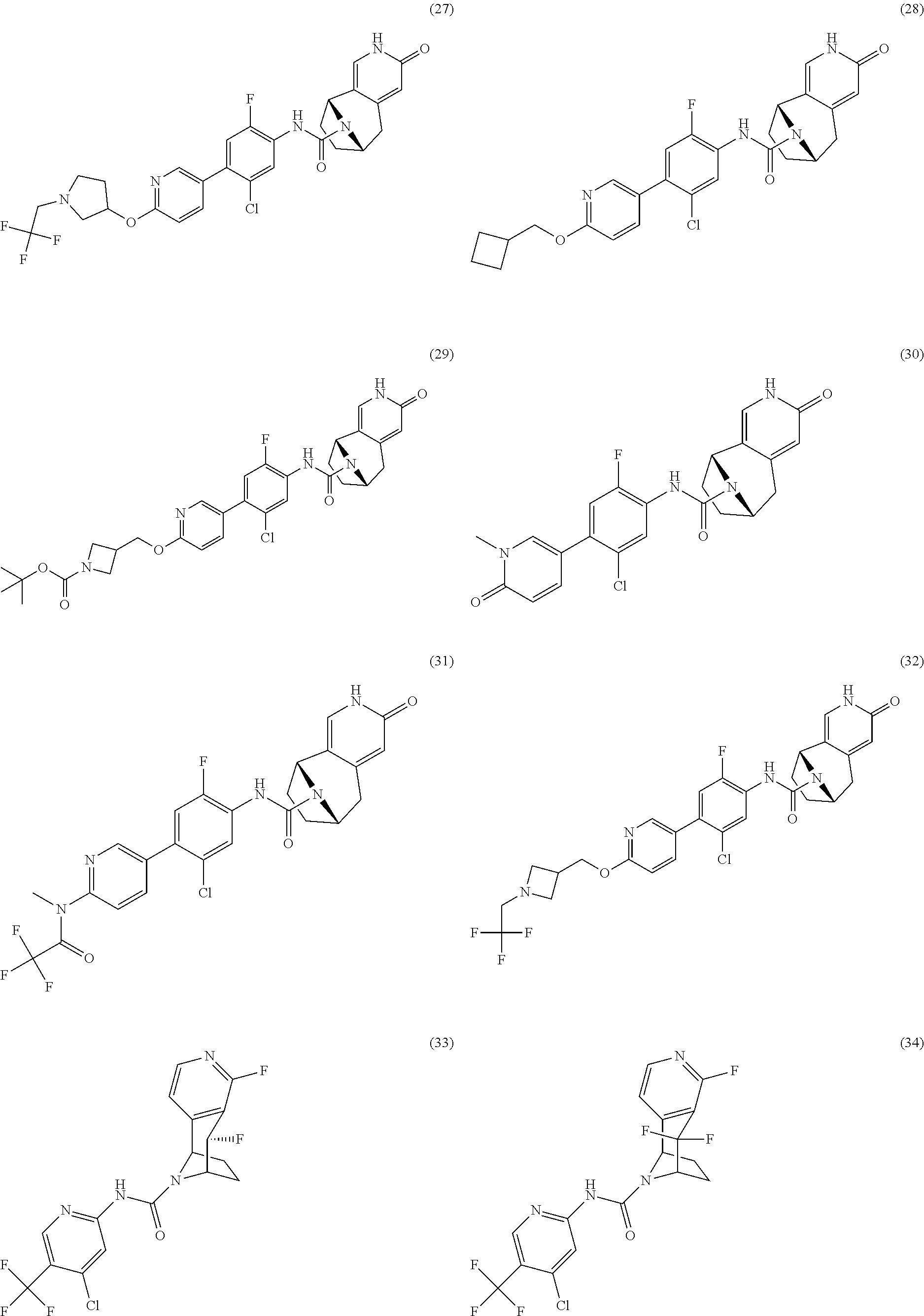 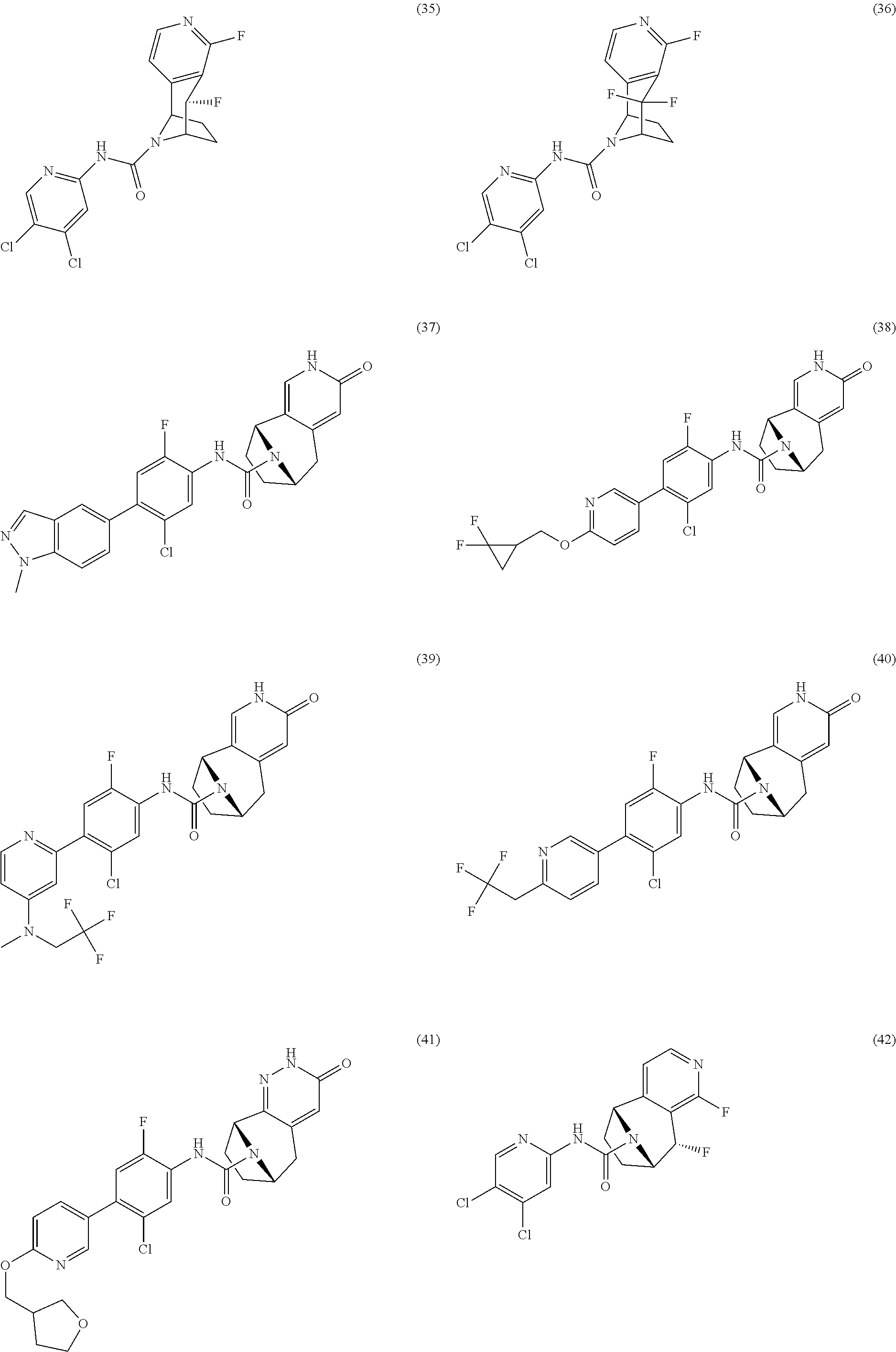 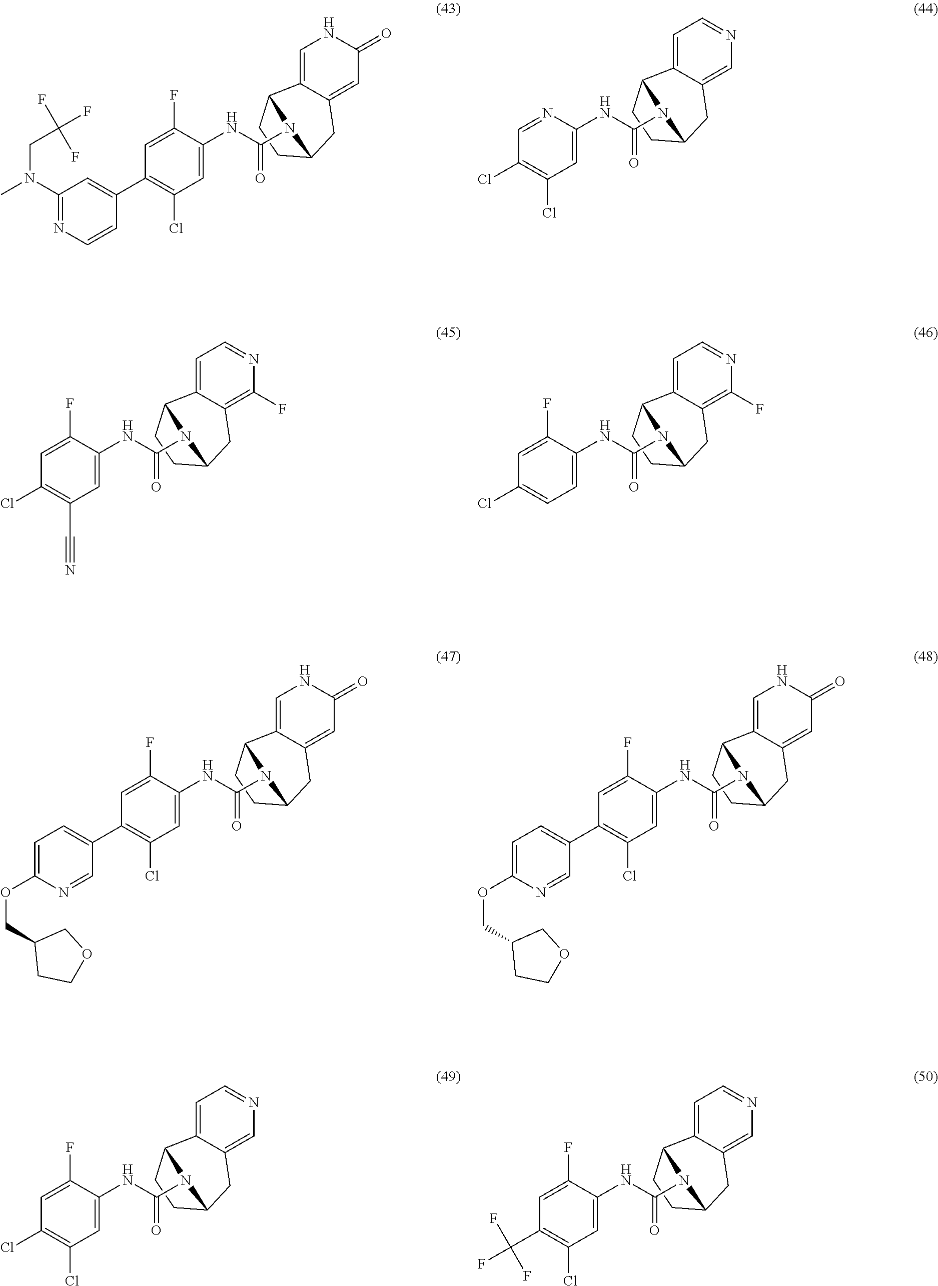 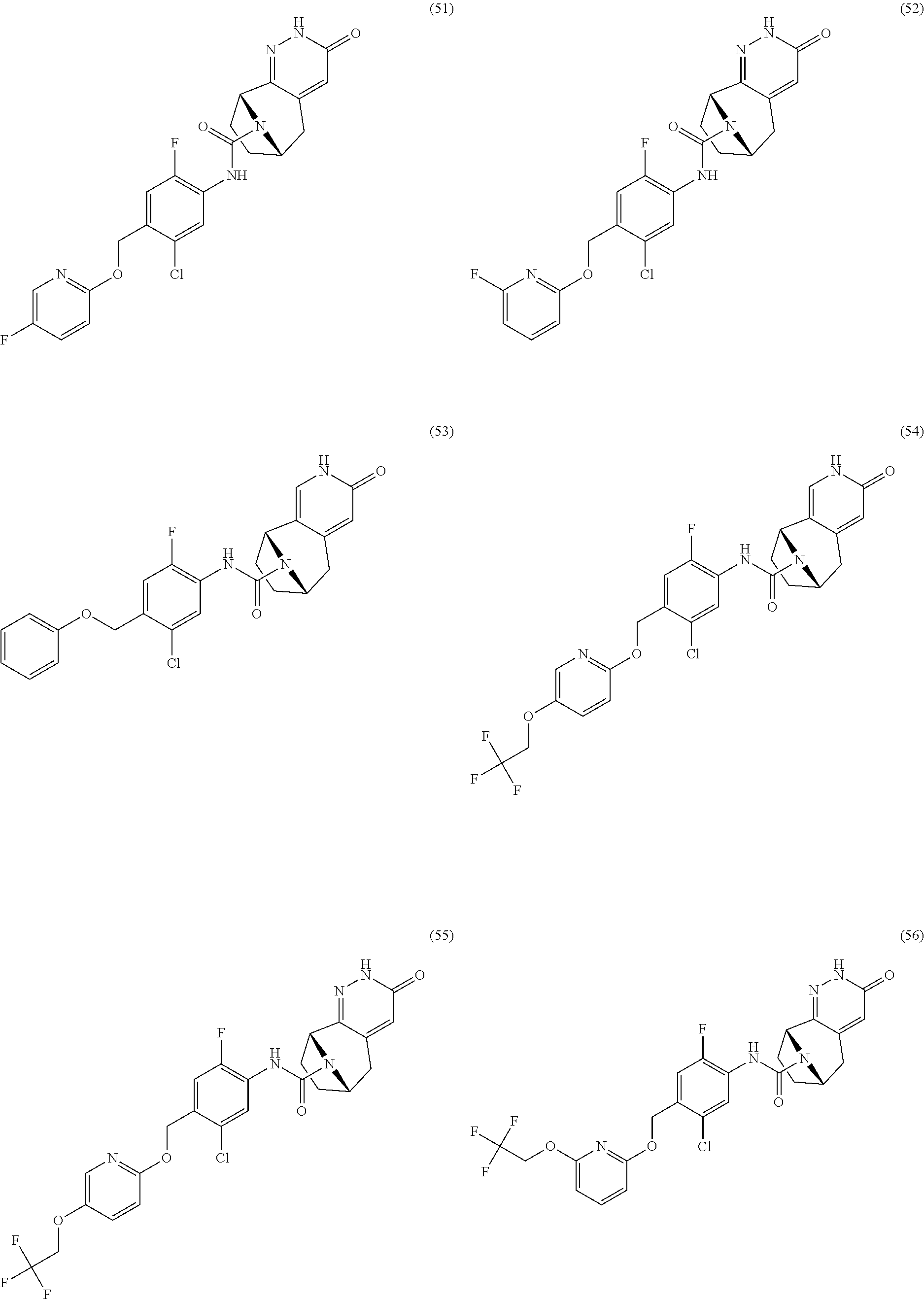 [SMILES: O=C(Nc1cc(Cl)c(COc2ccc(F)cn2)cc1F)N1[C@H]2CC[C@@H]1c1n[nH]c(=O)cc1C2 ; O=C(Nc1cc(Cl)c(COc2cccc(F)n2)cc1F)N1[C@H]2CC[C@@H]1c1n[nH]c(=O)cc1C2 ; O=C(Nc1cc(Cl)c(COc2ccccc2)cc1F)N1[C@H]2CC[C@@H]1c1c[nH]c(=O)cc1C2 ; O=C(Nc1cc(Cl)c(COc2ccc(OCC(F)(F)F)cn2)cc1F)N1[C@H]2CC[C@@H]1c1c[nH]c(=O)cc1C2 ; O=C(Nc1cc(Cl)c(COc2ccc(OCC(F)(F)F)cn2)cc1F)N1[C@H]2CC[C@@H]1c1n[nH]c(=O)cc1C2 ; O=C(Nc1cc(Cl)c(COc2cccc(OCC(F)(F)F)n2)cc1F)N1[C@H]2CC[C@@H]1c1n[nH]c(=O)cc1C2] 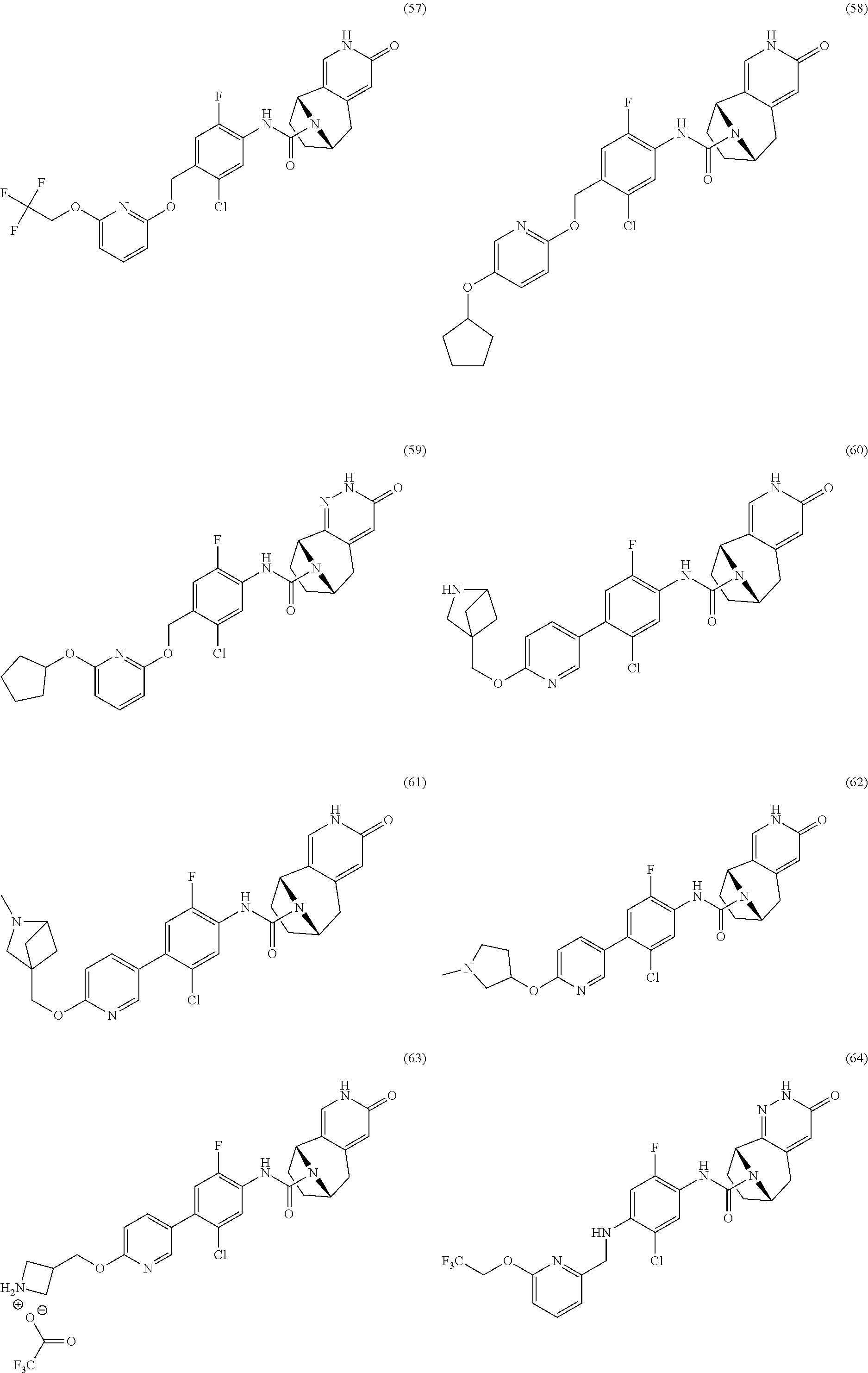 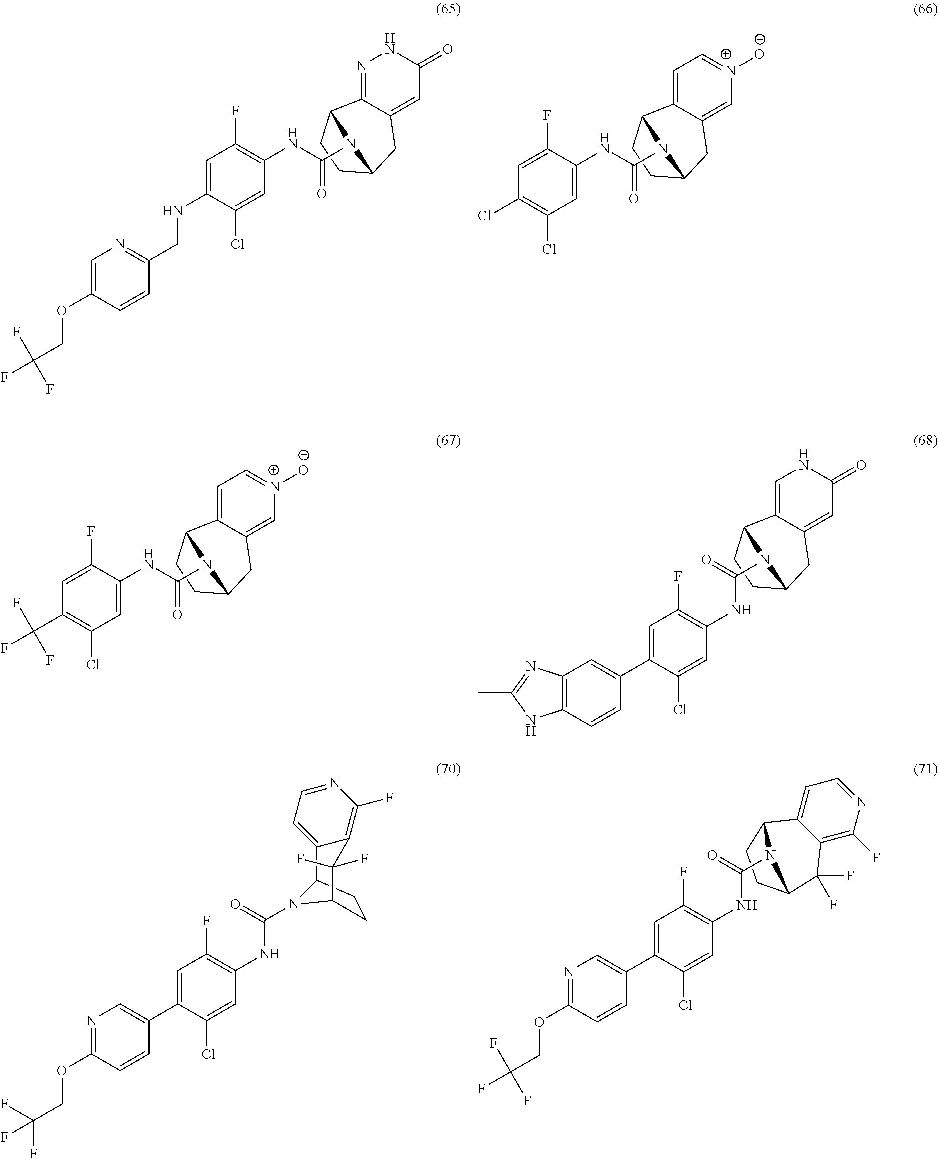 [SMILES: O=C(Nc1cc(Cl)c(NCc2ccc(OCC(F)(F)F)cn2)cc1F)N1[C@H]2CC[C@@H]1c1n[nH]c(=O)cc1C2 ; O=C(Nc1cc(Cl)c(Cl)cc1F)N1[C@H]2CC[C@@H]1c1cc[n+]([O-])cc1C2 ; O=C(Nc1cc(Cl)c(C(F)(F)F)cc1F)N1[C@H]2CC[C@@H]1c1cc[n+]([O-])cc1C2 ; Cc1nc2cc(-c3cc(F)c(NC(=O)N4[C@H]5CC[C@@H]4c4c[nH]c(=O)cc4C5)cc3Cl)ccc2[nH]1 ; O=C(Nc1cc(Cl)c(-c2ccc(OCC(F)(F)F)nc2)cc1F)N1C2CCC1C(F)(F)c1c2ccnc1F ; O=C(Nc1cc(Cl)c(-c2ccc(OCC(F)(F)F)nc2)cc1F)N1[C@@H]2CC[C@H]1C(F)(F)c1c2ccnc1F] 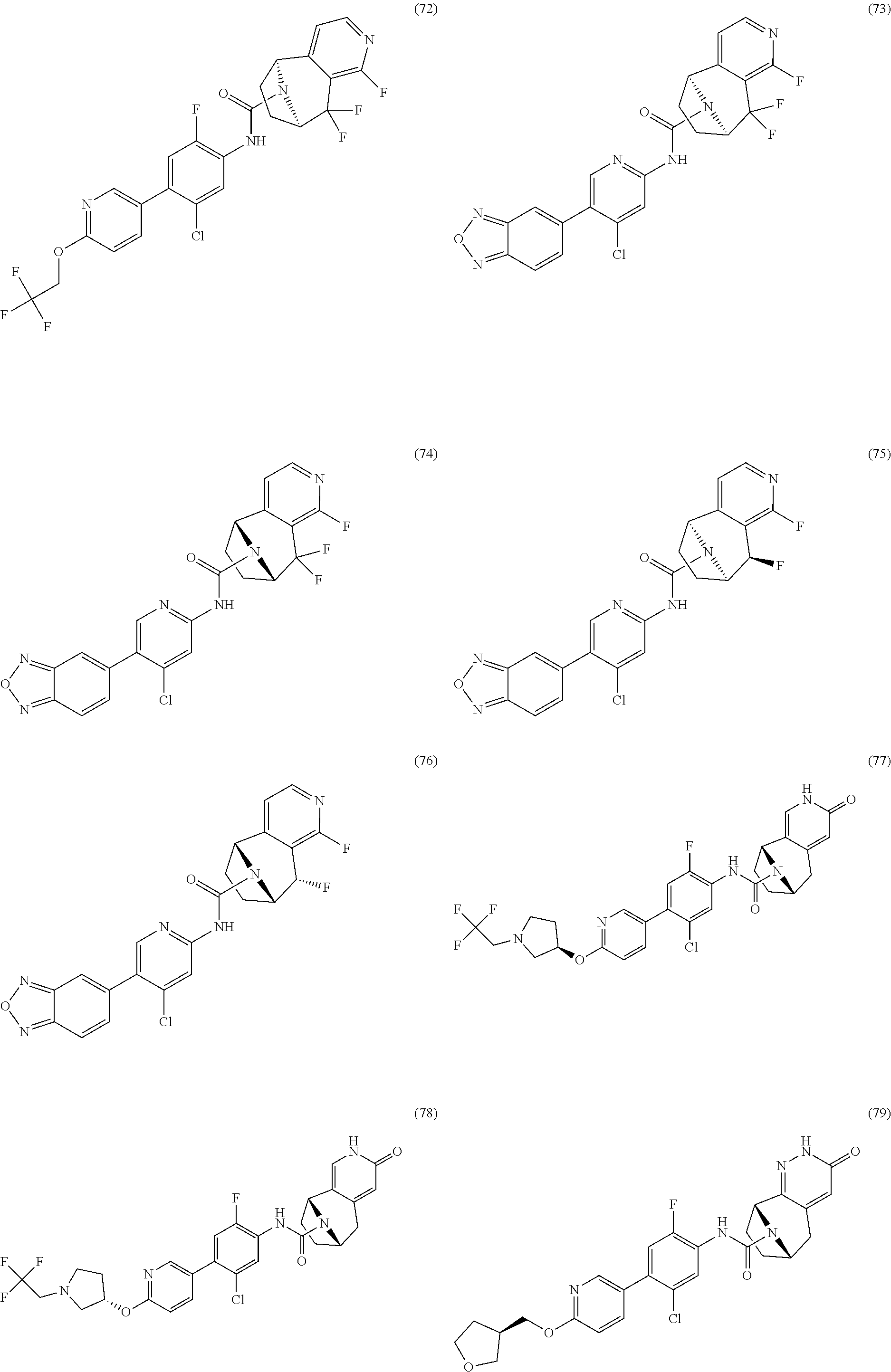 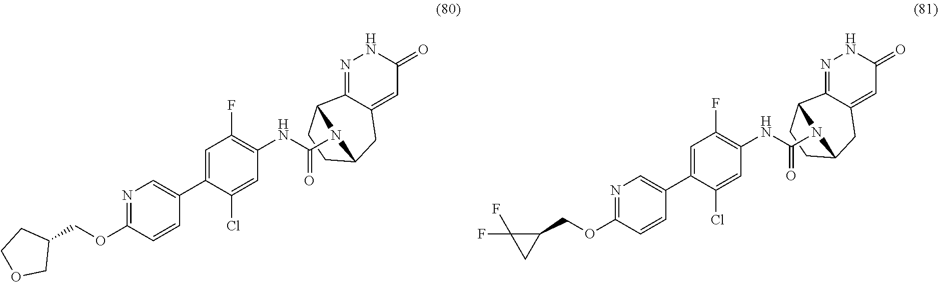 [SMILES: O=C(Nc1cc(Cl)c(-c2ccc(OC[C@H]3CCOC3)nc2)cc1F)N1[C@H]2CC[C@@H]1c1n[nH]c(=O)cc1C2 ; O=C(Nc1cc(Cl)c(-c2ccc(OC[C@H]3CC3(F)F)nc2)cc1F)N1[C@H]2CC[C@@H]1c1n[nH]c(=O)cc1C2] 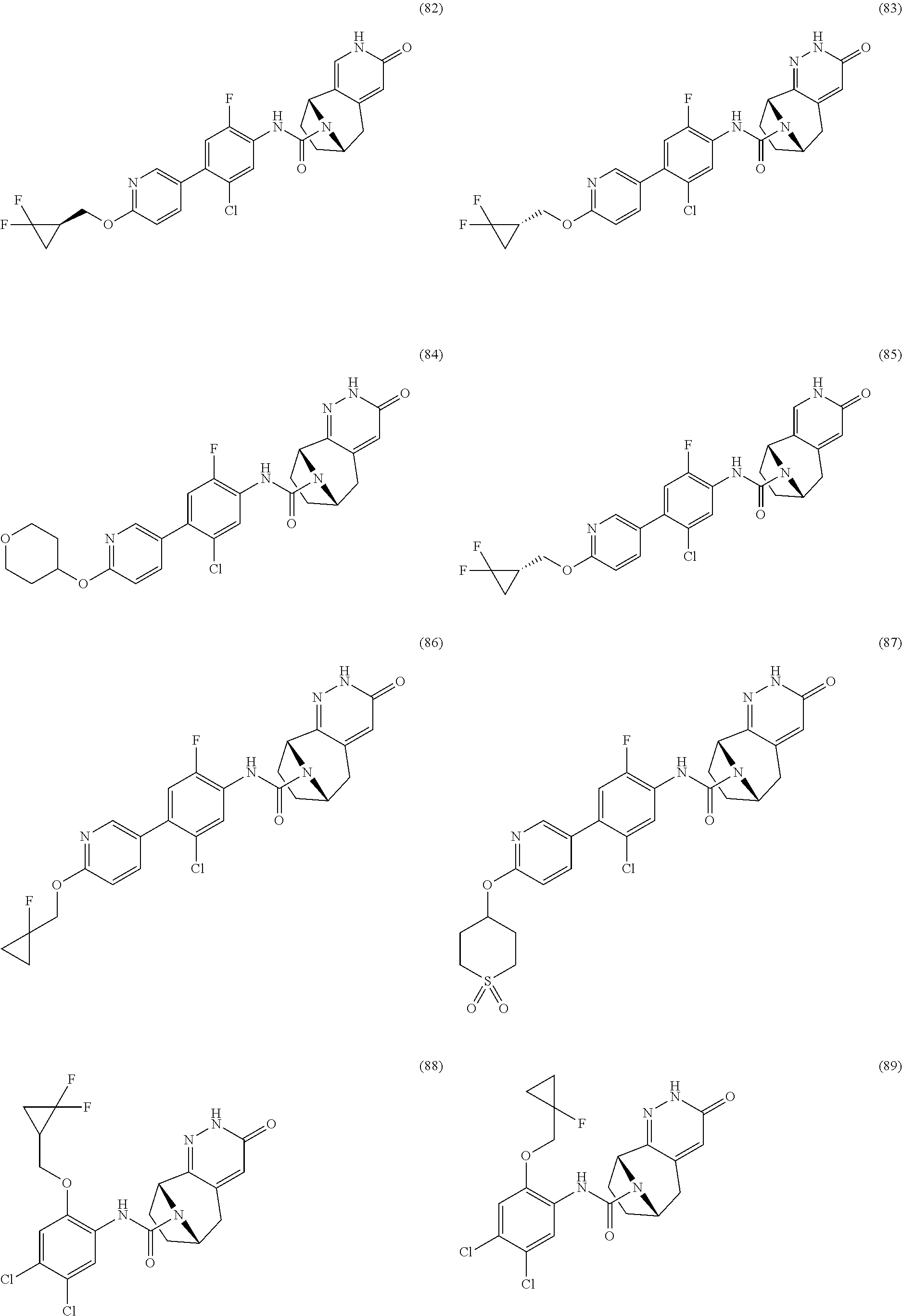 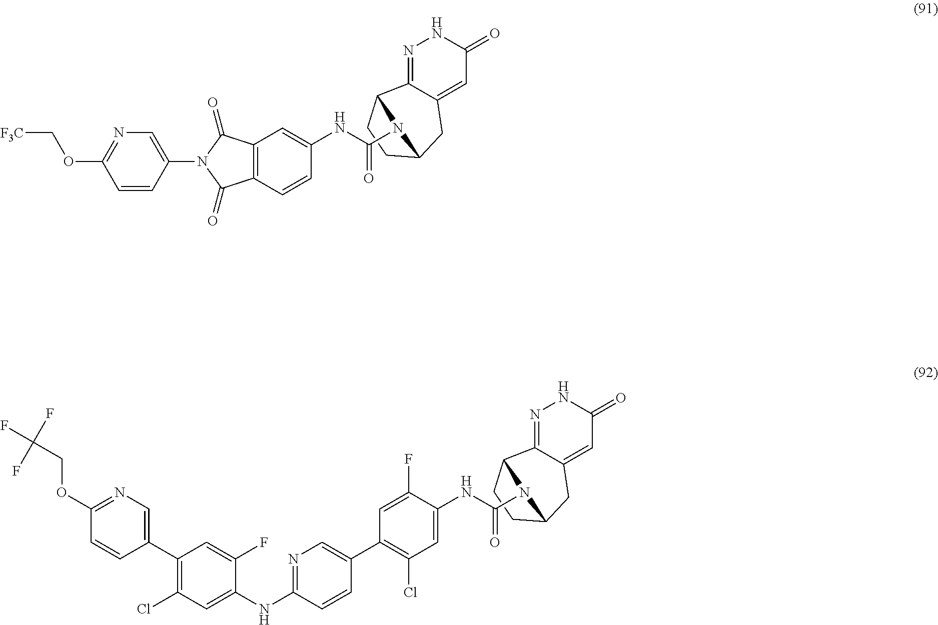 [SMILES: O=C1c2ccc(NC(=O)N3[C@H]4CC[C@@H]3c3n[nH]c(=O)cc3C4)cc2C(=O)N1c1ccc(OCC(F)(F)F)nc1 ; O=C(Nc1cc(Cl)c(-c2ccc(Nc3cc(Cl)c(-c4ccc(OCC(F)(F)F)nc4)cc3F)nc2)cc1F)N1[C@H]2CC[C@@H]1c1n[nH]c(=O)cc1C2]
and enantiomers thereof, and mixtures of enantiomers thereof, including racemic mixtures, and pharmaceutically acceptable salts and solvates thereof.

32. A pharmaceutical composition comprising a compound according to any of claims 1-31, and a pharmaceutically acceptable diluent, excipient, or carrier.

33. A compound according to any one of claims 1-31, or a pharmaceutical composition according to claim 32, for use as a medicament.

34. A compound according to any one of claims 1-31, or a pharmaceutical composition according to claim 32, for use in treating or preventing a disorder selected from a proliferative disorder, an immune disorder, asthma, chronic obstructive pulmonary disease (COPD) and acute respiratory distress syndrome (ARDS).

35. A compound or pharmaceutical composition for use according to claim 33 or claim 34, wherein the use comprises modulating GPR65, preferably wherein the use comprises inhibiting GPR65 signalling.

36. A compound or pharmaceutical composition for use according to claim 34 or claim 35, wherein the disorder is a proliferative disorder.

37. A compound or pharmaceutical composition for use according to claim 36, wherein the proliferative disorder is a cancer, and is preferably a solid tumour and/or metastases thereof.

38. A compound or pharmaceutical composition for use according to claim 37, wherein the proliferative disorder is a cancer selected from melanoma, renal cell carcinoma (RCC), gastric cancer, acute myeloid leukaemia (AML), triple negative breast cancer (TNBC), colorectal cancer, head and neck cancer, colorectal adenocarcinoma, pancreatic adenocarcinoma, sarcoma, lung cancer, ovarian cancer and gliomas, preferably glioblastoma (GBM).

39. A compound or pharmaceutical composition for use according to claim 34 or claim 35 wherein the disorder is an immune disorder.

40. A compound or pharmaceutical composition for use according to claim 39, wherein the immune disorder is an autoimmune disease.

41. A compound or pharmaceutical composition for use according to claim 40, wherein the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, and autoimmune polyendocrinopathies.

42. A compound or pharmaceutical composition for use according to claim 41, wherein the autoimmune disease is selected from psoriasis, psoriatic arthritis, ankylosing spondylitis, Crohn's disease, and multiple sclerosis (MS).

43. A compound or pharmaceutical composition for use according to claim 34 or claim 35, wherein the use comprises treating or preventing a disorder selected from asthma, chronic obstructive pulmonary disease (COPD) and acute respiratory distress syndrome (ARDS).

44. A method of treating a disorder as defined in any of claims 34 to 43, comprising administering to a subject a compound as defined in any of claims 1-31, or a pharmaceutical composition as defined in claim 32.

45. A compound as defined in any one of claims 1-31, or a pharmaceutically acceptable salt or solvate thereof, or a pharmaceutical composition according to claim 32, for use in treating or preventing a GPR65-associated disease or disorder.

46. Use of a compound as defined in any one of claims 1-31, or a pharmaceutically acceptable salt or solvate thereof, in the preparation of a medicament for treating or preventing a GPR65-associated disease or disorder in a subject.

47. Use of a compound as defined in any one of claims 1-31, or a pharmaceutically acceptable salt or solvate thereof, in the preparation of a medicament for treating or preventing a disorder selected from a proliferative disorder, an immune disorder, asthma, chronic obstructive pulmonary disease (COPD) and acute respiratory distress syndrome (ARDS).
